(19) United States
(12) Patent Application Publication
(10) Pub. No.: US 2025/0120975 A1
(43) Pub. Date: Apr. 17, 2025

(54) INTEGRIN INHIBITORS FOR TREATMENT OF PRIMARY SCLEROSING CHOLANGITIS

(71) Applicant: Pliant Therapeutics, Inc., South San Francisco, CA (US)

(72) Inventors: Eric LEFEBVRE, San Francisco, CA (US); Johanna Roberta SCHAUB, San Francisco, CA (US)

(21) Appl. No.: 18/895,181

(22) Filed: Sep. 24, 2024

Related U.S. Application Data
(60) Provisional application No. 63/540,341, filed on Sep. 25, 2023.
(60) Provisional application No. 63/597,660, filed on Nov. 9, 2023.
(60) Provisional application No. 63/549,492, filed on Feb. 3, 2024.
(60) Provisional application No. 63/549,934, filed on Feb. 5, 2024.
(60) Provisional application No. 63/671,199, filed on Jul. 13, 2024.

(51) Int. Cl. A61K 31/517; A61K 49/10; A61P 1/16

(52) U.S. Cl. CPC A61K 31/517; A61K 49/105; A61P 1/16

(57) ABSTRACT

The disclosure relates to methods of (i) treating a subject for a liver fibrotic disease, in particular, primary sclerosing cholangitis or (ii) modulating α_{V}β₆ integrin, α_{V}β₁ integrin, or both α_{V}β₆ integrin and α_{V}β₁ integrin in a subject in need thereof, comprising administration of (S)-4-((2-methoxyethyl)(4-(5,6,7,8-tetrahydro-1,8-naphthyridin-2-yl)butyl)amino)-2-(quinazolin-4- ylamino)butanoic acid, or a pharmaceutically acceptable salt thereof as described herein. Optionally, a second drug, such as ursodeoxycholic acid or a pharmaceutically acceptable salt thereof, can be co-administered. The compounds and pharmaceutical compositions thereof are α_{V}β₆ integrin inhibitors that are useful for treating fibrosis such as primary sclerosing cholangitis.

### CROSS-REFERENCE TO RELATED APPLICATIONS

This application claims priority benefit of U.S. Provisional Patent Application No. 63/540,341, filed on Sep. 25, 2023, U.S. Provisional Patent Application No. 63/597,660, filed on Nov. 9, 2023, U.S. Provisional Patent Application No. 63/549,492, filed on Feb. 3, 2024, U.S. Provisional Patent Application No. 63/549,934, filed on Feb. 5, 2024, and U.S. Provisional Patent Application No. 63/671,199, filed on Jul. 13, 2024, the contents of each of which are incorporated herein by reference in their entirety.

### FIELD OF THE INVENTION

The disclosure relates to use of inhibitors of integrins for treatment of the fibrotic disease primary sclerosing cholangitis, which affects tissues of the liver, gall bladder, and/or bile ducts.

### BACKGROUND OF THE INVENTION

Fibrosis, a pathologic feature of many diseases, is caused by a dysfunction in the body's natural ability to repair damaged tissues. If left untreated, fibrosis can result in scarring of vital organs causing irreparable damage and eventual organ failure.

Primary sclerosing cholangitis (PSC) involves inflammation of bile ducts in the liver, causing fibrosis that obliterates the bile ducts. The resulting impediment to the flow of bile to the intestines can lead to cirrhosis of the liver and subsequent complications such as liver failure and liver cancer. Bile duct surgery can temporarily open blocked bile ducts, but is not a long-term solution or cure. A liver transplant can result in a positive outcome for resolution of PSC. However, a suitable organ for transplantation may not be available for a given patient. Even when a donor liver can be obtained, a liver transplant is an expensive and arduous procedure, and PSC may recur in the transplanted liver. A liver transplant also requires lifelong use of immunosuppressant drugs to prevent organ rejection, which burdens the patient in other ways. Ursodeoxycholic acid (UDCA) has been used to treat PSC, and while some indicators of liver function are improved, it is uncertain whether UDCA has any effect on long-term outcomes (Goode EC and Rushbrook S M, Ther Adv Chronic Dis. 2016 January; 7 (1): 68-85).

The α_{V}β₆ integrin is expressed in epithelial cells and binds to the latency-associated peptide of transforming growth factor-β₁ (TGFβ₁) and mediates TGFβ₁ activation. Its expression level is significantly increased after injury to lung and cholangiocytes and plays a critical in vivo role in tissue fibrosis. Expression of α_{V}β₆ is elevated in the liver and bile duct of PSC patients.

The present disclosure provides for treatment of primary sclerosing cholangitis with the α_{V}β₆ integrin inhibitors.

### BRIEF SUMMARY OF THE INVENTION

Disclosed is a method of treating a fibrotic disease in an individual (such as a human) in need thereof comprising administering to the individual a therapeutically effective amount of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments, the fibrotic disease is liver fibrosis. In some embodiments, the fibrotic disease is primary sclerosing cholangitis (PSC).

In a further embodiment, provided is a method of delaying the onset and/or development of a fibrotic disease in an individual (such as a human) who is at risk for developing a fibrotic disease comprising administering to the individual a therapeutically effective amount of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In a further embodiment, provided is a method of delaying the onset and/or development of a fibrotic disease in an individual (such as a human) who is at risk for developing a fibrotic disease comprising administering to the individual a therapeutically effective amount of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments, the fibrotic disease is liver fibrosis. In some embodiments, the fibrotic disease is primary sclerosing cholangitis. In some embodiments, the individual at risk of developing a fibrotic disease has or is suspected of having primary sclerosing cholangitis.

Also provided is(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutical composition thereof, for the treatment of a fibrotic disease.

Also provided is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or a pharmaceutical composition comprising any of the foregoing, in the manufacture of a medicament for the treatment of a fibrotic disease. In some embodiments, the fibrotic disease is liver fibrosis. In some embodiments, the fibrotic disease is primary sclerosing cholangitis.

Further provided is a kit comprising(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments, the kit comprises instructions for use according to a method described herein, such as a method of treating a fibrotic disease in an individual. In some embodiments, the fibrotic disease is liver fibrosis. In some embodiments, the fibrotic disease is primary sclerosing cholangitis.

Further provided is a pharmaceutical composition comprising(S)-4-((2-methoxyethyl)(4-(5,6,7,8-tetrahydro-1,8-naphthyridin-2-yl)butyl)amino)-2-(quinazolin-4-ylamino)butanoic acid, or a salt thereof (e.g., a pharmaceutically acceptable salt thereof), and a pharmaceutically acceptable carrier or excipient, for use in treatment of a fibrotic disease. In some embodiments, the fibrotic disease is liver fibrosis. In some embodiments, the fibrotic disease is primary sclerosing cholangitis.

In a further embodiment, provided is a method of treating a fibrotic disease in an individual (such as a human) in need thereof comprising administering to the individual a therapeutically effective amount of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments, the fibrotic disease is liver fibrosis. In some embodiments, the fibrotic disease is primary sclerosing cholangitis.

In a further embodiment, provided is a method of delaying the onset and/or development of a fibrotic disease in an individual (such as a human) who is at risk for developing a fibrotic disease comprising administering to the individual a therapeutically effective amount of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In a further embodiment, provided is a method of delaying the onset and/or development of a fibrotic disease in an individual (such as a human) who is at risk for developing a fibrotic disease comprising administering to the individual a therapeutically effective amount of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments, the fibrotic disease is liver fibrosis. In some embodiments, the fibrotic disease is primary sclerosing cholangitis.

Also provided(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutical composition thereof, for the treatment of a fibrotic disease. In some embodiments, the fibrotic disease is liver fibrosis. In some embodiments, the fibrotic disease is primary sclerosing cholangitis.

Also provided is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or a pharmaceutical composition comprising any of the foregoing, in the manufacture of a medicament for the treatment of a fibrotic disease. In some embodiments, the fibrotic disease is liver fibrosis. In some embodiments, the fibrotic disease is primary sclerosing cholangitis.

Further provided is a kit comprising(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments, the kit comprises instructions for use according to a method described herein, such as a method of treating a fibrotic disease in an individual. In some embodiments, the fibrotic disease is liver fibrosis. In some embodiments, the fibrotic disease is primary sclerosing cholangitis.

In a further embodiment, provided is a method of treating a subject for a disease, comprising: administering to the subject a first drug comprising(S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof; and administering to the subject at least a second drug that is ursodeoxycholic acid (UDCA), or a pharmaceutically acceptable salt thereof, whereby the subject is treated for the disease. In some embodiments, the UDCA is administered at a dose of about 25 mg/kg/day or less. In some embodiments, the UDCA is administered at a dose of about 1 mg/kg/day to about 25 mg/kg/day. In some embodiments, the UDCA is administered at a dose of about 5 mg/kg/day to about 25 mg/kg/day. In some embodiments, the UDCA is administered at a dose of about 10 mg/kg/day to about 25 mg/kg/day.

In a further embodiment, provided is a method of reducing alkaline phosphatase (ALP) levels in a subject in need thereof, comprising administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, whereby the level of ALP is reduced. In a further embodiment, provided is a method of reducing alkaline phosphatase (ALP) levels in a subject in need thereof, comprising administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, whereby the level of ALP is reduced.

In a further embodiment, provided is a method of modulating α_{V}β₆ integrin, α_{V}β₁ integrin, or both α_{V}β₆ integrin and α_{V}β₁ integrin in a subject in need thereof, comprising: administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the administering is not accompanied by a serious adverse event. In a further embodiment, provided is a method of modulating α_{V}β₆ integrin, α_{V}β₁ integrin, or both α_{V}β₆ integrin and α_{V}β₁ integrin in a subject in need thereof, comprising:
administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the administering is not accompanied by a serious treatment-related adverse event.

In a further embodiment, provided is a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein said one or more genes are selected from COL1A1, COL1A2, COL3A1, or TGFβ1.

In a further embodiment, provided is a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid to the subject, wherein said one or more genes are selected from COL1A1, COL1A2, COL3A1, or TGFβ₁.

In a further embodiment, provided is a method of modulating the activity of at least one gene affecting fibrotic activity in a subject in need thereof, comprising (i) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or (ii) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid or a pharmaceutically acceptable salt thereof. In some embodiments, the method of modulating the activity of at least one gene affecting fibrotic activity in a subject in need thereof comprises modulating one or more genes selected from COL1A1, COL1A2, COL3A1, or TGFβ₁.

In a further embodiment, provided is a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein said one or more genes are selected from COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

In a further embodiment, provided is a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein said one or more genes are selected from COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

In a further embodiment, provided is a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein said one or more genes are selected from ADAMTS3, ANXA6, CCDC80, COL5A1, COL7A1, CXCL12, FBLN1, IGFBP7, or LOXL1.

In a further embodiment, provided is a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid to the subject, wherein said one or more genes are selected from COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

In a further embodiment, provided is a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid to the subject, wherein said one or more genes are selected from COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

In a further embodiment, provided is a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid to the subject, wherein said one or more genes are selected from ADAMTS3, ANXA6, CCDC80, COL5A1, COL7A1, CXCL12, FBLN1, IGFBP7, or LOXL1.

In a further embodiment, provided is a method of modulating the activity of at least one gene affecting fibrotic activity in a subject in need thereof, comprising (i) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or (ii) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid or a pharmaceutically acceptable salt thereof. In some embodiments, the method of modulating the activity of at least one gene affecting fibrotic activity in a subject in need thereof comprises modulating one or more genes selected from COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

In a further embodiment, provided is a method of modulating the activity of at least one gene affecting fibrotic activity in a subject in need thereof, comprising (i) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or (ii) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid or a pharmaceutically acceptable salt thereof. In some embodiments, the method of modulating the activity of at least one gene affecting fibrotic activity in a subject in need thereof comprises modulating one or more genes selected from COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

In a further embodiment, provided is a method of modulating the activity of at least one gene affecting fibrotic activity in a subject in need thereof, comprising (i) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or (ii) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid or a pharmaceutically acceptable salt thereof. In some embodiments, the method of modulating the activity of at least one gene affecting fibrotic activity in a subject in need thereof comprises modulating one or more genes selected from ADAMTS3, ANXA6, CCDC80, COL5A1, COL7A1, CXCL12, FBLN1, IGFBP7, or LOXL1.

In any of the embodiments disclosed herein, the compound for use in any of the methods, including methods of treatment of disease or methods of treating a subject in need thereof, can be a compound, salt, or polymorph disclosed in United States Patent Application Publication No. US 2022/0177468, International Patent Application No. WO 2022/109598, U.S. Pat. Nos. 10,793,564, 11,419,869, United States Patent Application Publication No. US 2023/0028658, U.S. Patent Application Publication No. 2019/0276449, and International Patent Application No. WO 2019/173653. The entire contents of each of the preceding patent documents are incorporated herein by reference.

In some embodiments, the compound for use in any of the methods, including methods of treatment of disease or methods of treating a subject in need thereof, is a phosphate salt. In some embodiments, the phosphate salt is crystalline. In some embodiments, the phosphate salt is the crystalline Form I phosphate salt. In some embodiments, the phosphate salt is selected from the group consisting of: crystalline Form IV phosphate salt, crystalline Form II fumarate salt, crystalline Form III naphthalenedisulfonic acid salt, a zwitterionic form, and an amorphous form.

### BRIEF DESCRIPTION OF THE FIGURES

FIG. 1 shows a graphical representation of plasma and liver concentrations of (S)-4-((2-methoxyethyl)(4-(5,6,7,8-tetrahydro-1,8-naphthyridin-2-yl)butyl)amino)-2-(quinazolin-4-ylamino)butanoic acid in BALB/c.Mdr2−/−mice at harvest, where data is mean±standard deviation).

FIG. 2 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on portal venous pressure of the subject.

FIG. 3 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on relative liver weight of the subject, where ** p<0.01.

FIG. 4 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on relative spleen weight of the subject, where ** p<0.01.

FIG. 5 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on colon length of the subject, where ** p<0.01.

FIG. 6 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on serum ALT, where ** p<0.01.

FIG. 7 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on serum alkaline phosphatase, where ** p<0.01.

FIG. 8 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on serum total bilirubin, where ** p<0.01.

FIG. 9 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on pSMAD3, where * p<0.05, *** p<0.001, and #p<0.05.

FIG. 10 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on relative hepatic collagen, where * p<0.05, *** p<0.001, and #p<0.05.

FIG. 11 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on total hepatic collagen, where * p<0.05, *** p<0.001, and #p<0.05.

FIG. 12 shows a picrosirius red histological stained image of the vehicle in the BALBc.Mdr2−/−model.

FIG. 13 shows a picrosirius red histological stained image associated with a subject treated with 100 mg/kg of (S)-4-((2-methoxyethyl)(4-(5,6,7,8-tetrahydro-1,8-naphthyridin-2-yl)butyl)amino)-2-(quinazolin-4-ylamino)butanoic acid in the BALBc.Mdr2−/−model.

FIG. 14 shows a picrosirius red histological stained image associated with a subject treated with 300 mg/kg of (S)-4-((2-methoxyethyl)(4-(5,6,7,8-tetrahydro-1,8-naphthyridin-2-yl)butyl)amino)-2-(quinazolin-4-ylamino)butanoic acid in the BALBc.Mdr2−/−model.

FIG. 15 shows a picrosirius red histological stained image associated with a subject treated with 1000 mg/kg of (S)-4-((2-methoxyethyl)(4-(5,6,7,8-tetrahydro-1,8-naphthyridin-2-yl)butyl)amino)-2-(quinazolin-4-ylamino)butanoic acid in the BALBc.Mdr2−/−model.

FIG. 16 shows a graphical representation of the quantification of picrosirius red staining by percent area for various concentrations of (S)-4-((2-methoxyethyl)(4-(5,6,7,8-tetrahydro-1,8-naphthyridin-2-yl)butyl)amino)-2-(quinazolin-4-ylamino)butanoic acid, where * p<0.05.

FIG. 17 shows a graphical representation of (S)-4-((2-methoxyethyl)(4-(5,6,7,8-tetrahydro-1,8-naphthyridin-2-yl)butyl)amino)-2-(quinazolin-4-ylamino)butanoic acid and OCA on COLIA1 expression in precision cut liver slices (PCLivS) from human PSC liver tissue, where data from n=3 pooled slices was used from one independent experiment, culture and treatment occurred for two days, and DMSO was used as the solvent and was utilized at a constant concentration (0.1%) across the different groups.

FIG. 18 shows a graphical representation of (S)-4-((2-methoxyethyl)(4-(5,6,7,8-tetrahydro-1,8-naphthyridin-2-yl)butyl)amino)-2-(quinazolin-4-ylamino)butanoic acid and OCA on COLIA2 expression in PCLivS from human PSC liver tissue, where data from n=3 pooled slices was used from one independent experiment, culture and treatment occurred for two days, and DMSO was used as the solvent and was utilized at a constant concentration (0.1%) across the different groups.

FIG. 19 shows a graphical representation of the effect of (S)-4-((2-methoxyethyl)(4-(5,6,7,8-tetrahydro-1,8-naphthyridin-2-yl)butyl)amino)-2-(quinazolin-4-ylamino)butanoic acid on pro-fibrotic gene expression (e.g., COLIA1 expression) in PCLivS from human liver tissue with biliary fibrosis, where data was taken from 3-6 slices from 10 independent experiments (n=6 PSC livers and n=4 PBC livers), culture and treatment occurred for two days, DMSO was used as the solvent and was utilized at a constant concentration (0.1%) across the different groups, * p<0.05, and **** p<0.0001.

FIG. 20 shows a graphical representation of the effect of (S)-4-((2-methoxyethyl)(4-(5,6,7,8-tetrahydro-1,8-naphthyridin-2-yl)butyl)amino)-2-(quinazolin-4-ylamino)butanoic acid on pro-fibrotic gene expression (e.g., TGF-1 expression) in PCLivS from human liver tissue with biliary fibrosis, where data was taken from 3-6 slices from 10 independent experiments (n=6 PSC livers and n=4 PBC livers), culture and treatment occurred for two days, DMSO was used as the solvent and was utilized at a constant concentration (0.1%) across the different groups, * p<0.05, and **** p<0.0001.

FIG. 21 shows a graphical representation of the effect of (S)-4-((2-methoxyethyl)(4-(5,6,7,8-tetrahydro-1,8-naphthyridin-2-yl)butyl)amino)-2-(quinazolin-4-ylamino)butanoic acid on pro-fibrotic gene expression (e.g., COLIA2 expression) in PCLivS from human liver tissue with biliary fibrosis, where data was taken from 3-6 slices from 10 independent experiments (n=6 PSC livers and n=4 PBC livers), culture and treatment occurred for two days, DMSO was used as the solvent and was utilized at a constant concentration (0.1%) across the different groups, * p<0.05, and **** p<0.0001.

FIG. 22 shows a graphical representation of the effect of (S)-4-((2-methoxyethyl)(4-(5,6,7,8-tetrahydro-1,8-naphthyridin-2-yl)butyl)amino)-2-(quinazolin-4-ylamino)butanoic acid on pro-fibrotic gene expression (e.g., COL3A1 expression) in PCLivS from human liver tissue with biliary fibrosis, where data was taken from 3-6 slices from 10 independent experiments (n=6 PSC livers and n=4 PBC livers), culture and treatment occurred for two days, DMSO was used as the solvent and was utilized at a constant concentration (0.1%) across the different groups, * p<0.05, and **** p<0.0001.

FIG. 23 shows a graph associated with the mean change in enhanced liver fibrosis (ELF) score from Baseline to Week 12 for several concentrations of (S)-4-((2-methoxyethyl)(4-(5,6,7,8-tetrahydro-1,8-naphthyridin-2-yl)butyl)amino)-2-(quinazolin-4-ylamino)butanoic acid in Example B-3. * p<0.05 vs placebo.

FIG. 24 shows a graph associated with the mean change in the enhanced liver fibrosis (ELF) score component, tissue inhibitor of metalloproteinases-1 (TIMP-1), from Baseline to Week 12 for several concentrations of (S)-4-((2-methoxyethyl)(4-(5,6,7,8-tetrahydro-1,8-naphthyridin-2-yl)butyl)amino)-2-(quinazolin-4-ylamino)butanoic acid in Example B-3.

FIG. 25 shows a graph associated with the mean change in the enhanced liver fibrosis (ELF) score component, procollagen III (PIIINP), from Baseline to Week 12 for several concentrations of (S)-4-((2-methoxyethyl)(4-(5,6,7,8-tetrahydro-1,8-naphthyridin-2-yl)butyl)amino)-2-(quinazolin-4-ylamino)butanoic acid in Example B-3.

FIG. 26 shows a graph associated with the mean change in the enhanced liver fibrosis (ELF) score component, hyaluronic acid (HA), from Baseline to Week 12 for several concentrations of (S)-4-((2-methoxyethyl)(4-(5,6,7,8-tetrahydro-1,8-naphthyridin-2-yl)butyl)amino)-2-(quinazolin-4-ylamino)butanoic acid in Example B-3.

FIG. 27 shows a graph associated with the percent mean change in neo-epitope pro-peptide of type III collagen formation (PROC-C3) change for several concentrations of (S)-4-((2-methoxyethyl)(4-(5,6,7,8-tetrahydro-1,8-naphthyridin-2-yl)butyl)amino)-2-(quinazolin-4-ylamino)butanoic acid over a sixteen-week time period in Example B-3.

FIG. 28 shows a graph associated with the percent mean change in ALP for several concentrations of (S)-4-((2-methoxyethyl)(4-(5,6,7,8-tetrahydro-1,8-naphthyridin-2-yl)butyl)amino)-2-(quinazolin-4-ylamino)butanoic acid at Week 12 in Example B-3.

FIG. 29 shows a graph associated with the percent mean change in contrast agent, Gadoxetate, for several concentrations of (S)-4-((2-methoxyethyl)(4-(5,6,7,8-tetrahydro-1,8-naphthyridin-2-yl)butyl)amino)-2-(quinazolin-4-ylamino)butanoic acid in MRI from Baseline at Week 12 in Example B-3.

FIG. 30 shows a graph associated with the percent mean change in Gadoxetate Time of Arrival in Bile Duct for several concentrations of (S)-4-((2-methoxyethyl)(4-(5,6,7,8-tetrahydro-1,8-naphthyridin-2-yl)butyl)amino)-2-(quinazolin-4-ylamino)butanoic acid at Week 12 in Example B-3.

FIG. 31 shows a graph associated with the mean change in itch numerical rating scale (NRS) for several concentrations of (S)-4-((2-methoxyethyl)(4-(5,6,7,8-tetrahydro-1,8-naphthyridin-2-yl)butyl)amino)-2-(quinazolin-4-ylamino)butanoic acid from Baseline at Week 12 in Example B-3.

FIG. 32 shows the amount of total (left graph) versus unbound (right graph) (S)-4-((2-methoxyethyl)(4-(5,6,7,8-tetrahydro-1,8-naphthyridin-2-yl)butyl)amino)-2-(quinazolin-4-ylamino)butanoic acid in plasma during the 12-week study period.

FIG. 33 shows mean (SE) change in enhanced liver fibrosis (ELF) score from Baseline for a safety population at Week 12 for 40 mg of bexotegrast (“40 mg”); 80 mg of bexotegrast (“80 mg”); 160 mg of bexotegrast (“160 mg”); 320 mg of bexotegrast (“320 mg”); bexotegrast at 40 mg, 80 mg, 160 mg, and 320 mg (“ALL BEXO”); and placebo of Example B-4.

FIG. 34 shows the percentage of mean (SE) change in neo-epitope pro-peptide of type III collagen formation (PRO-C3) for a safety population over a twelve-week period for 40 mg of bexotegrast (“40 mg”); 80 mg of bexotegrast (“80 mg”); 160 mg of bexotegrast (“160 mg”); 320 mg of bexotegrast (“320 mg”); and placebo of Example B-4, where * p<0.05 vs. placebo.

FIG. 35 shows the percentage of mean (SE) change in neo-epitope pro-peptide of type III collagen formation (PRO-C3) for a safety population over a twelve-week period for bexotegrast at 40 mg, 80 mg, 160 mg, and 320 mg (“ALL BEXO”) and placebo of Example B-4, where * p<0.05 vs. placebo and ** p<0.01 vs. placebo.

FIG. 36 shows the percentage of mean (SE) change in Gadoxetate relative enhancement (RE) for a sub-study safety population from Baseline at Week 12 for 40 mg of bexotegrast (“40 mg”); 80 mg of bexotegrast (“80 mg”); 160 mg of bexotegrast (“160 mg”); 320 mg of bexotegrast (“320 mg”); bexotegrast at 40 mg, 80 mg, 160 mg, and 320 mg (“ALL BEXO”); and placebo of Example B-4, where * p<0.05 vs. placebo and ** p<0.01 vs. placebo.

FIG. 37 shows the percentage of mean (SE) change in Gadoxetate time of arrival to common bile duct for a sub-study safety population at Week 12 in seconds for bexotegrast (“40 mg”); 80 mg of bexotegrast (“80 mg”); 160 mg of bexotegrast (“160 mg”); 320 mg of bexotegrast (“320 mg”); bexotegrast at 40 mg, 80 mg, 160 mg, and 320 mg (“ALL BEXO”); and placebo of Example B-4, where * p<0.05 vs. placebo and ** p<0.01 vs. placebo.

FIG. 38 shows the mean (SE) change in alkaline phosphatase (ALP) from baseline for a safety population (subjects with ALP>ULN at Baseline) at Week 12 for bexotegrast (“40 mg”); 80 mg of bexotegrast (“80 mg”); 160 mg of bexotegrast (“160 mg”); 320 mg of bexotegrast (“320 mg”); bexotegrast at 40 mg, 80 mg, 160 mg, and 320 mg (“ALL BEXO”); and placebo of Example B-4, where * p<0.05 vs. placebo.

FIG. 39 shows the mean (SE) change in itch numerical rating scale (NRS) of a safety population for bexotegrast (“40 mg”); 80 mg of bexotegrast (“80 mg”); 160 mg of bexotegrast (“160 mg”); 320 mg of bexotegrast (“320 mg”); bexotegrast at 40 mg, 80 mg, 160 mg, and 320 mg (“ALL BEXO”); and placebo of Example B-4, where * p<0.05 vs. placebo.

FIG. 40 shows mean (SE) change in an enhanced liver fibrosis (ELF) score component, tissue inhibitor of metalloproteinases-1 (TIMP-1), for a safety population from Baseline at Week 12 for 40 mg, 80, mg, 160 mg, and 320 mg of bexotegrast, all four doses (40 mg, 80 mg, 160 mg, and 320 mg) of bexotegrast pooled together (“ALL BEXO”), and placebo of Example B-4.

FIG. 41 shows mean (SE) change in an enhanced liver fibrosis (ELF) score component, procollagen III, N-terminal propeptide (PIIINP), for a safety population from Baseline at Week 12 for 40 mg, 80, mg, 160 mg, and 320 mg of bexotegrast, all four doses (40 mg, 80 mg, 160 mg, and 320 mg) of bexotegrast pooled together (“ALL BEXO”), and placebo of Example B-4.

FIG. 42 shows mean (SE) change in an enhanced liver fibrosis (ELF) score component, hyaluronic acid (HA), for a safety population from Baseline at Week 12 for 40 mg, 80, mg, 160 mg, and 320 mg of bexotegrast, all four doses (40 mg, 80 mg, 160 mg, and 320 mg) of bexotegrast pooled together (“ALL BEXO”), and placebo of Example B-4.

FIG. 43 shows data indicating that the Mean Itch Numerical Rating Scale (NRS) score did not increase over 24 weeks of treatment with bexotegrast, compared to an increase on placebo in Example B-5.

FIG. 44A shows that there was no increase in Enhanced Liver Fibrosis (ELF) scores observed with bexotegrast from weeks 12 to 24 in Example B-5.

FIG. 44B shows a reduction in ELF score at week 24 observed in the 320 mg bexotegrast treatment group, compared to an increase in the placebo group, in a high-risk subpopulation in Example B-5.

FIG. 45 shows a decrease in median liver stiffness in the bexotegrast-treated group, compared to an increase in the placebo group in Example B-5

FIG. 46A shows the change in alkaline phosphatase levels (ALP) over the course of the study in Example B-5 in all participants in the bexotegrast-treated group and the placebo group.

FIG. 46B shows alkaline phosphatase level (ALP) reductions observed in the Example B-5 study in participants with baseline ALP>ULN compared to increasing ALP on placebo. ULN=upper limit of normal.

FIG. 47 shows alkaline phosphatase level (ALP) reductions observed in all participants at Week 12 and Week 24 of the study in Example B-5.

FIG. 48A shows improvements in the MRI parameter relative enhancement (RE), using the contrast agent gadoxetate, observed at weeks 12 and 24 in the study in Example B-5.

FIG. 48B shows individual values for the MRI parameter relative enhancement (RE) corresponding to FIG. 48A.

FIG. 48C shows improvements in the MRI parameter time of arrival of gadoxetate to bile duct observed at weeks 12 and 24 in the study in Example B-5.

FIG. 48D shows individual values for the MRI parameter time of arrival of gadoxetate to bile duct corresponding to FIG. 48C.

### DETAILED DESCRIPTION OF THE INVENTION

The present disclosure provides, inter alia, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, pharmaceutical compositions comprising(S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof, and methods of using such compounds and compositions in treating fibrotic diseases. In some embodiments, the fibrotic disease is liver fibrosis. In some embodiments, the fibrotic disease is primary sclerosing cholangitis.

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

As used herein, a “small molecule” is an organic molecule characterized by a mass of less than 900 Daltons.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. Beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. Also encompassed by “treatment” is a reduction of pathological consequence of fibrosis. The methods disclosed herein contemplate any one or more of these aspects of treatment.

As used herein, the term “effective amount” intends such amount of a compound disclosed herein which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

A “therapeutically effective amount” refers to an amount of a compound or salt thereof (e.g., pharmaceutically acceptable salt thereof) sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like) and formulating the mixture according to the desired route of delivery (e.g., coated capsules, implantable reservoirs, injectable solutions containing biodegradable capsules, and the like).

As used herein, the term “composition” or “pharmaceutical composition” refers to the combination of an active agent with an excipient or a carrier, inert or active, making the composition especially suitable for diagnostic or therapeutic use in vivo or ex vivo.

Pharmaceutical compositions may be prepared by known pharmaceutical methods. Suitable compositions, excipients, or carriers can be found, e.g., in Remington: The Science and Practice of Pharmacy, Lippincott Williams & Wilkins, 21st ed. (2005), which is incorporated herein by reference in its entirety.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. See, e.g., Handbook of Pharmaceutical Salts Properties, Selection, and Use, International Union of Pure and Applied Chemistry, John Wiley & Sons (2008), hereby incorporated by reference in its entirety. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound disclosed herein in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound disclosed herein as an active ingredient. See, e.g., Handbook of Pharmaceutical Excipients. 6th Edition, Pharmaceutical Press (2008), hereby incorporated by reference in its entirety. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc= “directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

Unless otherwise stated, “substantially pure” intends a composition that contains no more than 10% impurity, such as a composition comprising less than 9%, 7%, 5%, 3%, 1%, 0.5% impurity.

It is understood that aspects and embodiments described herein as “comprising” include “consisting of” and “consisting essentially of” embodiments. The term “comprise” or variations such as “comprises” or “comprising,” will be understood to imply the inclusion of a stated element, integer or step, or group of elements, integers or steps, but not the exclusion of any other element, integer or step, or group of elements, integers or steps. It is understood that aspects and embodiments described herein as “comprising” include “consisting of” and “consisting essentially of” embodiments.

When a composition is described as “consisting essentially of” the listed components, the composition contains the components expressly listed, and may contain other components which do not substantially affect the disease or condition being treated such as trace impurities. However, the composition either does not contain any other components which do substantially affect the disease or condition being treated other than those components expressly listed; or, if the composition does contain extra components other than those listed which substantially affect the disease or condition being treated, the composition does not contain a sufficient concentration or amount of those extra components to substantially affect the disease or condition being treated. When a method is described as “consisting essentially of” the listed steps, the method contains the steps listed, and may contain other steps that do not substantially affect the disease or condition being treated, but the method does not contain any other steps which substantially affect the disease or condition being treated other than those steps expressly listed.

Blood level concentrations of drug substances and other substances in a subject can be measured in plasma or serum, as appropriate. Where a level of a substance is indicated as measured in plasma, the level can also be measured in serum if such a measurement is suitably accurate. Where a level of a substance is indicated as measured in serum, the level can also be measured in plasma if such a measurement is suitably accurate.

### Compounds

(S)-4-((2-methoxyethyl)(4-(5,6,7,8-tetrahydro-1,8-naphthyridin-2-yl)butyl)amino)-2-(quinazolin-4-ylamino)butanoic acid (bexotegrast) and pharmaceutically acceptable salts thereof are disclosed herein for use in the methods described herein. The synthesis of (S)-4-((2-methoxyethyl)(4-(5,6,7,8-tetrahydro-1,8-naphthyridin-2-yl)butyl)amino)-2-(quinazolin-4-ylamino)butanoic acid is disclosed in U.S. Pat. No. 10,793,564, which is incorporated by reference herein in its entirety, as Compound 5 of that application. Salts and polymorphs of (S)-4-((2-methoxyethyl)(4-(5,6,7,8-tetrahydro-1,8-naphthyridin-2-yl)butyl)amino)-2-(quinazolin-4-ylamino)butanoic acid are disclosed in US Patent Application Publication No. 2022/0177468, which is incorporated by reference herein in its entirety.

The disclosure also includes all salts of compounds referred to herein, such as pharmaceutically acceptable salts. The disclosure also includes any or all of the stereochemical forms, including any enantiomeric or diastereomeric forms, and any tautomers or other forms of the compounds described. Unless stereochemistry is explicitly indicated in a chemical structure or name, the structure or name is intended to embrace all possible stereoisomers of a compound depicted. In addition, where a specific stereochemical form is depicted, it is understood that other stereochemical forms are also described and embraced. All forms of the compounds are also embraced, such as crystalline or non-crystalline forms of the compounds. It is also understood that prodrugs, solvates and metabolites of the compounds are embraced by this disclosure. Compositions comprising a compound of the disclosure are also intended, such as a composition of substantially pure compound, including a specific stereochemical form thereof. Compositions comprising a mixture of compounds of the disclosure in any ratio are also embraced, including mixtures of two or more stereochemical forms of a compound in any ratio, such that racemic, non-racemic, enantioenriched and scalemic mixtures of a compound are embraced. Where one or more tertiary amine moiety is present in the compound, the N-oxides are also provided and described.

The disclosure also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, where one or more atoms are replaced by an isotope of the same element. Exemplary isotopes that can be incorporated into compounds disclosed include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl. Incorporation of heavier isotopes such as deuterium (²H or D) can afford certain therapeutic advantages resulting from greater metabolic stability, for example, increased in vivo half-life, or reduced dosage requirements and, hence may be preferred in some instances. As used herein, each instance of replacement of a hydrogen by deuterium is also a disclosure of replacing that hydrogen with tritium. As used herein, each instance of enrichment, substitution, or replacement of an atom with corresponding isotope of that atom encompasses isotopic enrichment levels of one of about: 50%, 60%, 70%, 80%, 90%, 95%, 96%, 97%, 98%, 99%, 99.5%, 99,6%, 99.7%, 99.8%, 99.9%, or 100%, or a range between any two of the preceding percentages.

Isotopically-labeled compounds of the present disclosure can generally be prepared by standard methods and techniques known to those skilled in the art or by procedures similar to those described in the accompanying Examples substituting appropriate isotopically-labeled reagents in place of the corresponding non-labeled reagent.

The disclosure also includes any or all metabolites of any of the compounds described. The metabolites may include any chemical species generated by a biotransformation of any of the compounds described, such as intermediates and products of metabolism of the compound.

Articles of manufacture comprising a compound as disclosed herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms.

### Pharmaceutical Compositions and Formulations

Pharmaceutical compositions of (S)-4-((2-methoxyethyl)(4-(5,6,7,8-tetrahydro-1,8-naphthyridin-2-yl)butyl)amino)-2-(quinazolin-4-ylamino)butanoic acid (bexotegrast) and pharmaceutically acceptable salts thereof are embraced by this disclosure. Thus, the disclosure includes pharmaceutical compositions comprising a compound as disclosed herein or a pharmaceutically acceptable salt thereof and a pharmaceutically acceptable carrier or excipient. In one embodiment, the pharmaceutically acceptable salt is an acid addition salt, such as a salt formed with an inorganic or organic acid. Pharmaceutical compositions according to the disclosure may take a form suitable for oral, buccal, parenteral, nasal, topical or rectal administration or a form suitable for administration by inhalation. In one embodiment, the pharmaceutical composition is a composition for controlled release of any of the compounds detailed herein.

A compound as detailed herein may in one embodiment be in a purified form and compositions comprising a compound in purified forms are detailed herein. In one embodiment, compositions may have no more than 35% impurity, wherein the impurity denotes a compound other than the compound comprising the majority of the composition or a pharmaceutically acceptable salt thereof, for example, (S)-4-((2-methoxyethyl)(4-(5,6,7,8-tetrahydro-1,8-naphthyridin-2-yl)butyl)amino)-2-(quinazolin-4-ylamino)butanoic acid (bexotegrast) and pharmaceutically acceptable salts thereof may contains no more than 35% impurity, wherein the impurity denotes a compound other than(S)-4-((2-methoxyethyl)(4-(5,6,7,8-tetrahydro-1,8-naphthyridin-2-yl)butyl)amino)-2-(quinazolin-4-ylamino)butanoic acid (bexotegrast) or a pharmaceutically acceptable salt thereof. In one embodiment, compositions may have no more than 35% impurity, wherein the impurity denotes a compound other than the compound comprising the majority of the composition or a pharmaceutically acceptable salt thereof, for example, a composition of a compound selected from(S)-4-((2-methoxyethyl)(4-(5,6,7,8-tetrahydro-1,8-naphthyridin-2-yl)butyl)amino)-2-(quinazolin-4-ylamino)butanoic acid (bexotegrast) and pharmaceutically acceptable salts thereof may contain no more than 35% impurity, wherein the impurity denotes a compound other than(S)-4-((2-methoxyethyl)(4-(5,6,7,8-tetrahydro-1,8-naphthyridin-2-yl)butyl)amino)-2-(quinazolin-4-ylamino)butanoic acid (bexotegrast), or a pharmaceutically acceptable salt thereof. In one embodiment, compositions may contain no more than 25% impurity. In one embodiment, compositions may contain no more than 20% impurity. In still further embodiments, compositions comprising a compound as detailed herein or a pharmaceutically acceptable salt thereof are provided as compositions of substantially pure compounds. “Substantially pure” compositions comprise no more than 10% impurity, such as a composition comprising less than 9%, 7%, 5%, 3%, 1%, or 0.5% impurity. In some embodiments, a composition containing a compound as detailed herein or a pharmaceutically acceptable salt thereof is in substantially pure form. In still another variation, a composition of substantially pure compound or a pharmaceutically acceptable salt thereof is provided wherein the composition contains or no more than 10% impurity. In a further variation, a composition of substantially pure compound or a pharmaceutically acceptable salt thereof is provided wherein the composition contains or no more than 9% impurity. In a further variation, a composition of substantially pure compound or a pharmaceutically acceptable salt thereof is provided wherein the composition contains or no more than 7% impurity. In a further variation, a composition of substantially pure compound or a pharmaceutically acceptable salt thereof is provided wherein the composition contains or no more than 5% impurity. In another variation, a composition of substantially pure compound or a pharmaceutically acceptable salt thereof is provided wherein the composition contains or no more than 3% impurity. In still another variation, a composition of substantially pure compound or a pharmaceutically acceptable salt thereof is provided wherein the composition contains or no more than 1% impurity. In a further variation, a composition of substantially pure compound or a pharmaceutically acceptable salt thereof is provided wherein the composition contains or no more than 0.5% impurity. In yet other variations, a composition of substantially pure compound means that the composition contains no more than 10% or preferably no more than 5% or more preferably no more than 3% or even more preferably no more than 1% impurity or most preferably no more than 0.5% impurity, which impurity may be the compound in a different stereochemical form. For instance, a composition of substantially pure(S) compound means that the composition contains no more than 10% or no more than 5% or no more than 3% or no more than 1% or no more than 0.5% of the I form of the compound.

In one variation, the compounds herein are synthetic compounds prepared for administration to an individual such as a human. In another variation, compositions are provided containing a compound in substantially pure form. In another variation, the disclosure embraces pharmaceutical compositions comprising a compound detailed herein and a pharmaceutically acceptable carrier or excipient. In another variation, methods of administering a compound are provided. The purified forms, pharmaceutical compositions and methods of administering the compounds are suitable for any compound or form thereof detailed herein.

A compound detailed herein or a pharmaceutically acceptable salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or a pharmaceutically acceptable salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

(S)-4-((2-methoxyethyl)(4-(5,6,7,8-tetrahydro-1,8-naphthyridin-2-yl)butyl)amino)-2-(quinazolin-4-ylamino)butanoic acid (bexotegrast) as described herein or a pharmaceutically acceptable salt thereof can be used in the preparation of a formulation, such as a pharmaceutical formulation, by combining the compound, or a pharmaceutically acceptable salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21st ed. (2005), which is incorporated herein by reference in its entirety.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

In one embodiment, the compounds can be administered in the liquid vehicle ORA-SWEET® from PERRIGO®, Allegan, Michigan, which is a syrup vehicle having ingredients of purified water, glycerin, sorbitol, sodium saccharin, xanthan gum, and flavoring, buffered with citric acid and sodium citrate, preserved with methylparaben (0.03%), potassium sorbate (0.1%), and propylparaben (0.008%); or in a mixture of ORA-SWEET® and water of any proportion, such as a 50:50 mixture of ORA-SWEET® to water. The water used should be a pharmaceutically acceptable grade of water, for example, sterile water.

Any of the compounds described herein can be formulated in a tablet in any dosage form described, for example, a compound as described herein or a pharmaceutically acceptable salt thereof can be formulated as a 10 mg tablet.

Compositions comprising a compound provided herein are also described. In one variation, the composition comprises a compound and a pharmaceutically acceptable carrier or excipient. In another variation, a composition of substantially pure compound is provided. In some embodiments, the composition is for use as a human or veterinary medicament. In some embodiments, the composition is for use in a method described herein. In some embodiments, the composition is for use in the treatment of a disease or disorder described herein.

### Methods of Use

Compounds and compositions as disclosed herein, such as a pharmaceutical composition containing a compound of any formula provided herein or a pharmaceutically acceptable salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays. The compounds and compositions are used to ameliorate diseases as disclosed herein. In some embodiments, amelioration can comprise an improvement in a measured parameter (such as a decrease in a parameter when a decrease of the parameter is desirable, or an increase in a parameter when an increase of the parameter is desirable), of about 5%, about 10%, about 20%, about 30%, about 40%, about 50%, about 60%, about 70%, about 80%, about 90%, or about 100% after administration of a compound or composition as disclosed herein, as compared to the measured parameter before the administration.

In one embodiment, provided is a method of ameliorating fibrosis in tissue of a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In one embodiment, provided is a method of ameliorating fibrosis in tissue of a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In one embodiment, provided is a method of ameliorating fibrosis in tissue of a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In one embodiment, provided is a method of ameliorating fibrosis in tissue of a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the tissue comprises liver tissue, gall bladder tissue, or bile duct tissue. In embodiments, the tissue comprises liver tissue and gall bladder tissue. In embodiments, the tissue comprises liver tissue and bile duct tissue. In embodiments, the tissue comprises gall bladder tissue and bile duct tissue. In embodiments, the tissue comprises liver tissue. In embodiments, the tissue comprises gall bladder tissue. In embodiments, the tissue comprises bile duct tissue. In embodiments, the human has liver fibrosis. In embodiments, the human is suspected of having liver fibrosis. In embodiments, the human has primary sclerosing cholangitis. In embodiments, the human is suspected of having primary sclerosing cholangitis. In embodiments, the human is at risk for moderate to severe fibrosis. In embodiments, the human has one or more of the following risk factors for fibrosis prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof: an enhanced liver fibrosis score greater than or equal to about 7.7; a transient elastography measurement between about 7.7 kPa and about 14.4 kPa; a magnetic resonance elastography measurement between about 2.4 kPa and about 4.9 kPa, and a historical liver biopsy indicating fibrosis. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including crystalline Form I phosphate salt, crystalline Form IV phosphate salt, crystalline Form II fumarate salt, and crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is Form I phosphate salt. Salts and polymorphs of (S)-4-((2-methoxyethyl)(4-(5,6,7,8-tetrahydro-1,8-naphthyridin-2-yl)butyl)amino)-2-(quinazolin-4-ylamino)butanoic acid are disclosed in US Patent Application Publication No. 2022/0177468, incorporated by reference herein in its entirety.

In any of the embodiments herein, “prior to” administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof can be within about three months or less before the administration, within about two months or less before the administration, within about one month or less before the administration, within about three weeks or less before the administration, within about two weeks or less before the administration, within about one week or less before the administration, within about 6 days or less before the administration, within about 5 days or less before the administration, within about 4 days or less before the administration, within about 3 days or less before the administration, within about 2 days or less before the administration, within about 1 day or less before the administration, or on the day of administration but prior to the administration. In some embodiments, “prior to” administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof is within about three months or less before the administration. In some embodiments, “prior to” administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof is within about two months or less before the administration. In some embodiments, “prior to” administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof is within about 6 days or less before the administration. In some embodiments, “prior to” administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof is within about 4 days or less before the administration. In some embodiments, “prior to” administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof is within about 3 days or less before the administration. In some embodiments, “prior to” administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof is within about 2 days or less before the administration. In some embodiments, “prior to” administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof is within about one day or less before the administration. In some embodiments, “prior to” administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof is on the day of administration but prior to the administration. The administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof can be a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

In one embodiment, provided is a method of treating a fibrotic disease in an individual in need thereof comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. The individual, such as human, may be in need of treatment, such as a human who has or is suspected of having a fibrotic disease.

In some embodiments, administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is added on to a patient's existing therapeutic regimen. In some embodiments, the patient is treatment-naïve prior to administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

In one embodiment is provided a method of ameliorating a fibrotic disease in a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In one embodiment is provided a method of ameliorating a fibrotic disease in a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the fibrotic disease in the human is ameliorated. The administering can be administering orally. The administering can be administering daily, such as once daily, twice daily, three times daily, or four times daily. In embodiments, the administering is for about 12 weeks. In embodiments, the administering is for at least about 12 weeks. In embodiments, the administering is for about 24 weeks. In embodiments, the administering is for at least about 24 weeks. In embodiments, the human has liver fibrosis. In embodiments, the human is suspected of having liver fibrosis. In embodiments, the human has primary sclerosing cholangitis. In embodiments, the human is suspected of having primary sclerosing cholangitis. In embodiments, the human is at risk for moderate to severe fibrosis. In embodiments, the human has one or more of the following risk factors for fibrosis prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof: an enhanced liver fibrosis score greater than or equal to about 7.7; a transient elastography measurement between about 7.7 kPa and about 14.4 kPa; a magnetic resonance elastography measurement between about 2.4 kPa and about 4.9 kPa, and a historical liver biopsy indicating fibrosis. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In a further embodiment, provided is a method of delaying the onset and/or development of a fibrotic disease in an individual (such as a human) who is at risk for developing a fibrotic disease, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In a further embodiment, provided is a method of delaying the onset and/or development of a fibrotic disease in an individual (such as a human) who is at risk for developing a fibrotic disease, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. It is appreciated that delayed development may encompass prevention in the event the individual does not develop the fibrotic disease. An individual at risk of developing a fibrotic disease in one aspect has or is suspected of having one or more risk factors for developing a fibrotic disease. Risk factors for fibrotic disease may include an individual's age (e.g., middle-age or older adults), the presence of inflammation, having one or more genetic component associated with development of a fibrotic disease, medical history such as treatment with a drug or procedure believed to be associated with an enhanced susceptibility to fibrosis (e.g., radiology) or a medical condition believed to be associated with fibrosis, a history of smoking, the presence of occupational and/or environmental factors such as exposure to pollutants associated with development of a fibrotic disease. In some embodiments, the individual at risk for developing a fibrotic disease is an individual who has or is suspected of having primary sclerosing cholangitis.

In some embodiments, the fibrotic disease is fibrosis of the liver.

In some embodiments, the fibrotic disease is primary sclerosing cholangitis.

In some embodiments, the fibrotic disease is a primary sclerosing cholangitis, or biliary fibrosis. In some embodiments, the fibrotic disease is primary biliary cholangitis (also known as primary biliary cirrhosis) or biliary atresia.

In some embodiments, the fibrotic disease is a liver fibrosis, e.g., infectious liver fibrosis (from pathogens such as HCV, HBV or parasites such as schistosomiasis), NASH, alcoholic steatosis induced liver fibrosis, and cirrhosis. In some embodiments, the liver fibrosis is nonalcoholic fatty liver disease (NAFLD). In some embodiments, the liver fibrosis is NASH. In some embodiments, the fibrotic disease is biliary tract fibrosis.

In some embodiments, methods may include modulating the activity of at least one integrin in a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. For example, the method may include modulating the activity of α_{V}β₆. The method may include modulating the activity of α_{V}β₁. The method may include modulating the activity of α_{V}β₁ and α_{V}β₆. Modulating the activity of the at least one integrin may include, e.g., inhibiting the at least one integrin. The method may include administering to the human an amount of the compound or a pharmaceutically acceptable salt thereof effective to modulate the activity of the at least one integrin in the subject, e.g., at least one of α_{V}β₁ and α_{V}β₆. The human in need of modulating the activity of at least one integrin may have any of the fibrotic disease or conditions described herein. For example, the fibrotic disease or condition may include primary sclerosing cholangitis, primary biliary cholangitis (also known as primary biliary cirrhosis), or biliary atresia. The method may include administering to the human an amount of the compound or a pharmaceutically acceptable salt thereof effective to modulate the activity of the at least one integrin in the human, e.g., at least one of α_{V}β₁ and α_{V}β₆, the human being in need of treatment for primary sclerosing cholangitis.

The fibrotic disease may be mediated by α_{V}β₁ and α_{V}β₆, for example, the fibrotic disease may include PSC or biliary atresia. Accordingly, the method may include modulating the activity of α_{V}β₁ and α_{V}β₆ to treat conditions mediated by both α_{V}β₁ and α_{V}β₆.

The compound may be a modulator, e.g., an inhibitor, of α_{V}β₁. The compound may be a modulator, e.g., an inhibitor, of α_{V}β₆. The compound may be a dual modulator, such as a dual inhibitor, e.g., dual selective inhibitor, of α_{V}β₁ and α_{V}β₆. (S)-4-((2-methoxyethyl)(4-(5,6,7,8-tetrahydro-1,8-naphthyridin-2-yl)butyl)amino)-2-(quinazolin-4-ylamino)butanoic acid inhibits α_{V}β₁ and α_{V}β₆, and may be considered a dual α_{V}Bi/α_{V}β₆ inhibitor.

Modulating or inhibiting the activity of one or both of α_{V}β₁ integrin and α_{V}β₆ integrin, thereby treating a subject with a fibrotic disease, indicates that α_{V}β₁ integrin, α_{V}β₆ integrin, or α_{V}β₁ integrin and α_{V}β₆ integrin are modulated or inhibited to a degree sufficient to treat the fibrotic disease in the subject. In some embodiments, the fibrotic disease is liver fibrosis. In some embodiments, the fibrotic disease is primary sclerosing cholangitis.

In one embodiment, provided is(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, for use in the treatment of a fibrotic disease. In some embodiments, the fibrotic disease is liver fibrosis. In some embodiments, the fibrotic disease is primary sclerosing cholangitis.

Also provided is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for the treatment of a fibrotic disease. In some embodiments, the fibrotic disease is liver fibrosis. In some embodiments, the fibrotic disease is primary sclerosing cholangitis.

In a further embodiment, provided herein is a method of treating a subject in need thereof, comprising administering to the subject a therapeutically effective amount of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or a dosage form disclosed herein, wherein the subject has at least one tissue in need of therapy and the tissue has at least one elevated level of: α_{V}β₁ integrin activity and/or expression; α_{V}β₆ integrin activity and/or expression; a pSMAD/SMAD value; new collagen formation or accumulation; total collagen; and Type I Collagen gene COL1A1 expression; and wherein the level is elevated compared to a healthy state of the tissue. In some embodiments, the at least one tissue in the subject comprises one or more of: liver tissue, gall bladder tissue, and bile duct tissue. In some embodiments, the at least one tissue in the subject is liver tissue. In some embodiments, the at least one tissue in the subject is gall bladder tissue. In some embodiments, the at least one tissue in the subject is bile duct tissue. In some embodiments, the tissue has an elevated pSMAD2/SMAD2 value or an elevated pSMAD3/SMAD3 value compared to the healthy state of the tissue.

Methods of determining the values of α_{V}β₁ integrin activity and/or expression; α_{V}β₆ integrin activity and/or expression; a pSMAD/SMAD value; new collagen formation or accumulation; total collagen; and Type I Collagen gene COL1A1 expression are known in the art and exemplary methods are disclosed in the Examples, such as antibody assays of tissue samples, such as a biopsy sample.

In some embodiments, the method reduces both α_{V}β₁ integrin and α_{V}β₆ integrin activity and/or expression compared to at least one other α_{V}-containing integrin in the subject. In some embodiments, the activity of α_{V}β₁ integrin in one or more fibroblasts is reduced in the subject. In some embodiments, the activity of α_{V}β₆ integrin in one or more epithelial cells is reduced in the subject.

In a further embodiment, provided herein is a method of treating a subject in need thereof, comprising administering to the subject a therapeutically effective amount of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or a dosage form disclosed herein, wherein the subject has at least one tissue in need of therapy and the tissue has at least one elevated level of: α_{V}β₁ integrin activity and/or expression; α_{V}β₆ integrin activity and/or expression; a pSMAD/SMAD value; new collagen formation or accumulation; total collagen; and Type I Collagen gene COL1A1 expression; and wherein the level is elevated compared to a healthy state of the tissue. In some embodiments, the at least one tissue in the subject comprises one or more of: liver tissue, gall bladder tissue, and bile duct tissue. In some embodiments, the at least one tissue in the subject is liver tissue. In some embodiments, the at least one tissue in the subject is gall bladder tissue. In some embodiments, the at least one tissue in the subject is bile duct tissue. In some embodiments, the tissue has an elevated pSMAD2/SMAD2 value or an elevated pSMAD3/SMAD3 value compared to the healthy state of the tissue.

Methods of determining the values of α_{V}β₁ integrin activity and/or expression; α_{V}β₆ integrin activity and/or expression; a pSMAD/SMAD value; new collagen formation or accumulation; total collagen; and Type I Collagen gene COL1A1 expression are known in the art, such as antibody assays of tissue samples, such as a biopsy sample.

In some embodiments, the method reduces both α_{V}β₁ integrin and α_{V}β₆ integrin activity and/or expression compared to at least one other α_{V}-containing integrin in the subject. In some embodiments, the activity of α_{V}β₁ integrin in one or more fibroblasts is reduced in the subject. In some embodiments, the activity of α_{V}β₆ integrin in one or more epithelial cells is reduced in the subject.

Also provided herein is a method of characterizing the antifibrotic activity of a small molecule in a subject, comprising: providing a first live cell sample from the subject, the first live cell sample characterized by the presence of at least one integrin capable of activating transforming growth factor β (TGF-β) from latency associated peptide-TGF-β; determining a first pSMAD/SMAD value in the first live cell sample; administering the small molecule to the subject; providing a second live cell sample from the subject, the second live cell sample being drawn from the same tissue in the subject as the first live cell sample; determining a second pSMAD/SMAD value in the second live cell sample; and characterizing the antifibrotic activity of the small molecule in the subject by comparing the second pSMAD/SMAD value to the first pSMAD/SMAD value. In some embodiments, the small molecule is a compound disclosed herein, such as(S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof, optionally in a dosage form disclosed herein.

In some embodiments, each live cell sample is a plurality of cells derived from a tissue of the subject, or a plurality of macrophages associated with the tissue of the subject. In some embodiments, the tissue comprises one of: liver tissue, gall bladder tissue, and bile duct tissue. In some embodiments, the at least one tissue in the subject is liver tissue. In some embodiments, the at least one tissue in the subject is gall bladder tissue. In some embodiments, the at least one tissue in the subject is bile duct tissue.

In some embodiments, the subject has a fibrotic disease selected from the group consisting of: nonalcoholic fatty liver disease (NAFLD), nonalcoholic steatohepatitis (NASH), alcoholic liver disease induced fibrosis, primary sclerosing cholangitis (PSC), primary biliary cholangitis, or biliary atresia. In some embodiments, the subject has the fibrotic disease primary sclerosing cholangitis (PSC).

In some embodiments, the subject is diagnosed with a fibrotic disease selected from the group consisting of: nonalcoholic fatty liver disease (NAFLD), nonalcoholic steatohepatitis (NASH), alcoholic liver disease induced fibrosis, primary sclerosing cholangitis (PSC), primary biliary cholangitis, or biliary atresia. In some embodiments, the subject is diagnosed with the fibrotic disease primary sclerosing cholangitis (PSC). In some embodiments, the subject is diagnosed with a fibrotic disease at the age of about 65 years or younger.

In some embodiments, the at least one integrin comprises av. In some embodiments, the at least one integrin comprises α_{V}β₁. In some embodiments, the at least one integrin comprises α_{V}β₆.

In some embodiments, determining the first pSMAD/SMAD value in the at least one live cell comprises determining a pSMAD2/SMAD2 value or a pSMAD3/SMAD3 value; and determining the second pSMAD/SMAD value in the at least one live cell after contacting the at least one live cell with the small molecule comprises determining a pSMAD2/SMAD2 value or a pSMAD3/SMAD3 value.

Also provided herein is a method of treating a fibrotic disease in a subject in need thereof, comprising: providing a first live cell sample from the subject, the first live cell sample having at least one integrin capable of activating transforming growth factor β (TGF-β) from latency associated peptide-TGF-B; determining a first pSMAD/SMAD value in the first live cell sample; administering a small molecule to the subject; providing a second live cell sample from the subject, the second live cell sample being drawn from the same tissue in the subject as the first live cell sample; determining a second pSMAD/SMAD value in the second live cell sample; comparing the second pSMAD/SMAD value to the first pSMAD/SMAD value; and administering the small molecule to the subject if the second pSMAD/SMAD value is lower than the first pSMAD/SMAD value. In some embodiments, the small molecule is a compound disclosed herein or a pharmaceutically acceptable salt thereof, optionally in a dosage form disclosed herein. In some embodiments, the first live cell sample is obtained from the subject prior to treatment with a small molecule.

In some embodiments, each live cell sample is a plurality of cells derived from a tissue of the subject, or a plurality of macrophages associated with the tissue of the subject. In some embodiments, the tissue comprises one of: liver tissue, gall bladder tissue, and bile duct tissue. In some embodiments, the tissue comprises liver tissue. In some embodiments, the tissue comprises gall bladder tissue. In some embodiments, the tissue comprises bile duct tissue.

In some embodiments, the subject is characterized by having a fibrotic disease selected from the group consisting of: nonalcoholic fatty liver disease (NAFLD), nonalcoholic steatohepatitis (NASH), alcoholic liver disease induced fibrosis, primary sclerosing cholangitis (PSC), primary biliary cholangitis, or biliary atresia. In some embodiments, the subject is characterized by having primary sclerosing cholangitis (PSC).

In some embodiments, the at least one integrin comprises av. In some embodiments, the at least one integrin comprises α_{V}β₁. In some embodiments, the at least one integrin comprises α_{V}β₆.

In some embodiments, determining the first pSMAD/SMAD value in the first live cell sample comprises determining a pSMAD2/SMAD2 value or a pSMAD3/SMAD3 value; and determining the second pSMAD/SMAD value in the at least one live cell after contacting the first live cell sample with the small molecule comprises determining a pSMAD2/SMAD2 value or a pSMAD3/SMAD3 value.

In a further embodiment, provided is a method of inhibiting α_{V}β₆ integrin in an individual comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Also provided is a method of inhibiting TGFβ activation in a cell comprising administering to the cell(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Also provided is a method of inhibiting α_{V}β₆ integrin in an individual in need thereof, comprising administering to the individual(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In one such method, the compound is a selective α_{V}β₆ integrin inhibitor.

Also provided is a method of modulating or inhibiting α_{V}β₆ integrin in an individual in need thereof without substantially increasing liver inflammation, comprising administering to the individual(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

In another embodiment is provided a method of modulating or inhibiting α_{V}β₆ integrin and α_{V}β₁ integrin without substantially increasing liver inflammation. In all such embodiments, in one aspect the method of inhibition is for an individual in need thereof, such as an individual who has or is suspected of having a fibrotic disease, and wherein the method comprises administering to the individual(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, without substantially increasing liver inflammation.

In any of the described methods, in one aspect the individual is a human, such as a human in need of the method. The individual may be a human who has been diagnosed with or is suspected of having a fibrotic disease. The individual may be a human who has been diagnosed with or is suspected of having primary sclerosing cholangitis. The individual may be a human who does not have detectable fibrotic disease but who has one or more risk factors for developing a fibrotic disease. The individual may be a human who does not have detectable primary sclerosing cholangitis but who has one or more risk factors for developing primary sclerosing cholangitis.

Also provided herein are dosage forms configured for daily administration, comprising a pharmaceutically acceptable carrier or excipient; and a unit dose(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. A dose amount corresponds to an amount of (S)-4-((2-methoxyethyl)(4-(5,6,7,8-tetrahydro-1,8-naphthyridin-2-yl)butyl)amino)-2-(quinazolin-4-ylamino)butanoic acid.

A unit dose, such as a unit dose for daily administration, can comprise about 1, 2.5, 5, 7.5, 10, 15, 20, 25, 30, 35, 40, 50, 75, 80, 85, 90, 95, 100, 105, 110, 115, 120, or 125 mg of the compound, or a range between any two of the preceding values, such as about 1-125, 1-5, 2.5-7.5, 5-15, 10-15, 10-20, 10-25, 10-30, 10-35, 10-40, 10-50, 10-75, 15-20, 15-25, 15-30, 15-35, 15-40, 15-50, 15-75, 20-25, 20-30, 20-35, 20-40, 20-50, 20-75, 25-30, 25-35, 25-40, 25-50, 25-75, 30-35, 30-40, 30-50, 30-75, 35-40, 35-50, 35-75, 40-50, 40-75, 50-75, 50-100, 60-85, 70-90, 70-100, 80-125, 90-125, or 100-125 mg.

A unit dose, such as a unit dose for daily administration, can comprise about 1, 2.5, 5, 7.5, 10, 15, 20, 25, 30, 35, 40, 50, 75, 80, 85, 90, 95, 100, 105, 110, 115, 120, 125, 150, 175, 200, 225, or 250 mg of the compound, or a range between any two of the preceding values, such as about 1-125, 1-250, 1-5, 2.5-7.5, 5-15, 10-15, 10-20, 10-25, 10-30, 10-35, 10-40, 10-50, 10-75, 15-20, 15-25, 15-30, 15-35, 15-40, 15-50, 15-75, 20-25, 20-30, 20-35, 20-40, 20-50, 20-75, 25-30, 25-35, 25-40, 25-50, 25-75, 30-35, 30-40, 30-50, 30-75, 35-40, 35-50, 35-75, 40-50, 40-75, 50-75, 50-100, 50-150, 50-250, 60-85, 70-90, 70-100, 80-125, 90-125, 100-125, 100-150, 100-200, 125-175, 100-225, 100-250, and 150-250 mg. For example, the unit dose may be 10 mg. The unit dose may be 15 mg. The unit dose may be 20 mg. The unit dose may be 30 mg. The unit dose may be 40 mg. The unit dose may be 50 mg. The unit dose may be 60 mg. The unit dose may be 70 mg. The unit dose may be 75 mg. The unit dose may be 80 mg. The unit dose may be 90 mg. The unit dose may be 100 mg. The unit dose may be 110 mg. The unit dose may be 120 mg. The unit dose may be 125 mg. The unit dose may be 150 mg. The unit dose may be 175 mg. The unit dose may be 200 mg. The unit dose may be 225 mg. The unit dose may be 250 mg.

Also provided herein are dosage forms configured for daily administration, comprising a pharmaceutically acceptable carrier or excipient; and a unit dose of (S)-4-((2-methoxyethyl)(4-(5,6,7,8-tetrahydro-1,8-naphthyridin-2-yl)butyl)amino)-2-(quinazolin-4-ylamino)butanoic acid, or a stereoisomer thereof, or a pharmaceutically acceptable salt thereof.

In various embodiments, a dose, e.g., a unit dose, such as a unit dose for daily administration, can include the compound in an amount of one of, or one of about: 1, 2.5, 5, 7.5, 10, 15, 20, 25, 30, 35, 40, 45, 50, 55, 60, 75, 80, 85, 90, 95, 100, 105, 110, 115, 120, 125, 130, 140, 150, 160, 170, 175, 180, 190, 200, 225, 240, 250, 275, 300, 320, 325, 350, 375, 400, 425, 450, 475, 480, 500, 525, 550, 560, 575, 600, 625, 640, 650, 675, 700, 720, 725, 750, 775, 800, 825, 850, 875, 880, 900, 925, 950, 960, 975, 1000, 1025, or 1040 milligrams. For example, a dose can include the compound in an amount of, or of about, 10 mg. A dose can include the compound in an amount of, or of about, 15 mg. A dose can include the compound in an amount of, or of about, 20 mg. A dose can include the compound in an amount of, or of about, 30 mg. A dose can include the compound in an amount of, or of about, 40 mg. A dose can include the compound in an amount of, or of about, 50 mg. A dose can include the compound in an amount of, or of about, 75 mg. A dose can include the compound in an amount of, or of about, 80 mg. A dose can include the compound in an amount of, or of about, 100 mg. A dose can include the compound in an amount of, or of about, 120 mg. A dose can include the compound in an amount of, or of about, 160 mg. A dose can include the compound in an amount of, or of about, 240 mg. A dose can include the compound in an amount of, or of about, 320 mg. A dose can include the compound in an amount of, or of about, 400 mg. A dose can include the compound in an amount of, or of about, 480 mg. A dose can include the compound in an amount of, or of about, 560 mg. A dose can include the compound in an amount of, or of about, 640 mg. A dose can include the compound in an amount of, or of about, 720 mg. A dose can include the compound in an amount of, or of about, 800 mg. A dose can include the compound in an amount of, or of about, 880 mg. A dose can include the compound in an amount of, or of about, 960 mg. A dose can include the compound in an amount of, or of about, 1040 mg.

In various embodiments, a dose, e.g., a unit dose, such as a unit dose for daily administration, can include the compound in an amount comprising an amount of the compound in mg of about one of about: 320, 400, 480, 560, 640, 720, 800, 880, 960, or 1040, or a range between any two of the preceding values.

In various embodiments, a dose, e.g., a unit dose, such as a unit dose for daily administration, can include the compound in an amount comprising an amount of the compound in mg of about one of about: 400, 480, 560, 640, 720, 800, 880, 960, or 1040.

In various embodiments, a dose, e.g., a unit dose, such as a unit dose for daily administration, can include the compound in an amount comprising an amount of the compound in mg of a range between about 320 and any one of about 400, 480, 560, 640, 720, 800, 880, 960, or 1040.

In various embodiments, a dose, e.g., a unit dose, such as a unit dose for daily administration, can include the compound in an amount comprising an amount of the compound in mg of about one of: 400, 480, 560, 640, 720, 800, 880, 960, or 1040, or a range between any two of the preceding values.

In some embodiments, the weight dosage of a pharmaceutically acceptable salt is adjusted to administer the same amount of active agent on a molar basis as would be administered if the non-salt compound were used. For example, if a dosage is indicated as 100 mg of a non-salt compound, such as the free base of an amine, or such as a zwitterion with no counterions, with a molecular weight of 500, which is a dosage of 0.2 mmol, and the hydrochloride salt of the same compound has a molecular weight of 536.5, then 107.3 mg of the hydrochloride salt would be administered in order to administer 0.2 mmol of active agent. As used herein, the amount of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, refers to the amount of (S)-4-((2-methoxyethyl)(4-(5,6,7,8-tetrahydro-1,8-naphthyridin-2-yl)butyl)amino)-2-(quinazolin-4-ylamino)butanoic acid free base equivalents present in the composition. The corresponding amount of pharmaceutically acceptable salt may be determined based on the form of (S)-4-((2-methoxyethyl)(4-(5,6,7,8-tetrahydro-1,8-naphthyridin-2-yl)butyl)amino)-2-(quinazolin-4-ylamino)butanoic acid used. For example, about 100 mg of the Form I phosphate salt of (S)-4-((2-methoxyethyl)(4-(5,6,7,8-tetrahydro-1,8-naphthyridin-2-yl)butyl)amino)-2-(quinazolin-4-ylamino)butanoic acid corresponds to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid free base equivalent.

In some embodiments, the unit dose may include the compound in a percentage range about any of the individual values in milligrams recited in the preceding paragraph, for example, any percentage range independently selected from one of, or one of about: ±1%, ±2%, ±2.5%, ±5%, ±7.5%, ±10%, ±15%, ±20%, ±25%, ±30%, ±40%, or ±50%. For example, the range may be, or be about, ±1%. The range may be, or be about, ±2%. The range may be, or be about, ±2.5%. The range may be, or be about, ±5%. The range may be, or be about, ±7.5%. The range may be, or be about, ±10%. The range may be, or be about, ±15%. The range may be, or be about, ±20%. The range may be, or be about, ±25%. The range may be, or be about, ±30%. The range may be, or be about, ±40%. The range may be, or be about, ±50%.

Further, for example, the unit dose may include the compound in an amount of one of: 10 mg±1%; 10 mg±2%; 10 mg±2.5%; 10 mg±5%; 10 mg±7.5%; 10 mg±10%; 10 mg±15%; 10 mg±20%; 10 mg±25%; 10 mg±30%; 10 mg±40%; or 10 mg±50%. The unit dose may include the compound in an amount of one of: 15 mg±1%; 15 mg±2%; 15 mg±2.5%; 15 mg±5%; 15 mg±7.5%; 15 mg±10%; 15 mg±15%; 15 mg±20%; 15 mg±25%; 15 mg±30%; 15 mg±40%; or 15 mg±50%. The unit dose may include the compound in an amount of one of: 20 mg±1%; 20 mg±2%; 20 mg±2.5%; 20 mg±5%; 20 mg±7.5%; 20 mg±10%; 20 mg±15%; 20 mg±20%; 20 mg±25%; 20 mg±30%; 20 mg±40%; or 20 mg±50%. The unit dose may include the compound in an amount of one of: 30 mg±1%; 30 mg±2%; 30 mg±2.5%; 30 mg±5%; 30 mg±7.5%; 30 mg±10%; 30 mg±15%; 30 mg±20%; 30 mg±25%; 30 mg±30%; 30 mg±40%; or 30 mg±50%. The unit dose may include the compound in an amount of one of: 40 mg±1%; 40 mg±2%; 40 mg±2.5%; 40 mg±5%; 40 mg±7.5%; 40 mg±10%; 40 mg±15%; 40 mg±20%; 40 mg±25%; 40 mg±30%; 40 mg±40%; or 40 mg±50%. The unit dose may include the compound in an amount of one of: 50 mg±1%; 50 mg±2%; 50 mg±2.5%; 50 mg±5%; 50 mg±7.5%; 50 mg±10%; 50 mg±15%; 50 mg±20%; 50 mg±25%; 50 mg±30%; 50 mg±40%; or 50 mg±50%. The unit dose may include the compound in an amount of one of: 60 mg±1%; 60 mg±2%; 60 mg±2.5%; 60 mg±5%; 60 mg±7.5%; 60 mg±10%; 60 mg±15%; 60 mg±20%; 60 mg±25%; 60 mg±30%; 60 mg±40%; or 60 mg±50%. The unit dose may include the compound in an amount of one of: 75 mg±1%; 75 mg±2%; 75 mg±2.5%; 75 mg±5%; 75 mg±7.5%; 75 mg±10%; 75 mg±15%; 75 mg±20%; 75 mg±25%; 75 mg±30%; 75 mg±40%; or 75 mg±50%. The unit dose may include the compound in an amount of one of: 80 mg±1%; 80 mg±2%; 80 mg±2.5%; 80 mg±5%; 80 mg±7.5%; 80 mg±10%; 80 mg±15%; 80 mg±20%; 80 mg±25%; 80 mg±30%; 80 mg±40%; or 80 mg±50%. The unit dose may include the compound in an amount of one of: 100 mg±1%; 100 mg±2%; 100 mg±2.5%; 100 mg±5%; 100 mg±7.5%; 100 mg±10%; 100 mg±15%; 100 mg±20%; 100 mg±25%; 100 mg±30%; 100 mg±40%; or 100 mg±50%. The unit dose may include the compound in an amount of one of: 120 mg±1%; 120 mg±2%; 120 mg±2.5%; 120 mg±5%; 120 mg±7.5%; 120 mg±10%; 120 mg±15%; 120 mg±20%; 120 mg±25%; 120 mg±30%; 120 mg±40%; or 120 mg±50%. The unit dose may include the compound in an amount of one of: 160 mg±1%; 160 mg±2%; 160 mg±2.5%; 160 mg±5%; 160 mg±7.5%; 160 mg±10%; 160 mg±15%; 160 mg±20%; 160 mg±25%; 160 mg±30%; 160 mg±40%; or 160 mg±50%. The unit dose may include the compound in an amount of one of: 240 mg±1%; 240 mg±2%; 240 mg±2.5%; 240 mg±5%; 240 mg±7.5%; 240 mg±10%; 240 mg±15%; 240 mg±20%; 240 mg±25%; 240 mg±30%; 240 mg±40%; or 240 mg±50%. The unit dose may include the compound in an amount of one of: 320 mg±1%; 320 mg±2%; 320 mg±2.5%; 320 mg±5%; 320 mg±7.5%; 320 mg±10%; 320 mg±15%; 320 mg±20%; 320 mg±25%; 320 mg±30%; 320 mg±40%; or 320 mg±50%. The unit dose may include the compound in an amount of one of: 400 mg±1%; 400 mg±2%; 400 mg±2.5%; 400 mg±5%; 400 mg±7.5%; 400 mg±10%; 400 mg±15%; 400 mg±20%; 400 mg±25%; 400 mg±30%; 400 mg±40%; or 400 mg±50%. The unit dose may include the compound in an amount of one of: 480 mg±1%; 480 mg±2%; 480 mg±2.5%; 480 mg±5%; 480 mg±7.5%; 480 mg±10%; 480 mg±15%; 480 mg±20%; 480 mg±25%; 480 mg±30%; 480 mg±40%; or 480 mg±50%. The unit dose may include the compound in an amount of one of: 560 mg±1%; 560 mg±2%; 560 mg±2.5%; 560 mg±5%; 560 mg±7.5%; 560 mg±10%; 560 mg±15%; 560 mg±20%; 560 mg±25%; 560 mg±30%; 560 mg±40%; or 560 mg±50%. The unit dose may include the compound in an amount of one of: 640 mg±1%; 640 mg±2%; 640 mg±2.5%; 640 mg±5%; 640 mg±7.5%; 640 mg±10%; 640 mg±15%; 640 mg±20%; 640 mg±25%; 640 mg±30%; 640 mg±40%; or 640 mg±50%. The unit dose may include the compound in an amount of one of: 720 mg±1%; 720 mg±2%; 720 mg±2.5%; 720 mg±5%; 720 mg±7.5%; 720 mg±10%; 720 mg±15%; 720 mg±20%; 720 mg±25%; 720 mg±30%; 720 mg±40%; or 720 mg±50%. The unit dose may include the compound in an amount of one of: 800 mg±1%; 800 mg±2%; 800 mg±2.5%; 800 mg±5%; 800 mg±7.5%; 800 mg±10%; 800 mg±15%; 800 mg±20%; 800 mg±25%; 800 mg±30%; 800 mg±40%; or 800 mg±50%. The unit dose may include the compound in an amount of one of: 880 mg±1%; 880 mg±2%; 880 mg±2.5%; 880 mg±5%; 880 mg±7.5%; 880 mg±10%; 880 mg±15%; 880 mg±20%; 880 mg±25%; 880 mg±30%; 880 mg±40%; or 880 mg±50%. The unit dose may include the compound in an amount of one of: 960 mg±1%; 960 mg±2%; 960 mg±2.5%; 960 mg±5%; 960 mg±7.5%; 960 mg±10%; 960 mg±15%; 960 mg±20%; 960 mg±25%; 960 mg±30%; 960 mg±40%; or 960 mg±50%. The unit dose may include the compound in an amount of one of: 1040 mg±1%; 1040 mg±2%; 1040 mg±2.5%; 1040 mg±5%; 1040 mg±7.5%; 1040 mg±10%; 1040 mg±15%; 1040 mg±20%; 1040 mg±25%; 1040 mg±30%; 1040 mg±40%; or 1040 mg±50%.

For numerical values indicated herein with “about,” the numerical value can be replaced by plus or minus 10%, plus or minus 5%, plus or minus 2%, or plus or minus 1%. For example, “about 100” can be replaced by 90-110, 95-105, 98-102, or 99-101.

The dosage form for daily administration can be administered to an individual in need thereof once daily. That is, the total amount of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, which is to be administered each day, can be administered all together at one time daily. Alternatively, if it is desirable that the total amount of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is to be administered in two or more portions daily, the dosage form containing the appropriate amount of compound can be administered two times or more daily, such as twice a day, three times a day, or four times a day.

The present application contemplates combination administration of the compound of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof with a second drug, for example, ursodeoxycholic acid (UDCA), or a pharmaceutically acceptable salt thereof.

In a further embodiment, method of treating a subject for a disease is provided, the method comprising: administering to the subject a first drug comprising(S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof; and administering to the subject at least a second drug comprising ursodeoxycholic acid, or a pharmaceutically acceptable salt thereof, whereby the subject is treated for the disease.

Administration of any drug can be associated with adverse events (AEs), which may rise to the level of serious adverse events (SAEs). An adverse event (AE) is considered a serious adverse event (SAE) when the patient outcome is death, life-threatening, hospitalization (initial or prolonged), disability or permanent damage, congenital anomaly/birth defect, required intervention to prevent permanent impairment or damage (due to the use of a medical product), or is otherwise considered an important medical event (see URL www.fda.gov/safety/reporting-serious-problems-fda/what-serious-adverse-event and Kizer K W, Stegun M B. Serious Reportable Adverse Events in Health Care. In: Henriksen K, Battles J B, Marks E S, Lewin DI, editors. Advances in Patient Safety: From Research to Implementation (Volume 4: Programs, Tools, and Products). Rockville (MD): Agency for Healthcare Research and Quality (US); 2005 February. PMID: 21250024, the entire contents of which are hereby incorporated by reference in their entirety). A serious adverse event includes an adverse event which is life-threatening (e.g., its occurrence places the subject at immediate risk of death); results in death; requires inpatient hospitalization (i.e., admission, overnight stay) or prolongs existing hospitalization; results in persistent or significant disability/incapacity; is a congenital anomaly/birth defect/miscarriage; or is an important medical event (e.g., is considered to be clinically significant and may jeopardize the subject, or when medical or surgical intervention may be required to prevent one of the outcomes listed above).

In some embodiments, the subject is undergoing concurrent treatment with standard of care therapy for primary sclerosing cholangitis. In some embodiments, the subject is undergoing concurrent treatment with another therapeutic for primary sclerosing cholangitis. In some embodiments, the subject is not undergoing concurrent treatment with standard of care therapy for primary sclerosing cholangitis. In some embodiments, the subject is not undergoing concurrent treatment with another therapeutic for primary sclerosing cholangitis. In some embodiments, standard of care therapy for primary sclerosing cholangitis comprises administration of ursodeoxycholic acid to the subject.

In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed at about 40 mg, about 80 mg, about 160 mg, or about 320 mg. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed at about 40 mg, about 80 mg, or about 160 mg. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed at about 40 mg. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed at about 80 mg. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed at about 160 mg. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed at about 320 mg. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed BID or QD. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed for at least 4 weeks. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed for at least 8 weeks. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed for at least 12 weeks. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed for at least 24 weeks. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is dosed for at least 24 weeks. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In some embodiments, the compound, or a pharmaceutically acceptable salt thereof, is dosed BID or QD. In some embodiments, the compound, or a pharmaceutically acceptable salt thereof, is dosed for at least about 4 weeks. In some embodiments, the compound, or a pharmaceutically acceptable salt thereof, is dosed for at least about 8 weeks. In some embodiments, the compound, or a pharmaceutically acceptable salt thereof, is dosed for at least about 12 weeks. In some embodiments, the compound, or a pharmaceutically acceptable salt thereof, is dosed for at least about 24 weeks. In some embodiments, the compound, or a pharmaceutically acceptable salt thereof, is dosed for between at least about 4 weeks to at least about 12 weeks. In some embodiments, the compound, or a pharmaceutically acceptable salt thereof, is dosed for between at least about 4 weeks to at least about 24 weeks.

In some embodiments, the individual does not require treatment for an adverse event up to 12 weeks. In some embodiments, the individual does not require treatment for an adverse event up to 24 weeks. In some embodiments, the severe adverse event comprises atrial flutter. In some embodiments, the individual does not require treatment for a gastrointestinal adverse event. In some embodiments, there is no dose relationship for adverse events.

In embodiments, a method of treating primary sclerosing cholangitis in a human in need thereof is provided, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein primary sclerosing cholangitis in the human is ameliorated. In embodiments, a method of treating primary sclerosing cholangitis in a human in need thereof is provided, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein primary sclerosing cholangitis in the human is ameliorated as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In a further embodiment, a method of treating primary sclerosing cholangitis in a human in need thereof is provided, the method comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human in a dose of about 40, 80, 160, or 320 mg, wherein primary sclerosing cholangitis in the human is ameliorated. In a further embodiment, a method of treating primary sclerosing cholangitis in a human in need thereof is provided, the method comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human in a dose of about 40, 80, 160, or 320 mg, wherein primary sclerosing cholangitis in the human is ameliorated as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments, the human is undergoing concurrent treatment with ursodeoxycholic acid. In some embodiments, the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid is dosed at about 40 mg. In some embodiments, the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid is dosed at about 80 mg. In some embodiments, the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid is dosed at about 160 mg. In some embodiments, the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid is dosed at about 320 mg. In some embodiments, the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid is dosed at about 40 or 160 mg. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the phosphate salt of (S)-4-((2-methoxyethyl)(4-(5,6,7,8-tetrahydro-1,8-naphthyridin-2-yl)butyl)amino)-2-(quinazolin-4-ylamino)butanoic acid is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In one embodiment is provided a method of modulating α_{V}β₆ integrin, α_{V}β₁ integrin, or both α_{V}β₆ integrin and α_{V}β₁ integrin in a subject in need thereof, comprising: administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the administering is not accompanied by a serious adverse event. In embodiments, the administering is not accompanied by a serious adverse event, wherein the serious adverse event is life-threatening (e.g., its occurrence places the subject at immediate risk of death); results in death; requires inpatient hospitalization (i.e., admission, overnight stay) or prolongs existing hospitalization; results in persistent or significant disability/incapacity; is a congenital anomaly/birth defect/miscarriage; or is an important medical event (e.g., is considered to be clinically significant and may jeopardize the subject, or when medical or surgical intervention may be required to prevent one of the outcomes listed above).

In one embodiment is provided a method of modulating α_{V}β₆ integrin, α_{V}β₁ integrin, or both α_{V}β₆ integrin and α_{V}β₁ integrin in a subject in need thereof, comprising: administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, wherein the administering is not accompanied by a serious adverse event. In embodiments, the administering is not accompanied by a serious adverse event, wherein the serious adverse event is life-threatening (e.g., its occurrence places the subject at immediate risk of death); results in death; requires inpatient hospitalization (i.e., admission, overnight stay) or prolongs existing hospitalization; results in persistent or significant disability/incapacity; is a congenital anomaly/birth defect/miscarriage; or is an important medical event (e.g., is considered to be clinically significant and may jeopardize the subject, or when medical or surgical intervention may be required to prevent one of the outcomes listed above).

The Enhanced Liver Fibrosis (ELF) score is a set of markers for extracellular matrix (ECM) activity. Three serum markers are measured, tissue inhibitor of metalloproteinases 1 (TIMP-1), amino-terminal propeptide of type III procollagen (PIIINP) and hyaluronic acid (HA). The measurements are combined in an algorithm to provide a score that correlates well with the degree of liver fibrosis (Sharma, C. et al., Journal of Gastroenterology and Hepatology, 36:1788-1802(2021), incorporated by reference herein in its entirety). A change in ELF score greater than or equal to 0.19 is indicative of clinical progression within two years (Muir A. J. et al., Hepatology, 2019 February; 69 (2): 684-698, incorporated by reference herein in its entirety). An ELF greater than or equal to 7.7 is considered moderate to severe fibrosis (Vesterhus M. et al., Hepatology, 2015 July; 62(1):188-97; incorporated by reference herein in its entirety).

In one embodiment is provided a method of decreasing the ELF score in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject. In one embodiment is provided a method of decreasing the ELF score in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, as compared to a subject who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or compared to a subject that has been administered a placebo. In one embodiment is provided a method of decreasing the ELF score in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, to the subject. In one embodiment is provided a method of decreasing the ELF score in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, to the subject as compared to a subject who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or compared to a subject that has been administered a placebo. In embodiments, the subject in need thereof has primary sclerosing cholangitis. In embodiments, the subject in need thereof is suspected of having primary sclerosing cholangitis. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In some embodiments, a method of decreasing a component of the ELF score in a subject in need thereof is described, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject. In some embodiments, a method of decreasing a component of the ELF score (e.g., tissue inhibitor of metalloproteinases-1 (TIMP-1)) in a subject in need thereof is described, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject. In some embodiments, a method of decreasing a component of the ELF score (e.g., procollagen III, N-terminal propeptide (PIIINP)) in a subject in need thereof is described, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject. In some embodiments, a method of decreasing a component of the ELF score (e.g., hyaluronic acid (HA)) in a subject in need thereof is described, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject. In some embodiments, a method of decreasing a component of the ELF score (e.g., TIMP-1) in a subject in need thereof is described, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, as compared to a subject who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or compared to a subject that has been administered a placebo. In some embodiments, a method of decreasing a component of the ELF score (e.g., PIIINP) in a subject in need thereof is described, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, as compared to a subject who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or compared to a subject that has been administered a placebo. In some embodiments, a method of decreasing a component of the ELF score (e.g., HA) in a subject in need thereof is described, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, as compared to a subject who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or compared to a subject that has been administered a placebo. In embodiments, the subject in need thereof has primary sclerosing cholangitis. In embodiments, the subject in need thereof is suspected of having primary sclerosing cholangitis. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof, is dosed in an amount of about 40 mg to about 400 mg, such as a daily dose of about 40 mg to about 400 mg. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof, is dosed in an amount of about 40 mg to about 320 mg, such as a daily dose of about 40 mg to about 320 mg. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof, is dosed in an amount of about 40 mg to about 200 mg, such as a daily doses of about 40 mg to about 200 mg. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof, is dosed in an amount of about 40 mg to about 160 mg, such as a daily doses of about 40 mg to about 160 mg. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof, is dosed in an amount of about 40 mg to about 80 mg, such as a daily doses of about 40 mg to about 80 mg. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 40 mg daily, or the pharmaceutically acceptable salt thereof is administered in an amount equivalent to about 40 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 80 mg daily, or the pharmaceutically acceptable salt thereof is administered in an amount equivalent to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 160 mg daily, or the pharmaceutically acceptable salt thereof is administered in an amount equivalent to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 320 mg daily, or the pharmaceutically acceptable salt thereof is administered in an amount equivalent to about 320 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In one embodiment is provided a method of ameliorating enhanced liver fibrosis score in a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the enhanced liver fibrosis score in the human is ameliorated. In one embodiment is provided a method of ameliorating enhanced liver fibrosis score in a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the enhanced liver fibrosis score in the human is ameliorated as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. As a higher enhanced liver fibrosis score is indicative of higher fibrosis and thus undesirable, the result of the administering can be a decrease in enhanced liver fibrosis score, a lack of increase of the enhanced liver fibrosis score, or an increase in enhanced liver fibrosis score due to administering that is less than the increase in enhanced liver fibrosis score in the absence of the administering. The administering can be administering orally. The administering can be administering daily, such as once daily, twice daily, three times daily, or four times daily. In embodiments, the administering is for about 12 weeks. In embodiments, the administering is for at least about 12 weeks. In embodiments, the amelioration of enhanced liver fibrosis score occurs within about 12 weeks following a first administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the amelioration of enhanced liver fibrosis score is measured at about 12 weeks following a first administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the administering is for about 24 weeks. In embodiments, the administering is for at least about 24 weeks. In embodiments, the amelioration of enhanced liver fibrosis score occurs within about 24 weeks following a first administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the amelioration of enhanced liver fibrosis score is measured at about 24 weeks following a first administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the human has liver fibrosis. In embodiments, the human is suspected of having liver fibrosis. In embodiments, the human has primary sclerosing cholangitis. In embodiments, the human is suspected of having primary sclerosing cholangitis. In embodiments, the human is at risk for moderate to severe fibrosis. In embodiments, the human has one or more of the following risk factors for fibrosis prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof: an enhanced liver fibrosis score greater than or equal to about 7.7; a transient elastography measurement between about 7.7 kPa and about 14.4 kPa; a magnetic resonance elastography measurement between about 2.4 kPa and about 4.9 kPa, and a historical liver biopsy indicating fibrosis. In embodiments, the amelioration of increase in enhanced liver fibrosis score is measured at or after about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, such as at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the enhanced liver fibrosis score increase is less than about 0.2 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the enhanced liver fibrosis score increase is less than about 0.1 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the amelioration of increase in enhanced liver fibrosis score is measured at or after about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, such as at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the enhanced liver fibrosis score increase is unchanged at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the enhanced liver fibrosis score increase is less than about 0.05 at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the enhanced liver fibrosis score increase is less than about 0.1 at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments, the human has an enhanced liver fibrosis score of about 9.8 or higher prior to the first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In another embodiment is provided a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from COL1A1, COL1A2, COL3A1, or TGFβ₁. In another embodiment is provided a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC. In another embodiment is provided a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1. In another embodiment is provided a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from ADAMTS3, ANXA6, CCDC80, COL5A1, COL7A1, CXCL12, FBLN1, IGFBP7, or LOXL1In another embodiment is provided a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, to the subject, wherein said one or more genes are selected from COL1A1, COL1A2, COL3A1, or TGFβ₁. In another embodiment is provided a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, to the subject, wherein said one or more genes are selected from COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC. In another embodiment is provided a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, to the subject, wherein said one or more genes are selected from COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1. In another embodiment is provided a method of decreasing the expression of one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, to the subject, wherein said one or more genes are selected from ADAMTS3, ANXA6, CCDC80, COL5A1, COL7A1, CXCL12, FBLN1, IGFBP7, or LOXL1. In another embodiment is provided a method of modulating the activity of at least one gene affecting fibrotic activity in a subject in need thereof, comprising (i) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or (ii) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, wherein the at least one gene is substantially modulated by said administering. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In another embodiment is provided a method of reducing alkaline phosphatase (ALP) levels in a human in need thereof, comprising administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, whereby the level of ALP is reduced. In another embodiment is provided a method of reducing alkaline phosphatase (ALP) levels in a human in need thereof, comprising administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, whereby the level of ALP is reduced as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. ALP ranges for healthy individuals are provided in Table M-1 below.

TABLE M-1

| Alkaline phosphatase (ALP) | Females | Males |
|---|---|---|
| 18 to 19 years of age | 45-87 U/L | 55-149 U/L |
| >19 years of age | 35-104 U/L | 40-129 U/L |

In embodiments, administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human results in an amelioration in alkaline phosphatase level in the human. In embodiments, administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human results in an amelioration in alkaline phosphatase level in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human results in a change in alkaline phosphatase level in the human. In embodiments, the alkaline phosphatase level is above an upper limit of normal prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the amelioration in alkaline phosphatase level in the human occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the change in alkaline phosphatase level occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the change in alkaline phosphatase level is from about 8.8 U/L to about 1.4 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the change in alkaline phosphatase level is from about 8.8 U/L to about-8.4 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the change in alkaline phosphatase level is from about 2.1 U/L to about-8.4 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the alkaline phosphatase level is decreased following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the change in alkaline phosphatase level is from about-8.4 U/L to about 0 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, alkaline phosphatase level is stable at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the amelioration in alkaline phosphatase level in the human occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the change in alkaline phosphatase level occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the change in alkaline phosphatase level is from about 4 U/L to about-30 U/L at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the change in alkaline phosphatase level is from about 2 U/L to about-26 U/L at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the change in alkaline phosphatase level is from about-30 U/L to about 0 U/L at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the change in alkaline phosphatase level is from about −40 U/L to about 0 U/L at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the alkaline phosphatase level is decreased following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, alkaline phosphatase level is stable at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the human has liver fibrosis. In embodiments, the human is suspected of having liver fibrosis. In embodiments, the human has primary sclerosing cholangitis. In embodiments, the human is suspected of having primary sclerosing cholangitis. In embodiments, the human is at risk for moderate to severe fibrosis. In embodiments, the human has one or more of the following risk factors for fibrosis prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof: an enhanced liver fibrosis score greater than or equal to about 7.7; a transient elastography measurement between about 7.7 kPa and about 14.4 kPa; a magnetic resonance elastography measurement between about 2.4 kPa and about 4.9 kPa, and a historical liver biopsy indicating fibrosis. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In embodiments, provided is a method of stabilizing or decreasing total bilirubin in a human in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human in need thereof. In embodiments, provided is a method of stabilizing or decreasing total bilirubin in a human in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human in need thereof, as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. The stabilizing or decreasing of total bilirubin can occur within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. The stabilizing or decreasing of total bilirubin can be from about-0.2 mg/dL to about 1 mg/dL at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. The stabilizing or decreasing of total bilirubin is decreased by about 0.5 mg/dL at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. The stabilizing or decreasing of total bilirubin can occur within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In embodiments, the human has liver fibrosis. In embodiments, the human is suspected of having liver fibrosis. In embodiments, the human has primary sclerosing cholangitis. In embodiments, the human is suspected of having primary sclerosing cholangitis. In embodiments, the human is at risk for moderate to severe fibrosis. In embodiments, the human has one or more of the following risk factors for fibrosis prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof: an enhanced liver fibrosis score greater than or equal to about 7.7; a transient elastography measurement between about 7.7 kPa and about 14.4 kPa; a magnetic resonance elastography measurement between about 2.4 kPa and about 4.9 kPa, and a historical liver biopsy indicating fibrosis. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

Transient elastography (TE) utilizes an ultrasound shear wave to assess the elasticity of the liver, that is, how “stiff” the liver tissue is. Fibrosis causes stiffening or hardening of tissue, and TE can be used to diagnose fibrosis and determine the degree of fibrosis. The FibroScan® device (FibroScan® is a registered trademark of ECHOSENS, Paris, France, for medical equipment and software) can be used for TE. (Kemp W, Roberts S., Aust Fam Physician. 2013 July; 42(7):468-71; and Ozturk A. et al., Abdom Radiol (NY), 2022 September; 47 (9): 3037-3050; each of which are hereby incorporated by reference in their entireties).

For cholestatic diseases, including primary sclerosing cholangitis, one particular scale (as provided by the Memorial Sloan Kettering Cancer Center, New York) is given in the following Table M-2.

TABLE M-2

| Liver Stiffness | Fibrosis Score | Clinical significance |
|---|---|---|
| 2 to 7 kPa | F0 to F1 | Liver is normal. |
| 7 to 9 kPa | F2 | Liver has moderate scarring. |
| 9 to 17 kPa | F3 | Liver has severe scarring. |
| 17 kPa or higher | F4 | Liver has cirrhosis. |

In one embodiment is provided a method of ameliorating the transient elastography measurement in a human in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In one embodiment is provided a method of ameliorating the transient elastography measurement in a human in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In one embodiment is provided a method of changing the transient elastography measurement in a human in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In one embodiment is provided a method of changing the transient elastography measurement in a human in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In one embodiment is provided a method of modulating the transient elastography measurement in a human in need thereof, wherein the transient elastography change occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In one embodiment is provided a method of modulating the transient elastography measurement in a human in need thereof, wherein the transient elastography change occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In one embodiment is provided a method of modulating the transient elastography measurement in a human in need thereof, wherein the transient elastography change is from about-0.56 kPa to about 0.85 kPa at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In one embodiment is provided a method of modulating the transient elastography measurement in a human in need thereof, wherein the transient elastography change occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In one embodiment is provided a method of modulating the transient elastography measurement in a human in need thereof, wherein the transient elastography change occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In one embodiment is provided a method of modulating the transient elastography measurement in a human in need thereof, wherein the transient elastography change is from about-0.4 kPa to about 0.4 kPa at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In embodiments, the human has liver fibrosis. In embodiments, the human is suspected of having liver fibrosis. In embodiments, the human has primary sclerosing cholangitis. In embodiments, the human is suspected of having primary sclerosing cholangitis.

In embodiments, the human is at risk for moderate to severe fibrosis. In embodiments, the human has one or more of the following risk factors for fibrosis prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof: an enhanced liver fibrosis score greater than or equal to about 7.7; a transient elastography measurement between about 7.7 kPa and about 14.4 kPa; a magnetic resonance elastography measurement between about 2.4 kPa and about 4.9 kPa, and a historical liver biopsy indicating fibrosis. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In one embodiment is provided a method of modulating the transient elastography measurement in a patient in need thereof, wherein the transient elastography change is about 0.85 kPa at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In one embodiment is provided a method of modulating the transient elastography measurement in a patient in need thereof, wherein the transient elastography change is about 0.4 kPa at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the human has liver fibrosis. In embodiments, the human is suspected of having liver fibrosis. In embodiments, the human has primary sclerosing cholangitis. In embodiments, the human is suspected of having primary sclerosing cholangitis. In embodiments, the human is at risk for moderate to severe fibrosis. In embodiments, the human has one or more of the following risk factors for fibrosis prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof: an enhanced liver fibrosis score greater than or equal to about 7.7; a transient elastography measurement between about 7.7 kPa and about 14.4 kPa; a magnetic resonance elastography measurement between about 2.4 kPa and about 4.9 kPa, and a historical liver biopsy indicating fibrosis. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In one embodiment is provided a method of reducing itch or pruritus (chronic itching). Itch can be quantified on a 0-10 scale using the Peak Pruritus Numerical Rating Scale (NRS), where 0 is “no itch” and 10 is “worst imaginable itch” over the past 24 hours (Yosipovitch et al., British Journal of Dermatology (2019) 181:761-769, incorporated by reference herein in its entirety). In embodiments, a method of reducing pruritus in a human who has fibrotic liver disease is provided, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, a method of reducing (e.g., ameliorating) pruritus in a human who has fibrotic liver disease (e.g., primary sclerosing cholangitis) is provided, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, a method of reducing pruritus in a human who has fibrotic liver disease is provided, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, a method of ameliorating pruritus in a human who has primary sclerosing cholangitis is provided, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In embodiments, the reduction of pruritus occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In further embodiments, pruritus is reduced as measured by a change in itch numerical rating scale from between about 0.8 and about-0.2 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In further embodiments, the change in itch numerical rating scale is less than about 0.2 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In further embodiments, the itch numerical rating for the human is not increased at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In embodiments, the reduction of pruritus occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. In further embodiments, pruritus is reduced as measured by a change in itch numerical rating scale from between about 1 and about-0.2 at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In further embodiments, the change in itch numerical rating scale is less than about 0.2 at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In further embodiments, the itch numerical rating for the human is not increased at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In one embodiment is provided a method of treating primary sclerosing cholangitis by modulating α_{V}β₆ integrin, α_{V}β₁ integrin, or both α_{V}β₆ integrin and α_{V}β₁ integrin in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the administering is not accompanied by a serious adverse event. In embodiments, the administering is not accompanied by a serious adverse event, wherein the serious adverse event is life-threatening (e.g., its occurrence places the subject at immediate risk of death); results in death; requires inpatient hospitalization (i.e., admission, overnight stay) or prolongs existing hospitalization; results in persistent or significant disability/incapacity; is a congenital anomaly/birth defect/miscarriage; or is an important medical event (e.g., is considered to be clinically significant and may jeopardize the subject, or when medical or surgical intervention may be required to prevent one of the outcomes listed above).

In one embodiment is provided a method of treating primary sclerosing cholangitis by modulating α_{V}β₆ integrin, α_{V}β₁ integrin, or both α_{V}β₆ integrin and α_{V}β₁ integrin in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, wherein the administering is not accompanied by a serious adverse event. In embodiments, the administering is not accompanied by a serious adverse event, wherein the serious adverse event is life-threatening (e.g., its occurrence places the subject at immediate risk of death); results in death; requires inpatient hospitalization (i.e., admission, overnight stay) or prolongs existing hospitalization; results in persistent or significant disability/incapacity; is a congenital anomaly/birth defect/miscarriage; or is an important medical event (e.g., is considered to be clinically significant and may jeopardize the subject, or when medical or surgical intervention may be required to prevent one of the outcomes listed above).

In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from COL1A1, COL1A2, COL3A1, or TGFβ₁. In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC. In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1. In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from ADAMTS3, ANXA6, CCDC80, COL5A1, COL7A1, CXCL12, FBLN1, IGFBP7, or LOXL1. In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from COL1A2, COL3A1, or TGFβ₁. In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC. In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1 . . . . In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the subject, wherein said one or more genes are selected from ADAMTS3, ANXA6, CCDC80, COL5A1, COL7A1, CXCL12, FBLN1, IGFBP7, or LOXL1.

In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, to the subject, wherein said one or more genes are selected from COL1A1, COL1A2, COL3A1, or TGFβ₁. In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, to the subject, wherein said one or more genes are selected from COL1A2, COL3A1, or TGFβ₁. In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, to the subject, wherein said one or more genes are selected from COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC. In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, to the subject, wherein said one or more genes are selected from COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1. In another embodiment is provided a method of treating primary sclerosing cholangitis by decreasing the expression of one or more genes in a subject in need thereof or downregulating one or more genes in a subject in need thereof, comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, to the subject, wherein said one or more genes are selected from ADAMTS3, ANXA6, CCDC80, COL5A1, COL7A1, CXCL12, FBLN1, IGFBP7, or LOXL1. In another embodiment is provided a method of treating primary sclerosing cholangitis by modulating the activity of at least one gene affecting fibrotic activity in a subject in need thereof, comprising (i) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or (ii) administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, and ursodeoxycholic acid, or a pharmaceutically acceptable salt thereof, wherein the at least one gene is substantially modulated by said administering. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In some embodiments is provided a method of slowing the clinical progression of primary sclerosing cholangitis in a human who has primary sclerosing cholangitis and is at risk for moderate to severe liver fibrosis, comprising administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments is provided a method of slowing the clinical progression of primary sclerosing cholangitis in a human who has primary sclerosing cholangitis and is at risk for moderate to severe liver fibrosis, comprising administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments is provided a method of slowing the clinical progression of primary sclerosing cholangitis in a human who has primary sclerosing cholangitis and is at risk for moderate to severe liver fibrosis, comprising administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or compared to a human who has been administered a placebo. In further embodiments, the clinical progression at two years following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human is slowed as compared to a subject who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In further embodiments, the slowing of clinical progression is measured by an enhanced liver fibrosis score increase of less than about 0.2 following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

In embodiments, provided is a method of reducing collagen synthesis in a human in need thereof. The reduction of collagen synthesis can occur within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. The reducing of collagen synthesis can be measured by a percent change in PRO-C3 from about-3.4% to about 2.9% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. The reduction of collagen synthesis can occur within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human.

In embodiments, provided is a method of reducing of PRO-C3 in a human in need thereof. The reducing of PRO-C3 can occur within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In embodiments, PRO-C3 is decreased by about 3.4% to about 0.5% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In embodiments, PRO-C3 is decreased by about 0.5% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In embodiments, mean change in PRO-C3 is about 0.6% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. The reducing of PRO-C3 can occur within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In any of the embodiments provided herein, hepatocyte function is improved. The improvement in hepatocyte function can occur within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. The improvement in hepatocyte function can be measured by contrast MRI at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In any of the embodiments provided herein, bile flow is improved. The improvement in bile flow can occur within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. The improvement in bile flow can be measured by contrast MRI at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is from about 3.4% to about 0.8% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about 3.4% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about 1.1% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about 2.1% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about 0.5% to about 1.0% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about 0.8% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about 2% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. The improvement in hepatocyte function can occur within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. The improvement in hepatocyte function can be measured by contrast MRI at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In any of the embodiments provided herein, bile flow is improved. The improvement in bile flow can occur within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. The improvement in bile flow can be measured by contrast MRI at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is from about 0.5% to about 5% at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about 5% at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about 4% at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about 3% at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about 2% to about 1% at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about 0.5% to about 1% at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, MRI relative enhancement is about unchanged at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In any of the embodiments provided herein, bile flow is improved. The improvement in bile flow can occur within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. The improvement in bile flow can be change in time of arrival to common bile duct measured by excretory function MRI at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, change in time of arrival to common bile duct is from about 35.1% to about-27.6% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, change in time of arrival to common bile duct is about-3.1% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, change in time of arrival to common bile duct is about 35.1% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, change in time of arrival to common bile duct is about-27.6% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, change in time of arrival to common bile duct is about-22.2% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, change in time of arrival to common bile duct is about-6.8% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. The improvement in bile flow can occur within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, to the human. The improvement in bile flow can be change in time of arrival to common bile duct measured by excretory function MRI at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, change in time of arrival to common bile duct is from about-10 seconds to about −150 seconds at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, change in time of arrival to common bile duct is about-20 seconds to about-130 seconds at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, change in time of arrival to common bile duct is about-20 seconds to about-100 seconds at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt.

In any of the methods disclosed herein, such as a method of treating liver fibrosis in a subject in need thereof or a method of treating primary sclerosing cholangitis (PSC) in a subject in need thereof, the method can comprise administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in an amount of about 40 mg to about 400 mg, such as a daily dose of about 40 mg to about 400 mg. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof, is dosed in an amount of about 40 mg to about 320 mg, such as a daily dose of about 40 mg to about 320 mg. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof, is dosed in an amount of about 40 mg to about 200 mg, such as a daily doses of about 40 mg to about 200 mg. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof, is dosed in an amount of about 40 mg to about 160 mg, such as a daily doses of about 40 mg to about 160 mg. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid, or pharmaceutically acceptable salt thereof, is dosed in an amount of about 40 mg to about 80 mg, such as a daily doses of about 40 mg to about 80 mg. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 40 mg daily, or the pharmaceutically acceptable salt thereof is administered in an amount equivalent to about 40 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 80 mg daily, or the pharmaceutically acceptable salt thereof is administered in an amount equivalent to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 160 mg daily, or the pharmaceutically acceptable salt thereof is administered in an amount equivalent to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 320 mg daily, or the pharmaceutically acceptable salt thereof is administered in an amount equivalent to about 320 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is for about a 12 week period. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 40 mg daily, or the pharmaceutically acceptable salt thereof is administered in an amount equivalent to about 40 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 80 mg daily, or the pharmaceutically acceptable salt thereof is administered in an amount equivalent to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 160 mg daily, or the pharmaceutically acceptable salt thereof is administered in an amount equivalent to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 320 mg daily, or the pharmaceutically acceptable salt thereof is administered in an amount equivalent to about 320 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of at least about 700 ng/mL. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,000 ng/mL plus or minus 200 ng/mL. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,600 ng/mL plus or minus 300 ng/mL. In some embodiments, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 2,700 ng/mL plus or minus 400 ng/mL. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is for at least about 4 weeks. In some embodiments, the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is for at least about 8 weeks. In some embodiments, the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is for at least about 12 weeks. In some embodiments, the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is for at least about 24 weeks. In some embodiments, the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is for about a 4-week period. In some embodiments, the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is for about an 8-week period. In some embodiments, the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is for about a 12 week period. In some embodiments, the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is for about a 24 week period. In some embodiments, the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is daily. In some embodiments, the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is once daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 40 mg daily, or the pharmaceutically acceptable salt thereof is administered in an amount equivalent to about 40 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 80 mg daily, or the pharmaceutically acceptable salt thereof is administered in an amount equivalent to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 160 mg daily, or the pharmaceutically acceptable salt thereof is administered in an amount equivalent to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid is administered in an amount of about 320 mg daily, or the pharmaceutically acceptable salt thereof is administered in an amount equivalent to about 320 mg of (S)-4-((2-methoxyethyl)(4-(5,6,7,8-tetrahydro-1,8-naphthyridin-2-yl)butyl)amino)-2-(quinazolin-4-ylamino)butanoic acid daily. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of at least about 700 ng/mL. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,000 ng/mL plus or minus 200 ng/mL. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 1,600 ng/mL plus or minus 300 ng/mL. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in an amount sufficient to provide mean plasma levels of (S)-4-((2-methoxyethyl)(4-(5,6,7,8-tetrahydro-1,8-naphthyridin-2-yl)butyl)amino)-2-(quinazolin-4-ylamino)butanoic acid of about 2,700 ng/mL plus or minus 400 ng/ml. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is administered in a therapeutically effective amount. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, has a fibrotic disease. In some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, has a fibrotic liver disease. In some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, has a fibrotic liver disease, wherein the fibrotic liver disease is primary sclerosing cholangitis (PSC). In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is a human. In some embodiments, the subject is being treated with an additional therapeutic. In embodiments, the additional therapeutic comprises an immunosuppressant, an antifibrotic agent, niacin, a statin, a fibrate, a bile acid, a bile salt, a bile acid sequestrant, or an antibiotic. In embodiments, the immunosuppressant is selected from the group consisting of prednisone, budesonide, methotrexate, azathioprine, mycophenolate mofetil, cyclosporine, tacrolimus, cladribine, pentoxifylline, and a corticosteroid. In embodiments, the antifibrotic agent is selected from the group consisting of colchicine, D-penicillamine, pirfenidone, nintedanib, and niacin. In embodiments, the antifibrotic agent is nintedanib. In embodiments, the additional therapeutic comprises a bile acid, such as a cholic acid, such as ursodeoxycholic acid or obeticholic acid. In embodiments, the bile acid sequestrant is cholestyramine. In embodiments, the antibiotic is selected from the group consisting of vancomycin, minocycline, and rifaximin. In some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is concurrently being treated with a standard medical therapy or a standard of care. In some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is concurrently being treated with a standard medical therapy or a standard of care, wherein the standard medical therapy or standard of care comprises administration of ursodeoxycholic acid (UDCA) or a pharmaceutically acceptable salt thereof. In some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, has not been previously treated with a standard medical therapy or a standard of care for a liver disorder. In some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, has not been previously treated with a standard medical therapy or a standard of care for a liver disorder, wherein the standard medical therapy or standard of care comprises administration of ursodeoxycholic acid (UDCA) or a pharmaceutically acceptable salt thereof. In some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is not being concurrently treated with an additional therapeutic. In some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is not being concurrently treated with a standard medical therapy or a standard of care. In some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is not being concurrently treated with a standard medical therapy or a standard of care, wherein the standard medical therapy or standard of care comprises administration of ursodeoxycholic acid (UDCA) or a pharmaceutically acceptable salt thereof. In some embodiments, the subject is not administered any treatment for a liver disorder other than(S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the method comprising administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is not accompanied by a serious adverse event. In any of the methods disclosed herein, in some embodiments, the method comprising administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is not accompanied by a serious adverse event, wherein the serious adverse event is life-threatening (e.g., its occurrence places the subject at immediate risk of death); results in death; requires inpatient hospitalization (i.e., admission, overnight stay) or prolongs existing hospitalization; results in persistent or significant disability/incapacity; is a congenital anomaly/birth defect/miscarriage; or is an important medical event (e.g., is considered to be clinically significant and may jeopardize the subject, or when medical or surgical intervention may be required to prevent one of the outcomes listed above). In some embodiments, the method comprises administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein a probability of a serious adverse event is less than about 20%. In some embodiments, the method comprising administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is not accompanied by a serious adverse event, wherein the serious adverse event is a gastrointestinal adverse event, cholangitis, cholecystitis, pancreatitis, or abdominal pain. In some embodiments, the method comprises administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein a probability of a serious adverse event is less than about 20%, and wherein the serious adverse event is a gastrointestinal adverse event, cholangitis, cholecystitis, pancreatitis, or abdominal pain. In some embodiments, the method comprises administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein an incidence of adverse events is lower than an incidence of adverse events for a standard medical therapy or a standard of care for a liver disorder. In some embodiments, the method comprises administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein an incidence of adverse events is lower than an incidence of adverse events for a standard medical therapy or a standard of care for a liver disorder, and wherein the standard medical therapy or standard of care comprises administration of ursodeoxycholic acid (UDCA) or a pharmaceutically acceptable salt thereof. In some embodiments, the method comprises administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein an incidence of adverse events is lower than an incidence of adverse events for a standard medical therapy or a standard of care for a liver disorder, and wherein the adverse events are gastrointestinal adverse events, cholangitis, cholecystitis, pancreatitis, or abdominal pain. In some embodiments, the method comprises administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein an incidence of adverse events is lower than an incidence of adverse events for a standard medical therapy or a standard of care for a liver disorder, wherein the standard medical therapy or standard of care comprises administration of ursodeoxycholic acid (UDCA) or a pharmaceutically acceptable salt thereof, and wherein the adverse events are gastrointestinal adverse events, cholangitis, cholecystitis, pancreatitis, or abdominal pain. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, liver inflammation is reduced following the administering of (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a phosphate salt, wherein the phosphate salt is crystalline. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a crystalline Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is selected from a crystalline Form IV phosphate salt, a crystalline Form II fumarate salt, a crystalline Form III naphthalenedisulfonic acid salt, a zwitterionic form, and an amorphous form.

In any of the methods disclosed herein, in some embodiments, bile duct inflammation is reduced following the administering of (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a phosphate salt, wherein the phosphate salt is crystalline. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a crystalline Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is selected from a crystalline Form IV phosphate salt, a crystalline Form II fumarate salt, a crystalline Form III naphthalenedisulfonic acid salt, a zwitterionic form, and an amorphous form.

Also provided in another embodiment is a method of modulating α_{V}β₆ integrin, α_{V}β₁ integrin, or both α_{V}β₆ integrin and α_{V}β₁ integrin in a subject in need thereof, comprising: administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the administering is not accompanied by a serious adverse event. In some embodiments, the method of modulating α_{V}β₆ integrin, α_{V}β₁ integrin, or both α_{V}β₆ integrin and α_{V}β₁ integrin comprising administering(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, further comprises inhibiting α_{V}β₆ integrin, α_{V}β₁ integrin, or both α_{V}β₆ integrin and α_{V}β₁ integrin. In some embodiments, liver inflammation is reduced following the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments, bile duct inflammation is reduced following the administering of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is concurrently being treated with a standard medical therapy or a standard of care, wherein the standard medical therapy or standard of care comprises administration of ursodeoxycholic acid or a pharmaceutically acceptable salt thereof. In some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, has not been previously treated with a standard medical therapy or a standard of care for a liver disorder, wherein the standard medical therapy or standard of care comprises administration of ursodeoxycholic acid or a pharmaceutically acceptable salt thereof. In some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, has not been previously treated with a standard medical therapy or a standard of care for a liver disorder or for primary sclerosing cholangitis. In some embodiments, the method comprises administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein an incidence of adverse events is lower than an incidence of adverse events for a standard medical therapy or a standard of care for a liver disorder, and wherein the standard medical therapy or standard of care comprises administration ursodeoxycholic acid or a pharmaceutically acceptable salt thereof. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In any of the methods disclosed herein, in some embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, has a fibrotic disorder. In any of the preceding embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, has a fibrotic liver disorder. In any of the preceding embodiments, the subject who has been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, has a fibrotic liver disorder, wherein the fibrotic liver disorder is primary sclerosing cholangitis. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a Form I phosphate salt. In some embodiments, the (S)-4-((2-methoxyethyl)(4-(5,6,7,8-tetrahydro-1,8-naphthyridin-2-yl)butyl)amino)-2-(quinazolin-4-ylamino)butanoic acid or pharmaceutically acceptable salt thereof is a phosphate salt, a polymorph (including a crystalline Form I phosphate salt, a crystalline Form IV phosphate salt, a crystalline Form II fumarate salt, and a crystalline Form III naphthalenedisulfonic acid salt), a zwitterionic form, or an amorphous form.

In embodiments, disclosed herein is(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, for use in ameliorating fibrosis in tissue of a human in need thereof, wherein the tissue comprises liver tissue, gall bladder tissue, or bile duct tissue.

In embodiments, disclosed herein is(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, for use in treating primary sclerosing cholangitis in a human in need thereof, wherein primary sclerosing cholangitis in the human is ameliorated.

In embodiments, disclosed herein is(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, for use in reducing pruritus in a human who has primary sclerosing cholangitis.

In embodiments, disclosed herein is(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, for use in slowing the clinical progression of primary sclerosing cholangitis in a human who has primary sclerosing cholangitis and is at risk for moderate to severe liver fibrosis.

In embodiments, disclosed herein is(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, for use in downregulating a gene in a human who has primary sclerosing cholangitis, wherein the gene is selected from the group consisting of COL1A1, COL1A2, COL3A1 and TFGb-1.

In embodiments, disclosed herein is(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, for use in downregulating a gene in a human who has primary sclerosing cholangitis, wherein the gene is selected from the group consisting of COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

In embodiments, disclosed herein is(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, for use in downregulating a gene in a human who has primary sclerosing cholangitis, wherein the gene is selected from the group consisting of COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

In embodiments, disclosed herein is(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, for use in downregulating a gene in a human who has primary sclerosing cholangitis, wherein the gene is selected from the group consisting of ADAMTS3, ANXA6, CCDC80, COL5A1, COL7A1, CXCL12, FBLN1, IGFBP7, or LOXL1.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in ameliorating fibrosis in tissue of a human in need thereof, wherein the tissue comprises liver tissue, gall bladder tissue, or bile duct tissue.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in treating primary sclerosing cholangitis in a human in need thereof, wherein primary sclerosing cholangitis in the human is ameliorated.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in reducing pruritus in a human who has primary sclerosing cholangitis.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in slowing the clinical progression of primary sclerosing cholangitis in a human who has primary sclerosing cholangitis and is at risk for moderate to severe liver fibrosis.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in slowing the clinical progression of primary sclerosing cholangitis in a human who has primary sclerosing cholangitis and is at risk for moderate to severe liver fibrosis as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, or compared to a human who has been administered a placebo.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of COL1A1, COL1A2, COL3A1 and TFGb-1.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of ADAMTS3, ANXA6, CCDC80, COL5A1, COL7A1, CXCL12, FBLN1, IGFBP7, or LOXL1.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for ameliorating fibrosis in tissue of a human in need thereof, wherein the tissue comprises liver tissue, gall bladder tissue, or bile duct tissue.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for treating primary sclerosing cholangitis in a human in need thereof, wherein primary sclerosing cholangitis in the human is ameliorated.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for reducing pruritus in a human who has primary sclerosing cholangitis.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for slowing the clinical progression of primary sclerosing cholangitis in a human who has primary sclerosing cholangitis and is at risk for moderate to severe liver fibrosis.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of COL1A1, COL1A2, COL3A1 and TFGb-1.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

In embodiments, disclosed herein is use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of ADAMTS3, ANXA6, CCDC80, COL5A1, COL7A1, CXCL12, FBLN1, IGFBP7, or LOXL1.

Further aspects of the disclosure are illustrated by the following exemplary embodiments.

Embodiment 1. A method of ameliorating fibrosis in a tissue of a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the tissue is liver tissue, gall bladder tissue, or bile duct tissue.

Embodiment 2. The method of embodiment 1, wherein the tissue is liver tissue and gall bladder tissue.

Embodiment 3. The method of embodiment 1, wherein the tissue is liver tissue and

bile duct tissue.

Embodiment 4. The method of embodiment 1, wherein the tissue is gall bladder tissue and bile duct tissue.

Embodiment 5. The method of embodiment 1, wherein the tissue is liver tissue.

Embodiment 6. The method of embodiment 1, wherein the tissue is gall bladder tissue.

Embodiment 7. The method of embodiment 1, wherein the tissue is bile duct tissue.

Embodiment 8. The method of any of embodiments 1-7, wherein the human has primary sclerosing cholangitis.

Embodiment 9. A method of treating primary sclerosing cholangitis in a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein primary sclerosing cholangitis in the human is ameliorated.

Embodiment 10. A method of reducing pruritus in a human who has primary sclerosing cholangitis comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment 11. The method of any of embodiments 1-10, wherein the human is at risk for moderate to severe fibrosis.

Embodiment 12. A method of slowing the clinical progression of primary sclerosing cholangitis in a human who has primary sclerosing cholangitis and is at risk for moderate to severe liver fibrosis comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment 13. The method of embodiment 12, wherein clinical progression at two years following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human is slowed as compared to a subject who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment 14. The method of embodiment 12 or 13, wherein the slowing of clinical progression is measured by an enhanced liver fibrosis score increase of less than about 0.2 following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 15. A method of downregulating a gene in a human who has primary sclerosing cholangitis, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the gene is selected from the group consisting of COL1A1, COL1A2, COL3A1 and TFGb-1.

Embodiment 16. The method of embodiment 15, wherein the gene is selected from the group consisting of COLLA2, COL3A1 and TFGb-1.

Embodiment 17. The method of any of embodiments 1-16, further comprising amelioration of an enhanced liver fibrosis score.

Embodiment 18. The method of any of embodiments 1-17, wherein the human has one or more of the following risk factors for fibrosis prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human:
a. enhanced liver fibrosis score greater than or equal to about 7.7,
b. transient elastography between about 7.7 kPa and about 14.4 kPa,
c. magnetic resonance elastography between about 2.4 kPa and about 4.9 kPa, and
d. historical biopsy.

Embodiment 19. The method of any of embodiments 1-18, wherein the human has an enhanced liver fibrosis score greater than or equal to about 7.7 prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 20. The method of any of embodiments 1-19, wherein the human has liver fibrosis.

Embodiment 21. The method of any of embodiments 17-20, wherein the amelioration of enhanced liver fibrosis score occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 22. The method of embodiment 21, wherein the amelioration of enhanced liver fibrosis score is measured at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 23. The method of embodiments 21 or 22, wherein the enhanced liver fibrosis score increase is less than about 0.2 at about 12 weeks following a first administration of (S)-4-((2-methoxyethyl)(4-(5,6,7,8-tetrahydro-1,8-naphthyridin-2- yl)butyl)amino)-2-(quinazolin-4-ylamino)butanoic acid, or a pharmaceutically acceptable salt thereof to the human.

Embodiment 24. The method of any of embodiments 21-23, wherein the enhanced liver fibrosis score increase is less than about 0.1 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 25. The method of any of embodiments 1-24, wherein the administering to the human is for at least about 12 weeks.

Embodiment 26. The method of any of embodiments 1-25, wherein the administering to the human is oral.

Embodiment 27. The method of any of embodiments 1-26, wherein the administering to the human is daily.

Embodiment 28. The method of any of embodiments 1-27, wherein the administering to the human is once daily.

Embodiment 29. The method of any of embodiments 1-28, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to from about 40 mg to about 400 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 30. The method of any of embodiments 1-29, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to from about 40 mg to about 320 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 31. The method of any of embodiments 1-30, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to from about 40 mg to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 32. The method of any of embodiments 1-31, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 40 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 33. The method of any of embodiments 1-31, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 34. The method of any of embodiments 1-31, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 35. The method of any of embodiments 1-31, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 320 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 36. The method of any of embodiments 1-9 or 11-35, further comprising reduction of pruritus.

Embodiment 37. The method of embodiments 10, 11 or 36, wherein the reduction of pruritus occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 38. The method of embodiments 36 or 37, wherein pruritus is reduced as measured by a change in itch numerical rating scale from between about 0.8 and about-0.2 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 39. The method of embodiment 38, wherein the change in itch numerical rating scale is less than about 0.2 at about 12 weeks following a first administration of (S)-4-((2-methoxyethyl)(4-(5,6,7,8-tetrahydro-1,8-naphthyridin-2-yl)butyl)amino)- 2-(quinazolin-4-ylamino)butanoic acid, or a pharmaceutically acceptable salt thereof to the human.

Embodiment 40. The method of any of embodiments 1-39, further comprising reduction in collagen synthesis in the human.

Embodiment 41. The method of embodiment 40, wherein reduction of collagen synthesis occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 42. The method of embodiments 40 or 41, wherein collagen synthesis is reduced as measured by a percent change in PRO-C3 from about-3.4% to about 2.9% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 43. The method of any of embodiments 1-42, further comprising reduction of PRO-C3 in the human.

Embodiment 44. The method of embodiment 43, wherein reduction of PRO-C3 occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 45. The method of embodiments 43 or 44, wherein PRO-C3 is decreased by about 3.4% to about 0.5% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 46. The method of any of embodiments 43-45, wherein PRO-C3 is decreased by about 0.5% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 47. The method of any of embodiments 1-46, wherein the administration results in a change in transient elastography.

Embodiment 48. The method of embodiment 47, wherein the change in transient elastography occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 49. The method of any of embodiments 1-48, wherein the administration results in a change in alkaline phosphatase level in the human.

Embodiment 50. The method of embodiment 49, wherein alkaline phosphatase level is above an upper limit of normal prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 51. The method of embodiments 49 or 50, wherein the change in alkaline phosphatase level occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 52. The method of any of embodiments 49-51, wherein the change in alkaline phosphatase level is from about 8.8 U/L to about-8.4 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 53. The method of any of embodiments 49-52, wherein the change in alkaline phosphatase level is from about 2.1 U/L to about-8.4 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 54. The method of any of embodiments 49-53, wherein the alkaline phosphatase level is decreased following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 55. The method of any of embodiments 49-54, wherein the change in alkaline phosphatase level is from about-8.4 U/L to about 0 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 56. The method of any of embodiments 1-55, wherein the administration results in stabilization or decrease in total bilirubin in the human.

Embodiment 57. The method of embodiment 56, wherein the stabilization or decrease in total bilirubin occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 58. The method of embodiments 56 or 57, wherein the stabilization or decrease in total bilirubin is from about-0.2 mg/dL to about 1 mg/dL at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 59. The method of any of embodiments 56-58, wherein total bilirubin is decreased by about 0.5 mg/dL at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 60. The method of any of embodiments 1-59, wherein the human is not concurrently being treated with another therapeutic for primary sclerosing cholangitis.

Embodiment 61. The method of any of embodiments 1-59, wherein the human is concurrently being treated with another therapeutic for primary sclerosing cholangitis.

Embodiment 62. The method of embodiment 61, wherein the other therapeutic comprises an immunosuppressant, an antifibrotic agent, nicotine, a statin, a bile acid, a bile salt, a bile acid sequestrant, or an antibiotic.

Embodiment 63. The method of embodiment 62, wherein the immunosuppressant is selected from the group consisting of prednisone, budesonide, methotrexate, azathioprine, mycophenolate mofetil, cyclosporine, tacrolimus, cladribine, pentoxifylline, and a corticosteroid.

Embodiment 64. The method of embodiment 62, wherein the antifibrotic agent is selected from the group consisting of colchicine, D-penicillamine, pirfenidone, and nicotine.

Embodiment 65. The method of embodiment 62, wherein the other therapeutic comprises a bile acid.

Embodiment 66. The method of embodiment 65, wherein the bile acid is a cholic acid.

Embodiment 67. The method of embodiment 66, wherein the cholic acid is ursodeoxycholic acid.

Embodiment 68. The method of embodiment 67, wherein the bile acid sequestrant is cholestyramine.

Embodiment 69. The method of embodiment 62, wherein the antibiotic is selected from the group consisting of vancomycin, minocycline, and rifaximin.

Embodiment 70. The method of any of embodiments 1-69, wherein the method is not accompanied by a serious adverse event.

Embodiment 71. The method of embodiment 70, wherein the serious adverse event comprises cholangitis, cholecystitis, pancreatitis, or abdominal pain.

Embodiment 72. The method of embodiment 71, wherein the serious adverse event is cholangitis.

Embodiment 73. The method of any of embodiments 1-72, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a phosphate salt.

Embodiment 74. The method of embodiment 73, wherein the phosphate salt is crystalline.

Embodiment 75. The method of any of embodiments 1-74, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is crystalline Form I phosphate salt.

Embodiment 76. The method of any of embodiments 1-72, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is selected from crystalline Form IV phosphate salt, crystalline Form II fumarate salt, crystalline Form III naphthalenedisulfonic acid salt, a zwitterionic form, and an amorphous form.

Embodiment 77. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in ameliorating fibrosis in tissue of a human in need thereof, wherein the tissue is liver tissue, gall bladder tissue, or bile duct tissue.

Embodiment 78. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in treating primary sclerosing cholangitis in a human in need thereof, wherein primary sclerosing cholangitis in the human is ameliorated.

Embodiment 79. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in reducing pruritus in a human who has primary sclerosing cholangitis.

Embodiment 80. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in slowing the clinical progression of primary sclerosing cholangitis in a human who has primary sclerosing cholangitis and is at risk for moderate to severe liver fibrosis.

Embodiment 81. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of COL1A1, COL1A2, COL3A1 and TFGb-1.

Embodiment 82. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for ameliorating fibrosis in a tissue of a human in need thereof, wherein the tissue is liver tissue, gall bladder tissue, or bile duct tissue.

Embodiment 83. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for treating primary sclerosing cholangitis in a human in need thereof, wherein primary sclerosing cholangitis in the human is ameliorated.

Embodiment 84. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for reducing pruritus in a human who has primary sclerosing cholangitis.

Embodiment 85. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for slowing the clinical progression of primary sclerosing cholangitis in a human who has primary sclerosing cholangitis and is at risk for moderate to severe liver fibrosis.

Embodiment 86. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of COL1A1, COL1A2, COL3A1 and TFGb-1.

Embodiment 87. The method or use of any of embodiments 1-86, wherein the human is an adult.

Embodiment 88. The method or use of any of embodiments 1-86, wherein the human is 18 years of age to about 75 years of age.

Embodiment 89. A method of ameliorating fibrosis in a tissue of a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the tissue is liver tissue, gall bladder tissue, or bile duct tissue.

Embodiment 90. The method of Embodiment 89, wherein the tissue is liver tissue and gall bladder tissue.

Embodiment 91. The method of Embodiment 89, wherein the tissue is liver tissue and bile duct tissue.

Embodiment 92. The method of Embodiment 89, wherein the tissue is gall bladder tissue and bile duct tissue.

Embodiment 93. The method of Embodiment 89, wherein the tissue is liver tissue.

Embodiment 94. The method of Embodiment 89, wherein the tissue is gall bladder tissue.

Embodiment 95. The method of Embodiment 89, wherein the tissue is bile duct tissue.

Embodiment 96. The method of any of Embodiments 89-95, wherein the human has primary sclerosing cholangitis.

Embodiment 97. A method of treating primary sclerosing cholangitis in a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein primary sclerosing cholangitis in the human is ameliorated.

Embodiment 98. A method of reducing pruritus in a human who has primary sclerosing cholangitis comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment 99. The method of any of Embodiments 89-98, wherein the human is at risk for moderate to severe fibrosis.

Embodiment 100. A method of slowing the clinical progression of primary sclerosing cholangitis in a human who has primary sclerosing cholangitis and is at risk for moderate to severe liver fibrosis comprising administering to the subject(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment 101. The method of Embodiment 100, wherein clinical progression at two years following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human is slowed as compared to a subject who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment 102. The method of Embodiment 100 or 101, wherein the slowing of clinical progression is measured by an enhanced liver fibrosis score increase of less than about 0.2 following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 103. A method of downregulating a gene in a human who has primary sclerosing cholangitis, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the gene is selected from the group consisting of COL1A1, COL1A2, COL3A1 and TFGb-1.

Embodiment 104. The method of Embodiment 103, wherein the gene is selected from the group consisting of COL1A2, COL3A1 and TFGb-1.

Embodiment 105. The method of any of Embodiments 89-104, further comprising amelioration of an enhanced liver fibrosis score.

Embodiment 106. The method of any of Embodiments 89-105, wherein the human has one or more of the following risk factors for fibrosis prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human: (a) enhanced liver fibrosis score greater than or equal to about 7.7, (b) transient elastography between about 7.7 kPa and about 14.4 kPa, (c) magnetic resonance elastography between about 2.4 kPa and about 4.9 kPa, and (d) historical biopsy.

Embodiment 107. The method of any of Embodiments 89-106, wherein the human has an enhanced liver fibrosis score greater than or equal to about 7.7 prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 108. The method of any of Embodiments 89-107, wherein the human has liver fibrosis.

Embodiment 109. The method of any of Embodiments 105-108, wherein the amelioration of enhanced liver fibrosis score occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 110. The method of Embodiment 109, wherein the amelioration of enhanced liver fibrosis score is measured at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 111. The method of Embodiment 109 or 110, wherein the enhanced liver fibrosis score increase is less than about 0.2 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 112. The method of any of Embodiments 109-111, wherein the enhanced liver fibrosis score increase is less than about 0.1 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 113. The method of any of Embodiments 89-112, wherein the administering to the human is for at least about 12 weeks.

Embodiment 114. The method of any of Embodiments 89-113, wherein the administering to the human is oral.

Embodiment 115. The method of any of Embodiments 89-114, wherein the administering to the human is daily.

Embodiment 116. The method of any of Embodiments 89-115, wherein the administering to the human is once daily.

Embodiment 117. The method of any of Embodiments 89-116, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to from about 40 mg to about 400 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 118. The method of any of Embodiments 89-117, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to from about 40 mg to about 320 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 119. The method of any of Embodiments 89-118, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to from about 40 mg to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 120. The method of any of Embodiments 89-119, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 40 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 121. The method of any of Embodiments 89-119, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 122. The method of any of Embodiments 89-119, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 123. The method of any of Embodiments 89-119, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 320 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 124. The method of any of Embodiments 89-97 or 99-123, further comprising reduction of pruritus.

Embodiment 125. The method of Embodiments 98, 99, or 124, wherein the reduction of pruritus occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 126. The method of Embodiments 124 or 125, wherein pruritus is reduced as measured by a change in itch numerical rating scale from between about 0.8 and about-0.2 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 127. The method of Embodiment 126, wherein the change in itch numerical rating scale is less than about 0.2 at about 12 weeks following a first administration of (S)-4-((2-methoxyethyl)(4-(5,6,7,8-tetrahydro-1,8-naphthyridin-2-yl)butyl)amino)- 2-(quinazolin-4-ylamino)butanoic acid, or a pharmaceutically acceptable salt thereof to the human.

Embodiment 128. The method of any of Embodiments 89-127, further comprising reduction in collagen synthesis in the human.

Embodiment 129. The method of Embodiment 128, wherein reduction of collagen synthesis occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 130. The method of Embodiments 128 or 129, wherein collagen synthesis is reduced as measured by a percent change in PRO-C3 from about-3.4% to about 2.9% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 131. The method of any of Embodiments 89-130, further comprising reduction of PRO-C3 in the human.

Embodiment 132. The method of Embodiment 131, wherein reduction of PRO-C3 occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 133. The method of Embodiments 131 or 132, wherein PRO-C3 is decreased by about 3.4% to about 0.5% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 134. The method of any of Embodiments 131-133, wherein PRO-C3 is decreased by about 0.5% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 135. The method of any of Embodiments 89-134, wherein the administration results in a change in transient elastography.

Embodiment 136. The method of Embodiment 135, wherein the change in transient elastography occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 137. The method of any of Embodiments 89-136, wherein the administration results in a change in alkaline phosphatase level in the human.

Embodiment 138. The method of Embodiment 137, wherein alkaline phosphatase level is above an upper limit of normal prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 139. The method of Embodiments 137 or 138, wherein the change in alkaline phosphatase level occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 140. The method of any of Embodiments 137-139, wherein the change in alkaline phosphatase level is from about 8.8 U/L to about-8.4 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 141. The method of any of Embodiments 137-140, wherein the change in alkaline phosphatase level is from about 2.1 U/L to about-8.4 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 142. The method of any of Embodiments 137-141, wherein the alkaline phosphatase level is decreased following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 143. The method of any of Embodiments 137-142, wherein the change in alkaline phosphatase level is from about-8.4 U/L to about 0 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 144. The method of any of Embodiments 89-143, wherein the administration results in stabilization or decrease in total bilirubin in the human.

Embodiment 145. The method of Embodiment 144, wherein the stabilization or decrease in total bilirubin occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 146. The method of Embodiments 144 or 145, wherein the stabilization or decrease in total bilirubin is from about-0.2 mg/dL to about 1 mg/dL at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 147. The method of any of Embodiments 144-146, wherein total bilirubin is decreased by about 0.5 mg/dL at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment 148. The method of any of Embodiments 89-147, wherein the human is not concurrently being treated with another therapeutic for primary sclerosing cholangitis.

Embodiment 149. The method of any of Embodiments 89-147, wherein the human is concurrently being treated with another therapeutic for primary sclerosing cholangitis.

Embodiment 150. The method of Embodiment 149, wherein the other therapeutic comprises an immunosuppressant, an antifibrotic agent, nicotine, a statin, a bile acid, a bile salt, a bile acid sequestrant, or an antibiotic.

Embodiment 151. The method of Embodiment 150, wherein the immunosuppressant is selected from the group consisting of prednisone, budesonide, methotrexate, azathioprine, mycophenolate mofetil, cyclosporine, tacrolimus, cladribine, pentoxifylline, and a corticosteroid.

Embodiment 152. The method of Embodiment 150, wherein the antifibrotic agent is selected from the group consisting of colchicine, D-penicillamine, pirfenidone, and nicotine.

Embodiment 153. The method of Embodiment 150, wherein the other therapeutic comprises a bile acid.

Embodiment 154. The method of Embodiment 153, wherein the bile acid is a cholic acid.

Embodiment 155. The method of Embodiment 154, wherein the cholic acid is ursodeoxycholic acid.

Embodiment 156. The method of Embodiment 150, wherein the bile acid sequestrant is cholestyramine.

Embodiment 157. The method of Embodiment 150, wherein the antibiotic is selected from the group consisting of vancomycin, minocycline, and rifaximin.

Embodiment 158. The method of any of Embodiments 89-157, wherein the method is not accompanied by a serious adverse event.

Embodiment 159. The method of Embodiment 158, wherein the serious adverse event comprises cholangitis, cholecystitis, pancreatitis, or abdominal pain.

Embodiment 160. The method of Embodiment 159, wherein the serious adverse event is cholangitis.

Embodiment 161. The method of any of Embodiments 89-160, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a phosphate salt.

Embodiment 162. The method of Embodiment 161, wherein the phosphate salt is crystalline.

Embodiment 163. The method of any of Embodiments 89-162, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is crystalline Form I phosphate salt.

Embodiment 164. The method of any of Embodiments 89-160, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is selected from crystalline Form IV phosphate salt, crystalline Form II fumarate salt, crystalline Form III naphthalenedisulfonic acid salt, a zwitterionic form, and an amorphous form.

Embodiment 165. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in ameliorating fibrosis in tissue of a human in need thereof, wherein the tissue is liver tissue, gall bladder tissue, or bile duct tissue.

Embodiment 166. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in treating primary sclerosing cholangitis in a human in need thereof, wherein primary sclerosing cholangitis in the human is ameliorated.

Embodiment 167. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in reducing pruritus in a human who has primary sclerosing cholangitis.

Embodiment 168. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in slowing the clinical progression of primary sclerosing cholangitis in a human who has primary sclerosing cholangitis and is at risk for moderate to severe liver fibrosis.

Embodiment 169. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of COL1A1, COL1A2, COL3A1 and TFGb-1.

Embodiment 170. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for ameliorating fibrosis in a tissue of a human in need thereof, wherein the tissue is liver tissue, gall bladder tissue, or bile duct tissue.

Embodiment 171. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for treating primary sclerosing cholangitis in a human in need thereof, wherein primary sclerosing cholangitis in the human is ameliorated.

Embodiment 172. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for reducing pruritus in a human who has primary sclerosing cholangitis.

Embodiment 173. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for slowing the clinical progression of primary sclerosing cholangitis in a human who has primary sclerosing cholangitis and is at risk for moderate to severe liver fibrosis.

Embodiment 174. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of COL1A1, COL1A2, COL3A1 and TFGb-1.

Embodiment 175. The method or use of any of Embodiments 89-174, wherein the human is an adult.

Embodiment 176. The method or use of any of Embodiments 89-175, wherein the human is 18 years of age to about 75 years of age.

Embodiment 177. A method of downregulating a gene in a human who has primary sclerosing cholangitis, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the gene is selected from the group consisting of COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

Embodiment 178. A method of downregulating a gene in a human who has primary sclerosing cholangitis, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the gene is selected from the group consisting of COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

Embodiment 179. A method of downregulating a gene in a human who has primary sclerosing cholangitis, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the gene is selected from the group consisting of ADAMTS3, ANXA6, CCDC80, COL5A1, COL7A1, CXCL12, FBLN1, IGFBP7, or LOXL1.

Embodiment 180. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

Embodiment 181. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

Embodiment 182. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of ADAMTS3, ANXA6, CCDC80, COL5A1, COL7A1, CXCL12, FBLN1, IGFBP7, or LOXL1.

Embodiment 183. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

Embodiment 184. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

Embodiment 185. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of ADAMTS3, ANXA6, CCDC80, COL5A1, COL7A1, CXCL12, FBLN1, IGFBP7, or LOXL1. Embodiment 185. The method or use of any of embodiments 1-185, wherein(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof comprises a crystalline form of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment 186. The method or use of any of embodiments 1-185, wherein(S)-4-((2-methoxyethyl)(4-(5,6,7,8-tetrahydro-1,8-naphthyridin-2-yl)butyl)amino)-2-(quinazolin-4-ylamino)butanoic acid comprises the Form I phosphate salt.

Embodiment 187. The method or use of any of embodiments 1-185, wherein(S)-4-((2-methoxyethyl)(4-(5,6,7,8-tetrahydro-1,8-naphthyridin-2-yl)butyl)amino)-2-(quinazolin-4-ylamino)butanoic acid comprises the Form II fumarate salt.

Embodiment 188. The method or use of any of embodiments 1-185, wherein(S)-4-((2-methoxyethyl)(4-(5,6,7,8-tetrahydro-1,8-naphthyridin-2-yl)butyl)amino)-2-(quinazolin-4-ylamino)butanoic acid comprises the Form III 1,5-naphthalenedisulfonate salt.

Embodiment 189. The method or use of any of embodiments 1-185, wherein(S)-4-((2-methoxyethyl)(4-(5,6,7,8-tetrahydro-1,8-naphthyridin-2-yl)butyl)amino)-2-(quinazolin-4-ylamino)butanoic acid comprises the Form IV mixed isopropyl alcohol and water phosphate salt.

Embodiment A1. A method of ameliorating fibrosis in a tissue of a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the tissue is liver tissue, gall bladder tissue, or bile duct tissue.

Embodiment A2. The method of embodiment A1, wherein the tissue is liver tissue and gall bladder tissue.

Embodiment A3. The method of embodiment A1, wherein the tissue is liver tissue and bile duct tissue.

Embodiment A4. The method of embodiment A1, wherein the tissue is gall bladder tissue and bile duct tissue.

Embodiment A5. The method of embodiment A1, wherein the tissue is liver tissue.

Embodiment A6. The method of embodiment A1, wherein the tissue is gall bladder tissue.

Embodiment A7. The method of embodiment A1, wherein the tissue is bile duct tissue.

Embodiment A8. The method of any of embodiments A1-A7, wherein the human has primary sclerosing cholangitis.

Embodiment A9. A method of treating primary sclerosing cholangitis in a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein primary sclerosing cholangitis in the human is ameliorated.

Embodiment A10. A method of ameliorating pruritus in a human who has fibrotic liver disease comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A11. The method of embodiment A10, wherein ameliorating pruritus comprises reducing pruritus in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A12. The method of embodiment A10 or embodiment A11, wherein the fibrotic liver disease is primary sclerosing cholangitis.

Embodiment A13. The method of any of embodiments A1-A12, wherein the human is at risk for moderate to severe fibrosis.

Embodiment A14. A method of slowing the clinical progression of primary sclerosing cholangitis in a human who has primary sclerosing cholangitis and is at risk for moderate to severe liver fibrosis comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A15. The method of embodiment A14, wherein the slowing of clinical progression is measured by an enhanced liver fibrosis score increase of less than about 0.2 following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A16. A method of downregulating a gene in a human who has primary sclerosing cholangitis, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the gene is selected from the group consisting of COL1A1, COL1A2, COL3A1 and TFGb-1.

Embodiment A17. The method of embodiment A16, wherein the gene is selected from the group consisting of COL1A2, COL3A1 and TFGb-1.

Embodiment A18. The method of any of embodiments A1-A17, further comprising amelioration of an enhanced liver fibrosis score.

Embodiment A19. The method of any of embodiments A1-A18, wherein the human has one or more of the following risk factors for fibrosis prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human:
a. an enhanced liver fibrosis score greater than or equal to about 7.7,
b. a transient elastography score between about 7.7 kPa and about 14.4 kPa,
c. a magnetic resonance elastography score between about 2.4 kPa and about 4.9 kPa, and
d. a historical biopsy.

Embodiment A20. The method of any of embodiments A1-A19, wherein the human has an enhanced liver fibrosis score greater than or equal to about 7.7 prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A21. The method of any of embodiments A1-A20, wherein the human has liver fibrosis.

Embodiment A22. The method of any of embodiments A18-A21, wherein the amelioration of enhanced liver fibrosis score occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A23. The method of embodiment A22, wherein the amelioration of enhanced liver fibrosis score is measured at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A24. The method of embodiments A22 or A23, wherein the enhanced liver fibrosis score increase is less than about 0.2 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A25. The method of any of embodiments A22-A24, wherein the enhanced liver fibrosis score increase is less than about 0.1 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A26. The method of any of embodiments A1-A25, wherein the administering to the human is for at least about 12 weeks.

Embodiment A27. The method of any of embodiments A18-A21, wherein the amelioration of enhanced liver fibrosis score occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A28. The method of embodiment A27, wherein the amelioration of enhanced liver fibrosis score is measured at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A29. The method of embodiments A27 or A28, wherein the enhanced liver fibrosis score increase is less than about 0.2 at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A30. The method of any of embodiments A27-A29, wherein the enhanced liver fibrosis score increase is less than about 0.1 at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A31. The method of any of embodiments A27-A29, wherein the enhanced liver fibrosis score is approximately unchanged at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A32. The method of any of embodiments A27-A29, wherein the enhanced liver fibrosis score decreases by about 0.05 at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A33. The method of any of embodiments A27-A29, wherein the enhanced liver fibrosis score decreases by about 0.1 at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A34. The method of any of embodiments A27-A33, wherein the human has an enhanced liver fibrosis score of about 9.8 or higher prior to the first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A35. The method of any of embodiments A1-A34, wherein the administering to the human is for at least about 24 weeks.

Embodiment A36. The method of any of embodiments A1-A35, wherein the administering to the human is oral.

Embodiment A37. The method of any of embodiments A1-A36, wherein the administering to the human is daily.

Embodiment A38. The method of any of embodiments A1-A37, wherein the administering to the human is once daily.

Embodiment A39. The method of any of embodiments A1-A38, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to from about 40 mg to about 400 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment A40. The method of any of embodiments A1-A39, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to from about 40 mg to about 320 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment A41. The method of any of embodiments A1-A40, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to from about 80 mg to about 320 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment A42. The method of any of embodiments A1-A41, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 40 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment A43. The method of any of embodiments A1-A41, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 80 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment A44. The method of any of embodiments A1-A41, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 160 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment A45. The method of any of embodiments A1-A41, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to about 320 mg of (S)-4-((2-methoxyethyl)(4-(5,6,7,8-tetrahydro-1,8-naphthyridin-2-yl)butyl)amino)-2-(quinazolin-4-ylamino)butanoic acid.

Embodiment A46. The method of any of embodiments A1-A9 or A13-A45, further comprising reduction of pruritus in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A47. The method of embodiments A10, A13 or A46, wherein the reduction of pruritus occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A48. The method of embodiments A46 or A47, wherein pruritus is reduced as measured by a change in itch numerical rating scale from between about 0.8 and about-0.2 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human relative to baseline itch rating prior to treatment.

Embodiment A49. The method of embodiment A48, wherein the change in itch numerical rating scale is less than about 0.2 at about 12 weeks following a first administration of (S)-4-((2-methoxyethyl)(4-(5,6,7,8-tetrahydro-1,8-naphthyridin-2-yl)butyl)amino)- 2-(quinazolin-4-ylamino)butanoic acid, or a pharmaceutically acceptable salt thereof to the human relative to baseline itch rating prior to treatment.

Embodiment A50. The method of embodiments A10, A13 or A46, wherein the reduction of pruritus occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A51. The method of embodiments A46 or A47, wherein pruritus is reduced as measured by a change in itch numerical rating scale from between about 1.0 and about-0.2 at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human relative to baseline itch rating prior to treatment.

Embodiment A52. The method of embodiment A51, wherein the change in itch numerical rating scale is less than about 0.2 at about 24 weeks following a first administration of (S)-4-((2-methoxyethyl)(4-(5,6,7,8-tetrahydro-1,8-naphthyridin-2-yl)butyl)amino)- 2-(quinazolin-4-ylamino)butanoic acid, or a pharmaceutically acceptable salt thereof to the human relative to baseline itch rating prior to treatment.

Embodiment A53. The method of any of embodiments A1-A52, further comprising reduction in collagen synthesis in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A54. The method of embodiment A53, wherein reduction of collagen synthesis occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A55. The method of embodiments A53 or A54, wherein collagen synthesis is reduced as measured by a percent change in PRO-C3 from about-3.4% to about 2.9% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A56. The method of embodiment A53, wherein reduction of collagen synthesis occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A57. The method of embodiment A53, wherein reduction of PRO-C3 occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A58. The method of embodiments A53 or A54, wherein PRO-C3 is decreased by about 3.4% to about 0.5% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A59. The method of embodiments A53 or A54, wherein PRO-C3 is decreased by about 0.5% at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A60. The method of embodiment A53, wherein reduction of PRO-C3 occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A61. The method of any of embodiments A1-A60, wherein the administration results in amelioration of a transient elastography score in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A62. The method of embodiment A61, wherein the amelioration in the transient elastography score occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A63. The method of embodiment A61, wherein the amelioration in the transient elastography score occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A64. The method of any of embodiments A1-A63, wherein the administration results in amelioration of an alkaline phosphatase level in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A65. The method of embodiment A64, wherein alkaline phosphatase level is above an upper limit of normal prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A66. The method of embodiments A64 or A65, wherein a change in the alkaline phosphatase level occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A67. The method of any of embodimentsA64-A66, wherein a change in the alkaline phosphatase level is from about 8.8 U/L to about-8.4 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A68. The method of any of embodiments A64-A67, wherein a change in the alkaline phosphatase level is from about 2.1 U/L to about-8.4 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A69. The method of embodiments A64 or A65, wherein a change in the alkaline phosphatase level occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A70. The method of any of embodiments A64-A66, wherein a change in the alkaline phosphatase level is from about 4 U/L to about-30 U/L at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A71. The method of any of embodiments A64-A67, wherein a change in the alkaline phosphatase level is from about 2 U/L to about-26 U/L at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A72. The method of any of embodiments A64-A68, wherein the alkaline phosphatase level is decreased following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A73. The method of any of embodiments A64-A69, wherein a change in the alkaline phosphatase level is from about-8.4 U/L to about 0 U/L at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A74. The method of any of embodiments A64-A69, wherein a change in the alkaline phosphatase level is from about-30 U/L to about 0 U/L at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A75. The method of any of embodiments A64-A69, wherein a change in the alkaline phosphatase level is from about-35 U/L to about 0 U/L at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A76. The method of any of embodiments A64-A69, wherein a change in the alkaline phosphatase level is from about-40 U/L to about 0 U/L at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A77. The method of embodiment A76, wherein the human has a alkaline phosphatase level above the upper limit of normal prior to the first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A78. The method of any of embodiments A1-A77 wherein the administration results in stabilization or decrease in total bilirubin in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A79. The method of embodiment A78, wherein the stabilization or decrease in total bilirubin occurs within about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A80. The method of embodiments A78 or A79, wherein the stabilization or decrease in total bilirubin is from about-0.2 mg/dL to about 1 mg/dL at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A81. The method of any of embodiments A78-A80, wherein total bilirubin is decreased by about 0.5 mg/dL at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A82. The method of embodiment A78, wherein the stabilization or decrease in total bilirubin occurs within about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A83. The method of embodiment A78, wherein the stabilization or decrease in total bilirubin is from about-0.2 mg/dL to about 1 mg/dL at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A84. The method of embodiment A78, wherein total bilirubin is decreased by about 0.5 mg/dL at about 24 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human.

Embodiment A85. The method of any of embodiments A1-A84, wherein the human is not concurrently being treated with another therapeutic for primary sclerosing cholangitis.

Embodiment A86. The method of any of embodiments A1-A84, wherein the human is concurrently being treated with another therapeutic for primary sclerosing cholangitis.

Embodiment A87. The method of embodiment A86, wherein the other therapeutic comprises an immunosuppressant, an antifibrotic agent, niacin, a statin, a bile acid, a bile salt, a bile acid sequestrant, or an antibiotic.

Embodiment A88. The method of embodiment A87, wherein the immunosuppressant is selected from the group consisting of prednisone, budesonide, methotrexate, azathioprine, mycophenolate mofetil, cyclosporine, tacrolimus, cladribine, pentoxifylline, and a corticosteroid.

Embodiment A89. The method of embodiment A87, wherein the antifibrotic agent is selected from the group consisting of colchicine, D-penicillamine, pirfenidone, nintedanib, and niacin.

Embodiment A90. The method of embodiment A62, wherein the other therapeutic comprises a bile acid.

Embodiment A91. The method of embodiment A90, wherein the bile acid is a cholic acid.

Embodiment A92. The method of embodiment A91 wherein the cholic acid is ursodeoxycholic acid.

Embodiment A93. The method of embodiment A87, wherein the bile acid sequestrant is cholestyramine.

Embodiment A94. The method of embodiment A87, wherein the antibiotic is selected from the group consisting of vancomycin, minocycline, and rifaximin.

Embodiment A95. The method of any of embodiments A1-A94, wherein the method is not accompanied by a serious treatment-related adverse event.

Embodiment A96. The method of embodiment A95, wherein the serious treatment-related adverse event comprises cholangitis, cholecystitis, pancreatitis, or abdominal pain.

Embodiment A97. The method of embodiment A95, wherein the serious treatment-related adverse event is cholangitis.

Embodiment A98. The method of any of embodiments A1-A97, wherein(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, is administered in an amount of about 40 mg, 80 mg, 160 mg or 320 mg.

Embodiment A99. The method of any of embodiments A1-A98, wherein hepatocyte function is improved in the human following administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A100. The method of embodiment A99, wherein hepatocyte function is measure by relative enhancement using a contrast agent.

Embodiment A101. The method of embodiment A100, wherein the contrast agent is gadoxetate.

Embodiment A102. The method of any of embodiments A1-A101 wherein bile flow is improved in the human following administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

Embodiment A103. The method of embodiment A102, wherein bile flow is measured by time of arrival of a contrast agent to a bile duct.

Embodiment A104. The method of embodiment A103, wherein the contrast agent is gadoxetate.

Embodiment A105. The method of any of embodiments A1-A104, wherein the human is an adult.

Embodiment A106. A method of downregulating a gene in a human who has primary sclerosing cholangitis, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the gene is selected from the group consisting of COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

Embodiment A107. A method of downregulating a gene in a human who has primary sclerosing cholangitis, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the gene is selected from the group consisting of COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

Embodiment A108. A method of downregulating a gene in a human who has primary sclerosing cholangitis, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the gene is selected from the group consisting of ADAMTS3, ANXA6, CCDC80, COL5A1, COL7A1, CXCL12, FBLN1, IGFBP7, or LOXL1.

Embodiment A109. The method of any of embodiments A1-A108, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a phosphate salt.

Embodiment A110. The method of embodiment A109, wherein the phosphate salt is crystalline.

Embodiment A111. The method of embodiment A110, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is crystalline Form I phosphate salt.

Embodiment A112. The method of embodiment A110, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is selected from the group consisting of: crystalline Form IV phosphate salt, crystalline Form II fumarate salt, crystalline Form III naphthalenedisulfonic acid salt, a zwitterionic form, and an amorphous form.

Embodiment A113. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in ameliorating fibrosis in tissue of a human in need thereof, wherein the tissue is liver tissue, gall bladder tissue, or bile duct tissue.

Embodiment A114. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in treating primary sclerosing cholangitis in a human in need thereof, wherein primary sclerosing cholangitis in the human is ameliorated.

Embodiment A115. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in reducing pruritus in a human who has primary sclerosing cholangitis.

Embodiment A116. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in slowing the clinical progression of primary sclerosing cholangitis in a human who has primary sclerosing cholangitis and is at risk for moderate to severe liver fibrosis.

Embodiment A117. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of COL1A1, COL1A2, COL3A1 and TFGb-1.

Embodiment A118. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for ameliorating fibrosis in a tissue of a human in need thereof, wherein the tissue is liver tissue, gall bladder tissue, or bile duct tissue.

Embodiment A119. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for treating primary sclerosing cholangitis in a human in need thereof, wherein primary sclerosing cholangitis in the human is ameliorated.

Embodiment A120. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for reducing pruritus in a human who has primary sclerosing cholangitis.

Embodiment A121. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for slowing the clinical progression of primary sclerosing cholangitis in a human who has primary sclerosing cholangitis and is at risk for moderate to severe liver fibrosis.

Embodiment A122. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of COL1A1, COL1A2, COL3A1 and TFGb-1.

Embodiment A123. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

Embodiment A124. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

Embodiment A125. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in downregulating a gene in a human who has primary sclerosing cholangitis, wherein the gene is selected from the group consisting of ADAMTS3, ANXA6, CCDC80, COL5A1, COL7A1, CXCL12, FBLN1, IGFBP7, or LOXL1.

Embodiment A126. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of COL16A1, BGN, FBLN1, LTBP2, AEBP1, SULF1, LOXL1, CCDC80, CXCL12, SRPX2, ADAMTS3, COL5A1, COL7A1, MGP, ANXA6, COL8A1, IGFBP7, or CTSC.

Embodiment A127. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of COL27A1, VWF, LAMA2, MMP2, SERPINE1, PDGFB, HTRA1, P3H1, PLAT, LAMB1, HSPG2, NID2, ADAMTS3, SRPX2, COL4A2, COL4A1, or CTHRC1.

Embodiment A128. Use of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, in the manufacture of a medicament for downregulating a gene in a human who has primary sclerosing cholangitis, wherein the gene is selected from the group consisting of ADAMTS3, ANXA6, CCDC80, COL5A1, COL7A1, CXCL12, FBLN1, IGFBP7, or LOXL1.

Embodiment A129. The use of any of embodiments A113-A128, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a phosphate salt.

Embodiment A130. The use of embodiment A129, wherein the phosphate salt is crystalline.

Embodiment A131. The use of embodiment A130, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is crystalline Form I phosphate salt.

Embodiment A132. The use of embodiment A130, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is selected from the group consisting of: crystalline Form IV phosphate salt, crystalline Form II fumarate salt, crystalline Form III naphthalenedisulfonic acid salt, a zwitterionic form, and an amorphous form.

Salts and Polymorphs of (S)-4-((2-methoxyethyl)(4-(5,6,7,8-tetrahydro-1,8-naphthyridin-2-yl)butyl)amino)-2-(quinazolin-4-ylamino)butanoic acid

Various salts and polymorphs of (S)-4-((2-methoxyethyl)(4-(5,6,7,8-tetrahydro-1,8-naphthyridin-2-yl)butyl)amino)-2-(quinazolin-4-ylamino)butanoic acid, can be used in the compositions and methods disclosed herein. (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof can be used in combination with ursodeoxycholic acid or a pharmaceutically acceptable salt thereof. (S)-4-((2-methoxyethyl)(4-(5,6,7,8-tetrahydro-1,8-naphthyridin-2-yl)butyl)amino)-2-(quinazolin-4-ylamino)butanoic acid is also known as bexotegrast.

Salts and polymorphs of (S)-4-((2-methoxyethyl)(4-(5,6,7,8-tetrahydro-1,8-naphthyridin-2-yl)butyl)amino)-2-(quinazolin-4-ylamino)butanoic acid are disclosed in US Patent Application Publication No. 2022/0177468, incorporated by reference herein in its entirety, and can be produced by the processes therein. In some embodiments, the methods herein employ a crystalline form of (S)-4-((2-methoxyethyl)(4-(5,6,7,8-tetrahydro-1,8-naphthyridin-2-yl)butyl)amino)-2-(quinazolin-4-ylamino)butanoic acid. Crystalline forms of (S)-4-((2-methoxyethyl)(4-(5,6,7,8-tetrahydro-1,8-naphthyridin-2-yl)butyl)amino)-2-(quinazolin-4-ylamino)butanoic acid include the Form I phosphate salt, the Form II fumarate salt, the Form III 1,5-naphthalenedisulfonate salt, and the Form IV mixed isopropyl alcohol and water phosphate salt. In some embodiments, the crystalline form is selected from the group consisting of the Form I phosphate salt, the Form II fumarate salt, the Form III 1,5-naphthalenedisulfonate salt, and the Form IV mixed isopropyl alcohol and water phosphate salt. In some embodiments, the crystalline form is the Form I or Form IV phosphate salt. In some embodiments, the crystalline form is selected from the group consisting of the Form I phosphate salt, the Form II fumarate salt, the Form III 1,5-naphthalenedisulfonate salt, and the Form IV mixed isopropyl alcohol and water phosphate salt. In some embodiments, the crystalline form is the Form I phosphate salt. In some embodiments, the crystalline form is the Form II fumarate salt. In some embodiments, the crystalline form is the Form III 1,5-naphthalenedisulfonate salt. In some embodiments, the crystalline form is the Form IV mixed isopropyl alcohol and water phosphate salt. US Patent Application Publication No. 2022/0177468 is hereby incorporated herein by reference in its entirety.

In some embodiments, (S)-4-((2-methoxyethyl)(4-(5,6,7,8-tetrahydro-1,8-naphthyridin-2-yl)butyl)amino)-2-(quinazolin-4-ylamino)butanoic acid is the only therapy specific for a liver disorder (that is, the only liver-specific therapy) administered to the subject, such as a subject with a fibrotic liver disease, for example, primary sclerosing cholangitis. For example, (S)-4-((2-methoxyethyl)(4-(5,6,7,8-tetrahydro-1,8-naphthyridin-2-yl)butyl)amino)-2-(quinazolin-4-ylamino)butanoic acid may be administered without administration of ursodeoxycholic acid or a pharmaceutically acceptable salt thereof. For example, (S)-4-((2-methoxyethyl)(4-(5,6,7,8-tetrahydro-1,8-naphthyridin-2-yl)butyl)amino)-2-(quinazolin-4-ylamino)butanoic acid may be administered without administration of ursodeoxycholic acid or a pharmaceutically acceptable salt thereof or any other therapy specific for a liver disorder. For example, (S)-4-((2-methoxyethyl)(4-(5,6,7,8-tetrahydro-1,8-naphthyridin-2-yl)butyl)amino)-2-(quinazolin-4-ylamino)butanoic acid may be administered without administration of ursodeoxycholic acid or a pharmaceutically acceptable salt thereof or any other therapy specific for a fibrotic liver disorder. For example, (S)-4-((2-methoxyethyl)(4-(5,6,7,8-tetrahydro-1,8-naphthyridin-2-yl)butyl)amino)-2-(quinazolin-4-ylamino)butanoic acid may be administered without administration of ursodeoxycholic acid or a pharmaceutically acceptable salt thereof or any other therapy specific for primary sclerosing cholangitis. Subjects may be taking medications for reasons other than treatment of liver disorders, or which are not specific for liver disorders, such as over-the counter treatments, including, but not limited to, vitamins, minerals, or ibuprofen, or prescription treatments such as drugs to treat diabetes, high blood pressure, or other disorders. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid is provided as a phosphate salt. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid is provided as Form I phosphate salt.

In some embodiments, (S)-4-((2-methoxyethyl)(4-(5,6,7,8-tetrahydro-1,8-naphthyridin-2-yl)butyl)amino)-2-(quinazolin-4-ylamino)butanoic acid is administered to a subject without serious adverse events. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid is administered to a subject without treatment-emergent adverse event. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid is administered to a subject with less than about a 20% probability, less than about a 10% probability, or less than about a 5% probability of serious adverse events. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid is administered to a subject with less than about a 20% probability, less than about a 10% probability, or less than about a 5% probability of treatment-emergent adverse events. Probability of a serious adverse event or of a treatment-emergent adverse event can be calculated from the percentage of such events in a group of patients treated with(S)-4-((2-methoxyethyl)(4-(5,6,7,8-tetrahydro-1,8-naphthyridin-2-yl)butyl)amino)-2-(quinazolin-4-ylamino)butanoic acid. In any of these embodiments, (S)-4-((2-methoxyethyl)(4-(5,6,7,8-tetrahydro-1,8-naphthyridin-2-yl) butyl)amino)-2-(quinazolin-4-ylamino)butanoic acid can be the only therapy specific for a liver disorder (that is, the only liver-specific therapy) administered to the subject, such as a subject with a fibrotic liver disease, for example, primary sclerosing cholangitis. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid is provided as a phosphate salt. In some embodiments, (S)-4-((2-methoxyethyl)(4-(5,6,7,8-tetrahydro-1,8-naphthyridin-2-yl)butyl)amino)-2-(quinazolin-4-ylamino)butanoic acid is provided as Form I phosphate salt.

### Kits

The disclosure further provides kits for carrying out the methods as disclosed herein, which comprises one or more compounds described herein, or a pharmaceutically acceptable salt thereof, or a pharmacological composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for use in the treatment of a fibrotic disease.

Kits generally comprise suitable packaging. The kits may comprise one or more containers comprising any compound described herein. Each component (if there is more than one component) can be packaged in separate containers or some components can be combined in one container where cross-reactivity and shelf-life permit. One or more components of a kit may be sterile and/or may be contained within sterile packaging.

The kits may be in unit dosage forms, bulk packages (e.g., multi-dose packages) or sub-unit doses. For example, kits may be provided that contain sufficient dosages of a compound as disclosed herein (e.g., a therapeutically effective amount) and/or a second pharmaceutically active compound useful for a disease detailed herein (e.g., fibrosis) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

The kits may optionally further comprise instructions for daily administration of the dosage form to an individual in need thereof, such as instructions for administration of the dosage form to an individual in need thereof one, two, three, or four times daily, for example, instructions for administration of the dosage form to an individual in need thereof once daily.

### Examples

The following Examples are set forth to enable this disclosure to be more fully understood. It should be understood that these Examples are for illustrative purposes only and are not to be construed as limiting this disclosure in any manner.

### Example B-1: Pre-clinical Evaluation of the Anti-Fibrotic Activity of (S)-4-((2-methoxyethyl)(4-(5,6,7,8-tetrahydro-1,8-naphthyridin-2-yl)butyl)amino)-2-(quinazolin-4-ylamino)butanoic acid (Bexotegrast) in the BALB/c.Mdr2−/− Mouse Model of Biliary Fibrosis

Primary sclerosing cholangitis (PSC) is a chronic liver disease with an unidentified etiology that is characterized by inflammation and fibrosis of bile ducts, which results in the formation of multifocal bile duct strictures. See R. Chapman, et al. (2010) Hepatology 51 (2): 660-678, the entire contents of which are hereby incorporated by reference in their entirety. PSC progression from biliary fibrosis to cirrhosis is associated with proliferation of adult bipotent hepatic progenitors and reactive cholangiocytes. These reactive cholangiocytes drive fibrogenesis through paracrine secretion of proinflammatory and profibrotic factors that act on hepatic stellate cells, the primary fibrotic effector cells.

TGF-β1 is ubiquitously expressed and in tissues is associated with the latency-associated protein (LAP), keeping it in a quiescent state. TGF-β₁ can be released in its activated form through an interaction between LAP and the α_{V}β₁ or α_{V}β₆ integrins. Additionally, α_{V}β₆ is an RGD-binding integrin that is almost exclusively restricted to the epithelium compartment and is directly up-regulated by TGF-β1. See D. Sheppard, et al. (1992) J Biol Chem 267 (24): 17409-17414, the entire contents of which are hereby incorporated by reference in their entirety. It has limited expression levels in healthy adult tissues but is highly upregulated in response to mechanical and biochemical injuries. See J. M. Breuss, et al. (1995) J Cell Sci 108 (Pt 6): 2241-2251, the entire contents of which are hereby incorporated by reference in their entirety. Accordingly, α_{V}β₆ expression is significantly upregulated in the liver of PSC patients and BALBc.Mdr2−/− mice and pharmacological inhibition in BALBc.Mdr2−/− mice reduces markers of fibrosis. See E. Patsenker, et al. (2008) Gastroenterology 135 (2): 660-670; and Y. Popov, et al. (2008) J Hepatol 48 (3): 453-464, the entire contents of which are hereby incorporated by reference in their entirety.

Current concepts strongly advocate that ongoing microinjury to hepatic epithelial cells induces a fibrotic environment and that growth factors secreted by the injured epithelial cells promote hepatic stellate cell activation, recruitment, and differentiation into pro-fibrotic myofibroblasts. See M. Parola, et al. (2008) Mol Aspects Med 29 (1-2): 58-66, the entire contents of which are hereby incorporated by reference in their entirety. As the disease progresses, actively proliferating fibroblasts and myofibroblasts organize into fibroblastic foci and produce excessive fibrillar collagen that results in scarring of the liver, which in turn leads to distortion of the tissue architecture and loss of function. Integrin α_{V}β₁ is expressed by hepatic stellate cells and myofibroblasts and could be the major integrin responsible for TGF-β activation by myofibroblasts. See N. C. Henderson, et al. (2013) Nat Med. 19 (12): 1617-1624, the entire contents of which are hereby incorporated by reference in their entirety. Targeted inhibition of α_{V}β₁ by a small molecule inhibitor resulted in the same degree of reversal of liver fibrosis as previously reported for deletion of all av integrins from PDGFR-positive cells including HSCs. See N. I. Reed, et al. (2015) Sci Transl Med 7 (288): 288ra27, the entire contents of which are hereby incorporated by reference in their entirety.

Since α_{V}β₁ and α_{V}β₆ leads to latent TGF-β1 activation directly contributing to the fibrotic response (see N. C. Henderson, et al. (2013) Nat Med. 19 (12): 1617-1624 and N. I. Reed, et al. (2015) Sci Transl Med. 7 (288): 288ra27, the entire contents of which are hereby incorporated by reference in their entirety) and their expression levels closely accompany the fibrotic scar, it is believed that inhibition of the α_{V}β₆/α_{V}β₁-TGF-β1 axis allows for a localized, and therefore safe, targeting of TGF-β1 signaling. See N. I. Reed, et al. (2015) Sci Transl Med. 7 (288): 288ra27, the entire contents of which are hereby incorporated by reference in their entirety.

Antifibrotic activity of (S)-4-((2-methoxyethyl)(4-(5,6,7,8-tetrahydro-1,8-naphthyridin-2-yl)butyl)amino)-2-(quinazolin-4-ylamino)butanoic acid was tested in the BALBc.Mdr2−/− murine model of liver biliary fibrosis. This animal model is similar to human PSC (see N. Ikenaga et al. (2015) Am J Pathol. 185 (2): 325-334, the entire contents of which are hereby incorporated by reference in their entirety) and demonstrates accelerated fibrosis compared to its parent FVB.Mdr−/− strain due to genetic resistance to fibrosis of FVB mice. BALBc.Mdr2−/− mice develop periductular fibrotic lesions at 4 weeks with rapid and progressive fibrotic progression. Bridging fibrosis and early cirrhosis is observed at 12 weeks along with severe portal hypertension and enhanced collagen deposition.

### Materials and Methods

Table B-1 includes information regarding the storage and formulation of the test substance.

TABLE B-1
Storage and Formulation

| | Vehicle/ | | |
|---|---|---|---|
| Test Material | Formulated | Route Given | Comments |
| (S)-4-((2-methoxyethyl)(4- | Phosphate- | Oral | Concentration: 20 |
| (5,6,7,8-tetrahydro-1,8- | buffered | administration | mg/mL, Dose at 5 mL/kg |
| naphthyridin-2- | saline | once daily | (100 mg/kg daily). |
| yl)butyl)amino)-2-(quinazolin- | (“PBS”) | (“PO/QD”) | pH: 6-7.5 |
| 4-ylamino)butanoic acid | | | |
| (S)-4-((2-methoxyethyl)(4- | PBS | PO/QD | Concentration: 60 |
| (5,6,7,8-tetrahydro-1,8- | | | mg/mL, Dose at 5 mL/kg |
| naphthyridin-2- | | | (300 mg/kg daily). |
| yl)butyl)amino)-2-(quinazolin- | | | pH: 6-7.5 |
| 4-ylamino)butanoic acid | | | |
| (S)-4-((2-methoxyethyl)(4- | PBS | PO/QD | Concentration: 200 |
| (5,6,7,8-tetrahydro-1,8- | | | mg/mL, Dose at 5 mL/kg |
| naphthyridin-2- | | | (1000 mg/kg daily). |
| yl)butyl)amino)-2-(quinazolin- | | | pH: 6-7.5 |
| 4-ylamino)butanoic acid | | | |

The tissue source is BALBc.Mdr2-/−male mice that were 6 weeks old. The Mdr2(abcb4)−/− mouse model of sclerosing cholangitis has been previously described and is a model of spontaneously progressive hepatic fibrosis in mice with deletion of the biliary phospholipid flippase on fibrosis-susceptible background (Mdr2(abcb4)^{-/-}. BALB/c mouse). See N. Ikenaga, et al. (2015) Am J Pathol. 185 (2): 325-334, the entire contents of which are hereby incorporated by reference in their entirety.

(S)-4-((2-methoxyethyl)(4-(5,6,7,8-tetrahydro-1,8-naphthyridin-2-yl)butyl)amino)-2-(quinazolin-4-ylamino)butanoic acid (or PBS) was dosed once daily orally at 100 mg/kg, 300 mg/kg or 1000 mg/kg (see Table B-2) starting at 6 weeks after birth of the subject and continuing for 6 weeks. All animals were euthanized and necropsied at the end of week 12.

TABLE B-2
Dosing Schedule

| Number | | | | | Harvest |
|---|---|---|---|---|---|
| of | | | Route | Dose | Schedule |
| Subjects | Test Article | Dose | Delivered | Schedule | Week |
| 12 | (S)-4-((2- | 100 | Oral | Week 7-12 | 12 |
| | methoxyethyl)(4- | mg/kg | administration | | |
| | (5,6,7,8-tetrahydro-1,8- | | once daily | | |
| | naphthyridin-2- | | (“PO/QD”) | | |
| | yl)butyl)amino)-2- | | | | |
| | (quinazolin-4- | | | | |
| | ylamino)butanoic acid | | | | |
| 12 | (S)-4-((2- | 300 | PO/QD | Week 7-12 | 12 |
| | methoxyethyl)(4- | mg/kg | | | |
| | (5,6,7,8-tetrahydro-1,8- | | | | |
| | naphthyridin-2- | | | | |
| | yl)butyl)amino)-2- | | | | |
| | (quinazolin-4- | | | | |
| | ylamino)butanoic acid | | | | |
| 9 | (S)-4-((2- | 1000 | PO/QD | Week 7-12 | 12 |
| | methoxyethyl)(4- | mg/kg | | | |
| | (5,6,7,8-tetrahydro-1,8- | | | | |
| | naphthyridin-2- | | | | |
| | yl)butyl)amino)-2- | | | | |
| | (quinazolin-4- | | | | |
| | ylamino)butanoic acid | | | | |
| 10 | PBS | — | PO/QD | Week 7-12 | 12 |

Concentrations of (S)-4-((2-methoxyethyl)(4-(5,6,7,8-tetrahydro-1,8-naphthyridin-2-yl)butyl)amino)-2-(quinazolin-4-ylamino)butanoic acid

Plasma samples were assayed by a qualified non-GLP bioanalytical method. Liver samples were homogenized in five volumes of ice-cold water. For plasma and liver homogenate, 20 μL of sample was extracted with 100 μL methanol: acetonitrile (5:95 v: v) containing 10 ng/mL internal standard. The solution was centrifuged at 4000 RPM for 5 minutes and 70 μL of supernatant transferred to injection plate containing 70 μl water. An aliquot of 10 μL was injected to a Sciex 4000 system. Concentrations of (S)-4-((2-methoxyethyl)(4-(5,6,7,8-tetrahydro-1,8-naphthyridin-2-yl)butyl)amino)-2-(quinazolin-4-ylamino)butanoic acid in mouse plasma and liver homogenate were determined by using Analyst 1.6 software (AB Sciex, Foster City, CA). Standard curves and quality control samples were run concurrently with the unknown samples. Using this bioanalytical method and four-times dilution of the samples, (S)-4-((2-methoxyethyl)(4-(5,6,7,8-tetrahydro-1,8-naphthyridin-2-yl)butyl)amino)-2-(quinazolin-4-ylamino)butanoic acid can be quantified in the range of 0.5-8000 ng/mL in plasma and liver homogenate.

### Direct (Invasive) Portal Venous Pressure Measurement

After laparotomy, the portal vein of the mouse subject was cannulated through an ileocolic vein, and portal pressure was measured directly by inserting a 1.2-Fr high-fidelity pressure catheter. Pressure signals were recorded at 2 kHz for 5 Min, and analyzed using PowerLab software. See N. Ikenaga, et al. (2015) Am J Pathol. 185 (2): 325-334, the entire contents of which are hereby incorporated by reference in their entirety.

### Assessment of Body and Organ Weights (Liver, Spleen)

Body, liver and spleen weights and colon length were determined at necropsy. Liver and plasma were snap frozen/preserved in liquid nitrogen. Two pieces of liver were fixed in formalin for histological assessment.

### Connective Tissue (Picrosirius Red) Histology

Connective tissue stains were carried out to morphometrically assess fibrosis, through analysis of 10 high power fields per animal.

### Liver and Kidney Biochemistries in Serum

Determination of ALT, ALP, and total bilirubin was performed as conventional liver and kidney function tests at the clinical biochemistry core (BIDMC).

### Determination of Relative and Total Liver Collagen

Liver collagen was determined biochemically as hydroxyproline (OHP). See N. Ikenaga, et al. (2015) Am J Pathol. 185 (2): 325-334, the entire contents of which are hereby incorporated by reference in their entirety. Two fresh liver pieces from the left and right liver lobe (120-160 mg each) were hydrolyzed in 2 mL 6N HCl at a temperature of 110° C. for 16 h. Moreover, 20 μL of hydrolysate was evaporated under N₂ and OHP was quantified using the chloramine-T/dimethylamino-benzaldehyde method. OHP levels were used to calculate relative (per 100 mg of wet liver) and total (obtained by multiplying liver weights with relative hepatic OHP) hepatic collagen content.

### Methods

Mouse liver tissues were lysed with RIPA buffer with protease inhibitor cocktail. Total protein concentration was determined by Micro BCA kit. MSD GOLD™ 96 Sm Spot SA Plate was washed three times with 150 μL of PBST and 1 μg/mL of biotinylated anti-SMAD2/3 was added to the plate, 25 μL per well (60 Min incubation under agitation). The plate was washed as before and 12.5 μL of unknown samples plus 12.5 μL of PBST were added in duplicates. After incubation and washing, 1 μg/mL of sulfo-tag labeled anti-pSMAD3 or anti-SMAD3 antibody was added (25 μL per well) and incubated for 60 Min under agitation. After wash, 150 μL of 1× read buffer was added and the plate was read using an MSD Sector Imager instrument.

### Statistical Analysis

Data are expressed as means±SEM unless otherwise noted and statistical analyses were performed using Microsoft EXCEL and GraphPad Prism. Multiple comparisons were performed by one-way analysis of variance (ANOVA). Planned comparisons were performed to the vehicle group using the Dunnett's post-test. Differences among experimental groups with p values lower than 0.05 were considered significant.

### Results

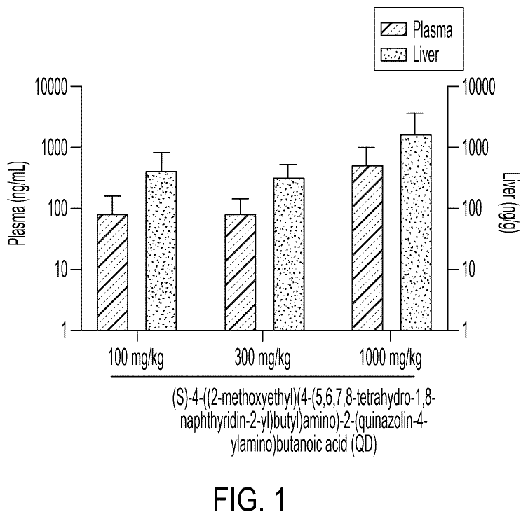

After 6 weeks of PO/QD dosing of (S)-4-((2-methoxyethyl)(4-(5,6,7,8-tetrahydro-1,8-naphthyridin-2-yl)butyl)amino)-2-(quinazolin-4-ylamino)butanoic acid, plasma and liver concentrations showed similar(S)-4-((2-methoxyethyl)(4-(5,6,7,8-tetrahydro-1,8-naphthyridin-2-yl)butyl)amino)-2-(quinazolin-4-ylamino)butanoic acid levels in the 100 mg/kg and 300 mg/kg groups and higher levels in the 1000 mg/kg group. Liver levels (ng/g) were approximately 3-5× the levels seen in the plasma (ng/mL) (see FIG. 1, which shows a graphical representation of plasma and liver concentrations of (S)-4-((2-methoxyethyl)(4-(5,6,7,8-tetrahydro-1,8-naphthyridin-2-yl)butyl)amino)-2-(quinazolin-4-ylamino)butanoic acid in BALB/c.Mdr2−/− mice at harvest, where data is meantstandard deviation). No deaths occurred in any group after the start of dosing.

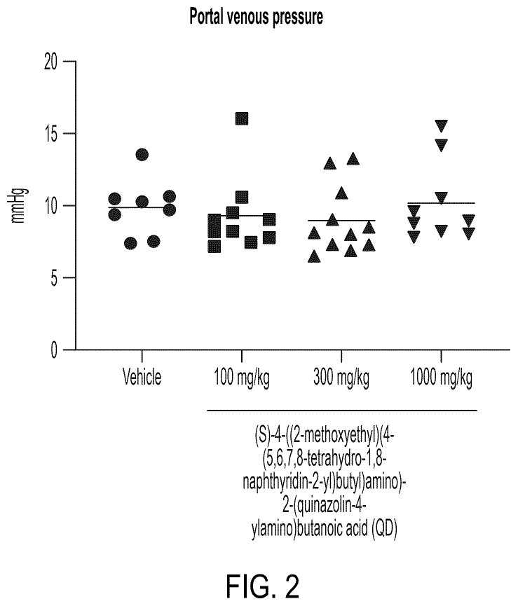

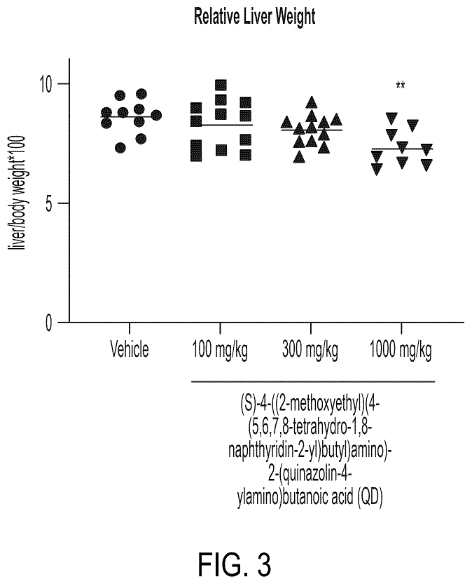

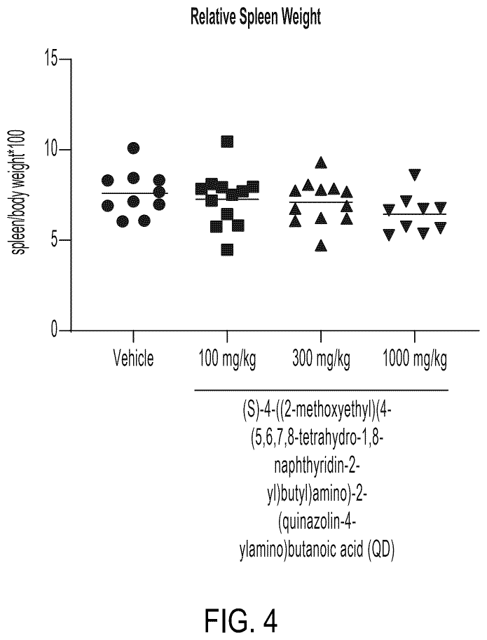

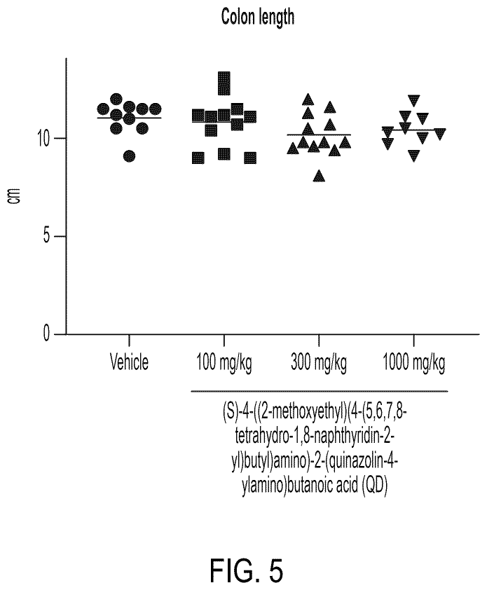

Specifically, FIG. 2 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on portal venous pressure of the subject. FIG. 3 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on relative liver weight of the subject, where ** p<0.01. FIG. 4 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on relative spleen weight of the subject, where ** p<0.01. FIG. 5 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on colon length of the subject, where ** p<0.01.

Over 6 weeks, treatment with(S)-4-((2-methoxyethyl)(4-(5,6,7,8-tetrahydro-1,8-naphthyridin-2-yl)butyl)amino)-2-(quinazolin-4-ylamino)butanoic acid did not induce any changes in portal venous pressure (see FIG. 2). About 1000 mg/kg/day(S)-4-((2-methoxyethyl)(4-(5,6,7,8-tetrahydro-1,8-naphthyridin-2-yl)butyl)amino)-2-(quinazolin-4-ylamino)butanoic acid decreased liver weight relative to vehicle control (see FIG. 3), however, spleen weights demonstrated no changes among all doses (see FIG. 4). Colon length did not change with compound treatment (see FIG. 5).

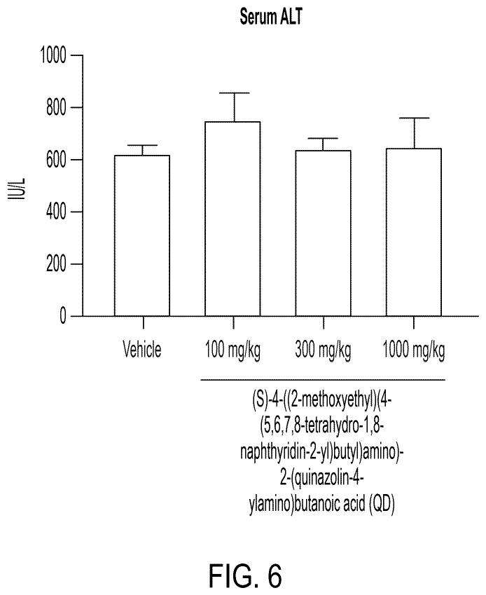

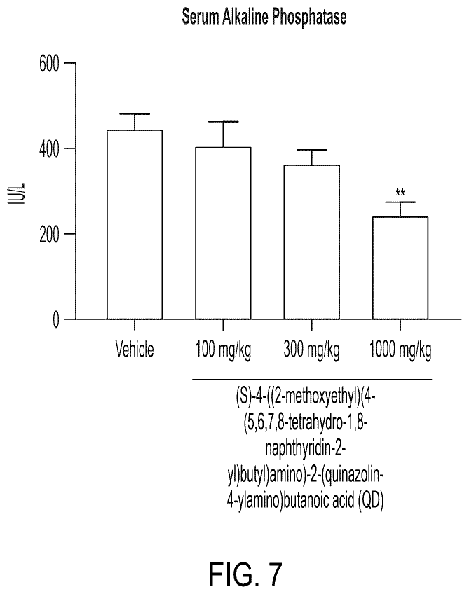

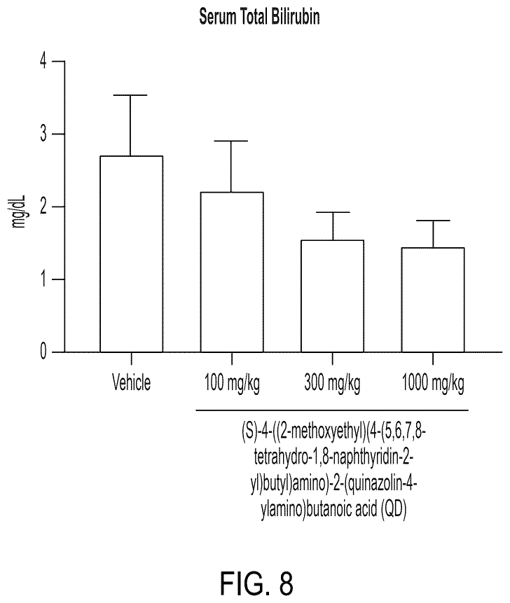

FIG. 6 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on serum ALT, where ** p<0.01. FIG. 7 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on serum alkaline phosphatase, where ** p<0.01. FIG. 8 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on serum total bilirubin, where ** p<0.01.

(S)-4-((2-methoxyethyl)(4-(5,6,7,8-tetrahydro-1,8-naphthyridin-2-yl)butyl)amino)-2-(quinazolin-4-ylamino)butanoic acid treatment did not change serum ALT levels (see FIG. 6). Serum alkaline phosphatase levels significantly decreased with 1000 mg/kg(S)-4-((2-methoxyethyl)(4-(5,6,7,8-tetrahydro-1,8-naphthyridin-2-yl)butyl)amino)-2-(quinazolin-4-ylamino)butanoic acid (see FIG. 7). Serum total bilirubin levels showed a dose dependent trend down with(S)-4-((2-methoxyethyl)(4-(5,6,7,8-tetrahydro-1,8-naphthyridin-2-yl)butyl)amino)-2-(quinazolin-4-ylamino)butanoic acid (see FIG. 8). Mice receiving oral treatment with 1000 mg/kg of (S)-4-((2-methoxyethyl)(4-(5,6,7,8-tetrahydro-1,8-naphthyridin-2-yl)butyl)amino)-2-(quinazolin-4-ylamino)butanoic acid showed a significant reduction in serum ALKP levels compared to vehicle control at time of collection.

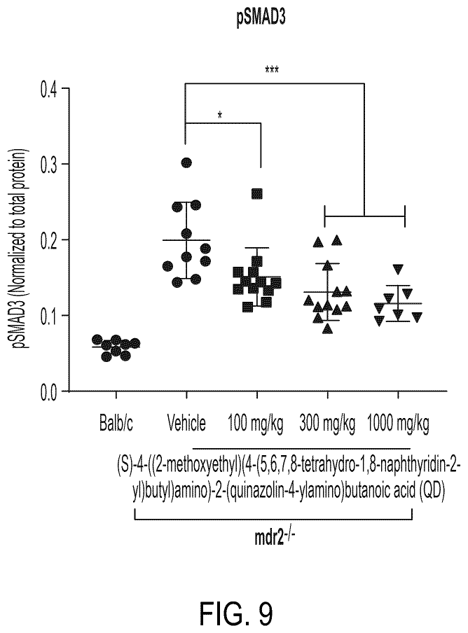

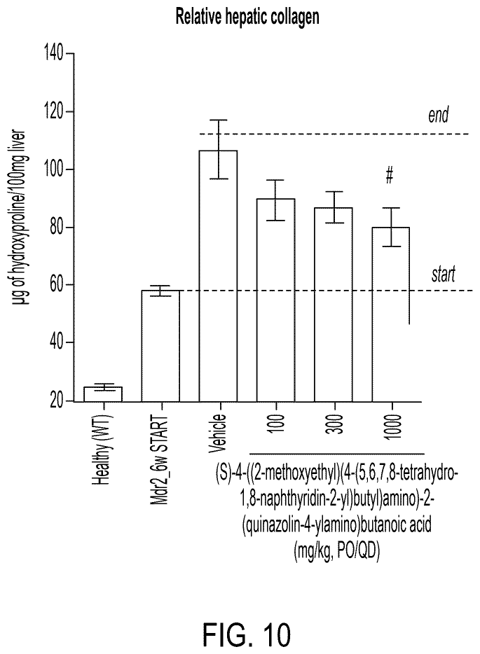

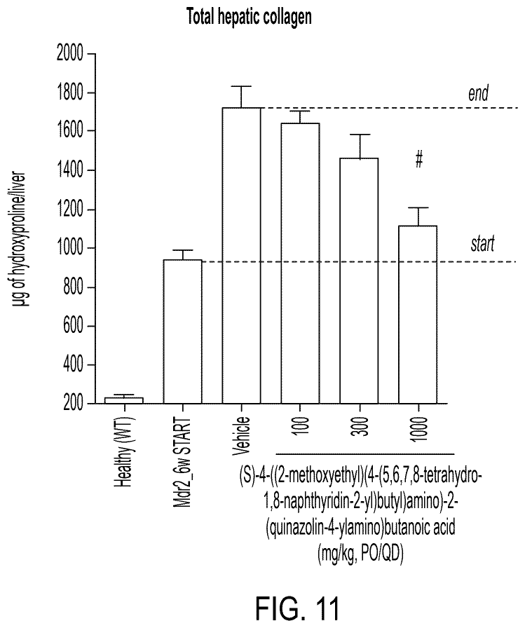

FIG. 9 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on pSMAD3, where * p<0.05, *** p<0.001, and #p<0.05. FIG. 10 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on relative hepatic collagen, where * p<0.05, *** p<0.001, and #p<0.05; Mdr2_6 w START indicates the six-week-old mice at the start of the experiment (i.e., baseline prior to treatment). FIG. 11 shows a graphical representation of the effect of various concentrations of (S)-4-((2-methoxyethyl)(4-(5,6,7,8-tetrahydro-1,8-naphthyridin-2-yl)butyl)amino)-2-(quinazolin-4-ylamino)butanoic acid on total hepatic collagen, where * p<0.05, *** p<0.001, and #p<0.05.

As shown in FIG. 9, pSMAD3 levels decreased with all doses of (S)-4-((2-methoxyethyl)(4-(5,6,7,8-tetrahydro-1,8-naphthyridin-2-yl)butyl)amino)-2-(quinazolin-4-ylamino)butanoic acid, indicating successful inhibition of release of active TGF-β₁. Both relative (normalized to liver weight) and total hepatic collagen content decreased with 1000 mg/kg of (S)-4-((2-methoxyethyl)(4-(5,6,7,8-tetrahydro-1,8-naphthyridin-2-yl)butyl)amino)-2-(quinazolin-4-ylamino)butanoic acid relative to the vehicle control (see FIG. 10 and FIG. 11).

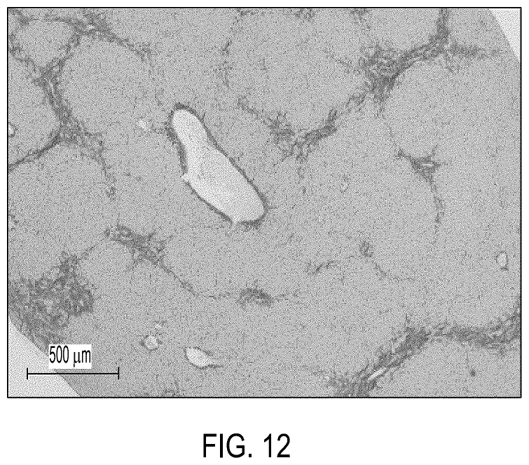

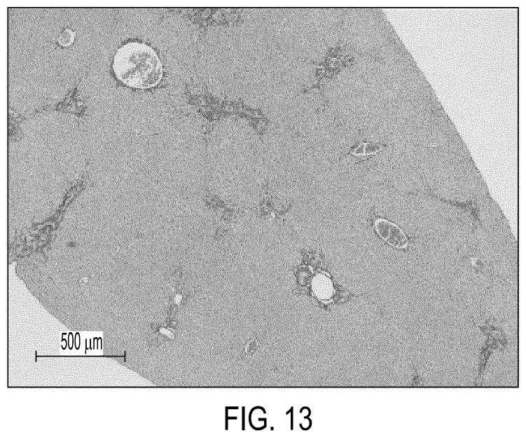

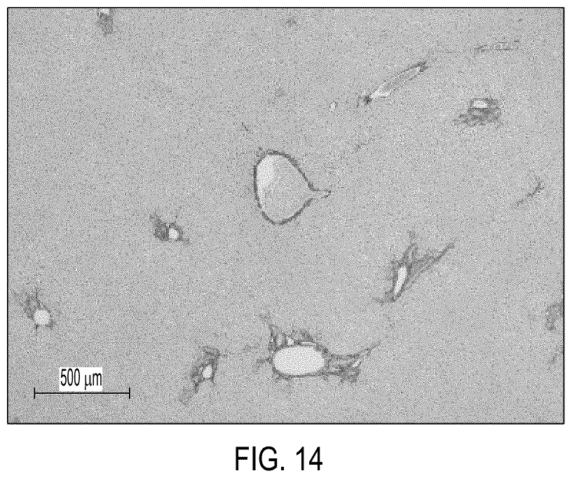

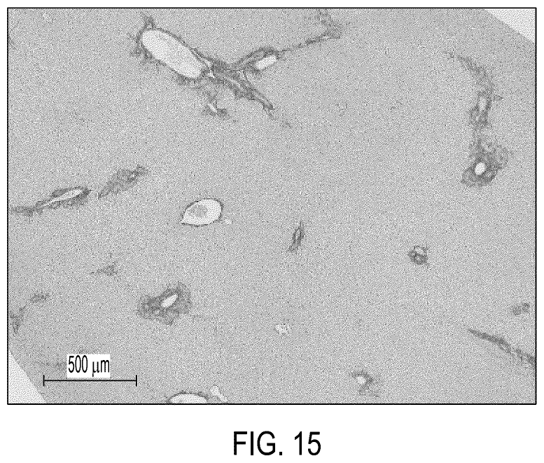

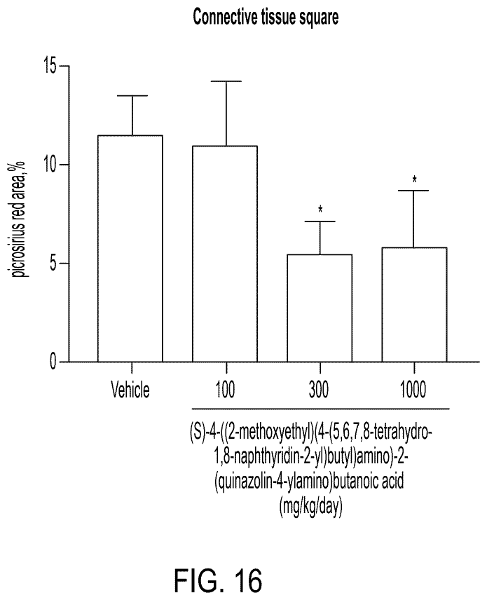

FIG. 12 shows a picrosirius red histological stained image of the vehicle in the BALBc.Mdr2-/−model. FIG. 13 shows a picrosirius red histological stained image associated with a subject treated with 100 mg/kg of (S)-4-((2-methoxyethyl)(4-(5,6,7,8-tetrahydro-1,8-naphthyridin-2-yl)butyl)amino)-2-(quinazolin-4-ylamino)butanoic acid in the BALBc.Mdr2−/− model. FIG. 14 shows a picrosirius red histological stained image associated with a subject treated with 300 mg/kg of (S)-4-((2-methoxyethyl)(4-(5,6,7,8-tetrahydro-1,8-naphthyridin-2-yl)butyl)amino)-2-(quinazolin-4-ylamino)butanoic acid in the BALBc.Mdr2-/−model. FIG. 15 shows a picrosirius red histological stained image associated with a subject treated with 1000 mg/kg of (S)-4-((2-methoxyethyl)(4-(5,6,7,8-tetrahydro-1,8-naphthyridin-2-yl)butyl)amino)-2-(quinazolin-4-ylamino)butanoic acid in the BALBc.Mdr2-/−model. FIG. 16 shows a graphical representation of the quantification of picrosirius red staining by percent area for various concentrations of (S)-4-((2-methoxyethyl)(4-(5,6,7,8-tetrahydro-1,8-naphthyridin-2-yl)butyl)amino)-2-(quinazolin-4-ylamino)butanoic acid, where * p<0.05.

The reduction in total hepatic collagen was approximately 75%. Picrosirius red histological staining revealed significant reductions in fibrosis in animals treated with 300 and 1000 mg/kg(S)-4-((2-methoxyethyl)(4-(5,6,7,8-tetrahydro-1,8-naphthyridin-2-yl)butyl)amino)-2-(quinazolin-4-ylamino)butanoic acid (see FIG. 14 and FIG. 15).

### DISCUSSION

Treatment of BALB/c.Mdr2−/− mice with 1000 mg/kg(S)-4-((2-methoxyethyl)(4-(5,6,7,8-tetrahydro-1,8-naphthyridin-2-yl)butyl)amino)-2-(quinazolin-4-ylamino)butanoic acid decreased liver weight, serum alkaline phosphatase levels, and liver collagen content after 6 weeks. Picrosirius red staining of liver sections demonstrated a significant reduction in fibrosis. Reduced pSMAD3 levels with treatment by(S)-4-((2-methoxyethyl)(4-(5,6,7,8-tetrahydro-1,8-naphthyridin-2-yl)butyl)amino)-2-(quinazolin-4-ylamino)butanoic acid indicates fibrotic reduction via TGF-β pathway inhibition.

### Conclusion

(S)-4-((2-methoxyethyl)(4-(5,6,7,8-tetrahydro-1,8-naphthyridin-2-yl)butyl)amino)-2-(quinazolin-4-ylamino)butanoic acid dose-dependently inhibited fibrosis in the BALB/c.Mdr2−/− mouse model of PSC liver, reducing the deposition of collagen and serum alkaline phosphatase levels.

### Example B-2. Evaluation of Anti-Fibrotic Activity of (S)-4-((2-methoxyethyl)(4-(5,6,7,8-tetrahydro-1,8-naphthyridin-2-yl)butyl)amino)-2-(quinazolin-4-ylamino)butanoic acid in a Human in vitro Model of Precision Cut Liver Slices

Common methods for testing efficacy of anti-fibrotic therapies include the use of isolated fibroblasts, sometimes in co-culture with epithelial cells, and/or in vivo animal models of fibrosis. The former provides a controlled system for monitoring cellular responses and the latter a physiological context for testing efficacy. Precision cut liver slices (PCLivS), thin slices of viable tissue from human explants or animal models that are cultured ex vivo, have been developed to bridge the gap between these cell and organism studies. In PCLivS the diverse cell and extracellular matrix composition and architecture of the native liver is preserved, providing a physiologically relevant platform in which to study aspects of liver disease progression and therapeutic response including fibrosis. See T. Luangmonkong, et al. (2018) Toxicol Appl Pharmacol. 355:127-37; I. M. Westra, et al. (2016) Toxicol In Vitro. 35:77-85; P. Olinga, et al. (2013) J Hepatol. 58 (6): 1252-3; I. M. Westra, et al. (2013) Xenobiotica. 43 (1): 98-112; and M. van de Bovenkamp, et al. (2008) Toxicol In Vitro. 22(3):771-8, the entire contents of which are hereby incorporated by reference in their entirety. Since PCLivS can be generated from liver tissue with biliary fibrosis including from PSC and primary biliary cirrhosis (PBC) explants, the ability of (S)-4-((2-methoxyethyl)(4-(5,6,7,8-tetrahydro-1,8-naphthyridin-2-yl)butyl)amino)-2-(quinazolin-4-ylamino)butanoic acid and obeticholic acid (OCA), a Farnesoid X Receptor (FXR) agonist in clinical trials for PSC, to inhibit expression of fibrotic genes in this model was assessed.

### Objectives

To determine the ex vivo anti-fibrotic activity of (S)-4-((2-methoxyethyl)(4-(5,6,7,8-tetrahydro-1,8-naphthyridin-2-yl)butyl)amino)-2-(quinazolin-4-ylamino)butanoic acid on precision cut slices from human PSC liver tissue.

### Tissue Source

A human liver tissue explant was obtained from a 30-year-old female PSC patient undergoing liver transplantation.

### Tissue Slicing and Culture

Slicing: To slice, 8-mm cores were punctured from the liver tissue and were stored in ice cold UW solution until slicing. A tissue core was glued onto the center of the plunger of the specimen syringe for slicing. The core was embedded in 2% low gelling temperature agarose made up in milliQ water. The thickness of the slice was adjusted to 400 μm and was sectioned into oxygenated KHB occurs. The slices were stored in cold UW solution on ice until all slices were cut.

Culture: The compounds were diluted to 1000× concentration in 100% DMSO. The compounds were diluted in a medium with a final concentration of 0.1% DMSO before transferring the slices and making a fresh medium for each 24-H medium change. Before being used for slice culture, all media was equilibrated in the incubator at culture conditions for 30 minutes. After all slices have been cut, all slices were transferred to wells prefilled with oxygenated, warmed, and equilibrated WEGG. The slices were incubated for 1 hour in the incubator at a temperature of 37° C., 5% CO₂, 80% 02, 100% humidity on shaking platform set at 55 rpm. After this one-hour recovery, the slices were transferred to wells prefilled with oxygenated, warmed, and equilibrated WEGG with appropriate treatments. The slices were distributed into the treatment groups to account for inter-core variability. The slices were cultured at 37° C., 80% 02, 5% CO₂, 100% humidity on shaking platform set at 55 rpm. The medium was changed after 24 hours.

### Collection of Samples

The slices were removed from the well and rinsed in PBS. Excess liquid was removed. The slices were transferred to a microtube pooling all three slices from the same treatment into one tube. The pooled slices were snap frozen in liquid nitrogen and placed on dry ice until all samples were collected and then transferred to −80° C. for storage. For media collection, 140 μL of medium was pooled from each well within a treatment group and transferred to a 96-well Axygen plate. After sealing the plate, spin down occurred at 4000×g for 10 minutes at 4° C. Then, 130 μL aliquots were transferred into 3 new plates. The plates were sealed and stored at −80° C.

### Treatment Groups

(S)-4-((2-methoxyethyl)(4-(5,6,7,8-tetrahydro-1,8-naphthyridin-2-yl)butyl)amino)-2-(quinazolin-4-ylamino)butanoic acid was tested at 100 nM, 1 μM, and 10 μM. OCA, an FXR agonist in clinical trials for PSC, was used at 250 nM and 2.5 μM reflecting Cₘₐₓ levels observed in clinical trials. An ALK5 inhibitor (R268712) was used at 1 μM (400 times its IC₅₀ per manufacturer) to target max inhibition of the ALK5 receptor.

### Study Design

### Lysate Generation

Materials; Frozen precision cut liver slices, Buffer RLT, RNAse-free water, and Proteinase K.

Methods: Lysis buffer was made with one third RLT buffer in RNAse-free water and 2 mg/mL proteinase K. About 100-200 μL lysis buffer was added to each pool of 3 liver slices. Samples were incubated at 55° C. in the bead bath for 2 hours and 45 minutes, vortexing every 30 minute. Samples were centrifuged and the supernatant aliquoted in 15 μL aliquots into 96-well Axygen plates.

### Hybridization and NanoString

Materials: NanoString PlexSet 24, hybridization buffer, custom probe A mix (IDT), custom probe B mix (IDT), TE-Tween, and Proteinase K.

Methods: nCounter PlexSet was run according to standard protocol. Briefly, probe A and probe B were diluted with TE-tween to Working Probe Pools and mixed with hybridization buffer. This master mix was added to the A-H PlexSets and mixed. Each PlexSet was added across a row to a 96 well qPCR plate and proteinase K was added to a final concentration of 2 mg/mL. Lysate from samples was added to appropriate wells and mixed. The plate was sealed, spun, and placed in thermocycler at 67° C. for 16 hours. Hybridized samples were pooled down columns and 35 μL were loaded on a Sprint Cartridge and run on the nCounter Sprint. Data analysis was performed in nSolver using background thresholding based on negative controls, positive control normalization, reference sample normalization and normalization to housekeeping genes (GUSB, HPRT1, POLR1B, RPLP0, SRFS4, and/or UBC).

Precision cut liver slices were generated from PSC and PBC patients undergoing transplant surgery and cultured based on previously reported methods. See I. A. de Graaf, et al. (2010) Nat Protoc. 5 (9): 1540-51, the entire contents of which are hereby incorporated by reference in their entirety. Specifically, 8-mm biopsy cores were punched from a piece of explanted liver tissue and 400-μm thick slices were cut from the cores and transferred to ice cold solution until culture. After all slices were collected, slices were transferred to 12-well plates containing 1.3 mL of media with the indicated compounds and placed at 37° C., 80% 02, 5% CO₂ with gentle agitation on an orbital shaker. Slices from each individual core were distributed among the treatment groups to control for regional variability. Media were changed after 24 hours. After 2 days in culture, the slices were washed, snap frozen and transferred to −80° C., and media were collected and frozen at −80° C. RNA-compatible tissue lysate was generated from slices and gene expression was determined using a custom fibrosis panel on a NanoString nCounter system. mRNA counts were normalized to housekeeping genes (GUSB, HPRT1, POLR1B, RPLP0, SRFS4, and/or UBC) and expression relative to vehicle controls were reported.

### Statistical Analysis

Relative expression levels were compared via one-way ANOVA with Dunnett's multiple comparisons test performed between each test group and the vehicle-treated group, unless otherwise stated.

### Results

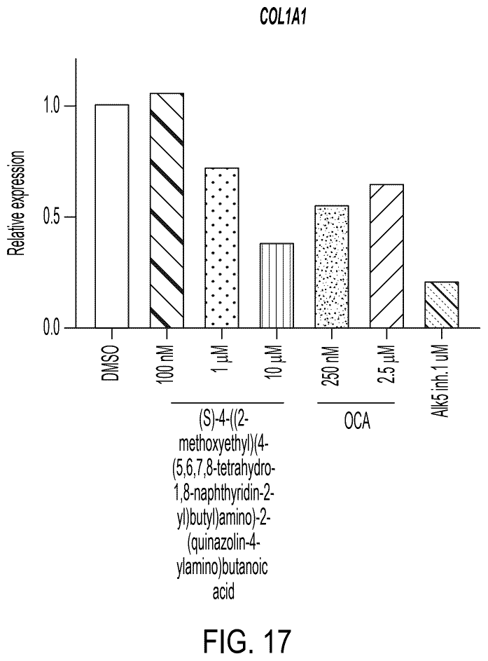

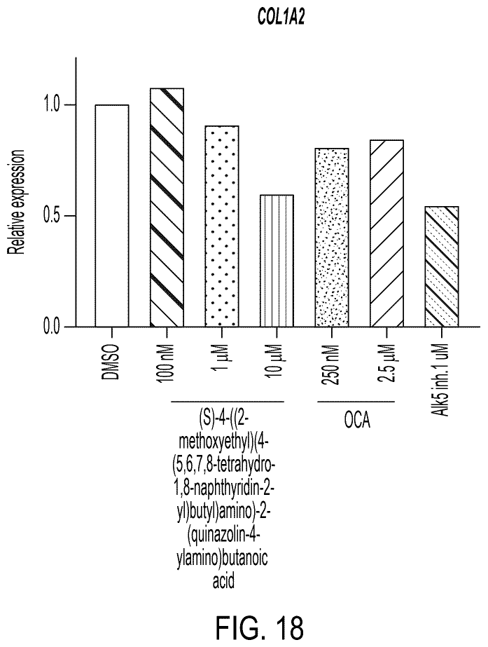

Specifically, FIG. 17 shows a graphical representation of (S)-4-((2-methoxyethyl)(4-(5,6,7,8-tetrahydro-1,8-naphthyridin-2-yl)butyl)amino)-2-(quinazolin-4-ylamino)butanoic acid and OCA on COLIA1 expression in PCLivS from human PSC liver tissue, where data from n=3 pooled slices was used from one independent experiment, culture and treatment occurred for two days, and DMSO was used as the solvent and was utilized at a constant concentration (0.1%) across the different groups. FIG. 18 shows a graphical representation of (S)-4-((2-methoxyethyl)(4-(5,6,7,8-tetrahydro-1,8-naphthyridin-2-yl)butyl)amino)-2-(quinazolin-4-ylamino)butanoic acid and OCA on COLIA2 expression in PCLivS from human PSC liver tissue, where data from n=3 pooled slices was used from one independent experiment, culture and treatment occurred for two days, and DMSO was used as the solvent and was utilized at a constant concentration (0.1%) across the different groups.

PCLivS from a single PSC liver tissue was cultured for two days with(S)-4-((2-methoxyethyl)(4-(5,6,7,8-tetrahydro-1,8-naphthyridin-2-yl)butyl)amino)-2-(quinazolin-4-ylamino)butanoic acid, OCA, or an ALK5 inhibitor, used as a positive control for inhibition of all TGF-β signaling. COLIA1 expression was decreased by 27% and 61% with 1 μM and 10 μM of (S)-4-((2-methoxyethyl)(4-(5,6,7,8-tetrahydro-1,8-naphthyridin-2-yl)butyl)amino)-2-(quinazolin-4-ylamino)butanoic acid, respectively. OCA decreased COLIA1 expression by 44% and 35% at 250 nM and 2.5 μM, respectively. COLIA2 expression was decreased by 9% and 41% with 1 μM and 10 μM of (S)-4-((2-methoxyethyl)(4-(5,6,7,8-tetrahydro-1,8-naphthyridin-2-yl)butyl)amino)-2-(quinazolin-4-ylamino)butanoic acid, respectively. OCA decreased COLIA2 expression by 19% and 15% at 250 nM and 2.5 μM, respectively (see Table B-3, FIG. 17, and FIG. 18).

TABLE B-3
Inhibition of collagen gene expression in PSC PCLivS

| | | | % Inhibition | |
|---|---|---|---|---|
| | | | of expression | |
| Compound | Concentration | | COL1A1 | COL1A2 |
| (S)-4-((2-methoxyethyl)(4- | 100 | nM | −6 | −8 |
| (5,6,7,8-tetrahydro-1,8- | 1 | μM | 27 | 9 |
| naphthyridin-2-yl)butyl)amino)- | 10 | μM | 61 | 41 |
| 2-(quinazolin-4- | | | | |
| ylamino)butanoic acid | | | | |
| OCA | 250 | nM | 44 | 19 |
| | 2.5 | μM | 35 | 15 |
| Alk5 Inh. | 1 | μM | 79 | 46 |

Nine additional livers with biliary fibrosis (five PSC liver and four PBC livers) were used to generate PCLivS that were cultured for two days with 100 nM, 1 μM and 10 μM of (S)-4-((2-methoxyethyl)(4-(5,6,7,8-tetrahydro-1,8-naphthyridin-2-yl)butyl)amino)-2-(quinazolin-4-ylamino)butanoic acid or an ALK5 inhibitor.

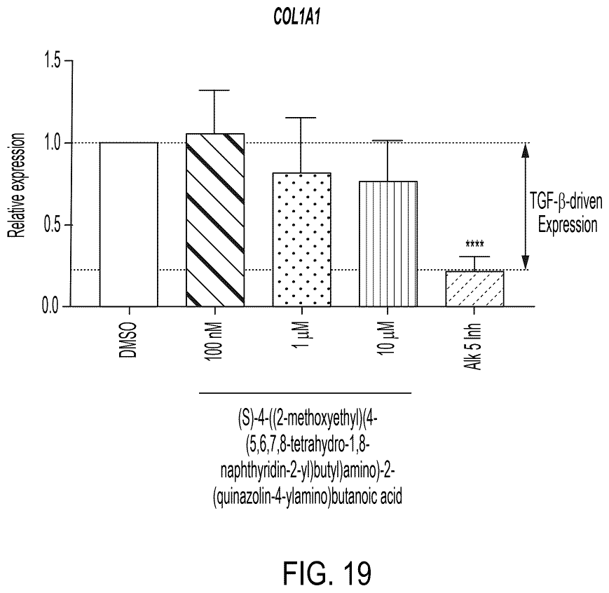

FIG. 19 shows a graphical representation of the effect of (S)-4-((2-methoxyethyl)(4-(5,6,7,8-tetrahydro-1,8-naphthyridin-2-yl)butyl)amino)-2-(quinazolin-4-ylamino)butanoic acid on pro-fibrotic gene expression (e.g., COLIA1 expression) in PCLivS from human liver tissue with biliary fibrosis, where data was taken from 3-6 slices from 10 independent experiments (n=6 PSC livers and n=4 PBC livers), culture and treatment occurred for two days, DMSO was used as the solvent and was utilized at a constant concentration (0.1%) across the different groups, * p<0.05, and **** p<0.0001.

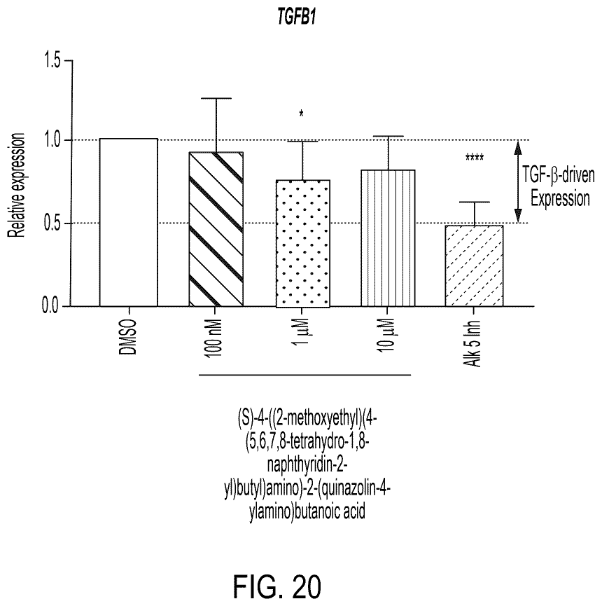

FIG. 20 shows a graphical representation of the effect of (S)-4-((2-methoxyethyl)(4-(5,6,7,8-tetrahydro-1,8-naphthyridin-2-yl)butyl)amino)-2-(quinazolin-4-ylamino)butanoic acid on pro-fibrotic gene expression (e.g., TGF-81 expression) in PCLivS from human liver tissue with biliary fibrosis, where data was taken from 3-6 slices from 10 independent experiments (n=6 PSC livers and n=4 PBC livers), culture and treatment occurred for two days, DMSO was used as the solvent and was utilized at a constant concentration (0.1%) across the different groups, * p<0.05, and **** p<0.0001.

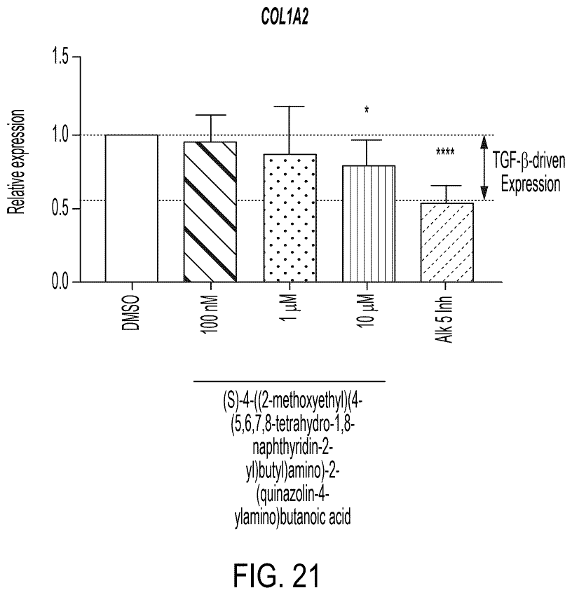

FIG. 21 shows a graphical representation of the effect of (S)-4-((2-methoxyethyl)(4-(5,6,7,8-tetrahydro-1,8-naphthyridin-2-yl)butyl)amino)-2-(quinazolin-4-ylamino)butanoic acid on pro-fibrotic gene expression (e.g., COLIA2 expression) in PCLivS from human liver tissue with biliary fibrosis, where data was taken from 3-6 slices from 10 independent experiments (n=6 PSC livers and n=4 PBC livers), culture and treatment occurred for two days, DMSO was used as the solvent and was utilized at a constant concentration (0.1%) across the different groups, * p<0.05, and **** p<0.0001.

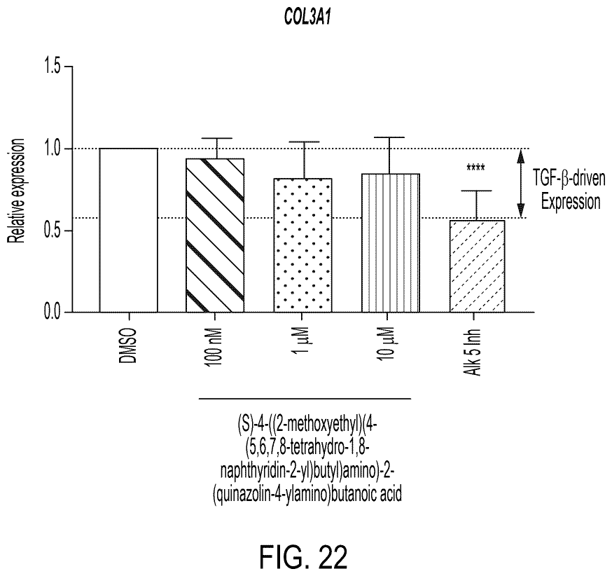

FIG. 22 shows a graphical representation of the effect of (S)-4-((2-methoxyethyl)(4-(5,6,7,8-tetrahydro-1,8-naphthyridin-2-yl)butyl)amino)-2-(quinazolin-4-ylamino)butanoic acid on pro-fibrotic gene expression (e.g., COL3A1 expression) in PCLivS from human liver tissue with biliary fibrosis, where data was taken from 3-6 slices from 10 independent experiments (n=6 PSC livers and n=4 PBC livers), culture and treatment occurred for two days, DMSO was used as the solvent and was utilized at a constant concentration (0.1%) across the different groups, * p<0.05, and **** p<0.0001.

Combining these data with the original PSC liver (n=10 total), there was a consistent trend down in overall expression of COLIA1 (22%) and COL3A1 (15%) and significant reductions in expression of TGF-81 (24%) and COLIA2(19%) with ≥1 μM of (S)-4-((2-methoxyethyl)(4-(5,6,7,8-tetrahydro-1,8-naphthyridin-2-yl)butyl)amino)-2-(quinazolin-4-ylamino)butanoic acid (see FIG. 19, FIG. 20, FIG. 21, and FIG. 22). The level of inhibition of expression achieved with the high dose of an ALK5 inhibitor in these livers can be used to determine the amount TGF-β-driven expression in this ex vivo system. Of the TGF-β-driven expression, (S)-4-((2-methoxyethyl)(4-(5,6,7,8-tetrahydro-1,8-naphthyridin-2-yl)butyl)amino)-2-(quinazolin-4-ylamino)butanoic acid inhibited up to 28% of COLIA1 expression, 43% of COLIA2 expression, 41% of COL3A1 expression and 48% of TGF-81 expression.

After two days in culture and incubation with(S)-4-((2-methoxyethyl)(4-(5,6,7,8-tetrahydro-1,8-naphthyridin-2-yl)butyl)amino)-2-(quinazolin-4-ylamino)butanoic acid, COLIA1 and COLIA2 mRNA expression in PCLivS from human PSC liver tissue was reduced in a dose dependent manner. OCA also reduced collagen gene expression, but did not show a dose dependent response at the concentrations tested. Expanding our analysis to 10 human liver tissues from patients with biliary fibrosis (PSC and PBC) showed a consistent decrease in collagen and TGFB-1 gene expression across tissues. The anti-fibrotic efficacy of (S)-4-((2-methoxyethyl)(4-(5,6,7,8-tetrahydro-1,8-naphthyridin-2-yl)butyl)amino)-2-(quinazolin-4-ylamino)butanoic acid in PCLivS from human liver tissue with biliary fibrosis suggests dual inhibition of α_{V}β₆ and α_{V}β₁ may be an effective approach to reducing fibrosis in patients with PSC.

### DISCUSSION

PCLivS was generated from a single PSC liver tissue and cultured in the presence of (S)-4-((2-methoxyethyl)(4-(5,6,7,8-tetrahydro-1,8-naphthyridin-2-yl)butyl)amino)-2-(quinazolin-4-ylamino)butanoic acid or OCA. (S)-4-((2-methoxyethyl)(4-(5,6,7,8-tetrahydro-1,8-naphthyridin-2-yl)butyl)amino)-2-(quinazolin-4-ylamino)butanoic acid inhibited the expression of COLIA1 and COLIA2 in a dose dependent manner. The primary therapeutic goal of OCA is improvement of bile acid homeostasis, not improvement in fibrosis, so while OCA also inhibited expression of these pro-fibrogenic genes, inhibition plateaued at lower concentrations of OCA. These data suggest a greater anti-fibrotic effect may be achieved clinically through inhibition of α_{V}β₁ and α_{V}β₆ integrins with(S)-4-((2-methoxyethyl)(4-(5,6,7,8-tetrahydro-1,8-naphthyridin-2-yl)butyl)amino)-2-(quinazolin-4-ylamino)butanoic acid. Testing of (S)-4-((2-methoxyethyl)(4-(5,6,7,8-tetrahydro-1,8-naphthyridin-2-yl)butyl)amino)-2-(quinazolin-4-ylamino)butanoic acid in PCLivS experiments from a total of ten human liver tissues with biliary fibrosis (six PSC and four PBC) showed a consistent reduction in profibrotic gene expression across multiple livers.

### Conclusion

(S)-4-((2-methoxyethyl)(4-(5,6,7,8-tetrahydro-1,8-naphthyridin-2-yl)butyl)amino)-2-(quinazolin-4-ylamino)butanoic acid is a potent inhibitor of fibrosis in PCLivS from human liver tissue with biliary fibrosis, including PSC, decreasing expression of collagen and TGF-β mRNA.

### Example B-3-Randomized, Double-Blind, Dose-Ranging, Placebo-Controlled, Phase 2a Evaluation of the Safety, Tolerability, and Pharmacokinetics of (S)-4-((2-Methoxyethyl)(4-(5,6,7,8-Tetrahydro-1,8-Naphthyridin-2-yl)Butyl) Amino)-2-(Quinazolin-4-ylamino) Butanoic Acid in Subjects with Primary Sclerosing Cholangitis (PSC) and Suspected Liver Fibrosis (INTEGRIS-PSC)

PSC is a rare, idiopathic, cholestatic liver disease that is characterized by biliary inflammation and progressive fibrosis. Over time, this biliary and hepatic inflammation progresses to serious and often fatal liver complications, such as cirrhosis, portal hypertension, and end-stage liver disease. See E. C. Goode, eta 1. (2016) Ther Adv Chronic Dis. 7 (1): 68-85 and E. Toy, et al. (2011) BMC Gastroenterol. 11:83, the entire contents of which are hereby incorporated by reference in their entirety. More than 50% of subjects require liver transplantation within 10 to 15 years after diagnosis. However, disease recurrence after transplantation is common. See G. M. Hirschfield, et al. (2013) Lancet. 382(9904):1587-99, the entire contents of which are hereby incorporated by reference in their entirety.

Patients with PSC are at greater risk of developing certain cancers in the hepatobiliary regions, with cholangiocarcinoma (CCA), the most prevalent form, having a lifetime risk ranging from 5% to 20%. See J. L. Horsley-Silva, et al. (2017) Liver Int. 37 (8): 1103-9 and K. N. Lazaridis, et al. (2016) N Engl J Med. 375 (12): 1161-70, the entire contents of which are hereby incorporated by reference in their entirety. Once diagnosed with CCA, the 5-year overall survival is poor, ranging from 20% to 68%. See A. H. Ali, et al. (2018) Hepatology. 67 (6) 2338-51, the entire contents of which are hereby incorporated by reference in their entirety. Although the progression of PSC is generally slow, the disease exhibits a highly variable natural history associated with age at diagnosis, sex, and ductal and inflammatory bowel disease (IBD) subtypes. See T. J. Weismüller, et al. (2017) Gastroenterology. 152(8):1975-84, the entire contents of which are hereby incorporated by reference in their entirety.

Although the etiology of PSC is largely unknown, strong associations have been made with both environmental and genetic risk factors. The characteristic biliary inflammation and injury seen in PSC may be the result of environmental exposures and gut microbial trauma triggering predisposed genetic pathways, which contribute to persistent injury of cholangiocytes, the cells that line the bile ducts. See K. N. Lazaridis (2016) N Engl J Med. 375 (12): 1161-70, the entire contents of which are hereby incorporated by reference in their entirety.

Concurrent autoimmune disease in patients with PSC is also common. The majority of cases of PSC are associated with IBD, mainly ulcerative colitis, and IBD is a major risk factor for the development of PSC. See E. C. Goode (2016) Ther Adv Chronic Dis. 7 (1): 68-85, the entire contents of which are hereby incorporated by reference in their entirety. In an analysis of high-density genotype data from tens of thousands of individuals of European ancestry, many of the genetic risk variants for PSC were found to be shared with ulcerative colitis. See D. Ellinghaus, et al. (2016) Nat Genet. 48 (5): 510-8, the entire contents of which are hereby incorporated by reference in their entirety. Patients with PSC also have a high lifetime risk of developing gastrointestinal malignancies. See K. Bambha, et al. (2003) Gastroenterology 125 (5): 1364-9, the entire contents of which are hereby incorporated by reference in their entirety.

There are currently no widely approved medical treatments for PSC. Disease management is confined to supportive measures, which fail to address disease progression. Ursodeoxycholic acid (UDCA), an established treatment for primary biliary cirrhosis (PBC), is commonly used for the management of PSC. However, clinical studies of its use in patients with PSC have produced meager and inconclusive results. See J. E. Eaton, et al. (2013) Gastroenterology 145 (3): 521-36, the entire contents of which are hereby incorporated by reference in their entirety. Moreover, long-term use of UDCA is controversial due to increased rates of serious adverse events (SAEs), including death and need for liver transplantation when given at high doses. See R. Chapman, et al. (2010) Hepatology 51 (2): 660-78, the entire contents of which are hereby incorporated by reference in their entirety. High-dose UDCA has also been associated with the development of colorectal neoplasia in patients with ulcerative colitis or PSC. See J. E. Eaton, et al. (2013) Gastroenterology 145 (3): 521-36, the entire contents of which are hereby incorporated by reference in their entirety.

The profibrotic cytokine transforming growth factor beta (TGF-β) is synthesized as a precursor molecule that is associated with LAP, a protein derived from the same gene product which keeps it in a quiescent state. The binding of integrins, transmembrane proteins responsible for cell-extracellular matrix protein interactions, to the arginine-glycine-aspartate amino acid sequence present in LAP has been identified as a major TGF-β₁ activation pathway in PSC, driving progression of fibrotic tissue remodeling.

(S)-4-((2-methoxyethyl)(4-(5,6,7,8-tetrahydro-1,8-naphthyridin-2-yl)butyl)amino)-2-(quinazolin-4-ylamino)butanoic acid is a small molecule and a selective dual inhibitor of α_{V}β₆ and α_{V}β₁ integrins (50% inhibitory concentration [IC_{50] 5.7} nM and 3.4 nM, respectively). Additionally, integrin-ligand binding assays have demonstrated that(S)-4-((2-methoxyethyl)(4-(5,6,7,8-tetrahydro-1,8-naphthyridin-2-yl)butyl)amino)-2-(quinazolin-4-ylamino)butanoic acid is highly selective for α_{V}β₆ and α_{V}β₁ over other integrin receptors, including those leukocyte-expressed integrins where therapeutic inhibition has previously been associated with significant toxicities (e.g., vedolizumab [α₄β₇ inhibitor; increased risk of infections, potential risk of progressive multifocal leukoencephalopathy (PML); and natalizumab [α_{V}β₁ and α_{V}β₇ inhibitor; increased risk of PML). Moreover, (S)-4-((2-methoxyethyl)(4-(5,6,7,8-tetrahydro-1,8-naphthyridin-2-yl)butyl)amino)-2-(quinazolin-4-ylamino)butanoic acid reduced collagen synthesis and improved liver parameters in a mouse model of sclerosing cholangitis. In ex vivo human PSC liver tissue (precision cut liver slices), treatment with(S)-4-((2-methoxyethyl)(4-(5,6,7,8-tetrahydro-1,8-naphthyridin-2-yl)butyl)amino)-2-(quinazolin-4-ylamino)butanoic acid dose-dependently decreased collagen gene expression, providing an indication of antifibrotic activity in PSC. Mice completely deficient for α_{V}β₆ function live a normal lifespan, suggesting that even full inhibition of such integrins is well tolerated.

The objectives of the Phase 2a study included assessing the safety, tolerability, and pharmacokinetics of (S)-4-((2-methoxyethyl)(4-(5,6,7,8-tetrahydro-1,8-naphthyridin-2-yl)butyl)amino)-2-(quinazolin-4-ylamino)butanoic acid in subjects with PSC and suspected liver fibrosis.

### Study Design

The study is a Phase 2a, multicenter, 3-part, randomized, double-blind, dose-ranging, placebo-controlled, parallel-group study that evaluated the safety, tolerability, and PK of once-daily treatment with(S)-4-((2-methoxyethyl)(4-(5,6,7,8-tetrahydro-1,8-naphthyridin-2-yl)butyl)amino)-2-(quinazolin-4-ylamino)butanoic acid in subjects aged 18 to 75 years with an established diagnosis of large duct PSC and suspected liver fibrosis. Subjects with stable inflammatory bowel disease (IBD) were eligible for the study.

Each study part included an up to 42-day screening period, followed by a treatment period of either 12-weeks (Parts 1 and 2) or at least 24 weeks (Part 3), and finally a 4-week post-treatment follow-up period. The treatment period for Part 3 was at least 24 weeks and up to 48 weeks.

In the study, 85 eligible subjects were randomized (3:1 ratio) to receive 40 mg (n=21), 80 mg (n=21), or 160 mg (n=21) of (S)-4-((2-methoxyethyl)(4-(5,6,7,8-tetrahydro-1,8-naphthyridin-2-yl)butyl)amino)-2-(quinazolin-4-ylamino)butanoic acid once daily or matching placebo once daily (n=21) for a time period of 12 weeks. Patients were screened on Day-28 and Day 1 was the first day of treatment. Patients were stratified for the use of Ursodeoxychcolic acid (UDCA). The primary and secondary endpoints were safety, tolerability, and pharmacokinetics (PK) of the bexotegrast dosage. The exploratory endpoints included changes in liver fibrosis markers (for example, ELF score and PRO-C), changes in liver biochemistry, and changes in liver imaging. Inclusion criteria was that a patient be at-risk for moderate/severe fibrosis defined by at least one criterion of ELF≥7.7; TE≥8 but≤14.4 kPa; magnetic resonance elastography (MRE)≥2.4 but≤4.9 kPa; or historical biopsy. Of the 179 subjects that were screened, 85 subjects were randomized (3:1 ratio) and treated. A total of 64 subjects received bexotegrast treatment and 4 (6.3%) discontinued treatment, 3 as a result of adverse events (in the 40 mg, 80 mg, and 160 mg cohorts) and 1 as a results of protocol deviation (40 mg cohort). A total of 42(65.6%) of the bexotegrast treatment subjects were stratified for the use of UDCA. A total of 21 subjects received treatment with placebo and 2(9.5%) discontinued treatment as a result of adverse events. A total of 13 (61.9%) subjects in the placebo group were stratified for the use of UDCA.

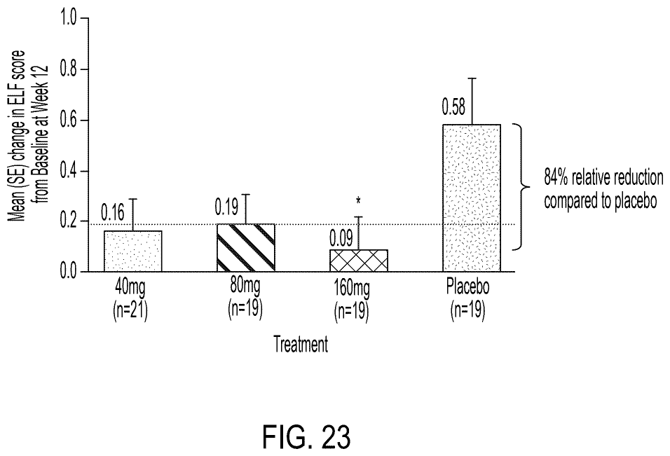

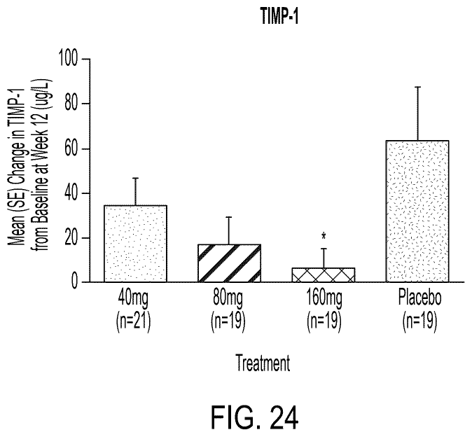

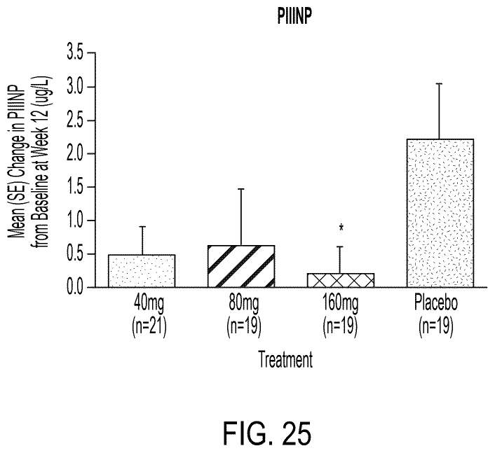

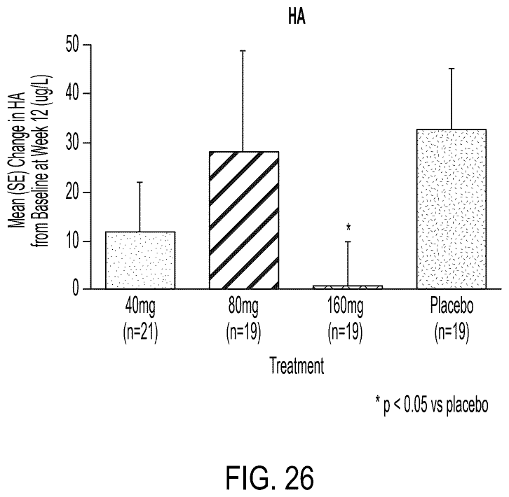

Bexotegrast was well-tolerated over 12 weeks of treatment in participants with PSC. Adverse events rates were comparable to placebo with all drug-related TEAEs mild or moderate in severity. There were no treatment-related SAEs. There was a low rate of discontinuation due to AEs. No notable effects on liver biochemistry or IBD activity status was observed. Patients with IBD experienced no clinically-relevant changes in IBD symptoms. Bexotegrast total and unbound plasma concentrations increased with dose. Bexotegrast demonstrated antifibrotic activity in a PSC population with suspected moderate to severe liver fibrosis. Bexotegrast reduced ELF score relative to placebo at all doses with a statistically significant difference for 160 mg (FIG. 23). Dose 160 mg achieved statistical significance across all components of the ELF score: Tissue inhibitor of metalloproteinases-1 (TIMP-1), Hyaluronic acid (HA), and Procollagen III, N-terminal propeptide (PIIINP); see FIG. 24, FIG. 25, and FIG. 26 (p<0.05 vs. placebo). All doses reduced collagen synthesis (PRO-C3) relative to placebo with statistical significance for the 160 mg dose. Bexotegrast demonstrated dose dependent reductions in itch relative to placebo with statistical significance for the 160 mg dose. Additionally, MRI imaging parameters were improved relative to placebo at Week 12. Dose dependent reductions in itch were reported with statistical significance for the 160 mg dose relative to placebo treatment. Liver biochemistry parameters were improved relative to placebo at Week 12.

Approximately 112 subjects were enrolled in Parts 1 (N=28), 2(N=56), and 3 (N=28). Subjects were centrally randomized to each of the treatment arms at the baseline visit using an interactive response technology (IRT) system in a blinded manner, according to a computer-generated randomization scheme. Randomization occurred immediately prior to dosing on the morning of the first dose (Day 1). Randomization was stratified by use of UDCA (yes/no).

The treatment periods were as follows:

The Part 1 treatment period included 28 eligible subjects in Cohort 1 that were randomized (3:1 ratio) to receive 40 mg of (S)-4-((2-methoxyethyl)(4-(5,6,7,8-tetrahydro-1,8-naphthyridin-2-yl)butyl)amino)-2-(quinazolin-4-ylamino)butanoic acid once daily (n=21) or matching placebo once daily (n=7) for a time period of 12 weeks. Upon review and approval of the data safety monitoring board (DSMB), the treatment period advances to Part 2.

The Part 2 treatment period included randomizing approximately 28 eligible subjects per cohort (56 in total) in a 3:1 ratio (active: placebo) who were administered 80 mg and 160 mg of (S)-4-((2-methoxyethyl)(4-(5,6,7,8-tetrahydro-1,8-naphthyridin-2-yl)butyl)amino)-2-(quinazolin-4-ylamino)butanoic acid for a time period of 12 weeks in sequential treatment cohorts. Cohort 3 (160 mg of (S)-4-((2-methoxyethyl)(4-(5,6,7,8-tetrahydro-1,8-naphthyridin-2-yl)butyl)amino)-2-(quinazolin-4-ylamino)butanoic acid and placebo) treatment assignments were initiated after Cohort 2(80 mg of (S)-4-((2-methoxyethyl)(4-(5,6,7,8-tetrahydro-1,8-naphthyridin-2-yl)butyl)amino)-2-(quinazolin-4-ylamino)butanoic acid and placebo) had completed enrollment and approved by the DSMB.

The Part 3 treatment period will include administering 320 mg of (S)-4-((2-methoxyethyl)(4-(5,6,7,8-tetrahydro-1,8-naphthyridin-2-yl)butyl)amino)-2-(quinazolin-4-ylamino)butanoic acid to the subject in Cohort 4 based on the following criteria:
Part 2 has been completely enrolled (i.e., 56 subjects had been randomized)
The data safety monitoring board (DSMB) reviews and provides a favorable opinion on the following data:
   All available safety and PK data from Parts 1 and 2; and
   Safety and PK data from a completed Phase 1 study evaluating the safety, tolerability, and pharmacokinetics of (S)-4-((2-methoxyethyl)(4-(5,6,7,8-tetrahydro-1,8-naphthyridin-2-yl)butyl)amino)-2-(quinazolin-4-ylamino)butanoic acid at multiple doses ranging from 80 to 320 mg in healthy subjects.

When Part 3 is initiated, approximately 28 eligible subjects will be randomized in a 3:1 ratio (320 mg(S)-4-((2-methoxyethyl)(4-(5,6,7,8-tetrahydro-1,8-naphthyridin-2-yl)butyl)amino)-2-(quinazolin-4-ylamino)butanoic acid: placebo) on Day 1 (Visit 3). Study treatment will be administered for at least 24 weeks. Treatment will continue for all subjects in Part 3 until the last subject enrolled in Part 3 reaches Week 24. At this time, all subjects will be contacted to return and complete the end of treatment (EOT) visit. The maximum treatment duration for the study will be 48 weeks.

Baseline demographics for the study of Example B-3 are shown in Table A-1.

TABLE A-1

| | Bexotegrast | Bexotegrast | Bexotegrast | Bexotegrast | |
|---|---|---|---|---|---|
| | 40 mg | 80 mg | 160 mg | All | Placebo |
| Characteristic | (n = 24)* | (n = 20)* | (n = 20)* | (n = 64) | (n = 21) |
| Male sex, n (%) | 17 (70.8) | 16 (80.0) | 14 (70.0) | 47 (73.4) | 17 (81.0) |
| Age (yr), mean (SD) | 46.9 (15.06) | 40.5 (15.32) | 45.1 (12.65) | 44.3 (14.46) | 45.6 (12.48) |

Race, n (%)

| | | | | | |
|---|---|---|---|---|---|
| White | 20 (83.3) | 16 (80.0) | 18 (90.0) | 54 (84.4) | 18 (85.7) |
| Black | 2 (8.3) | 2 (10.0) | 1 (5.0) | 5 (7.8) | 1 (4.8) |
| Asian | 2 (8.3) | 1 (5.0) | 1 (5.0) | 4 (6.3) | 1 (4.8) |
| Other/Not Reported/ | 0 | 1 (5.0) | 0 | 1 (1.6) | 1 (4.8) |
| Unknown | | | | | |
| Body-mass index | 28.2 (5.17) | 26.5 (4.51) | 26.8 (5.95) | 27.2 (5.21) | 26.4 (3.92) |
| (kg/m²), mean (SD) | | | | | |
| Time since diagnosis | 11.1 (8.15) | 8.3 (7.97) | 7.8 (6.78) | 9.2 (7.72) | 10 (7.95) |
| of PSC (yr), mean | | | | | |
| (SD) | | | | | |
| Concomitant UDCA | 14 (58.3) | 15 (75.0) | 13 (65.0) | 42 (65.6) | 13 (61.9) |
| use, n (%) | | | | | |
| Duration of UDCA | 8.3 (4.55) | 7.8 (10.84) | 4.6 (4.03) | 6.9 (7.35) | 5.3 (6.56) |
| use, n (%) | | | | | |
| IBD, n (%) | 18 (75.0) | 12 (60.0) | 11 (55.0) | 34 (53.1) | 12 (57.1) |
| Crohn's disease | 6 (25.0) | 4 (20.0) | 2 (10.0) | 12 (18.8) | 4 (19.0) |
| Ulcerative colitis | 11 (45.8) | 6 (30.0) | 7 (35.0) | 24 (37.5) | 7 (33.3) |
| IBD Other | 3 (12.5) | 2 (10.0) | 2 (10.0) | 7 (10.9) | 1 (4.8) |
| Partial Mayo Score, | 0.7 (1.08) | 1.6 (2.54) | 1.1 (1.27) | 1.1 (1.70) | 0.7 (1.56) |
| mean (SD) | | | | | |
| Itch NRS, mean | 1.8 (2.54) | 2.1 (2.63) | 1.4 (1.50) | 1.7 (2.27) | 1.1 (1.58) |
| (SD) | | | | | |

Duration since diagnosis at screening is calculated from the first reported date for preferred terms of PSC.
Partial Mayo score only reported for those with active IBD at Baseline
BMI = Body Mass Index;
IBD = inflammatory bowel diseases;
NRS = numerical Rating scale;
SD = Standard deviation
*Two participants (80 mg and 160 mg) were dispensed incorrect number of tablets and provided incorrect dosing instructions for the full treatment period due to an error at a single site. The participants' daily dose corresponded to a ≤40 mg dose. These 2 participants are grouped in the 40 mg dose group for all summaries.

Baseline disease activity markers for the study of Example B-3 are shown in Table A-2.

TABLE A-2

| | | | | |
|---|---|---|---|---|
| Bexotegrast | Bexotegrast | Bexotegrast | Bexotegrast | |
| 40 mg | 80 mg | 160 mg | All | Placebo |
| (n = 24) | (n = 20) | (n = 20) | (n = 64) | (n = 21) |

Serum Liver
tests, mean (SD)

| Alkaline | 315.1 | (140.26) | 199.2 | (81.03) | 273.8 | (165.63) | 266.0 | (140.68) | 259.7 | (185.76) |
|---|---|---|---|---|---|---|---|---|---|---|
| phosphatase (U/L) | | | | | | | | | | |
| Alanine | 91.5 | (62.08) | 67.6 | (63.15) | 98.4 | (73.11) | 86.2 | (66.25) | 67.5 | (49.19) |
| aminotransferase | | | | | | | | | | |
| (U/L) | | | | | | | | | | |
| Aspartate | 67.2 | (49.34) | 46.4 | (30.12) | 69.0 | (39.62) | 61.3 | (41 70) | 48.8 | (30.57) |
| aminotransferase | | | | | | | | | | |
| (U/L) | | | | | | | | | | |
| Total Bilirubin | 0.66 | (0.307) | 0.79 | (0.493) | 0.88 | (0.396) | 0.77 | (0.405) | 0.84 | (0.357) |
| (mg/dL) | | | | | | | | | | |
| Direct bilirubin | 0.27 | (0.164) | 0.26 | (0.188) | 0.31 | (0.166) | 0.28 | (0.171) | 0.30 | (0.189) |
| (mg/dL) | | | | | | | | | | |
| Markers of Fibrosis, | | | | | | | | | | |
| mean (SD) | | | | | | | | | | |
| ELF Score | 9.6 | (0.77) | 9.2 | (1.01) | 9.4 | (0.79) | 9.4 | (0.86) | 9.2 | (1.08) |
| PRO-C3 (ng/mL) | 49.96 | (13.844) | 48.84 | (42.790) | 46.12 | (11.670) | 48.39 | (25.904) | 43.24 | (10.828) |
| Transient | 10.1 | (2.62) | 9.1 | (2.99) | 8.2 | (3.16) | 9.2 | (2.98) | 8.5 | (2.86) |
| Elastography (kPa) | | | | | | | | | | |

ELF: Enhanced Liver Fibrosis; PROC-C3: neo-epitope pro-peptide of type III collagen formation.
PRO-C3 quantified using Roche COBAS platform (assay reports approximately 2x higher concentrations than previous generation PRO-C3 ELISA)

A safety summary of adverse events of Example B-3 is shown in Table A-3.

TABLE A-3

| AE, n (%) of | Bexotegrast | | Bexotegrast | | Bexotegrast | | Bexotegrast | | | |
|---|---|---|---|---|---|---|---|---|---|---|
| Participants | 40 mg | | 80 mg | | 160 mg | | All | | Placebo | |
| Reporting | (n = 24) | | (n = 20) | | (n = 20) | | (n = 64) | | (n = 21) | |
| TEAE | 10 | (41.7) | 16 | (80.0) | 15 | (75.0) | 41 | (64.1) | 16 | (76.2) |
| Related to | 1 | (4.2) | 6 | (30.0) | 4 | (20.0) | 11 | (17.2) | 7 | (33.3) |
| study drug | | | | | | | | | | |
| Serious TEAE | 1 | (4.2) | 1 | (5.0) | 0 | | 2 | (3.1) | 0 | |
| Related to | 0 | | 0 | | 0 | | 0 | | 0 | |
| study drug | | | | | | | | | | |
| TEAE of CTCAE | 1 | (4.2) | 2 | (10.0) | 1 | (5.0) | 4 | (6.3) | 3 | (14.3) |
| Grade 3 or | | | | | | | | | | |
| Higher | | | | | | | | | | |
| Related to | 0 | | 0 | | 0 | | 0 | | 2 | (9.5) |
| study drug | | | | | | | | | | |
| TEAE Leading to | 1 | (4.2)¹ | 0 | | 0 | | 1 | (1.6)¹ | 0 | |
| Interruption | | | | | | | | | | |
| of Study Drug | | | | | | | | | | |
| TEAE Leading to | 1 | (4.2)² | 1 | (5.0)³ | 1 | (5.0)⁴ | 3 | (4.7)^{2, 3, 4} | 2 | (9.5)^{5, 6} |
| Withdrawal of | | | | | | | | | | |
| Study Drug | | | | | | | | | | |

| | | | | | | | |
|---|---|---|---|---|---|---|---|
| TEAE Leading to | 0 | 0 | 1 | (5.0)⁴ | 1 | (1.6)¹ | 0 |
| Early Termination | | | | | | | |
| from Study | | | | | | | |

| | | | | | |
|---|---|---|---|---|---|
| TEAE Leading | 0 | 0 | 0 | 0 | 0 |
| to Death | | | | | |

¹chills/constipation/fatigue/nausea/pyrexia/vomiting;
²COVID-19/dyspnoea/nasal congestion;
³Hepatic enzyme increase/Pruritus;
⁴Fatigue;
⁵fatigue; cough; oropharyngeal pain; increased ALT;
⁶increased ALP, ALT and AST;
⁷abdominal pain upper/fatigue/ocular icterus/pruritus;
⁸cardiomegaly/dyspnoea/malaise; headache
AE = Adverse Event; TEAE = Treatment Emergent Adverse Event; SAE = Serious Adverse Events. Adverse events coded using MedDRA v. 24.0.
TEAE is defined as any AE starting (or worsening) on or after the date of first dose.

The total number of subjects enrolled in Parts 1 and 2 and treated for 12 weeks was approximately 84, with approximately 63 subjects receiving(S)-4-((2-methoxyethyl)(4-(5,6,7,8-tetrahydro-1,8-naphthyridin-2-yl)butyl)amino)-2-(quinazolin-4-ylamino)butanoic acid and 21 subjects receiving placebo. Approximately 28 additional subjects will be enrolled in Part 3, with approximately 21 subjects receiving(S)-4-((2-methoxyethyl)(4-(5,6,7,8-tetrahydro-1,8-naphthyridin-2-yl)butyl)amino)-2-(quinazolin-4-ylamino)butanoic acid and 7 subjects receiving placebo.

(S)-4-((2-methoxyethyl)(4-(5,6,7,8-tetrahydro-1,8-naphthyridin-2-yl)butyl)amino)-2-(quinazolin-4-ylamino)butanoic acid was administered on Day 1, at Weeks 4 and 12(Parts 1 and 2), and additionally will be administered at Week 12 and Week 24 in Part 3. Subjects self-administer(S)-4-((2-methoxyethyl)(4-(5,6,7,8-tetrahydro-1,8-naphthyridin-2-yl)butyl)amino)-2-(quinazolin-4-ylamino)butanoic acid on an outpatient basis on all other days. In Parts 1 and 2, subjects returned to the study site for on-treatment evaluations on Day 1 and at Weeks 2, 4, 8, and 12, as shown in Table B-4. In Part 3, subjects will return to the study site for on-treatment evaluations on Day 1 and at Weeks 2, 4, 8, 12, 18, 24, and every 8 weeks after Week 24 until the last subject enrolled in Part 3 completes this Week 24 visit.

TABLE B-4
Schedule of Events: Part 1 and Part 2
Schedule of Events - Protocol: Parts 1 and 2

| | | Treatment | | | | | EoS/ET |
|---|---|---|---|---|---|---|---|
| Screening | | | Visit 4 | Visit 5 | Visit 6 | Visit 7 | Visit 8 |
| Visit 1 | Visit 2 | | Week | Week | Week | Week | Week |

| | | | | | | | |
|---|---|---|---|---|---|---|---|
| | (Visit 2 separated by ≥2 | Visit 3 | 2 | 4 | 8 | 12 | 16 |
| | weeks from Visit 1) | Baseline | Day | Day | Day | Day | Day |
| Evaluation | Day −42 to −1 | Day 1 | 14 ± 2 | 28 ± 3 | 56 ± 3 | 84 ± 3 | 112 ± 3 |

Entrance and General Assessments

| | | | | | | |
|---|---|---|---|---|---|---|
| Informed consent | X | | | | | |
| Inclusion/exclusion criteria | X | X | | | | |
| Randomization | | X | | | | |
| Demographic information | X | | | | | |
| Medical history | X | | | | | |
| Height and weight | X | | | | | |
| Urine drug screen | X | | | | | |
| Urine pregnancy test | X | X | X | X | X | X |
| (women of childbearing | | | | | | |
| potential)^{a} | | | | | | |
| FSH test (women only) | X | | | | | |
| ELF Test | X | | | | | |
| Serology (HbsAg, HCVAb, | X | | | | | |
| HIV) | | | | | | |
| Concomitant medications | | | | | | |

Safety Assessments

| | | | | | | | | |
|---|---|---|---|---|---|---|---|---|
| Complete physical | X | | | | | | | |
| examination | | | | | | | | |
| Targeted physical | | | X | X | X | X | X | X |
| examination | | | | | | | | |
| Vital signs (blood pressure, | X | | X | X | X | X | X | X |
| pulse rate, temperature) | | | | | | | | |
| Triplicate 12-lead ECG^{g} | X | | X | X | X | X | X | X |
| Hematology^{b} | X | | X | X | X | X | X | X |
| Coagulation (INR only)^{b} | X | | X | X | X | X | X | X |
| Serum chemistry^{b} | X | X | X | X | X | X | X | X |
| Urinalysis (macro panel)^{b, c} | X | | X | X | X | X | X | X |
| Adverse events (AEs) | | | | | | | | |

Pharmacokinetic Assessments

| | | | | |
|---|---|---|---|---|
| Plasma sample × 2 (predose | X | X | X | X^{f} |
| and at least 2 hours | | | | |
| postdose)^{d, e} | | | | |

Pharmacodynamic Assessments

| | | | | | | | | |
|---|---|---|---|---|---|---|---|---|
| MR-based liver imaging^{h} | | X | | | | | X | |
| FibroScan ® (to be done after | X | | | | | | X | |
| at least a 4 hour fast) | | | | | | | | |
| Patient-Reported Outcomes | | | X | | X | | X | |
| (PROs)ⁱ | | | | | | | | |
| Plasma biomarker sample | | | X | | X | X | X | X |
| Serum biomarker sample | | | X | X | X | X | X | X |
| Urine biomarker sample | | | X | | X | | X | |

Pharmacogenomics

| | |
|---|---|
| Whole blood PaxGene | X |
| sample | |

(S)-4-((2-methoxyethyl)(4-(5,6,7,8-tetrahydro-1,8-naphthyridin-2-yl)butyl)amino)-2-(quinazolin-4-
ylamino)butanoic acid Administration and Compliance

| | | | | |
|---|---|---|---|---|
| (S)-4-((2-methoxyethyl)(4- | X | X | | X |
| (5,6,7,8-tetrahydro-1,8- | | | | |
| naphthyridin-2- | | | | |
| yl)butyl)amino)-2- | | | | |
| (quinazolin-4- | | | | |
| ylamino)butanoic acid | | | | |
| administration onsite | | | | |
| Dispense (S)-4-((2- | X | X | X | |
| methoxyethyl)(4-(5,6,7,8- | | | | |
| tetrahydro-1,8-naphthyridin- | | | | |
| 2-yl)butyl)amino)-2- | | | | |
| (quinazolin-4- | | | | |
| ylamino)butanoic acid | | | | |
| Treatment compliance | | X | X | X |

ECG = electrocardiogram,
EoS = end of study,
ET = early termination,
FSH = follicle-stimulating hormone,
HbsAg = hepatitis B surface antigen,
HCVAb = hepatitis C virus antibody,
HIV = human immunodeficiency virus,
INR = international normalized ratio,
PK = pharmacokinetics
^{a}Positive urine pregnancy tests will be confirmed with a serum pregnancy test.
^{b}Samples will be collected after at least an 8-hour fast.
^{c}A microscopic examination will be done if the results of the macro panel analysis are abnormal.
^{d}In addition to the specified time points, a PK sample must be obtained for any subject who experiences elevations in liver-related tests that meet the thresholds or ECG abnormality.
^{e}The actual time of sample collection and dosing will be recorded. Subjects will record the time of their last meal before taking their dose of (S)-4-((2-methoxyethyl)(4-(5,6,7,8-tetrahydro-1,8-naphthyridin-2-yl)butyl)amino)-2-(quinazolin-4-ylamino)butanoic acid and the time of their first meal after taking their dose of (S)-4-((2-methoxyethyl)(4-(5,6,7,8-tetrahydro-1,8-naphthyridin-2-yl)butyl)amino)-2-(quinazolin-4-ylamino)butanoic acid in their dosing diary.
^{f}A plasma PK sample should be obtained at early termination, if possible, for any subject who prematurely discontinues from the study. A plasma PK sample is not required at EoS.
^{g}Subjects should be supinely resting in an undisturbed environment at least 10 minutes prior to the ECG collection. Triplicate ECGs should be recorded within ~5 to 10 minutes. The triplicate ECGs will be averaged, and the mean values per time point (average of the 3 assessments) used to interpret the ECG tracings. When time points for ECG collection, vital sign assessments, and blood draws coincide, procedures should be carried out with blood draws last. ECG will be collected prior to a time-matched PK sample at least 2 hours postdose at Baseline, Weeks 4 and 12.
^{h}Optional and may be completed after Visit 1 for eligible subjects only. Only for subjects who have completed an MRI scan at Visit 2.
ⁱPROs will be administered at Baseline, Weeks 4, and 12 and include the Chronic Liver Disease Questionnaire (CLDQ), the Itch Numeric Rating Scale (NRS), and the Partial Mayo Score (PMS), when available for use.

At Week 12, all subjects returned and completed the EOT visit, as shown in Table B-5 and Table B-6. The Schedule of Events for Part 3 is presented in two sections: Screening through the Week 12 Visit (Table B-5) and the Week 18 Visit through End of Study (Table B-6).

TABLE B-5
Schedule of Events for Part 3: Screening through the Week 12 Visit
Schedule of Events - Protocol Part 3 (Screening through Week 12 Visit)

| Screening | | Treatment (continued) | | | | |
|---|---|---|---|---|---|---|
| Visit 1 | Visit 2 | | Visit 4 | Visit 5 | Visit 6 | Visit 7 |

| | | | | | | |
|---|---|---|---|---|---|---|
| | (Visit 2 separated | Visit 3 | Week | Week | Week | Week |
| | by ≥2 weeks from | Base- | 2 | 4 | 8 | 12 |
| | Visit 1) | line | Day | Day | Day | Day |
| Evaluation | Day −42 to −1 | Day 1 | 14 ± 2 | 28 ± 3 | 56 ± 3 | 84 ± 3 |

Entrance and General Assessments

| | | | | | |
|---|---|---|---|---|---|
| Informed consent | X | | | | |
| Inclusion/exclusion criteria | X | X | | | |
| Randomization | | X | | | |
| Demographic information | X | | | | |
| Medical history | X | | | | |
| Height and weight | X | | | | |
| Urine drug screen | X | | | | |
| Urine pregnancy test (women of | X | X | X | X | X |
| childbearing potential)^{a} | | | | | |
| FSH test (women only) | X | | | | |
| ELF Test | X | | | | |
| Serology (HbsAg, HCVAb, HIV) | X | | | | |
| Concomitant medications | | | | | |

Safety Assessments

| | | | | | | | |
|---|---|---|---|---|---|---|---|
| Complete physical examination | X | | | | | | |
| Targeted physical examination | | | X | X | X | X | X |
| Vital signs (blood pressure, pulse rate, | X | | X | X | X | X | X |
| temperature) | | | | | | | |
| Triplicate 12-lead ECG^{f} | X | | X | X | X | X | X |
| Hematology^{b} | X | | X | X | X | X | X |
| Coagulation (INR only)^{b} | X | | X | X | X | X | X |
| Serum chemistry^{b} | X | X | X | X | X | X | X |
| Urinalysis (macro panel)^{b, c} | X | | X | X | X | X | X |
| Adverse events (AEs) | | | | | | | |

Pharmacokinetic Assessments

| | | | |
|---|---|---|---|
| Plasma sample × 2 (predose and at least | X | X | X |
| 2 hours postdose)^{d, e} | | | |

Pharmacodynamic Assessments

| | | | | | | | |
|---|---|---|---|---|---|---|---|
| MR-based liver imaging^{g} | | X | | | | | X |
| FibroScan ® (to be done after at least a 4 | X | | | | | | X |
| hour fast) | | | | | | | |
| Patient-Reported Outcomes (PROs)^{h} | | | X | | X | | X |
| Plasma biomarker sample | | | X | | X | X | X |
| Serum biomarker sample | | | X | X | X | X | X |

Pharmacogenomics

| | |
|---|---|
| Whole blood PaxGene sample | X |

(S)-4-((2-methoxyethyl)(4-(5,6,7,8-tetrahydro-1,8-naphthyridin-2-yl)butyl)amino)-2-(quinazolin-4-
ylamino)butanoic acid Administration and Compliance

| | | | | |
|---|---|---|---|---|
| (S)-4-((2-methoxyethyl)(4-(5,6,7,8- | X | X | | X |
| tetrahydro-1,8-naphthyridin-2- | | | | |
| yl)butyl)amino)-2-(quinazolin-4- | | | | |
| ylamino)butanoic acid administration | | | | |
| onsite | | | | |
| Dispense (S)-4-((2-methoxyethyl)(4- | X | X | X | |
| (5,6,7,8-tetrahydro-1,8-naphthyridin-2- | | | | |
| yl)butyl)amino)-2-(quinazolin-4- | | | | |
| ylamino)butanoic acid | | | | |
| Treatment compliance | | X | X | X |

ECG = electrocardiogram,
FSH = follicle-stimulating hormone,
HbsAg = hepatitis B surface antigen,
HCVAb = hepatitis C virus antibody,
HIV = human immunodeficiency virus,
INR = international normalized ratio,
PK = pharmacokinetics
^{a}Positive urine pregnancy tests will be confirmed with a serum pregnancy test.
^{b}Samples will be collected after at least an 8-hour fast.
^{c}A microscopic examination will be done if the results of the macro panel analysis are abnormal.
^{d}In addition to the specified time points, a PK sample must be obtained for any subject who experiences elevations in liver-related tests that meet the thresholds or ECG abnormality.
^{e}The actual time of sample collection and dosing will be recorded. Subjects will record the time of their last meal before taking their dose of (S)-4-((2-methoxyethyl)(4-(5,6,7,8-tetrahydro-1,8-naphthyridin-2-yl)butyl)amino)-2-(quinazolin-4-ylamino)butanoic acid and the time of their first meal after taking their dose of (S)-4-((2-methoxyethyl)(4-(5,6,7,8-tetrahydro-1,8-naphthyridin-2-yl)butyl)amino)-2-(quinazolin-4-ylamino)butanoic acid in their dosing diary.
^{f}Subjects should be supinely resting in an undisturbed environment at least 10 minutes prior to the ECG collection. Triplicate ECGs should be recorded within ~5 to 10 minutes. The triplicate ECGs will be averaged, and the mean values per time point (average of the 3 assessments) used to interpret the ECG tracings. When time points for ECG collection, vital sign assessments, and blood draws coincide, procedures should be carried out with blood draws last. ECG will be collected prior to a time-matched PK sample at least 2 hours postdose at Baseline, Weeks 4 and 12.
^{g}Optional and may be completed after Visit 1 for eligible subjects only. Only for subjects who have completed an MRI scan at Visit 2.
^{h}PROs will include the Chronic Liver Disease Questionnaire (CLDQ), the Itch Numeric Rating Scale (NRS), and the Partial Mayo Score (PMS), when available for use.

TABLE B-6
Schedule of Events for Part 3: Week 18 Visit through End of Study
Schedule of Events - Protocol Part 3 (Week 18 Visit through End of Study)

| | Treatment | | | | | |
|---|---|---|---|---|---|---|
| | | | | | EoT^{a} | EoS/ET |
| | | | | | Week 48/ | EoS |
| | | | | | Unscheduled/ | End of Study |
| | | | | | End of | Visit |
| | Visit 8 | Visit 9 | Visit 10^{a} | Visit 11^{a} | Treatment | EoT Visit ±4 |
| continued | Week 18 | Week 24 | Week 32 | Week 40 | Visit | weeks |
| Evaluation | Day 126 ± 3 | Day 168 ± 3 | Day 224 ± 5 | Day 280 ± 5 | EoT Visit | (±3 days) |

General Assessments

| | | | | | | |
|---|---|---|---|---|---|---|
| Urine pregnancy | X | X | X | X | X | X |
| test (women of | | | | | | |
| childbearing | | | | | | |
| potential)^{b} | | | | | | |
| Concomitant | | | | | | |
| medications | | | | | | |

Safety Assessments

| | | | | | | |
|---|---|---|---|---|---|---|
| Targeted physical | X | X | X | X | X | X |
| examination | | | | | | |
| Vital signs (blood | X | X | X | X | X | X |
| pressure, pulse | | | | | | |
| rate, temperature) | | | | | | |
| Triplicate 12-lead | X | X | X | X | X | X |
| ECG^{h} | | | | | | |
| Hematology^{c} | X | X | X | X | X | X |
| Coagulation (INR | X | X | X | X | X | X |
| only)^{c} | | | | | | |
| Serum chemistry^{c} | X | X | X | X | X | X |
| Urinalysis (macro | X | X | X | X | X | X |
| panel)^{c, d} | | | | | | |
| Adverse events | | | | | | |
| (AEs) | | | | | | |

Pharmacokinetic Assessments

| | | |
|---|---|---|
| Plasma sample × 2 | X | X^{g} |
| (predose and at | | |
| least 2 hours | | |
| postdose)^{e, f} | | |

Pharmacodynamic Assessments

| | | | |
|---|---|---|---|
| MR-based liver | X | | |
| imagingⁱ | | | |
| FibroScan ® (to be | X | | |
| done after at least a | | | |
| 4 hour fast) | | | |
| Patient-Reported | X | | |
| Outcomes (PROs)^{j} | | | |
| Plasma biomarker | X | X | X |
| sample | | | |
| Serum biomarker | X | X | X |
| sample | | | |

(S)-4-((2-methoxyethyl)(4-(5,6,7,8-tetrahydro-1,8-naphthyridin-2-yl)butyl)amino)-2-(quinazolin-4-
ylamino)butanoic acid Administration and Compliance

| | | | | | |
|---|---|---|---|---|---|
| (S)-4-((2- | | X | | | |
| methoxyethyl)(4- | | | | | |
| (5,6,7,8-tetrahydro- | | | | | |
| 1,8-naphthyridin-2- | | | | | |
| yl)butyl)amino)-2- | | | | | |
| (quinazolin-4- | | | | | |
| ylamino)butanoic | | | | | |
| acid | | | | | |
| administration | | | | | |
| onsite | | | | | |
| Dispense (S)-4-((2- | X | X | X | X | |
| methoxyethyl)(4- | | | | | |
| (5,6,7,8-tetrahydro- | | | | | |
| 1,8-naphthyridin-2- | | | | | |
| yl)butyl)amino)-2- | | | | | |
| (quinazolin-4- | | | | | |
| ylamino)butanoic | | | | | |
| acid | | | | | |
| Treatment | X | X | X | X | X |
| compliance | | | | | |

ECG = electrocardiogram,
EoS = end of study,
EoT = end of treatment,
ET = early termination,
INR = international normalized ratio,
PK = pharmacokinetics
^{a}All subjects will complete visits every 8 weeks after the Week 24 visit until such time as the last subject enrolled has completed the Week 24 visit. At this time, all subjects will be contacted to return and complete the EoT Visit.
^{b}Positive urine pregnancy tests will be confirmed with a serum pregnancy test.
^{c}Samples will be collected after at least an 8-hour fast.
^{d}A microscopic examination will be done if the results of the macro panel analysis are abnormal.
^{e}In addition to the specified time points, a PK sample must be obtained for any subject who experiences elevations in liver-related tests that meet the thresholds or ECG abnormality.
^{f}The actual time of sample collection and dosing will be recorded. Subjects will record the time of their last meal before taking their dose of (S)-4-((2-methoxyethyl)(4-(5,6,7,8-tetrahydro-1,8-naphthyridin-2-yl)butyl)amino)-2-(quinazolin-4-ylamino)butanoic acid and the time of their first meal after taking their dose of (S)-4-((2-methoxyethyl)(4-(5,6,7,8-tetrahydro-1,8-naphthyridin-2-yl)butyl)amino)-2-(quinazolin-4-ylamino)butanoic acid in their dosing diary.
^{g}A plasma PK sample should be obtained at early termination, if possible, for any subject who prematurely discontinues from the study. A plasma PK sample is not required at EoS.
^{h}Subjects should be supinely resting in an undisturbed environment at least 10 minutes prior to the ECG collection. Triplicate ECGs should be recorded within ~5 to 10 minutes. The triplicate ECGs will be averaged, and the mean values per time point (average of the 3 assessments) used to interpret the ECG tracings. When time points for ECG collection, vital sign assessments, and blood draws coincide, procedures should be carried out with blood draws last. ECG will be collected prior to a time-matched PK sample at least 2 hours postdose at Baseline, Weeks 4, 12, and 24.
ⁱOptional and may be completed after Visit 1 for eligible subjects only. Only for subjects who have completed an MRI scan at Visit 2.
^{j}PROs will include the Chronic Liver Disease Questionnaire (CLDQ), the Itch Numeric Rating Scale (NRS), and the Partial Mayo Score (PMS), when available for use.

The maximum treatment duration for the study will be 48 weeks. Blood and urine specimens for safety laboratory assessments is collected after at least an 8 hour fast. A final study visit is conducted 4 weeks after the last dose of (S)-4-((2-methoxyethyl)(4-(5,6,7,8-tetrahydro-1,8-naphthyridin-2-yl)butyl)amino)-2-(quinazolin-4-ylamino)butanoic acid is administered to the subject. Subjects who discontinued(S)-4-((2-methoxyethyl)(4-(5,6,7,8-tetrahydro-1,8-naphthyridin-2-yl)butyl)amino)-2-(quinazolin-4-ylamino)butanoic acid for safety reasons prior to completion of 12 weeks (Parts 1 and 2) or at least 24 weeks (Part 3) of treatment will be asked to remain in the study to complete all remaining assessments.

The DSMB will assess subject safety at predetermined intervals during the study, including prior to initiating Part 3, following the enrollment of the last subject in Part 3, and as needed. Potential cases of serious liver-related adverse events (AEs) and laboratory abnormalities and/or cases of suspected drug induced liver injury (DILI) were assessed.

### Study Population

Approximately 112 subjects with PSC who met the following eligibility criteria were enrolled in the study.

### Inclusion Criteria

1. Aged 18 to 75 years, inclusive.

2. Female subjects of childbearing potential that had used a contraceptive method with a failure rate of <1% per year (including bilateral tubal ligation, hormonal contraceptives that inhibit ovulation (initiated >3 months from Day 1), hormone-releasing intrauterine devices (initiated >3 months from Day 1), or copper intrauterine devices (initiated >3 months from Day 1)) or remain abstinent (refrain from heterosexual intercourse) during the treatment period and for 1 month after the last dose of (S)-4-((2-methoxyethyl)(4-(5,6,7,8-tetrahydro-1,8-naphthyridin-2-yl)butyl)amino)-2-(quinazolin-4-ylamino)butanoic acid. Male subjects with female partners of childbearing potential that had used contraceptive measures or remain abstinent (refrain from heterosexual intercourse) during screening and the treatment period and for at least 3 months after the last dose of (S)-4-((2-methoxyethyl)(4-(5,6,7,8-tetrahydro-1,8-naphthyridin-2-yl)butyl)amino)-2-(quinazolin-4-ylamino)butanoic acid.

3. Female subjects of nonchildbearing potential that were surgically sterile or postmenopausal.

4. Subjects agree to abstain from sperm or egg donation for the duration of the study, through 3 months or 1 month, respectively, and after administration of the last dose of (S)-4-((2-methoxyethyl)(4-(5,6,7,8-tetrahydro-1,8-naphthyridin-2-yl)butyl)amino)-2-(quinazolin-4-ylamino)butanoic acid.

5. Able to understand the purpose and procedures that were involved in the study and are willing to sign a written informed consent form.

### Primary Sclerosing Cholangitis Diagnosis

1. Established clinical diagnosis of large duct PSC based on an abnormal cholangiography as assessed by magnetic resonance cholangiopancreatography (MRCP), endoscopic retrograde cholangiopancreatography (ERCP), and/or percutaneous transhepatic cholangiopancreatography (PTC) in the context of elevated cholestatic liver chemistries.

2. Serum alkaline phosphatase concentration within normal ranges or >1× the upper limit of normal (ULN).

3. Serum aspartate aminotransferase (AST) and serum alanine aminotransferase (ALT) concentration≤5×ULN.

4. Serum total bilirubin≤1.5×ULN, in the absence of hemolysis. Subjects with serum total bilirubin >1.5×ULN were enrolled if they had Gilbert's Syndrome and a direct bilirubin≤0.6 mg/dL. In subjects with Gilbert's syndrome, stable direct bilirubin during the screening period was confirmed by measurements >2 weeks apart with a ≤30% increase observed with the follow-up value. This was only applied to values that were outside the normal range at Screening Visit 2.

5. Suspected liver fibrosis, as defined by any of the following:
Liver stiffness measurement (LSM)≥8 kPa but≤14.4 kPa, assessed by FibroScan®, or
Enhanced Liver Fibrosis (ELF) Score ≥7.7 at Screening, or
Historical liver biopsy showing fibrosis without cirrhosis (by any scoring system), or
Magnetic resonance elastography (MRE)≥2.4 kPa but≤4.9 kPa.

6. Platelet count ≥140,000/mm³.

7. Albumin ≥3.3 g/dL.

8. International normalized ratio (INR)≤1.3 in the absence of anticoagulant therapy.

9. Serum carbohydrate antigen 19-9 (CA19-9) value≤130 U/mL.

### Allowed Prior and Concomitant Medications

1. Prior and concomitant use of the following is allowed:
Biologics (including TNF-α inhibitors and/or vedolizumab), immunosuppressive agents, or corticosteroids for the treatment of IBD, if the dose was stable for at least 3 months before screening and was expected to remain stable from screening through the end of study visit.
UDCA at a dose of <25 mg/kg/day, at a stable dose for at least 3 months before screening and is expected to remain stable from screening through the end of study visit.
Supportive therapies for PSC (e.g., antibiotics, antihistamines) or for comorbid conditions (e.g., depression, anxiety), unless the therapy is included among the prohibited classes of medications.

### Medical History and Comorbid Conditions

1. Subjects with IBD must have had a colonoscopy showing no evidence of dysplasia within no more than 18 months before screening.

2. Subjects with IBD must have no evidence of active disease and a partial Mayo score of <2, with a score of <1 on the Rectal Bleeding domain, between screening through Day 1.

3. Subjects with IBD who received treatment with biologics, including tumor necrosis factor-alpha (TNF-α) inhibitors and/or vedolizumab, immunosuppressive agents, or corticosteroids must have been receiving a stable dose for at least 3 months before screening. The dose must remain stable from screening through Day 1 (baseline) and must be expected to remain stable for the duration of the study.

4. Estimated glomerular filtration rate ≥60 mL/min, according to the Cockcroft-Gault equation.

### Exclusion Criteria

### Primary Sclerosing Cholangitis Diagnosis

1. Other causes of liver disease, including secondary sclerosing cholangitis or viral, metabolic, or alcoholic liver disease, as assessed clinically.

2. Known or suspected overlapping clinical and histologic diagnosis of autoimmune hepatitis.

3. Small duct PSC with no evidence of large duct involvement (evidence of PSC on historical liver histology, with normal bile ducts on cholangiography).

### Liver Disease Status

1. Presence of a clinically significant dominant stricture based on the combination of radiological, biochemical, and clinical features.

2. Presence of a percutaneous drain or bile duct stent.

3. Serum alkaline phosphatase (ALP) concentration >10 times ULN.

4. Worsening of liver disease, defined as 2 consecutive ALP, ALT, or AST measurements obtained >2 weeks apart during the screening period that increase by >30%. This will only be applied to values that were outside of the normal range at Screening Visit 2.

5. Ascending cholangitis within 60 days of screening, as assessed clinically or use of antibiotics for acute cholangitis within 60 days of screening.

6. IgG4-associated cholangitis.

7. Positive anti-mitochondrial antibody.

8. Presence of liver cirrhosis as assessed by historical liver histology, ultrasound-based liver stiffness measurement (FibroScan® value >14.4 kPa), MRE >4.9 kPa, and/or signs and symptoms of hepatic decompensation (including, but not limited to, jaundice, ascites, variceal hemorrhage, and/or hepatic encephalopathy).

9. Presence of hepatic impairment, end-stage liver disease, and/or a model for end-stage liver disease (MELD) score ≥15.

10. Prior or planned liver transplantation during the study.

Medical History and Comorbid Conditions 1. Presence of end-stage renal disease that required dialysis.

2. History, current clinical or radiological suspicion, or diagnosis of cholangiocarcinoma, other hepatobiliary malignancy, colorectal cancer, or other abdominal malignancy at any time.

3. Human immunodeficiency virus (HIV), hepatitis A virus, hepatitis B virus, and/or hepatitis C virus infection, with the exception of those who had been successfully treated for hepatitis C infection and had achieved sustained virologic response for ≥1 year.

4. History of malignancy within the past 5 years or ongoing malignancy other than basal cell carcinoma, resected noninvasive cutaneous squamous cell carcinoma, or treated cervical carcinoma in situ.

5. Clinical evidence of active bacterial, viral, or fungal infection within 30 days before screening.

6. History of unstable or deteriorating cardiac disease within the previous 6 months, including, but not limited to:
a) Unstable angina pectoris or myocardial infarction,
b) Congestive heart failure requiring hospitalization,
c) Uncontrolled clinically significant arrhythmias, and/or
d) Clinically significant electrocardiogram (ECG) abnormalities, including but not limited to, QT interval corrected for heart rate using Fridericia's formula (QTcF)>450 msec for males or >460 msec for females at Screening Visit 1 or prior to administration of the initial dose of (S)-4-((2-methoxyethyl)(4-(5,6,7,8-tetrahydro-1,8-naphthyridin-2-yl)butyl)amino)- 2-(quinazolin-4-ylamino)butanoic acid.

7. Surgery within the 4 weeks before administration of (S)-4-((2-methoxyethyl)(4-(5,6,7,8-tetrahydro-1,8-naphthyridin-2-yl)butyl)amino)-2-(quinazolin-4-ylamino)butanoic acid.

### Prior and Concomitant Medications

1. Use of any of the following will be prohibited:
a) Rifampin (a potent inducer of CYP3A4) as it may result in reduced(S)-4-((2-methoxyethyl)(4-(5,6,7,8-tetrahydro-1,8-naphthyridin-2-yl)butyl)amino)-2-(quinazolin-4-ylamino)butanoic acid concentrations,
b) Grapefruit or grapefruit-containing foods and beverage (potent inhibitors of CYP3A4) that may increase the concentrations of (S)-4-((2-methoxyethyl)(4-(5,6,7,8-tetrahydro-1,8-naphthyridin-2-yl)butyl)amino)-2-(quinazolin-4-ylamino)butanoic acid, and
c) St. John's Wort extract (a potent inducer of CYP3A4) that may decrease the concentrations of (S)-4-((2-methoxyethyl)(4-(5,6,7,8-tetrahydro-1,8-naphthyridin-2-yl)butyl)amino)-2-(quinazolin-4-ylamino)butanoic acid.
Potent (i.e., strong) inhibitors or inducers BCRP or OATP1B1/1B3 transporters.
Other investigational drugs, including those under evaluation for treatment of PSC (e.g., vancomycin, fibrates, or obeticholic acid).
Immunomodulating agents (such as interleukins and interferons).
Cytotoxic or chemotherapeutic agents or radiation therapy.
Treatment with a disallowed medication, other investigational drug within 5 half-lives or 30 days before screening, whichever time is longer, or the use of an investigational device within 30 days before screening is prohibited.

### Screening Assessments

1. Pregnancy or breastfeeding or male subject whose female partner is pregnant.

2. History of weekly alcohol consumption >21 units for male subjects or >14 units for female subjects (1 unit=1 oz/30 mL of alcohol contained in 12 oz/360 mL of beer, 4 oz/120 mL of wine, or 1 oz/30 mL of 40% proof alcohol).

3. Positive urine drug screen at screening (Table B-7) unless the positive result is due to a medical treatment for a comorbid condition.

4. Participation in an earlier part of the current study.

TABLE B-7
List of Laboratory tests

| Hematology | Serum Chemistry |
|---|---|
| Hematocrit (Hct) | Alanine aminotransferase (ALT) |
| Hemoglobin (Hgb) | Albumin |
| Mean corpuscular hemoglobin | Alkaline phosphatase (ALP) |
| (MCH) | Alpha-1-acid-glucoprotein (AAG) |
| Mean corpuscular hemoglobin | Aspartate aminotransferase (AST) |
| concentration (MCHC) | Bilirubin (total and direct) |
| Mean corpuscular volume (MCV) | Amylase (reflex lipase if amylase |
| Platelet count | ≥1.5 × upper limit of normal) |
| Red blood cell (RBC) count | Blood urea nitrogen (BUN) |
| White blood cell (WBC) count | Calcium |
| with differential (absolute | Chloride |
| counts only) | Creatinine |
| Basophils | Creatine kinase |
| Eosinophils | Gamma-glutamyl transferase (GGT) |
| Lymphocytes | Glucose |
| Monocytes | High-sensitivity C-reactive |
| Neutrophils | protein (hs-CRP) |
| | Lactate dehydrogenase (LDH) |
| | Phosphorus |
| | Potassium |
| | Sodium |
| | Total cholesterol |
| | Total protein |
| | Triglycerides |
| | Uric acid |
| Coagulation | Other |
| International normalized ratio | Urine pregnancy test - women of |
| (INR) | childbearing potential only |
| Prothrombin time (PT) | (confirm positive test with a |
| | serum test) |
| Urinalysis | Screening Only |
| Appearance | Anti-mitochondrial antibody |
| Bilirubin | CA-19-9 |
| Color | ELF Test |
| Glucose | Follicle-stimulating hormone (FSH) |
| Ketones | to confirm postmenopausal status |
| Nitrite | Hepatitis A IgM antibody (HAV-IgM Ab) |
| Occult blood | Hepatitis B surface antigen (HbsAg) |
| pH | Hepatitis C virus antibody (HCVAb) |
| Protein | Human immunodeficiency virus (HIV) |
| Specific gravity | Immunoglobulin G4 (IgG4) levels |
| Urobilinogen | Urine drug screen, including, but |
| Microscopic examination of | not limited to: |
| sediment (if macro panel | amphetamines |
| results are abnormal) | barbiturates |
| | benzodiazepines |
| | cocaine |
| | methadone |
| | phencyclidine |
| | opiates |

### Test Product, Dose, and Mode of Administration

Part 1 included orally administering 40 mg of (S)-4-((2-methoxyethyl)(4-(5,6,7,8-tetrahydro-1,8-naphthyridin-2-yl)butyl)amino)-2-(quinazolin-4-ylamino)butanoic acid immediate release tablet or matching placebo once daily to the subject for a time period of 12 weeks. Part 2 included orally administering 80 mg or 160 mg of (S)-4-((2-methoxyethyl)(4-(5,6,7,8-tetrahydro-1,8-naphthyridin-2-yl)butyl)amino)-2-(quinazolin-4-ylamino)butanoic acid immediate release tablet or matching placebo once daily to the subject for a time period of 12 weeks. Part 1 and Part 2 of the study used the zwitterion of (S)-4-((2-methoxyethyl)(4-(5,6,7,8-tetrahydro-1,8-naphthyridin-2-yl)butyl)amino)-2-(quinazolin-4-ylamino)butanoic acid.

Part 3 will include orally administering 320 mg of (S)-4-((2-methoxyethyl)(4-(5,6,7,8-tetrahydro-1,8-naphthyridin-2-yl)butyl)amino)-2-(quinazolin-4-ylamino)butanoic acid or matching placebo once daily to the subject for a time period of at least 24 weeks and up to a maximum of 48 weeks. In Part 3, 80 mg immediate release tablets of the phosphate form of (S)-4-((2-methoxyethyl)(4-(5,6,7,8-tetrahydro-1,8-naphthyridin-2-yl)butyl)amino)-2-(quinazolin-4-ylamino)butanoic acid will be used.

Doses of (S)-4-((2-methoxyethyl)(4-(5,6,7,8-tetrahydro-1,8-naphthyridin-2-yl)butyl)amino)-2-(quinazolin-4-ylamino)butanoic acid are taken at visits on which scheduled PK blood samples are obtained (Baseline, Weeks 4 and 12 in Parts 1 and 2; additionally at Week 12 and Week 24 in Part 3). All other doses are taken on an outpatient basis.

Subjects were instructed to take(S)-4-((2-methoxyethyl)(4-(5,6,7,8-tetrahydro-1,8-naphthyridin-2-yl)butyl)amino)-2-(quinazolin-4-ylamino)butanoic acid once daily at approximately the same time each day (at approximately 24-hour intervals). Dosing of (S)-4-((2-methoxyethyl)(4-(5,6,7,8-tetrahydro-1,8-naphthyridin-2-yl)butyl)amino)-2-(quinazolin-4-ylamino)butanoic acid solution in the fed state decreased Cₘₐₓ by 52% and area under the drug concentration-time curve from time zero extrapolated to infinity (AUC_{inf}) by 37%, relative to the fasted state; therefore, subjects were instructed to take the (S)-4-((2-methoxyethyl)(4-(5,6,7,8-tetrahydro-1,8-naphthyridin-2-yl)butyl)amino)-2-(quinazolin-4-ylamino)butanoic acid on an empty stomach (no food for 2 hours before or for 2 hours after taking the (S)-4-((2-methoxyethyl)(4-(5,6,7,8-tetrahydro-1,8-naphthyridin-2-yl)butyl)amino)-2-(quinazolin-4-ylamino)butanoic acid). Subjects were instructed to drink approximately 240 mL (˜1 cup) of water after swallowing the (S)-4-((2-methoxyethyl)(4-(5,6,7,8-tetrahydro-1,8-naphthyridin-2-yl)butyl)amino)-2-(quinazolin-4-ylamino)butanoic acid.

A dose was considered missed if the subject could not the dose within 12 hours of their regular dosing time. If the subject missed a dose of (S)-4-((2-methoxyethyl)(4-(5,6,7,8-tetrahydro-1,8-naphthyridin-2-yl)butyl)amino)-2-(quinazolin-4-ylamino)butanoic acid and was still within 12 hours of the time the dose was usually taken, the subject was instructed to take a dose of the missed drug as soon as possible and then continue the usual dosing schedule. If the subject missed a dose of (S)-4-((2-methoxyethyl)(4-(5,6,7,8-tetrahydro-1,8-naphthyridin-2-yl)butyl)amino)-2-(quinazolin-4-ylamino)butanoic acid more than 12 hours after the time it was usually taken, the subject was instructed not take the missed dose and was instructed to resume the usual dosing at the next scheduled time. The subject was instructed not to take a double dose to make up for a missed dose.

### Reference Therapy, Dose, and Mode of Administration

The placebo was a tablet identical in appearance to the (S)-4-((2-methoxyethyl)(4-(5,6,7,8-tetrahydro-1,8-naphthyridin-2-yl)butyl)amino)-2-(quinazolin-4-ylamino)butanoic acid tablet and was taken as described.

### Duration of Treatment

The duration of Parts 1 and 2 of the study are up to 22 weeks each (up to 6 weeks of screening, 12 weeks of treatment with(S)-4-((2-methoxyethyl)(4-(5,6,7,8-tetrahydro-1,8-naphthyridin-2-yl)butyl)amino)-2-(quinazolin-4-ylamino)butanoic acid, and 4 weeks of follow-up). The duration for Part 3 of the study will be variable depending on the study enrollment rate; i.e., the time between enrolling the first and last subject. The duration of participation will be up to approximately 34 weeks for the last subject enrolled (up to 6 weeks of screening, 24 weeks of treatment with the (S)-4-((2-methoxyethyl)(4-(5,6,7,8-tetrahydro-1,8-naphthyridin-2-yl)butyl)amino)-2-(quinazolin-4-ylamino)butanoic acid, and 4 weeks of follow up) and up to approximately 58 weeks for the first subject enrolled (up to 6 weeks of screening, up to 48 weeks of treatment with the (S)-4-((2-methoxyethyl)(4-(5,6,7,8-tetrahydro-1,8-naphthyridin-2-yl)butyl)amino)-2-(quinazolin-4-ylamino)butanoic acid, and 4 weeks of follow up). The end of Part 3 will commence once the last subject reached 24 weeks of treatment.

### Safety Assessments

Safety assessments included analyzing adverse events (AES), as determined by open ended questioning, laboratory parameters, vital sign measurements, safety electrocardiograms (ECGs), and physical examinations. (S)-4-((2-methoxyethyl)(4-(5,6,7,8-tetrahydro-1,8-naphthyridin-2-yl)butyl)amino)-2-(quinazolin-4-ylamino)butanoic acid was permanently discontinued in any subject who experienced confirmed elevations in liver enzymes meeting the criteria for suspected DILI or confirmed Grade ³ or Grade ⁴ QTc prolongation.

### Pharmacokinetic, Pharmacodynamic, and Pharmacogenomic Assessments

The pharmacokinetic, pharmacodynamic, and pharmacogenomic assessments included the following:
a) PK: plasma samples for PK analysis (total and unbound(S)-4-((2-methoxyethyl)(4-(5,6,7,8-tetrahydro-1,8-naphthyridin-2-yl)butyl)amino)-2-(quinazolin-4-ylamino)butanoic acid concentrations) were obtained.
b) Biomarkers: plasma, serum (Parts 1, 2, and 3), and urine (Parts 1 and 2) samples were obtained.
c) Pharmacogenomics: a whole blood sample for deoxyribonucleic acid (DNA) isolation was collected before dosing on Day 1 from each subject in the study.

### STUDY PROCEDURES

### Ultrasound-Based Transient Elastography (FibroScan®)

Each subject underwent an ultrasound-based transient elastography (FibroScan®) assessment at the screening and Week 12 study visits (Parts 1 and 2) and will undergo an ultrasound-based transient elastography (FibroScan®) assessment at the screening, at Week 12 study visits, and at Week 24 (Part 3 only). FibroScan® is a noninvasive technique that uses ultrasound-based transient elastography (TE) to assess liver stiffness measurement (LSM) and determine likelihood of clinically meaningful liver fibrosis. In the TE assessment, a controlled 50-Hz frequency shear wave was mechanically induced, and the propagation speed of the shear wave was measured with ultrasound.

The LSM, as assessed by TE, is expressed in kilopascals (kPa). Larger values (normal range approximately between 2 and 7 kPa) indicate a greater likelihood of clinically meaningful liver fibrosis. See H. Ehlken, et al. (2016) PloS ONE 11 (10): e0164224 and C. Corpechot, et al. (2014) Gastroenterology 146:970-979, the entire contents of which are hereby incorporated by reference in their entirety. Subjects with LSM values >8 kPa at screening were eligible for the study if all other eligibility criteria were met. Subjects with LSM values >14.4 kPa at screening were not eligible for the study due to the increased likelihood of having liver cirrhosis. See A. J. Muir, et al. (2019) Hepatology 69 (2): 684-699, the entire contents of which are hereby incorporated by reference in their entirety.

### Liver Imaging

Imaging of the whole liver was performed by MRI prior to Baseline and at Week 12 (Parts 1 and 2) and will be performed at Baseline, at Week 12, and at Week 24 (Part 3 only), and will be read centrally utilizing dynamic liver imaging in combination with the contrast agent Gadoxetate disodium and a 4-hour fast (no food or drink, except water). Any clinically significant incidental findings are evaluated as potential AEs.

### Patient-Reported Outcomes (PROs)

Questionnaires to assess PROs will be administered at Baseline, Weeks 4 and 12(Parts 1, 2, and 3) and at Week 24 (Part 3 only), and will include the Chronic Liver Disease Questionnaire (CLDQ), the Itch Numeric Rating Scale (NRS), and the Partial Mayo Score (PMS), when available for use. See A. N. Naegeli, et al. (2015) International Journal of Dermatology 54 (6): 715-722 and Z. M. Younossi, et al. (1999) Gut 45:295-300, the entire contents of which are hereby incorporated by reference in their entirety.

### Physical Examinations

A complete physical examination was performed at the screening visit, which included evaluating general appearance, including the head, the ears, the eyes, the nose, the throat, and the dentition; evaluating the thyroid; evaluating the chest (heart and lungs); evaluating the abdomen; evaluating the skin; conducting a neurological examination; evaluating the extremities; evaluating the back and neck; conducting a musculoskeletal examination; and evaluating the lymph nodes.

Targeted physical examinations were, or will be, performed at the time points specified in the Schedule of Events (see Table B-4, Table B-5, and Table B-6). A targeted physical examination was performed based on prior findings in the general examination, as well as evaluation of any pertinent system based on any previous findings.

### Vital Signs

Vital signs (blood pressure, pulse rate and body temperature (preferably ear)) were or will be recorded at each clinic visit. Blood pressure and pulse rate were or will be obtained after the subject had rested supine for at least 3 minutes.

### Electrocardiograms

Triplicate 12-lead ECGs were or will be obtained at each visit. ECG collection during the study is performed using standardized equipment and centrally read. ECGs are collected prior to a time-matched PK sample at least 2 hours post-dose at Baseline, Weeks 4 and 12(Parts 1, 2, and 3) and at Week 24 (Part 3 only). Additional 12-lead ECGs are obtained at other times using unscheduled visits, if clinically indicated. All ECGs are obtained after the subject had rested in the supine position for at least 10 minutes with the triplicate ECGs recorded within ˜ 5 to 10 minutes. The triplicate ECGs is averaged and the mean values per time point (average of the 3 assessments) are used to interpret the ECG tracings. QT correction utilizes the Fridericia Method, which is a formula for calculating the QT interval on an ECG according to hear rate. Clinically significant changes from baseline in ECG findings are recorded as AEs.

### Clinical Laboratory Parameters

Fasting blood and urine specimens for safety laboratory assessments were or will be obtained at the time points specified in the Schedule of Events (see Table B-4, Table B-5, and Table B-6). Specimens are obtained after at least an 8-hour fast. Subjects are in a seated or supine position during blood collection. The required clinical laboratory tests are listed in Table B-7. Creatinine clearance is calculated using the Cockcroft-Gault equation.

### Adverse Event (AE) Assessments

AEs are collected from the time the subjected signed the informed consent form until the last study visit. An AE is any event, side effect, or other untoward medical occurrence that occurs in conjunction with the use of a(S)-4-((2-methoxyethyl)(4-(5,6,7,8-tetrahydro-1,8-naphthyridin-2-yl)butyl)amino)-2-(quinazolin-4-ylamino)butanoic acid in humans, whether or not considered to have a causal relationship to this treatment. An AE can, therefore, be any unfavorable and unintended sign (that could include a clinically significant abnormal laboratory finding), symptom, or disease temporally associated with the use of a(S)-4-((2-methoxyethyl)(4-(5,6,7,8-tetrahydro-1,8-naphthyridin-2-yl)butyl)amino)-2-(quinazolin-4-ylamino)butanoic acid, whether or not considered related to the (S)-4-((2-methoxyethyl)(4-(5,6,7,8-tetrahydro-1,8-naphthyridin-2-yl)butyl)amino)-2-(quinazolin-4-ylamino)butanoic acid.

Events meeting the definition of an AE include:
a) Exacerbation of a chronic or intermittent pre-existing condition including either an increase in frequency and/or intensity of the condition.
b) New conditions detected or diagnosed after(S)-4-((2-methoxyethyl)(4-(5,6,7,8-tetrahydro-1,8-naphthyridin-2-yl)butyl)amino)-2-(quinazolin-4-ylamino)butanoic acid administration even though it may have been present prior to the start of the study.
c) Signs, symptoms, or the clinical sequelae of a suspected interaction.
d) Signs, symptoms, or the clinical sequelae of a suspected overdose of either(S)-4-((2-methoxyethyl)(4-(5,6,7,8-tetrahydro-1,8-naphthyridin-2-yl)butyl)amino)-2-(quinazolin-4-ylamino)butanoic acid or a concomitant medication (overdose per se will not be reported as an AE/SAE).

Events that do not meet the definition of an AE include:
a) Medical or surgical procedure (e.g., endoscopy, appendectomy); the condition that leads to the procedure is an AE.
b) Situations in which an untoward medical occurrence did not occur (e.g., social and/or convenience admission to a hospital).
c) Anticipated day-to-day fluctuations of preexisting disease(s) or condition(s) present or detected at the start of the study that do not worsen.

If there is evidence of an AE through report or observation, the following information is analyzed and recorded:
a) Time of onset and resolution
b) Severity
c) Causality/relation to study treatment
d) Action taken regarding(S)-4-((2-methoxyethyl)(4-(5,6,7,8-tetrahydro-1,8-naphthyridin-2-yl)butyl)amino)-2-(quinazolin-4-ylamino)butanoic acid
e) Outcome

Grading the severity of AEs occurred using the Common Terminology Criteria for Adverse Events (CTCAE) grading system, Version 5.0.
Grade ^{1a} Mild; asymptomatic or mild symptoms; clinical or diagnostic observations only; intervention not indicated
Grade ^{2a} Moderate; minimal, local or noninvasive intervention indicated; limiting age-appropriate instrumental ADL^{b}
Grade ^{3a} Severe or medically significant but not immediately life-threatening; hospitalization or prolongation of hospitalization indicated; disabling; limiting self-care ADL^{c}
Grade ^{4a} Life-threatening consequences; urgent intervention indicated
Grade ^{5a} Death related to AE
a. A semi-colon indicates ‘or’ within the description of the grade.
b. Instrumental activities of daily living (ADL) refer to preparing meals, shopping for groceries or clothes, using the telephone, managing money, etc.
c. Self-care ADL refer to bathing, dressing and undressing, feeding self, using the toilet, taking medications, and not bedridden.

The relationship between(S)-4-((2-methoxyethyl)(4-(5,6,7,8-tetrahydro-1,8-naphthyridin-2-yl)butyl)amino)-2-(quinazolin-4-ylamino)butanoic acid and the occurrence of each AE is analyzed. The relationship of each AE to(S)-4-((2-methoxyethyl)(4-(5,6,7,8-tetrahydro-1,8-naphthyridin-2-yl)butyl)amino)-2-(quinazolin-4-ylamino)butanoic acid is recorded. Alternative causes, such as medical history, concomitant therapy, or other risk factors, and the temporal relationship of the event to(S)-4-((2-methoxyethyl)(4-(5,6,7,8-tetrahydro-1,8-naphthyridin-2-yl)butyl)amino)-2-(quinazolin-4-ylamino)butanoic acid are considered and investigated, if appropriate. The relationship or association of the AE to(S)-4-((2-methoxyethyl)(4-(5,6,7,8-tetrahydro-1,8-naphthyridin-2-yl)butyl)amino)-2-(quinazolin-4-ylamino)butanoic acid is assessed using clinical judgment and the following considerations:
No (not related): Evidence exists that the adverse event has an etiology other than the (S)-4-((2-methoxyethyl)(4-(5,6,7,8-tetrahydro-1,8-naphthyridin-2-yl)butyl)amino)-2-(quinazolin-4-ylamino)butanoic acid. For SAEs, an alternative causality must be provided (e.g., preexisting condition, underlying disease, intercurrent illness, or concomitant medication).
Yes (related): A temporal relationship exists between the AE onset and administration of the (S)-4-((2-methoxyethyl)(4-(5,6,7,8-tetrahydro-1,8-naphthyridin-2-yl)butyl)amino)-2-(quinazolin-4-ylamino)butanoic acid that cannot be readily explained by the subject's clinical state or concomitant therapies. Furthermore, the AE appears with some degree of certainty to be related, based on the known therapeutic and pharmacologic actions or AE profile of the (S)-4-((2-methoxyethyl)(4-(5,6,7,8-tetrahydro-1,8-naphthyridin-2-yl)butyl)amino)-2-(quinazolin-4-ylamino)butanoic acid. In case of cessation or reduction of the dose, the AE abates or resolves and reappears upon re-challenge.

The relationship to study procedures (such as venipuncture) is assessed using the following considerations:
No (not related): Evidence exists that the AE has an etiology other than the study procedure.
Yes (related): The AE occurred as a result of protocol-mandated procedures.

The outcome of an AE is recorded as follows:
Recovered/Resolved
Recovering/Resolving
Recovered/Resolved with Sequelae
Not Recovered/Not Resolving
Fatal
Unknown

### Pregnancy

A pregnancy was not considered an AE. If the pregnancy resulted in a congenital anomaly/birth defects/miscarriage, this constitutes an SAE.

### Abnormal Laboratory Findings

Abnormal laboratory findings that are deemed clinically significant or that were associated with signs and/or symptoms are recorded as AEs if they meet the definition of an AE.

### Serious Adverse Event (SAE)

An SAE is any untoward medical occurrence that at any dose:
Is life-threatening (e.g., its occurrence places the subject at immediate risk of death),
Results in death,
Requires inpatient hospitalization (i.e., admission, overnight stay) or prolongs existing hospitalization,
Results in persistent or significant disability/incapacity,
Is a congenital anomaly/birth defect/miscarriage, or
Is an important medical event (e.g., is considered to be clinically significant and may jeopardize the subject, or when medical or surgical intervention may be required to prevent one of the outcomes listed above).

Table C-1 shows most frequent treatment emergent adverse events (TEAEs) for any causality associated with Example B-3. Table C-2 shows that no severe adverse events (SAEs) were related to(S)-4-((2-methoxyethyl)(4-(5,6,7,8-tetrahydro-1,8-naphthyridin-2-yl)butyl)amino)-2-(quinazolin-4-ylamino)butanoic acid for Example B-3. Table C-3 shows the TEAEs leading to withdrawal of (S)-4-((2-methoxyethyl)(4-(5,6,7,8-tetrahydro-1,8-naphthyridin-2-yl)butyl)amino)-2-(quinazolin-4-ylamino)butanoic acid in Example B-3.

TABLE C-1

| | | | | | |
|---|---|---|---|---|---|
| TEAE, n (%) of | Bexotegrast | Bexotegrast | Bexotegrast | Bexotegrast | |
| Participants | 40 mg | 80 mg | 160 mg | All | Placebo |
| Reporting | (n = 24) | (n = 20) | (n = 20) | (n = 64) | (n = 21) |

Most frequent
TEAEs
(n ≥ 3 in at
least one arm)

| | | | | | | | | | | |
|---|---|---|---|---|---|---|---|---|---|---|
| Pruritus¹ | 2 | (8.3) | 4 | (20.0) | 3 | (15.0) | 9 | (14.1) | 5 | (23.8) |
| Fatigue | 3 | (12.5) | 2 | (10.0) | 4 | (20.0) | 9 | (14.1) | 2 | (9.5) |
| Headache | 1 | (4.2) | 2 | (10.0) | 3 | (15.0) | 6 | (9.4) | 4 | (19.0) |

| | | | | | | | | | |
|---|---|---|---|---|---|---|---|---|---|
| Nausea | 1 | (4.2) | 2 | (10.0) | 3 | (15.0) | 6 | (9.4) | 0 |

| | | | | | | | | | |
|---|---|---|---|---|---|---|---|---|---|
| COVID-19 | 2 | (8.3) | 1 | (5.0) | 0 | 3 | (4.7) | 3 | (14.3) |

| Frequent bowel | 0 | 3 | (15.0) | 0 | | 3 | (4.7) | 3 | (14.3) |
|---|---|---|---|---|---|---|---|---|---|
| movements | | | | | | | | | |
| Cholangitis | 0 | 1 | (5.0) | 1 | (5.0) | 2 | (3.1) | 3 | (14.3) |

¹Pruritus includes preferred terms for pruritus and cholestatic pruritus
TEAE = Treatment Emergent Adverse Event; Adverse events coded using MedDRA version 24.0.
TEAE is defined as any AE starting (or worsening) on or after the date of first dose

TABLE C-2

| | | | | Any | | |
|---|---|---|---|---|---|---|
| | | | | alternative | | |
| | SAE | Standard | | cause or | | |
| Treatment | Preferred | Toxicity | Treatment | confounding | | |
| Group | Term | Grade | Related | factors? | Action Taken | Outcome |
| 40 mg | Cholecytitis/ | Grade 3 (all) | No | ERCP (post- | Hospitalization; | Recovered/ |
| | Abdominal | (Severe) | | procedure) | Event in | Resolved |
| | pain/ | | | | follow-up | |
| | Pancreatitis | | | | Period (3-4 | |
| | | | | | weeks post last | |
| | | | | | dose) | |
| 80 mg | Cholangitis | Grade 3 | No | No¹ | Hospitalization; | Recovered/ |
| | | (Severe) | | | Dose not | Resolved |
| | | | | | changed | |

¹Patient has medical history of cholangitis
SAE = Serious Adverse Events.
Adverse events coded using MedDRA version 24.0

TABLE C-3

| | | | | Any | | |
|---|---|---|---|---|---|---|
| | | | | alternative | | |
| | AE | Standard | | cause or | | |
| Treatment | Preferred | Toxicity | Treatment | confounding | Action | |
| Group | Term | Grade | Related | factors? | Taken | Outcome |
| 40 mg | COVID-19/ | Grade 1 (Mild) | No | COVID-19 | Drug | Recovered/ |
| | Nasal | | | | withdrawn | Resolved |
| | congestion I | | | | | |
| | Dyspnoea | | | | | |
| 80 mg | Hepatic | Grade 1 (Mild) | Yes | Variation in | Drug | Recovered/ |
| | enzyme | | | PSC/ | withdrawn | Resolved |
| | increased I | | | Aggravation of | | |
| | Pruritus | | | PSC | | |
| 160 mg | Fatigue | Grade 2 | Yes | No | Drug | Recovered/ |
| | | (Moderate) | | | withdrawn | Resolved |
| Placebo | Dyspnoea I | Grade 2 | Yes | No | Drug | Recovered/ |
| | Malaise/ | (Moderate)/ | | | withdrawn | Resolved |
| | Cardiomegaly | Grade 3 | | | | |
| | | (Severe)/ | | | | |
| | | Grade 1 (Mild) | | | | |
| Placebo | Headache | Grade 1 (Mild) | Yes | Fasting before | Drug | Recovered/ |
| | | | | drug | withdrawn | Resolved |
| | | | | administration | | |

TEAE = Treatment Emergent Adverse Even~Adverse events coded using MedDRA version 24.0.
TEAE is defined as any AE starting (or worsening) on or after the date of first dose

### Pharmacokinetic Assessments

Blood samples for determination of plasma concentrations of (S)-4-((2-methoxyethyl)(4-(5,6,7,8-tetrahydro-1,8-naphthyridin-2-yl)butyl)amino)-2-(quinazolin-4-ylamino)butanoic acid are obtained before and at least 2 hours after administration of (S)-4-((2-methoxyethyl)(4-(5,6,7,8-tetrahydro-1,8-naphthyridin-2-yl)butyl)amino)-2-(quinazolin-4-ylamino)butanoic acid on Day 1, at Weeks 4 and 12(Parts 1, 2, and 3), Week 24 (Part 3 only), and at early termination (if possible, for any subject who prematurely discontinues from the study). The actual time dosing and the actual time of blood collection is recorded. The subject also records the time of their last meal before taking their dose of (S)-4-((2-methoxyethyl)(4-(5,6,7,8-tetrahydro-1,8-naphthyridin-2-yl)butyl)amino)-2-(quinazolin-4-ylamino)butanoic acid and the time of their first meal after taking the dose of (S)-4-((2-methoxyethyl)(4-(5,6,7,8-tetrahydro-1,8-naphthyridin-2-yl)butyl)amino)-2-(quinazolin-4-ylamino)butanoic acid. Aliquots of these samples are used to measure concentrations of commonly used medications for PSC in subjects who received such medications concomitantly.

In addition to the specified time points, a PK sample is obtained for any subject who experienced elevations in liver enzymes. A PK sample is also obtained from any subject who prematurely discontinued the study or at the early termination visit.

### Pharmacodynamic Assessments

In Parts 1 and 2, plasma, serum, and urine biomarker samples were obtained at predetermined times as indicated in the Schedule of Events (see Table B-4). In Part 3, plasma and serum biomarker samples were obtained at predetermined times as indicated in the Schedule of Events (see Table B-5 and Table B-6). These samples were used to measure specific proteins, nucleotides and/or metabolites that were elevated in subjects with fibrosis, and/or pharmacodynamic markers that could change with inhibition of integrins α_{V}β₆ and α_{V}β₁. The samples were also used to perform exploratory investigations using metabolomic or proteomic methods for use in future studies in subjects with fibrotic diseases.

The main purpose of the study was to confirm that(S)-4-((2-methoxyethyl)(4-(5,6,7,8-tetrahydro-1,8-naphthyridin-2-yl)butyl)amino)-2-(quinazolin-4-ylamino)butanoic acid is well tolerated by subjects with PSC and that drug concentrations are similar to those previously found in healthy subjects. In addition, the study tested, in an exploratory manner, a panel of biomarkers with potential to assess drug effects. Bexotegrast total and unbound plasma concentrations increased with dose. Bexotegrast reduced ELF score relative to placebo at all doses with a statistically significant difference at the 160 mg dose at Week 12. 160 mg dose achieved statistical significance across all components of the ELF score (TIMP-1, PIIINP, HA). All doses reduced collagen synthesis (PRO-C3) relative to placebo with statistical significance for the 40 and 160 mg doses at Week 12. Bexotegrast demonstrated dose dependent reductions in itch relative to placebo with statistical significance for the 160 mg dose at Week 12.

A table of exploratory biomarkers of interest and evidence for their potential prognostic or pharmacodynamic value is presented in Table B-8.

TABLE B-8
PSC Exploratory Biomarkers

| | Evidence of Prognostic |
|---|---|
| Biomarker | or Pharmacodynamic Value |
| Collagen neo-epitope | PSC patients with high baseline serum levels |
| serum markers: (e.g., | of PRO-C3 and PRO-C5) had shorter survival |
| PRO-C3 and PRO-C5) | compared to patients with low baseline serum |
| | levels¹; Significant reductions in PRO-C3 |
| | observed in the NGM282 treatment groups |
| | compared with the placebo². |
| ELF (TIMP-1, PIIINP, | ELF levels predict transplant-free survival |
| HA) | in patients with PSC³; NGM282 significantly |
| | reduced serum ELF levels in PSC |
| | patients compared with placebo². |
| 7α-hydroxy-4- | NGM282 significantly reduced levels of |
| cholesten-3-one (C4) | 7alpha-hydroxy-4-cholesten-3-one (C4, a |
| | serum marker of hepatic CYP7A1 activity |
| | indicative of target engagement) compared |
| | with placebo². |
| Total, primary, | Treatment with NGM282 resulted in decreases |
| secondary bile acids | in circulating bile acids, and secondary bile |
| | acids (GDCA), compared with placebo-treated |
| | patients². |

¹M. J. Nielsen, et al. (2018) Aliment Pharmacol Ther. 48: 179-189, the entire contents of which are hereby incorporated by reference in their entirety.
² G. M. Hirschfield, et al. (2019) J Hepatol. 70(3): 483-493, the entire contents of which are hereby incorporated by reference in their entirety.
³ E. M. G. de Vries, et al. (2017) Liver Int. 37(10): 1554-1561, the entire contents of which are hereby incorporated by reference in their entirety.

### Pharmacogenomics

A whole blood PaxGene sample for DNA isolation is collected from each subject before dosing on Day 1. The DNA samples is analyzed to evaluate drug metabolic enzyme and transporter polymorphisms that may contribute to variability in(S)-4-((2-methoxyethyl)(4-(5,6,7,8-tetrahydro-1,8-naphthyridin-2-yl)butyl)amino)-2-(quinazolin-4-ylamino)butanoic acid PK.

### Monitoring and Management of Elevated Liver Biochemistry Tests

Reference ranges for key liver-related parameters are shown in Table B-9.

TABLE B-9
Reference Ranges for Central Laboratory Key Liver Tests

| Parameter | Females | | Males | |
|---|---|---|---|---|
| Alanine aminotransferase (ALT) | 4-43 | U/L | 5-48 | U/L |
| Aspartate aminotransferase (AST) | 8-40 | U/L | 8-40 | U/L |

Alkaline phosphatase (ALP)

| | | | | |
|---|---|---|---|---|
| 18 to 19 years of age | 45-87 | U/L | 55-149 | U/L |
| >19 years of age | 35-104 | U/L | 40-129 | U/L |
| Total bilirubin | 0.2-1.2 | mg/dL | 0.2-1.2 | mg/dL |
| | 3-21 | μmol/L | 3-21 | μmol/L |

Subjects who experienced confirmed elevations in serum ALT, AST, or ALP that reached the thresholds specified in Table B-10, Table B-11, or Table B-12 were managed as described in the tables. Subjects returned to the study site for reevaluation within 48 to 72 hours after the initial abnormal laboratory results became available for confirmatory testing serum levels of ALP, ALT, AST, total and direct bilirubin, and lactate dehydrogenase (LDH) and INR by the central laboratory. Central laboratory values for INR that were >1.5 were repeated by the local laboratory in addition to the central laboratory. A PK sample was obtained at the same time that the repeat clinical laboratory samples were drawn, and the date and time of the PK draw and the last dose of (S)-4-((2-methoxyethyl)(4-(5,6,7,8-tetrahydro-1,8-naphthyridin-2-yl)butyl)amino)-2-(quinazolin-4-ylamino)butanoic acid was recorded.

If prompt evaluation was not possible within 48 to 72 hours after receipt of the abnormal laboratory results, (S)-4-((2-methoxyethyl)(4-(5,6,7,8-tetrahydro-1,8-naphthyridin-2-yl)butyl)amino)-2-(quinazolin-4-ylamino)butanoic acid was interrupted immediately (date of last dose of the (S)-4-((2-methoxyethyl)(4-(5,6,7,8-tetrahydro-1,8-naphthyridin-2-yl)butyl)amino)-2-(quinazolin-4-ylamino)butanoic acid was recorded), and the subject returned to the study site as soon as possible for reevaluation. Laboratory abnormalities and any clinical symptoms were reported within 48 hours of the availability of laboratory results and/or assessment of clinical symptoms.

TABLE B-10
Management of Subjects Who Had Normal Baseline ALT or
AST Values and a Suspected Hepatocellular DILI Signal

| Confirmed treatment- | | | |
|---|---|---|---|
| emergent ALT or | | | |
| AST elevation | Bilirubin | Symptoms ^{a} | Management Recommendation |
| ALT or AST ≥ | Normal or, for subjects | None | Blood tests should be repeated |
| 5 × ULN | with Gilbert's syndrome or | | at least once a week ^{b} |
| | hemolysis, no change in | | Follow-up for symptoms |
| | baseline total bilirubin | | |
| ALT or AST ≥ | Normal or elevated | None or | Interrupt (S)-4-((2- |
| 8 × ULN | | present | methoxyethyl)(4-(5,6,7,8- |
| | | | tetrahydro-1,8-naphthyridin-2- |
| | | | yl)butyl)amino)-2-(quinazolin- |
| | | | 4-ylamino)butanoic acid |
| | | | Blood tests should be repeated |
| | | | at least once a week ^{b} |
| | | | Initiate close monitoring and |
| | | | workup for competing etiologies |
| | | | (S)-4-((2-methoxyethyl)(4- |
| | | | (5,6,7,8-tetrahydro-1,8- |
| | | | naphthyridin-2-yl)butyl)amino)- |
| | | | 2-(quinazolin-4-ylamino)butanoic |
| | | | acid can be restarted only if |
| | | | another etiology is identified and |
| | | | liver enzymes return to baseline. |
| | | | Drug cannot be restarted if |
| | | | hepatic decompensation occurs. ^{c} |
| ALT or AST ≥ | Total bilirubin ≥ 2 × | None or | Interrupt (S)-4-((2- |
| 3 × ULN | baseline or, for subjects | present | methoxyethyl)(4-(5,6,7,8- |
| | with Gilbert's syndrome | | tetrahydro-1,8-naphthyridin-2- |
| | or hemolysis, direct | | yl)butyl)amino)-2-(quinazolin- |
| | bilirubin > 2 × baseline | | 4-ylamino)butanoic acid |
| | if baseline > 0.5 mg/dL | | Blood tests should be repeated |
| | | | at least once a week ^{b} |
| | | | Initiate close monitoring and |
| | | | workup for competing etiologies |
| | | | (S)-4-((2-methoxyethyl)(4- |
| | | | (5,6,7,8-tetrahydro-1,8- |
| | | | naphthyridin-2-yl)butyl)amino)- |
| | | | 2-(quinazolin-4-ylamino)butanoic |
| | | | acid can be restarted only if |
| | | | another etiology is identified and |
| | | | abnormalities return to baseline. |
| | | | Drug cannot be restarted if |
| | | | hepatic decompensation occurs. ^{c} |
| ALT or AST ≥ | Normal or elevated | Present | Interrupt (S)-4-((2- |
| 5 × ULN | | | methoxyethyl)(4-(5,6,7,8- |
| | | | tetrahydro-1,8-naphthyridin-2- |
| | | | yl)butyl)amino)-2-(quinazolin- |
| | | | 4-ylamino)butanoic acid |
| | | | Blood tests should be repeated |
| | | | at least once a week ^{b} |
| | | | Initiate close monitoring and |
| | | | workup for competing etiologies |
| | | | (S)-4-((2-methoxyethyl)(4- |
| | | | (5,6,7,8-tetrahydro-1,8- |
| | | | naphthyridin-2-yl)butyl)amino)- |
| | | | 2-(quinazolin-4-ylamino)butanoic |
| | | | acid can be restarted only if |
| | | | another etiology is identified and |
| | | | abnormalities return to baseline. |
| | | | Drug cannot be restarted if |
| | | | hepatic decompensation occurs. ^{c} |

ALT = alanine aminotransferase;
AST = aspartate aminotransferase;
ULN = upper limit of normal
^{a} Liver-related symptoms (e.g., severe fatigue, nausea, new onset of or worsening or pruritus, right upper quadrant pain); immunologic reaction (e.g., rash, >5% eosinophilia); new onset of or increase of pruritus; or hepatic decompensation.
^{b} The specific interval between tests should also be determined based on the subject's clinical condition.
^{c} The decision to restart (S)-4-((2-methoxyethyl)(4-(5,6,7,8-tetrahydro-1,8-naphthyridin-2-yl)butyl)amino)-2-(quinazolin-4-ylamino)butanoic acid must be discussed and approved in consultation with the Medical Monitor and Sponsor Study Director prior to re-initiation. If applicable, the subject will require close follow-up monitoring to exclude recurrence of liver injury after restarting the (S)-4-((2-methoxyethyl)(4-(5,6,7,8-tetrahydro-1,8-naphthyridin-2-yl)butyl)amino)-2-(quinazolin-4-ylamino)butanoic acid.

TABLE B-11
Management of Subjects who had Elevated Baseline ALT
or AST^{a} and a Suspected Hepatocellular DILI Signal

| Confirmed treatment- | | | |
|---|---|---|---|
| emergent ALT or | | | |
| AST elevation | Bilirubin | Symptoms ^{b} | Management Recommendation |
| ALT or AST ≥ 3 × | Normal or, for subjects | None | Blood tests should be repeated |
| baseline or ≥ 300 | with Gilbert's syndrome or | | at least once a week ^{c} |
| U/L (whichever | hemolysis, no change in | | Follow-up for symptoms |
| occurs first) | baseline total bilirubin | | |
| ALT or AST ≥ 5 × | Normal or elevated | None or | Interrupt (S)-4-((2- |
| baseline or ≥ 500 | | present | methoxyethyl)(4-(5,6,7,8- |
| U/L (whichever | | | tetrahydro-1,8-naphthyridin-2- |
| occurs first) | | | yl)butyl)amino)-2-(quinazolin- |
| | | | 4-ylamino)butanoic acid |
| | | | Blood tests should be repeated |
| | | | at least once a week ^{c} |
| | | | Initiate close monitoring and |
| | | | workup for competing etiologies |
| | | | (S)-4-((2-methoxyethyl)(4- |
| | | | (5,6,7,8-tetrahydro-1,8- |
| | | | naphthyridin-2-yl)butyl)amino)- |
| | | | 2-(quinazolin-4-ylamino)butanoic |
| | | | acid can be restarted only if |
| | | | another etiology is identified and |
| | | | liver abnormalities return to |
| | | | baseline. Drug cannot be restarted |
| | | | if hepatic decompensation occurs. ^{d} |
| ALT or AST ≥ 2 × | Total bilirubin ≥ 2 × | None or | Interrupt (S)-4-((2- |
| baseline or ≥ 300 | baseline or, for subjects | present | methoxyethyl)(4-(5,6,7,8- |
| U/L (whichever | with Gilbert's syndrome | | tetrahydro-1,8-naphthyridin-2- |
| occurs first) | or hemolysis, direct | | yl)butyl)amino)-2-(quinazolin- |
| | bilirubin > 2 × baseline | | 4-ylamino)butanoic acid |
| | if baseline > 0.5 mg/dL | | Blood tests should be repeated |
| | | | at least once a week ^{c} |
| | | | Initiate close monitoring and |
| | | | workup for competing etiologies |
| | | | (S)-4-((2-methoxyethyl)(4- |
| | | | (5,6,7,8-tetrahydro-1,8- |
| | | | naphthyridin-2-yl)butyl)amino)- |
| | | | 2-(quinazolin-4-ylamino)butanoic |
| | | | acid can be restarted only if |
| | | | another etiology is identified and |
| | | | abnormalities return to baseline. |
| | | | Drug cannot be restarted if |
| | | | hepatic decompensation occurs. ^{d} |
| ALT or AST ≥ 2 × | Normal or elevated | Present | Interrupt (S)-4-((2- |
| baseline or ≥ 300 | | | methoxyethyl)(4-(5,6,7,8- |
| U/L (whichever | | | tetrahydro-1,8-naphthyridin-2- |
| occurs first) | | | yl)butyl)amino)-2-(quinazolin- |
| | | | 4-ylamino)butanoic acid |
| | | | Blood tests should be repeated |
| | | | at least once a week ^{c} |
| | | | Initiate close monitoring and |
| | | | workup for competing etiologies |
| | | | (S)-4-((2-methoxyethyl)(4- |
| | | | (5,6,7,8-tetrahydro-1,8- |
| | | | naphthyridin-2-yl)butyl)amino)- |
| | | | 2-(quinazolin-4-ylamino)butanoic |
| | | | acid can be restarted only if |
| | | | another etiology is identified and |
| | | | abnormalities return to baseline. |
| | | | Drug cannot be restarted if |
| | | | hepatic decompensation occurs. ^{d} |

ALT = alanine aminotransferase;
AST= aspartate aminotransferase;
ULN = upper limit of normal
^{a}Elevated baseline ALT or AST is ≥ 1.5 × ULN.
^{b} Liver-related symptoms (e.g., severe fatigue, nausea, new onset of or worsening or pruritus, right upper quadrant pain); immunologic reaction (e.g., rash, >5% eosinophilia); new onset of or increase of pruritus; or hepatic decompensation.
^{c} The specific interval between tests should also be determined based on the subject's clinical condition.
^{d} The decision to restart (S)-4-((2-methoxyethyl)(4-(5,6,7,8-tetrahydro-1,8-naphthyridin-2-yl)butyl)amino)-2-(quinazolin-4-ylamino)butanoic acid must be discussed and approved in consultation with the Medical Monitor and Sponsor Study Director prior to re-initiation. If applicable, the subject will require close follow-up monitoring to exclude recurrence of liver injury after restarting the (S)-4-((2-methoxyethyl)(4-(5,6,7,8-tetrahydro-1,8-naphthyridin-2-yl)butyl)amino)-2-(quinazolin-4-ylamino)butanoic acid.

TABLE B-12
Management of Subjects who had a Suspected Cholestatic DILI Signal

| Confirmed treatment- | | | |
|---|---|---|---|
| emergent ALP | | | |
| elevation | Bilirubin | Symptoms^{a} | Management Recommendation |
| ALP ≥ 2 × baseline | Normal or, for subjects | None | Blood tests should be repeated |
| without alternative | with Gilbert's syndrome or | | at least once a week ^{b} |
| explanation | hemolysis, no change in | | Follow-up for symptoms |
| | baseline total bilirubin | | |
| ALP ≥ 2 × baseline | Total bilirubin ≥ 2 × | None or | Interrupt (S)-4-((2- |
| without alternative | baseline or, for subjects | present | methoxyethyl)(4-(5,6,7,8- |
| explanation | with Gilbert's syndrome | | tetrahydro-1,8-naphthyridin-2- |
| | or hemolysis, direct | | yl)butyl)amino)-2-(quinazolin- |
| | bilirubin > 2 × baseline | | 4-ylamino)butanoic acid |
| | if baseline > 0.5 mg/dL | | Blood tests should be repeated |
| | | | at least once a week ^{b} |
| | | | Initiate close monitoring and |
| | | | workup for competing etiologies |
| | | | (S)-4-((2-methoxyethyl)(4- |
| | | | (5,6,7,8-tetrahydro-1,8- |
| | | | naphthyridin-2-yl)butyl)amino)- |
| | | | 2-(quinazolin-4-ylamino)butanoic |
| | | | acid can be restarted only if |
| | | | another etiology is identified and |
| | | | liver abnormalities return to |
| | | | baseline. Drug cannot be restarted |
| | | | if hepatic decompensation occurs. ^{c} |
| ALP ≥ 2 × baseline | Normal or elevated | Present | Interrupt (S)-4-((2- |
| without alternative | | | methoxyethyl)(4-(5,6,7,8- |
| explanation | | | tetrahydro-1,8-naphthyridin-2- |
| | | | yl)butyl)amino)-2-(quinazolin- |
| | | | 4-ylamino)butanoic acid |
| | | | Blood tests should be repeated |
| | | | at least once a week ^{b} |
| | | | Initiate close monitoring and |
| | | | workup for competing etiologies |
| | | | (S)-4-((2-methoxyethyl)(4- |
| | | | (5,6,7,8-tetrahydro-1,8- |
| | | | naphthyridin-2-yl)butyl)amino)- |
| | | | 2-(quinazolin-4-ylamino)butanoic |
| | | | acid can be restarted only if |
| | | | another etiology is identified and |
| | | | abnormalities return to baseline. |
| | | | Drug cannot be restarted if |
| | | | hepatic decompensation occurs. ^{c} |
| ALP ≥ 3 × baseline | Normal or elevated | None or | Interrupt (S)-4-((2- |
| without alternative | | present | methoxyethyl)(4-(5,6,7,8- |
| explanation | | | tetrahydro-1,8-naphthyridin-2- |
| | | | yl)butyl)amino)-2-(quinazolin- |
| | | | 4-ylamino)butanoic acid |
| | | | Blood tests should be repeated |
| | | | at least once a week ^{b} |
| | | | Initiate close monitoring and |
| | | | workup for competing etiologies |
| | | | (S)-4-((2-methoxyethyl)(4- |
| | | | (5,6,7,8-tetrahydro-1,8- |
| | | | naphthyridin-2-yl)butyl)amino)- |
| | | | 2-(quinazolin-4-ylamino)butanoic |
| | | | acid can be restarted only if |
| | | | another etiology is identified and |
| | | | abnormalities return to baseline. |
| | | | Drug cannot be restarted if |
| | | | hepatic decompensation occurs. ^{c} |

ALP = alkaline phosphatase;
ULN = upper limit of normal
^{a}Liver-related symptoms (e.g., severe fatigue, nausea, new onset of or worsening or pruritus, right upper quadrant pain); immunologic reaction (e.g., rash, >5% eosinophilia); new onset of or increase of pruritus; or hepatic decompensation.
^{b} The specific interval between tests should also be determined based on the subject's clinical condition.
^{c} The decision to restart (S)-4-((2-methoxyethyl)(4-(5,6,7,8-tetrahydro-1,8-naphthyridin-2-yl)butyl)amino)-2-(quinazolin-4-ylamino)butanoic acid must be discussed and approved in consultation with the Medical Monitor and Sponsor Study Director prior to re-initiation. If applicable, the subject will require close follow-up monitoring to exclude recurrence of liver injury after restarting the (S)-4-((2-methoxyethyl)(4-(5,6,7,8-tetrahydro-1,8-naphthyridin-2-yl)butyl)amino)-2-(quinazolin-4-ylamino)butanoic acid.

(S)-4-((2-methoxyethyl)(4-(5,6,7,8-tetrahydro-1,8-naphthyridin-2-yl)butyl)amino)-2-(quinazolin-4-ylamino)butanoic acid is permanently discontinued in the event of confirmed elevations in serum ALT, AST or ALP that meet the following criteria for suspected acute DILI, based on the Consensus Guidelines on Best Practices for Detection, Assessment and Management of Suspected Acute Drug-Induced Liver Injury Occurring during Clinical Trials in Adults with Chronic Cholestatic Liver Disease. See M. Palmer, et al. (2020) Aliment Pharmacol Ther. 51 (1): 90-109, the entire contents of which are hereby incorporated by reference in their entirety. Special attention is given to Baseline values for ALT, AST, ALP, bilirubin, and INR; and presence or absence or liver-related symptoms. Liver-related symptoms include: new, worsening, or severe fatigue; nausea, anorexia, vomiting; new onset of or worsening of pruritus; abdominal or right upper quadrant pain; immunologic reaction (e.g., rash, fever, >5% eosinophilia); hepatic decompensation. Baseline direct bilirubin levels are considered for the management of confirmed elevations in serum ALT, AST or ALP for subjects with Gilbert's syndrome or hemolysis, as described in Table B-10, Table B-11, or Table B-12.

Criteria for permanent discontinuation of (S)-4-((2-methoxyethyl)(4-(5,6,7,8-tetrahydro-1,8-naphthyridin-2-yl)butyl)amino)-2-(quinazolin-4-ylamino)butanoic acid for confirmed ALT or AST elevations:

For subjects with normal Baseline ALT or AST values:
Confirmed ALT or AST ≥3×ULN with total bilirubin ≥2× Baseline value, with or without liver-related symptoms, if no other etiology is suspected/confirmed and liver enzymes are sustained following interruption of (S)-4-((2-methoxyethyl)(4-(5,6,7,8-tetrahydro-1,8-naphthyridin-2-yl)butyl)amino)-2-(quinazolin-4-ylamino)butanoic acid,
Confirmed ALT or AST ≥5×ULN with normal or elevated bilirubin accompanied by liver-related symptoms, if no other etiology is suspected/confirmed and liver enzymes are sustained following interruption of (S)-4-((2-methoxyethyl)(4-(5,6,7,8-tetrahydro-1,8-naphthyridin-2-yl)butyl)amino)-2-(quinazolin-4-ylamino)butanoic acid, or
Confirmed ALT or AST >8×ULN with normal or elevated bilirubin, with or without liver-related symptoms, if no other etiology is suspected/confirmed and liver enzymes are sustained following interruption of (S)-4-((2-methoxyethyl)(4-(5,6,7,8-tetrahydro-1,8-naphthyridin-2-yl)butyl)amino)-2-(quinazolin-4-ylamino)butanoic acid.

For subjects with elevated Baseline ALT or AST values:
Confirmed ALT or AST ≥2× Baseline value (or ≥300 U/L, whichever occurs first) with normal or elevated bilirubin accompanied by liver-related symptoms, if no other etiology is suspected/confirmed and liver enzymes are sustained following interruption of (S)-4-((2-methoxyethyl)(4-(5,6,7,8-tetrahydro-1,8-naphthyridin-2-yl)butyl)amino)-2-(quinazolin-4-ylamino)butanoic acid,
Confirmed ALT or AST >2× Baseline value (or ≥300 U/L, whichever occurs first) with total bilirubin >2× Baseline value OR with INR increased by >0.2 relative to Baseline value, with or without liver-related symptoms, if no other etiology is suspected/confirmed and liver enzymes are sustained following interruption of (S)-4-((2-methoxyethyl)(4-(5,6,7,8-tetrahydro-1,8-naphthyridin-2-yl)butyl)amino)-2-(quinazolin-4-ylamino)butanoic acid,
If Baseline measurements were <2×ULN: Confirmed ALT or AST ≥5× Baseline value (or ≥500 U/L, whichever occurs first) with normal or elevated bilirubin, with or without liver-related symptoms, and if no other etiology is suspected/confirmed and liver enzymes are sustained following interruption of (S)-4-((2-methoxyethyl)(4-(5,6,7,8-tetrahydro-1,8-naphthyridin-2-yl)butyl)amino)-2-(quinazolin-4-ylamino)butanoic acid, or
If Baseline measurements were >2×ULN but <5×ULN: Confirmed ALT or AST >3× Baseline value with normal or elevated bilirubin, with or without liver-related symptoms, and if no other etiology is suspected/confirmed and liver enzymes are sustained following interruption of (S)-4-((2-methoxyethyl)(4-(5,6,7,8-tetrahydro-1,8-naphthyridin-2-yl)butyl)amino)-2-(quinazolin-4-ylamino)butanoic acid.

Criteria for permanent discontinuation for(S)-4-((2-methoxyethyl)(4-(5,6,7,8-tetrahydro-1,8-naphthyridin-2-yl)butyl)amino)-2-(quinazolin-4-ylamino)butanoic acid for confirmed ALP elevations:
Confirmed ALP ≥2× Baseline value with total bilirubin >2× Baseline value, with or without liver-related symptoms, and if no other etiology is suspected/confirmed and liver enzymes are sustained following interruption of (S)-4-((2-methoxyethyl)(4-(5,6,7,8-tetrahydro-1,8-naphthyridin-2-yl)butyl)amino)-2-(quinazolin-4-ylamino)butanoic acid,
Confirmed ALP ≥2× Baseline value with normal or elevated bilirubin accompanied by liver-related symptoms, and if no other etiology is suspected/confirmed and liver enzymes are sustained following interruption of (S)-4-((2-methoxyethyl)(4-(5,6,7,8-tetrahydro-1,8-naphthyridin-2-yl)butyl)amino)-2-(quinazolin-4-ylamino)butanoic acid, or
Confirmed ALP ≥3× Baseline value with normal or elevated bilirubin, if no other etiology is suspected/confirmed and liver enzymes are sustained following interruption of (S)-4-((2-methoxyethyl)(4-(5,6,7,8-tetrahydro-1,8-naphthyridin-2-yl)butyl)amino)-2-(quinazolin-4-ylamino)butanoic acid.

All elevations in serum ALT, AST or ALP that are confirmed upon repeat testing and that meet the criteria for suspected DILI (as listed above) are reported within 48 hours. The subject permanently discontinues(S)-4-((2-methoxyethyl)(4-(5,6,7,8-tetrahydro-1,8-naphthyridin-2-yl)butyl)amino)-2-(quinazolin-4-ylamino)butanoic acid immediately and continues to be followed for close monitoring and causality and clinical evaluation as follows:
Repeat liver enzyme and serum bilirubin tests at least once a week until abnormalities stabilize (i.e., levels are clinically comparable to baseline values or baseline grade of abnormality) and the subject is asymptomatic,
Obtain a detailed history of symptoms and prior or concomitant diseases,
Obtain a history of concomitant drug use (including nonprescription or over-the-counter medications and herbal and dietary supplement preparations), alcohol use, recreational drug use, and special diets,
Rule out acute viral hepatitis types A, B, C, D, and E; alcoholic hepatitis; hypoxic/ischemic hepatopathy; and biliary tract disease,
Obtain immunoglobulin G1 (IgG1), antinuclear antibody (ANA), anti-smooth muscle antibody (anti-SMA), anti-liver-kidney microsome type 1 (anti-LKM1), and anti-liver cytosolic antigen type 1 (anti-LC1) levels to be compared with baseline values, to rule out autoimmune hepatitis,
Obtain a history of exposure to environmental chemical agents, and
Consider a gastroenterology or hepatology consultation, which may include a liver biopsy, for definitive diagnosis.

For subjects with confirmed elevations in liver enzymes (see Table B-10, Table B-11, or Table B-12) warranting temporary interruption of (S)-4-((2-methoxyethyl)(4-(5,6,7,8-tetrahydro-1,8-naphthyridin-2-yl)butyl)amino)-2-(quinazolin-4-ylamino)butanoic acid, but not meeting permanent drug discontinuation criteria as stated above, frequent hepatic laboratory testing and clinical assessments using unscheduled visits, including a thorough causality evaluation, are performed according to the tables above.

Re-initiation of (S)-4-((2-methoxyethyl)(4-(5,6,7,8-tetrahydro-1,8-naphthyridin-2-yl)butyl)amino)-2-(quinazolin-4-ylamino)butanoic acid is only be considered if another etiology is identified and confirmed and if liver abnormalities return to baseline values. The(S)-4-((2-methoxyethyl)(4-(5,6,7,8-tetrahydro-1,8-naphthyridin-2-yl)butyl)amino)-2-(quinazolin-4-ylamino)butanoic acid cannot be restarted if hepatic decompensation occurs.

Subjects who permanently discontinue(S)-4-((2-methoxyethyl)(4-(5,6,7,8-tetrahydro-1,8-naphthyridin-2-yl)butyl)amino)-2-(quinazolin-4-ylamino)butanoic acid due to potential liver toxicity are followed for close monitoring until abnormalities stabilize to baseline levels or baseline grade of abnormality and the subjects asymptomatic. Long-term follow-up information is provided for the subjects who permanently discontinue(S)-4-((2-methoxyethyl)(4-(5,6,7,8-tetrahydro-1,8-naphthyridin-2-yl)butyl)amino)-2-(quinazolin-4-ylamino)butanoic acid due to potential liver toxicity, including information on immunosuppressive therapy and new liver histology.

### Monitoring and Management of ECG-Related Abnormalities

The definition of a cardiac dysrhythmia characterized by a CTCAE Version 5 grading for electrocardiogram QT corrected interval prolonged is presented in Table B-13.

TABLE B-13
CTCAE Version 5 Grading for Electrocardiogram QT Corrected Interval Prolonged

| CTCAE Term | Grade 1 | Grade 2 | Grade 3 | Grade 4 |
|---|---|---|---|---|
| Electrocardiogram | Average QTc | Average QTc | Average QTc | Torsade de pointes; |
| (ECG) QT | 450-480 msec | 481-500 msec | ≥501 msec; or | polymorphic |
| Corrected Interval | | | >60 msec | ventricular |
| Prolonged | | | change from | tachycardia; |
| | | | baseline | signs/symptoms of |
| | | | | serious arrhythmia |

Any subject who experiences a treatment-emergent QTc prolongation of Grade ² or higher returns to the study site for prompt clinical evaluation after the abnormal ECG results became available and to repeat the safety ECG assessment. A PK sample is obtained at the same visit that the repeat ECG assessment was performed, and the date and time of the PK draw and the last dose of (S)-4-((2-methoxyethyl)(4-(5,6,7,8-tetrahydro-1,8-naphthyridin-2-yl)butyl)amino)-2-(quinazolin-4-ylamino)butanoic acid are recorded.
Grade 2 QTcF interval >480 but≤500 msec; or increased >30 to ≤60 msec from baseline value-> The subject must return to the study site within 48 to 72 hours
Grade 3: QTcF interval >500 msec or increased >60 msec from baseline value-> The subject must return to the study site within 24 hours or, if not feasible, to a local clinic or hospital for immediate clinical evaluation.

Clinical evaluation included the following:
Ruling out comorbidities and potential risk factors (e.g., congestive heart failure, bradyarrhythmias),
Evaluation of electrolyte disturbances (e.g., potassium, calcium, and magnesium), and
Review of potential concomitant medications known to prolong the QTc interval (e.g., serotonin receptor antagonists, anti-emetics).

If prompt evaluation is not feasible, (S)-4-((2-methoxyethyl)(4-(5,6,7,8-tetrahydro-1,8-naphthyridin-2-yl)butyl)amino)-2-(quinazolin-4-ylamino)butanoic acid is interrupted immediately (the date of last dose of (S)-4-((2-methoxyethyl)(4-(5,6,7,8-tetrahydro-1,8-naphthyridin-2-yl)butyl)amino)-2-(quinazolin-4-ylamino)butanoic acid was recorded), and the subject returns to the study site as soon as possible for reevaluation.

(S)-4-((2-methoxyethyl)(4-(5,6,7,8-tetrahydro-1,8-naphthyridin-2-yl)butyl)amino)-2-(quinazolin-4-ylamino)butanoic acid is permanently discontinued in the event of QTc prolongation meeting the following criteria:
Confirmed Grade 3 abnormality: QTcF interval >500 msec or increased >60 msec from baseline value, and
Grade 4 abnormality (does not require confirmation): torsade de pointes; polymorphic ventricular tachycardia; signs/symptoms of serious arrhythmia.

All treatment-emergent QTc abnormalities meeting the criteria for permanent discontinuation are reported within 48 hours. The subject permanently discontinues(S)-4-((2-methoxyethyl)(4-(5,6,7,8-tetrahydro-1,8-naphthyridin-2-yl)butyl)amino)-2-(quinazolin-4-ylamino)butanoic acid immediately and continues to be followed for close monitoring and causality and clinical evaluation as follows:
Repeat central ECG at least once weekly until abnormalities stabilize (i.e., QTc interval is comparable to baseline value or baseline grade of abnormality, if applicable) and the subject is asymptomatic,
Correction of any electrolyte abnormalities, if applicable,
Obtain a detailed history of symptoms and comorbidities,
Obtain a history of concomitant drug use (including nonprescription or over-the-counter medications and herbal and dietary supplement preparations), and
Consider a cardiology consultation.

For subjects with confirmed QTc prolongation warranting temporary interruption of (S)-4-((2-methoxyethyl)(4-(5,6,7,8-tetrahydro-1,8-naphthyridin-2-yl)butyl)amino)-2-(quinazolin-4-ylamino)butanoic acid (as noted below), but not meeting permanent drug discontinuation criteria, more frequent central ECG testing and clinical assessments (at least once weekly) are performed using unscheduled visits, including a thorough causality evaluation.
Confirmed Grade 2 QTcF interval >480 but≤500 msec
Confirmed increased >30 to ≤60 msec from baseline value

Re-initiation of (S)-4-((2-methoxyethyl)(4-(5,6,7,8-tetrahydro-1,8-naphthyridin-2-yl)butyl)amino)-2-(quinazolin-4-ylamino)butanoic acid is only considered if a more likely etiology is identified and if ECG abnormalities returns to baseline values. More frequent clinical monitoring and central ECG assessments (at least once weekly) after re-initiation of (S)-4-((2-methoxyethyl)(4-(5,6,7,8-tetrahydro-1,8-naphthyridin-2-yl)butyl)amino)-2-(quinazolin-4-ylamino)butanoic acid, if applicable, are performed through use of unscheduled visits. If the QTc prolongation reoccurs upon re-challenge, (S)-4-((2-methoxyethyl)(4-(5,6,7,8-tetrahydro-1,8-naphthyridin-2-yl)butyl)amino)-2-(quinazolin-4-ylamino)butanoic acid is permanently discontinued.

Any subject who experiences a QTc prolongation not confirmed upon repeat central ECG assessment continues or resumes administration of (S)-4-((2-methoxyethyl)(4-(5,6,7,8-tetrahydro-1,8-naphthyridin-2-yl)butyl)amino)-2-(quinazolin-4-ylamino)butanoic acid and follows the scheduled study visits. More frequent clinical evaluation and central ECG monitoring through use of unscheduled visits is allowed, if clinically warranted.

### Early Discontinuation of Study or Individual Subjects

A subject may be discontinued from(S)-4-((2-methoxyethyl)(4-(5,6,7,8-tetrahydro-1,8-naphthyridin-2-yl)butyl)amino)-2-(quinazolin-4-ylamino)butanoic acid for any of the following reasons:
Noncompliance with protocol procedures, including those relating to administration of (S)-4-((2-methoxyethyl)(4-(5,6,7,8-tetrahydro-1,8-naphthyridin-2-yl)butyl)amino)-2-(quinazolin-4-ylamino)butanoic acid,
Occurrence of an SAE or intolerable AE,
Occurrence of a clinically significant change in a laboratory parameter,
Confirmed elevations in serum ALT, AST, or ALP meeting criteria for suspected acute DILI,
Prolonged ECG QTc interval meeting criteria for permanent discontinuation, and/or
Infection with COVID-19 that precludes safe participation in the study.

A subject may elect to discontinue(S)-4-((2-methoxyethyl)(4-(5,6,7,8-tetrahydro-1,8-naphthyridin-2-yl)butyl)amino)-2-(quinazolin-4-ylamino)butanoic acid or withdraw from the study at any time. Subjects who discontinue(S)-4-((2-methoxyethyl)(4-(5,6,7,8-tetrahydro-1,8-naphthyridin-2-yl)butyl)amino)-2-(quinazolin-4-ylamino)butanoic acid for safety reasons before completing 12 weeks (Parts 1 and 2) or at least 24 weeks (Part 3) of treatment are asked to remain in the study to complete all remaining assessments. If this was not feasible, the subject is asked to return to the clinic for an early termination visit. All assessments that are required at the last study visit (EoS), as shown in the Schedule of Events for Parts 1, 2, and 3, are completed for any subject who discontinues(S)-4-((2-methoxyethyl)(4-(5,6,7,8-tetrahydro-1,8-naphthyridin-2-yl)butyl)amino)-2-(quinazolin-4-ylamino)butanoic acid and does not agree to complete all remaining assessments.

### Primary Endpoint

The primary endpoint is the nature and proportion of treatment-emergent adverse events (TEAEs) between(S)-4-((2-methoxyethyl)(4-(5,6,7,8-tetrahydro-1,8-naphthyridin-2-yl)butyl)amino)-2-(quinazolin-4-ylamino)butanoic acid and placebo groups.

Safety data from all subjects who received at least one dose of (S)-4-((2-methoxyethyl)(4-(5,6,7,8-tetrahydro-1,8-naphthyridin-2-yl)butyl)amino)-2-(quinazolin-4-ylamino)butanoic acid are incorporated into the final safety analysis.

AEs are coded using the Medical Dictionary for Regulatory Activities (MedDRA®). All AEs are graded for severity per the CTCAE grading scale and listed by subject and summarized by last treatment taken at onset of AE. All AEs are listed by subject and summarized by last treatment taken at onset of AE. The incidence of AEs, the incidence of TEAEs, the incidence of treatment-related AEs, and the severity of AEs are summarized by system organ class, preferred term, and maximum severity. In cases where a subject reported multiple occurrences of the same event, the greatest severity is included in the summary. The number and percentage of subjects with SAEs and treatment-related SAEs and subjects who withdrew prematurely due to an AE are tabulated by study treatment and dose.

Clinical laboratory test parameters are graded using the CTCAE grading scale for individual subjects and values outside the reference ranges were flagged. The incidence of treatment-emergent laboratory abnormalities are summarized by severity and treatment group. For each parameter, summary statistics are calculated for each measure and summarized by treatment and dose.

Individual ECG results are listed for each subject. Summaries of ECGs by treatment and dose include changes from baseline for each parameter.

Vital sign measurements, other laboratory tests, concomitant medications, medical history and changes in physical examinations at each time point are listed by subject. The number and percentage of subjects with abnormal ECGs are summarized by treatment and dose.

### Secondary Pharmacokinetic Endpoints

Plasma(S)-4-((2-methoxyethyl)(4-(5,6,7,8-tetrahydro-1,8-naphthyridin-2-yl)butyl)amino)-2-(quinazolin-4-ylamino)butanoic acid concentrations (total and unbound concentrations) at each sampling timepoint are presented in listings and descriptive summary statistics by dose and visit.

### Exploratory Endpoints

Absolute and relative changes from Baseline in liver fibrosis biomarkers (including PRO-C3 and ELF) and in ALP are presented in numerical and graphical forms by treatment and dose utilizing data from the timepoints specified in the Schedule of Events. Changes from Baseline to Week 12(Parts 1 and 2) and Week 24 (Part 3) in magnetic resonance (MR)-based liver imaging are also evaluated, as well as changes in PROs.

FIG. 23 shows a graph associated with the mean change in enhanced liver fibrosis (ELF) score from Baseline to Week 12 for several concentrations of (S)-4-((2-methoxyethyl)(4-(5,6,7,8-tetrahydro-1,8-naphthyridin-2-yl)butyl)amino)-2-(quinazolin-4-ylamino)butanoic acid in Example B-3. All participants had baseline ELF≥7.7 (moderate to severe liver fibrosis; Vesterhus M. et al., Hepatology. 2015 July; 62(1):188-97). As shown in FIG. 23, bexotegrast reduced ELF relative to placebo at all doses with statistical significance for the 160 mg dose. (marked by an asterisk, *; p<0.05 versus placebo) The 160 mg dose demonstrated an 84% reduction relative to placebo. An ELF score change ≥0.19 at 12 weeks has been shown to be predictive of clinical progression within 2 years (Vesterhus M. et al., Hepatology. 2015 July; 62(1):188-97).

FIG. 24 shows a graph associated with the mean change in the enhanced liver fibrosis (ELF) score component, tissue inhibitor of metalloproteinases-1 (TIMP-1), from Baseline to Week 12 for several concentrations of (S)-4-((2-methoxyethyl)(4-(5,6,7,8-tetrahydro-1,8-naphthyridin-2-yl)butyl)amino)-2-(quinazolin-4-ylamino)butanoic acid in Example B-3. FIG. 25 shows a graph associated with the mean change in the enhanced liver fibrosis (ELF) score component, procollagen III (PIIINP), from Baseline to Week 12 for several concentrations of (S)-4-((2-methoxyethyl)(4-(5,6,7,8-tetrahydro-1,8-naphthyridin-2-yl)butyl)amino)-2-(quinazolin-4-ylamino)butanoic acid in Example B-3. FIG. 26 shows a graph associated with the mean change in the enhanced liver fibrosis (ELF) score component, hyaluronic acid (HA), from Baseline to Week 12 for several concentrations of (S)-4-((2-methoxyethyl)(4-(5,6,7,8-tetrahydro-1,8-naphthyridin-2-yl)butyl)amino)-2-(quinazolin-4-ylamino)butanoic acid in Example B-3. As shown in FIG. 24, FIG. 25, and FIG. 26, the effect of Bexotegrast on all ELF score components was statistically significant for the 160 mg dose.

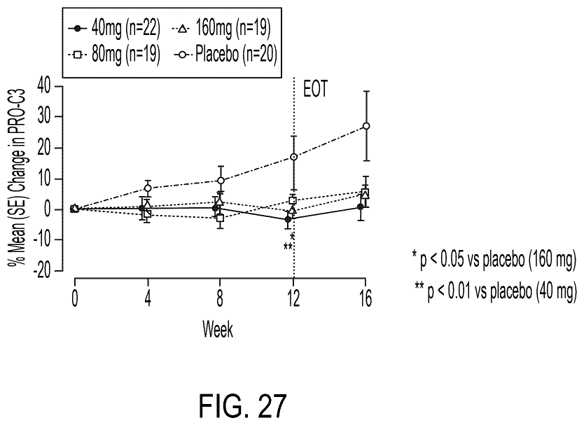

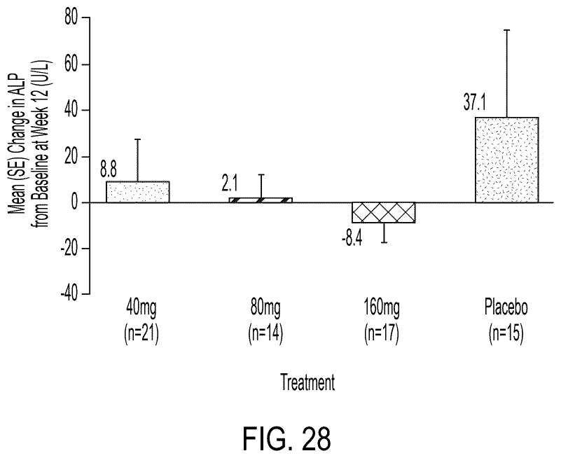

FIG. 27 shows a graph associated with the percent mean change in neo-epitope pro-peptide of type III collagen formation (PROC-C3) change for several concentrations of (S)-4-((2-methoxyethyl)(4-(5,6,7,8-tetrahydro-1,8-naphthyridin-2-yl)butyl)amino)-2-(quinazolin-4-ylamino)butanoic acid over a sixteen-week time period in Example B-3. As shown in FIG. 27, all doses reduced PRO-C3 relative to placebo, with statistically significant differences from placebo at Week 12 for 40 mg (p<0.01 vs. placebo) and 160 mg doses (p<0.05 vs. placebo). Only participants with both a baseline and post baseline are summarized. FIG. 28 shows a graph associated with the percent mean change in ALP for several concentrations of (S)-4-((2-methoxyethyl)(4-(5,6,7,8-tetrahydro-1,8-naphthyridin-2-yl)butyl)amino)-2-(quinazolin-4-ylamino)butanoic acid at Week 12 in Example B-3. As shown in FIG. 28, a dose-dependent trend for reduction in ALP relative to placebo was observed with bexotegrast.

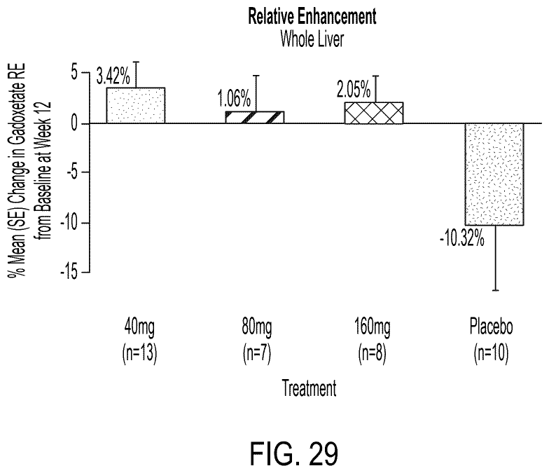

FIG. 29 shows a graph associated with the percent mean change in MRI relative enhancement in whole liver under different doses of (S)-4-((2-methoxyethyl)(4-(5,6,7,8-tetrahydro-1,8-naphthyridin-2-yl)butyl)amino)-2-(quinazolin-4-ylamino)butanoic acid in MRI from Baseline at Week 12 in Example B-3. All bexotegrast doses demonstrated improvement in relative enhancement compared to a reduction in placebo, suggesting improved hepatocyte function. All bexotegrast doses demonstrated improvement in hepatocyte function/density, as evidenced by contrast MRI scans, as compared to a reduction in placebo.

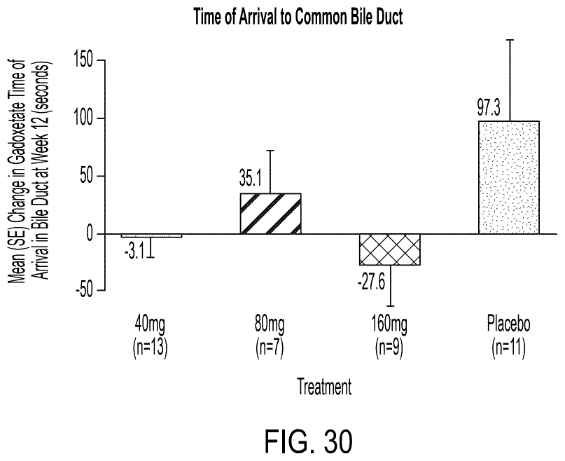

FIG. 30 shows a graph associated with the percent mean change in Gadoxetate Time of Arrival in Bile Duct for several concentrations of (S)-4-((2-methoxyethyl)(4-(5,6,7,8-tetrahydro-1,8-naphthyridin-2-yl)butyl)amino)-2-(quinazolin-4-ylamino)butanoic acid at Week 12 in Example B-3. The change in time of arrival was shorter in all bexotegrast doses compared to placebo, suggesting improved bile flow.

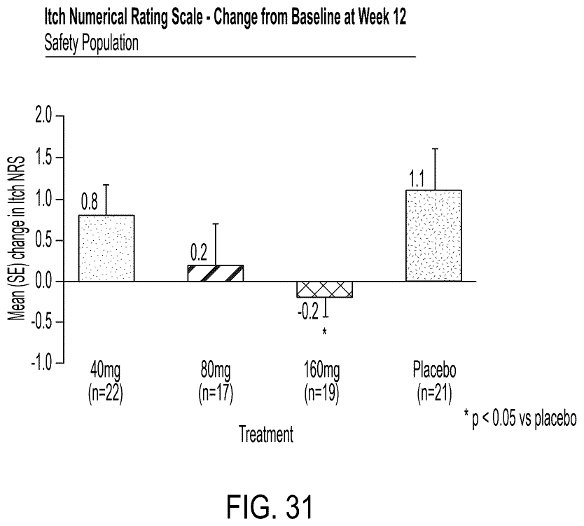

FIG. 31 shows a graph associated with the mean change in itch numerical rating scale (NRS) for several doses of (S)-4-((2-methoxyethyl)(4-(5,6,7,8-tetrahydro-1,8-naphthyridin-2-yl)butyl)amino)-2-(quinazolin-4-ylamino)butanoic acid from Baseline at Week 12 in Example B-3. As shown in FIG. 31, bexotegrast demonstrated dose-dependent changes in itch with statistical significance relative to the placebo for the 160 mg dose (p<0.05 vs. placebo) NRS=numerical rating scale. Itch NRS is a patient-reported outcome which assesses severity of itch over the past 24 hours on a scale of 0 (no itch) to 10 (worst imaginable itching).

Urine (Parts 1 and 2 only), plasma and serum samples were analyzed for biomarkers (presence or actual concentration). These samples were used to determine the levels of these markers in subjects and the relationship between these markers. Results were presented by listings, descriptive summary statistics and in graphical form by treatment and dose and expressed as the relative change (and or absolute) for each subject.

### Interim Analysis

Interim analyses (safety and tolerability) are conducted at the time points indicated below:
Following full enrollment and completion of the 12-week treatment duration with 40, 80, or 160 mg(S)-4-((2-methoxyethyl)(4-(5,6,7,8-tetrahydro-1,8-naphthyridin-2-yl)butyl)amino)-2-(quinazolin-4-ylamino)butanoic acid (completion of Parts 1 and 2), and
Following full enrollment and completion of the 12-week treatment duration with 320 mg(S)-4-((2-methoxyethyl)(4-(5,6,7,8-tetrahydro-1,8-naphthyridin-2-yl)butyl)amino)-2-(quinazolin-4-ylamino)butanoic acid (Part 3).

### Total Versus Unbound Bexotegrast in Plasma

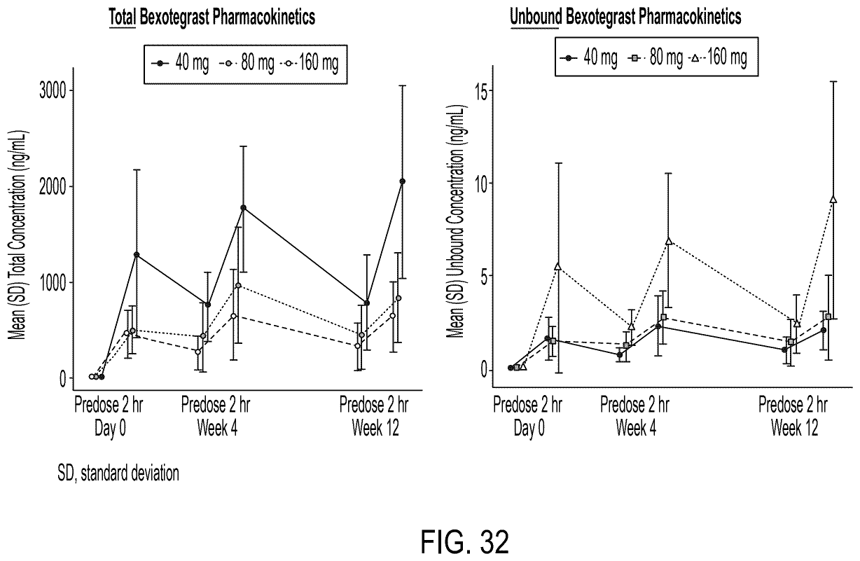

FIG. 32 shows a comparison of the levels of total bexotegrast in plasma (in ng/mL) (left graph) versus unbound bexotegrast in plasma (right graph). Levels are shown for two timepoints: pre-dose and two hours post-dose, at three different times over the course of the study: at the beginning of the study, at week 4, and at week 12. The lower curve shows levels for the 40 mg group, the middle curve shows levels for the 80 mg group, and the upper curve shows levels for the 160 mg group. The y-axis shows mean total bexotegrast concentration for the left graph and mean unbound bexotegrast concentration for the right graph, with bars showing standard deviation. Levels of total bexotegrast are approximately 200 to 300 times the level of unbound bexotegrast.

### Final Analysis

Final analyses (safety and tolerability) will be conducted once the last randomized subject in Part 3 have completed 24 weeks of treatment, including the follow up visit.

Bexotegrast demonstrated a favorable safety and tolerability profile in a PSC patient population with suspected moderate to severe liver fibrosis. Bexotegrast showed antifibrotic activity (ELF and PRO-C3) with statistically significant differences relative to placebo observed at Week 12 for the 160 mg dose. Liver biochemistry and imaging parameters were improved relative to placebo at Week 12. Bexotegrast demonstrated dose dependent reductions in itch numerical rating scale at Week 12 relative to placebo with statistical significance for the 160 mg dose.

As discussed above, bexotegrast is an oral, once-daily, inhibitor of integrins α_{V}β₆ and α_{V}β₁ in development for PSC. The Phase 2a study described above is an ongoing, double-blind, dose-ranging, randomized, placebo-controlled study of bexotegrast in patients with PSC and evidence of liver fibrosis evaluating safety, tolerability, pharmacokinetics, and effects on markers of fibrosis (enhanced liver fibrosis [ELF] and N-terminal type III collagen propeptide [PRO-C3]). The study had a 3:1 (bexotegrast: placebo) randomization and stratification by ursodeoxycholic acid (UDCA) use; with doses of 40 mg, 80 mg or 160 mg evaluated over a 12-week treatment period. 64 patients were enrolled in the active arms and 21 patients were enrolled in the placebo arm. The trial was enriched for patients having suspected liver fibrosis based on liver stiffness, ELF Score or historical liver biopsy. Baseline characteristics of the trial population reflected enrichment for patients at risk for moderate to severe fibrosis. A 320 mg dose cohort is ongoing and will be evaluated over 24-48 weeks. Key entry criteria included: large duct PSC with stable inflammatory bowel disease, if present, and liver fibrosis (without cirrhosis) as evidenced by ≥1: ELF≥7.7, liver stiffness by transient elastography (TE)≥8 kPa or magnetic resonance elastography ≥2.4 kPa or historical biopsy.

To summarize the results, at baseline (n=85), the mean age was 45 years, 75% male, 65% on concomitant UDCA for the entire cohort. Baseline characteristics (mean (SD)) included: ALP 264 (152) U/L, ALT 82(63) U/L, bilirubin 0.8 (0.4) mg/dL, TE 9.0 (2.9) kPa, ELF 9.4 (0.92) and PRO-C3 47.1 (23.12) ug/mL; bexotegrast-treated and placebo groups were comparable across baseline parameters. The incidence of treatment emergent adverse events (TEAE) and study discontinuations were not significantly different between any of the bexotegrast-treated or placebo groups. The most common TEAEs were (bexotegrast/placebo %) pruritus (14/24), fatigue (14/9), headache (9/19) and nausea (9/0). Rates of cholangitis were lower with bexotegrast (3/14). No serious TEAEs were deemed related to bexotegrast. At Week 12, the mean change in ELF score (FIG. 23) with the 160 mg group was 84% lower compared to placebo (p<0.05). Similar findings were observed for PRO-C3 (FIG. 27). At this 12-week interim analysis bexotegrast's safety and tolerability in PSC were similar to placebo. Bexotegrast was well tolerated at all three doses tested. Of the 64 patients treated with bexotegrast, 60 (94%) completed 12 weeks of treatment with no deaths or drug-related serious adverse events (SAE) reported. Most treatment emergent adverse events (TEAEs) were mild or moderate in severity and consistent with PSC disease symptoms. Patients in the trial who had concomitant inflammatory bowel disease (IBD) saw no change in their IBD symptoms as measured by partial Mayo Score while on treatment. Bexotegrast displayed a favorable pharmacokinetic profile with total and unbound plasma concentrations of bexotegrast increasing with dose.

Increases in ELF scores and PRO-C3 observed in the placebo group, in this fibrosis-enriched population, were attenuated by bexotegrast at all doses with statistical significance observed with 160 mg at 12 weeks. Results at the initial three doses tested showed bexotegrast reduced both ELF scores and PRO-C3 levels at Week 12 at all doses relative to placebo with statistically significant differences at the 160 mg dose. The bexotegrast 160 mg dose group demonstrated an 84% reduction of the change in ELF score relative to placebo at Week 12. Importantly, at the 160 mg dose group, statistically significant relative reductions were observed across all three components of the ELF score (tissue inhibitor of metalloproteinase 1 (TIMP-1), procollagen III N-terminal propeptide (PIIINP) and hyaluronic acid (HA)). Similarly, the bexotegrast 160 mg dose showed a statistically significant reduction in PRO-C3 level relative to placebo at Week 12. PRO-C3 is a biomarker of active fibrogenesis with higher levels associated with greater disease activity. Patients with PSC tend to have elevated liver biochemistry levels. Patients treated with bexotegrast showed stabilization of liver chemistry levels, including alanine aminotransferase (ALT) and aspartate aminotransferase (AST), as well as total and direct bilirubin, at all doses versus increases in placebo at Week 12. Additionally, patients treated with bexotegrast showed a dose-dependent trend in reduction of ALP levels, relative to placebo at Week 12. In addition, preliminary MRI imaging results suggest possible improved hepatocyte function and bile flow with bexotegrast 160 mg. Preliminary imaging results of the Gadoxetate contrast MRCP sub-study showed small increases in MRI relative enhancement from baseline across all dose groups, compared to a decrease in the placebo group to Week 12. MRI relative enhancement using Gadoxetate contrast is a measure of hepatocyte function, with increased enhancement suggesting improved hepatocyte function. Similarly, the change in time of arrival of Gadoxetate into the bile duct from baseline to Week 12 was shorter in the bexotegrast arms compared to the placebo group. Time to arrival is a measure of bile flow with shorter times suggestive of improved bile flow.

### Example B-4-Randomized, Double-Blind, Dose-Ranging, Placebo-Controlled, Phase 2a Evaluation of the Safety, Tolerability, and Pharmacokinetics of (S)-4-((2-Methoxyethyl)(4-(5,6,7,8-Tetrahydro-1,8-Naphthyridin-2-Yl)Butyl)Amino)-2-(Quinazolin-4-Ylamino) Butanoic Acid in Subjects with Primary Sclerosing Cholangitis (PSC) and Suspected Liver Fibrosis (INTEGRIS-PSC)

This example shows week 12 topline results for 40, 80, 160, and 320 mg vs. placebo of the INTEGRIS-PSC study in Example B-3. The objectives of the Phase 2a study included assessing the safety, tolerability, and pharmacokinetics of (S)-4-((2-methoxyethyl)(4-(5,6,7,8-tetrahydro-1,8-naphthyridin-2-yl)butyl)amino)-2-(quinazolin-4-ylamino)butanoic acid in subjects with PSC and suspected liver fibrosis.

### Study Design

The study is a Phase 2a, multicenter, 3-part, randomized, double-blind, dose-ranging, placebo-controlled, parallel-group study that evaluated the safety, tolerability, and PK of once-daily treatment with(S)-4-((2-methoxyethyl)(4-(5,6,7,8-tetrahydro-1,8-naphthyridin-2-yl)butyl)amino)-2-(quinazolin-4-ylamino)butanoic acid (bexotegrast) in subjects aged 18 to 75 years with an established diagnosis of large duct PSC and suspected liver fibrosis. Subjects with stable inflammatory bowel disease (IBD) were eligible for the study.

In this study, 121 eligible subjects were randomized (3:1 ratio) to receive 40 mg (n=21), 80 mg (n=21), 160 mg (n=21), or 320 mg (n=27) of (S)-4-((2-methoxyethyl)(4-(5,6,7,8-tetrahydro-1,8-naphthyridin-2-yl)butyl)amino)-2-(quinazolin-4-ylamino)butanoic acid once daily or matching placebo once daily (n=30) for a time period of 12 weeks. These initial groups are the safety population for both analysis of safety and efficacy. Patients were screened on Day −42 and Day 1 was the first day of treatment. Patients were stratified for the use of Ursodeoxychcolic acid (UDCA).

The total number of subjects screened was 205. The total number of subjects randomized and treated was 121, with 91 subjects receiving(S)-4-((2-methoxyethyl)(4-(5,6,7,8-tetrahydro-1,8-naphthyridin-2-yl)butyl)amino)-2-(quinazolin-4-ylamino)butanoic acid and 30 subjects receiving placebo. Out of the 91 subjects that received(S)-4-((2-methoxyethyl)(4-(5,6,7,8-tetrahydro-1,8-naphthyridin-2-yl)butyl)amino)-2-(quinazolin-4-ylamino)butanoic acid, 60 subjects (65.9%) were stratified for the use of UDCA and 8 subjects (8.8%) discontinued treatment (4 due to adverse events, 1 due to protocol deviation, 2 due to withdrawal by subject, 1 due to other reasons). Out of the subjects that received the placebo, 19 subjects (63.3%) were stratified for the use of UDCA and 2 subjects (6.7%) discontinued treatment due to adverse events. The 320 mg of the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a phosphate salt.

Table B-14 shows baseline demographics, where the Partial Mayo score is only reported for those with active IBD at Baseline, BMI=Body Mass Index; IBD=inflammatory bowel diseases; NRS-numerical Rating scale; and SD=Standard deviation.

TABLE B-14
Baseline Demographics

| | | | | | | | | | Bexotegrast | | | |
|---|---|---|---|---|---|---|---|---|---|---|---|---|
| | | | | | | | | | (40, 80, | | | |
| | Bexotegrast | | Bexotegrast | | Bexotegrast | | Bexotegrast | | 160, and | | | |
| | (40 mg) | | (80 mg) | | (160 mg) | | (320 mg) | | 320 mg) | | Placebo | |
| Characteristic | (n = 24)* | | (n = 20)* | | (n = 20)* | | (n = 27) | | (n = 91) | | (n = 30) | |
| Male sex, n | 17 | (70.8) | 16 | (80.0) | 14 | (70.0) | 13 | (48.1) | 60 | (65.9) | 24 | (80.0) |
| (%) | | | | | | | | | | | | |
| Age (yr.), | 46.9 | (15.06) | 40.5 | (15.32) | 45.1 | (12.65) | 47.1 | (14.47) | 45.2 | (14.44) | 45.2 | (13.75) |
| mean (SD) | | | | | | | | | | | | |
| Race, n (%) | | | | | | | | | | | | |
| White | 20 | (83.3) | 16 | (80.0) | 18 | (90.0) | 26 | (96.3) | 80 | (87.9) | 25 | (83.3) |
| Black | 2 | (8.3) | 2 | (10.0) | 1 | (5.0) | 0 | | 5 | (5.5) | 2 | (6.7) |
| Asian | 2 | (8.3) | 1 | (5.0) | 1 | (5.0) | 1 | (3.7) | 5 | (5.5) | 1 | (3.8) |
| Other/Not | 0 | | 1 | (5.0) | 0 | | 0 | | 1 | (1.1) | 2 | (6.7) |
| Reported/ | | | | | | | | | | | | |
| Unknown | | | | | | | | | | | | |
| Time since | 11.1 | (8.15) | 8.3 | (7.97) | 7.8 | (6.78) | 9.7 | (11.56) | 9.3 | (8.89) | 9.1 | (7.45) |
| diagnosis of | | | | | | | | | | | | |
| PSC (yr.), | | | | | | | | | | | | |
| mean (SD)** | | | | | | | | | | | | |
| Concomitant | 14 | (58.3) | 15 | (75.0) | 13 | (65.0) | 18 | (66.7) | 60 | (65.9) | 19 | (63.3) |
| UDCA use, n | | | | | | | | | | | | |
| (%) | | | | | | | | | | | | |
| IBD, n (%) | 18 | (75.0) | 12 | (60.0) | 11 | (55.0) | 13 | (48.1) | 54 | (59.3) | 17 | (56.7) |
| Ulcerative | 11 | (45.8) | 6 | (30.0) | 7 | (35.0) | 6 | (22.2) | 30 | (33.0) | 10 | (33.3) |
| colitis | | | | | | | | | | | | |
| Crohn's | 6 | (25.0) | 4 | (20.0) | 2 | (10.0) | 8 | (29.6) | 20 | (22.0) | 6 | (20.0) |
| disease | | | | | | | | | | | | |
| IBD Other | 3 | (12.5) | 2 | (10.0) | 2 | (10.0) | 0 | | 7 | (7.7) | 1 | (3.3) |
| Partial Mayo | 0.7 | (1.08) | 1.6 | (2.54) | 1.1 | (1.27) | 0.8 | (1.17) | 1.0 | (1.57) | 0.5 | (1.36) |
| Score, mean | | | | | | | | | | | | |
| (SD) | | | | | | | | | | | | |
| Itch NRS, | 1.8 | (2.54) | 2.1 | (2.63) | 1.4 | (1.50) | 0.9 | (1.77) | 1.5 | (2.15) | 1.0 | (1.43) |
| mean (SD) | | | | | | | | | | | | |

*Two participants (80 mg and 160 mg of bexotegrast) were dispensed incorrect number of tablets and provided incorrect dosing instructions for the full treatment period due to an error at a single site. The subjects daily dose corresponded to a ≤40 mg dose of bexotegrast. These 2 subjects are grouped in the 40 mg bexotegrast dose group for all summaries.
**Duration since diagnosis at screening is calculated from the first reported date for preferred terms of PSC.

Table B-15 shows the safety summary, where AE=Adverse Event; TEAE=Treatment Emergent Adverse Event; and SAE=Serious Adverse Events. AEs were coded using MedDRA v. 24.0.

TABLE B-15
Safety Summary

| | | | | | | | | | Bexotegrast | | | |
|---|---|---|---|---|---|---|---|---|---|---|---|---|
| AE, n (%) of | Bexotegrast | | Bexotegrast | | Bexotegrast | | Bexotegrast | | (40, 80, 160, | | | |
| Participants | (40 mg) | | (80 mg) | | (160 mg) | | (320 mg) | | and 320 mg) | | Placebo | |
| Reporting | (n = 24) | | (n = 20) | | (n = 20) | | (n = 27) | | (n = 91) | | (n = 30) | |
| TEAE* | 10 | (41.7) | 16 | (80.0) | 15 | (75.0) | 20 | (74.1) | 61 | (67.0) | 20 | (66.7) |
| Related to | 1 | (4.2) | 6 | (30.0) | 4 | (20.0) | 0 | | 11 | (12.1) | 7 | (23.3) |
| bexotegrast | | | | | | | | | | | | |
| Serious TEAE | 1 | (4.2) | 1 | (5.0) | 0 | | 0 | | 2 | (2.2) | 0 | |
| Related to | 0 | | 0 | | 0 | | 0 | | 0 | | 0 | |
| bexotegrast | | | | | | | | | | | | |
| TEAE of CTCAE | 1 | (4.2) | 2 | (10.0) | 1 | (5.0) | 1 | (3.7) | 5 | (5.5) | 3 | (10.0) |
| Grade 3 or Higher | | | | | | | | | | | | |
| Related to | 0 | | 0 | | 0 | | 0 | | 0 | | 2 | (6.7) |
| bexotegrast | | | | | | | | | | | | |
| TEAE Leading to | 1 | (4.2)¹ | 0 | | 0 | | 4 | (14.8)⁵ | 5 | (5.5) | 1 | (3.3)⁷ |
| Interruption of | | | | | | | | | | | | |
| bexotegrast | | | | | | | | | | | | |
| TEAE Leading to | 1 | (4.2)² | 1 | (5.0)³ | 1 | (5.0)⁴ | 1 | (3.7)⁶ | 4 | (4.4) | 2 | (6.7)⁸ |
| Withdrawal of | | | | | | | | | | | | |
| bexotegrast | | | | | | | | | | | | |
| TEAE Leading to | 0 | | 0 | | 1 | (5.0)⁴ | 0 | | 1 | (1.1) | 0 | |
| Early Termination | | | | | | | | | | | | |
| from Study | | | | | | | | | | | | |
| TEAE Leading to | 0 | | 0 | | 0 | | 0 | | 0 | | 0 | |
| Death | | | | | | | | | | | | |

*TEAE is defined as any AE starting (or worsening) on or after the date of first dose.
¹Chills/constipation/fatigue/nausea/pyrexia/vomiting
²COVID-19/dyspnoea/nasal congestion
³Hepatic enzyme increase/Pruritus
⁴Fatigue
⁵Fatigue; cough; oropharyngeal pain; increased ALT
⁶Increased ALP, ALT and AST
⁷Abdominal pain upper/fatigue/ocular icterus/pruritus
⁸Cardiomegaly/dyspnoea/malaise; headache

Table B-16 shows the most frequent TEAEs, where AEs were coded using MedDRA version 24.0.

TABLE B-16
Most Frequent TEAEs

| | | | | | | | | | Bexotegrast | | | |
|---|---|---|---|---|---|---|---|---|---|---|---|---|
| | | | | | | | | | (40, 80, | | | |
| TEAE,* n (%) | Bexotegrast | | Bexotegrast | | Bexotegrast | | Bexotegrast | | 160, and | | | |
| of Participants | (40 mg) | | (80 mg) | | (160 mg) | | (320 mg) | | 320 mg) | | Placebo | |
| Reporting | (n = 24) | | (n = 20) | | (n = 20) | | (n = 27) | | (n = 91) | | (n = 30) | |
| Most frequent | | | | | | | | | | | | |
| TEAEs | | | | | | | | | | | | |
| (n ≥ 3 in at least | | | | | | | | | | | | |
| one arm) | | | | | | | | | | | | |
| Fatigue | 3 | (12.5) | 2 | (10.0) | 4 | (20.0) | 3 | (11.1) | 12 | (13.2) | 4 | (13.3) |
| Pruritus¹ | 2 | (8.3) | 4 | (20.0) | 3 | (15.0) | 2 | (7.4) | 11 | (12.1) | 6 | (20.0) |
| Headache | 1 | (4.2) | 2 | (10.0) | 3 | (15.0) | 2 | (7.4) | 8 | (8.8) | 4 | (13.3) |
| COVID-19 | 2 | (8.3) | 1 | (5.0) | 0 | | 4 | (14.8) | 7 | (7.7) | 3 | (10.0) |
| Nausea | 1 | (4.2) | 2 | (10.0) | 3 | (15.0) | 1 | (3.7) | 7 | (7.7) | 0 | |
| Frequent bowel | 0 | | 3 | (15.0) | 0 | | 0 | | 3 | (3.3) | 3 | (10.0) |
| movements | | | | | | | | | | | | |

| | | | | | | | | | | |
|---|---|---|---|---|---|---|---|---|---|---|
| Cholangitis | 0 | 1 | (5.0) | 1 | (5.0) | 0 | 2 | (2.2) | 4 | (13.3) |

| | | | | | | | | | |
|---|---|---|---|---|---|---|---|---|---|
| Pyrexia | 1 | (4.2) | 0 | 0 | 0 | 1 | (1.1) | 3 | (10.0) |

| | | | | | | | |
|---|---|---|---|---|---|---|---|
| Dyspepsia | 0 | 0 | 0 | 0 | 0 | 3 | (10.0) |
| Ocular icterus | 0 | 0 | 0 | 0 | 0 | 3 | (10.0) |

*TEAE is defined as any AE starting (or worsening) on or after the date of first dose.
¹Pruritus includes preferred terms for pruritus and cholestatic pruritus

There were no safety concerns identified overall, including with the 320 mg dose of bexotegrast. Bexotegrast was well-tolerated with no dose relationship for AEs. There was a low rate of discontinuation due to the AEs. As noted in Table B-16, the most frequent TEAEs were fatigue and pruritus consistent with PSC. Further, the most common AEs were observed at lower rates for pooled bexotegrast as compared to the placebo. No treatment-related SAEs were observed on bexotegrast. No notable changes in liver biochemistry parameters were detected with bexotegrast. Additionally, the IBD status remained stable.

### Pharmacodynamic Assessments

Table B-17 shows baseline disease activity markers, where ELF=Enhanced Liver Fibrosis and PROC-C3=neo-epitope pro-peptide of type III collagen formation. PRO-C3 was quantified using Roche COBAS platform (assay reports approximately 2× higher concentrations than previous generation PRO-C3 ELISA).

TABLE B-17
Baseline Disease Activity Markers

| | | | | | | | | | Bexotegrast | | | |
|---|---|---|---|---|---|---|---|---|---|---|---|---|
| | Bexotegrast | | Bexotegrast | | Bexotegrast | | Bexotegrast | | (40, 80, 160, | | | |
| | (40 mg) | | (80 mg) | | (160 mg) | | (320 mg) | | and 320 mg) | | Placebo | |
| | (n = 24) | | (n = 20) | | (n = 20) | | (n = 27) | | (n = 91) | | (n = 30) | |
| Liver | | | | | | | | | | | | |
| Biochemistry, | | | | | | | | | | | | |
| mean (SD) | | | | | | | | | | | | |
| Alkaline | 315.1 | (140.26) | 199.2 | (81.03) | 273.8 | (165.63) | 190.6 | (91.29) | 243.6 | (132.13) | 277.4 | (215.88) |
| phosphatase (U/L) | | | | | | | | | | | | |
| Alanine | 91.5 | (62.08) | 67.6 | (63.15) | 98.4 | (73.11) | 60.4 | (37.76) | 78.5 | (60.20) | 73.1 | (59.84) |
| aminotransferase | | | | | | | | | | | | |
| (U/L) | | | | | | | | | | | | |
| Aspartate | 67.2 | (49.34) | 46.4 | (30.12) | 69.0 | (39.62) | 44.6 | (24.69) | 56.3 | (38.10) | 51.6 | (37.13) |
| aminotransferase | | | | | | | | | | | | |
| (U/L) | | | | | | | | | | | | |
| Total Bilirubin | 0.66 | (0.307) | 0.79 | (0.493) | 0.88 | (0.396) | 0.53 | (0.208) | 0.70 | (0.373) | 0.82 | (0.373) |
| (mg/dL) | | | | | | | | | | | | |
| Direct bilirubin | 0.27 | (0.164) | 0.26 | (0.188) | 0.31 | (0.166) | 0.16 | (0.062) | 0.24 | (0.156) | 0.31 | (0.238) |
| (mg/dL) | | | | | | | | | | | | |
| Markers of | | | | | | | | | | | | |
| Fibrosis, mean | | | | | | | | | | | | |
| (SD) | | | | | | | | | | | | |
| ELF Score | 9.6 | (0.77) | 9.2 | (1.01) | 9.4 | (0.79) | 9.0 | (0.84) | 9.3 | (0.87) | 9.3 | (1.03) |
| PRO-C3 (ng/mL) | 49.96 | (13.844) | 48.84 | (42.790) | 46.12 | (11.670) | 46.48 | (19.536) | 47.81 | (24.058) | 48.50 | (24.329) |
| Transient | 10.1 | (2.62) | 9.1 | (2.99) | 8.2 | (3.16) | 8.7 | (3.14) | 9.0 | (3.02) | 8.6 | (2.8) |
| Elastography (kPa) | | | | | | | | | | | | |

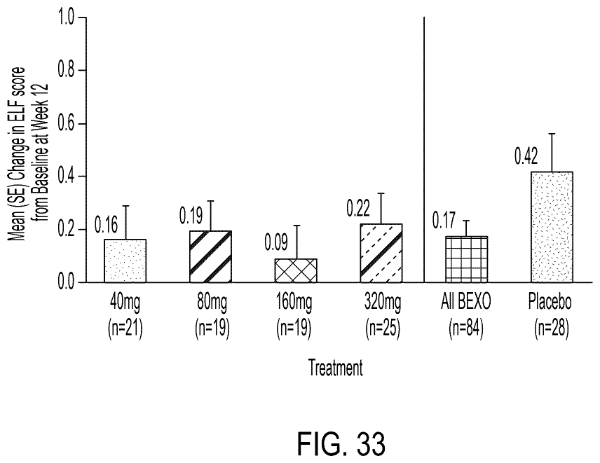

FIG. 33 shows mean (SE) change in enhanced liver fibrosis (ELF) score from Baseline for a safety population at Week 12 for 40 mg of bexotegrast (“40 mg”); 80 mg of bexotegrast (“80 mg”); 160 mg of bexotegrast (“160 mg”); 320 mg of bexotegrast (“320 mg”); bexotegrast at 40 mg, 80 mg, 160 mg, and 320 mg (“ALL BEXO”); and placebo of Example B-4. As shown in FIG. 33, bexotegrast reduced ELF scores relative to placebo at all doses.

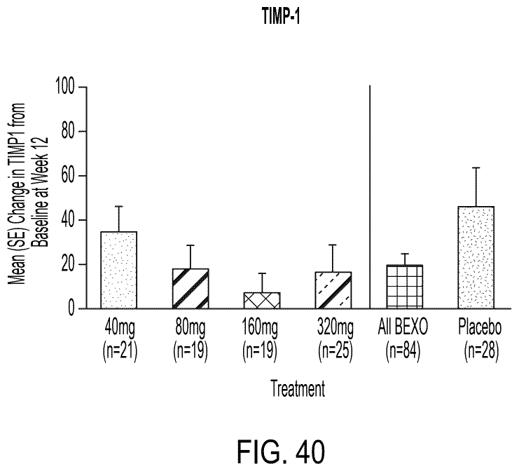

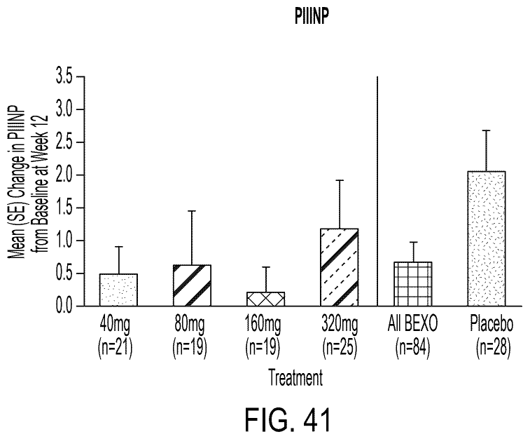

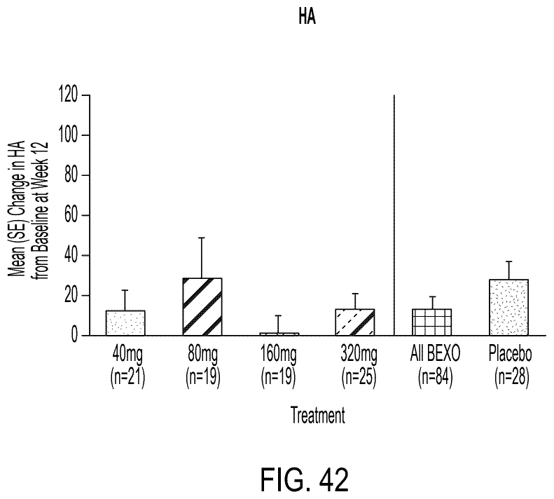

FIG. 40 shows mean (SE) change in an enhanced liver fibrosis (ELF) score component, tissue inhibitor of metalloproteinases-1 (TIMP-1), for a safety population from Baseline at Week 12 for 40 mg, 80, mg, 160 mg, and 320 mg of bexotegrast, all four doses (40 mg, 80 mg, 160 mg, and 320 mg) of bexotegrast pooled together (“ALL BEXO”), and placebo of Example B-4. FIG. 41 shows mean (SE) change in an enhanced liver fibrosis (ELF) score component, procollagen III, N-terminal propeptide (PIIINP), for a safety population from Baseline at Week 12 for 40 mg, 80, mg, 160 mg, and 320 mg of bexotegrast, all four doses (40 mg, 80 mg, 160 mg, and 320 mg) of bexotegrast pooled together (“ALL BEXO”), and placebo of Example B-4. FIG. 42 shows mean (SE) change in an enhanced liver fibrosis (ELF) score component, hyaluronic acid (HA), for a safety population from Baseline at Week 12 for 40 mg, 80, mg, 160 mg, and 320 mg of bexotegrast, all four doses (40 mg, 80 mg, 160 mg, and 320 mg) of bexotegrast pooled together (“ALL BEXO”), and placebo of Example B-4. As shown in FIG. 40, FIG. 41, and FIG. 42, bexotegrast generally reduced all ELF score components (TMP-1, PIIINP, and HA) compared to the placebo.

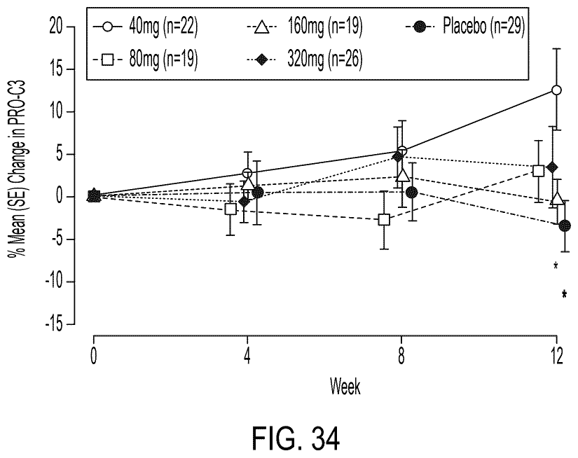

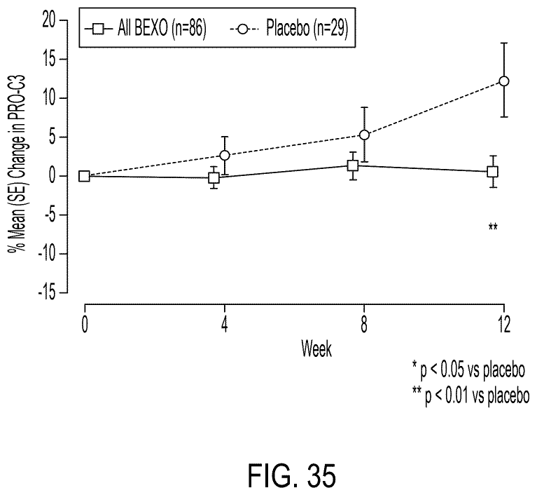

FIG. 34 shows the percentage of mean (SE) change in neo-epitope pro-peptide of type III collagen formation (PRO-C3) for a safety population over a twelve-week period for 40 mg of bexotegrast (“40 mg”); 80 mg of bexotegrast (“80 mg”); 160 mg of bexotegrast (“160 mg”); 320 mg of bexotegrast (“320 mg”); and placebo of Example B-4, where * p<0.05 vs. placebo. FIG. 35 shows the percentage of mean (SE) change in neo-epitope pro-peptide of type III collagen formation (PRO-C3) for a safety population over a twelve-week period for bexotegrast at 40 mg, 80 mg, 160 mg, and 320 mg (“ALL BEXO”) and placebo of Example B-4, where * p<0.05 vs. placebo and ** p<0.01 vs. placebo. In FIG. 34 and FIG. 35, only subjects with both a baseline and post-baseline were summarized. As shown in FIG. 34 and FIG. 35, all doses of bexotegrast reduced PRO-C3 relative to the placebo with statistical significance at Week 12 for 40 mg of bexotegrast, and 160 mg of bexotegrast, and the pooled bexotegrast groups.

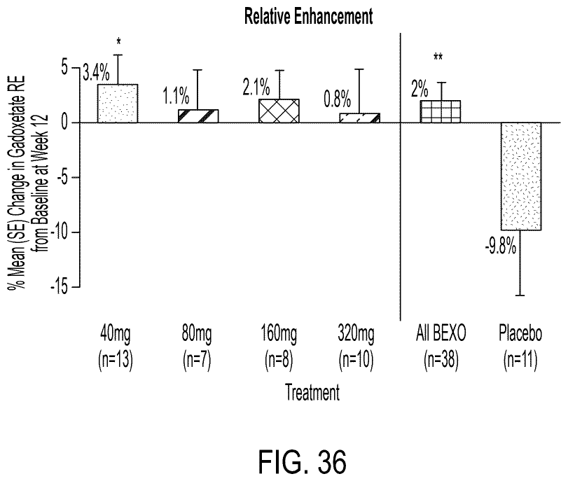

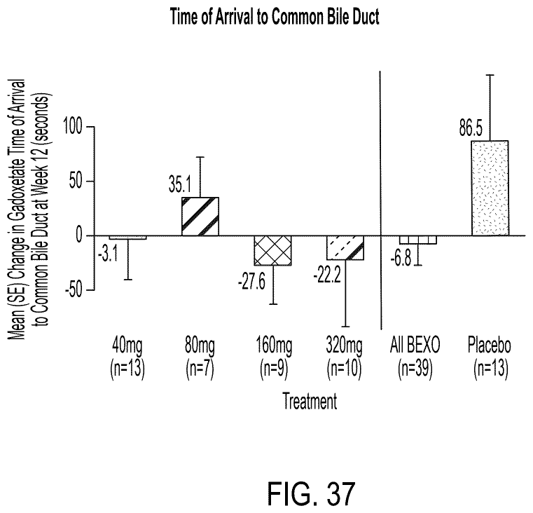

FIG. 36 and FIG. 37 show the change from Baseline at Week 12 for MRI parameters of the sub-study safety population. FIG. 36 shows the percentage of mean (SE) change in Gadoxetate relative enhancement (RE) for a sub-study safety population from Baseline at Week 12 for 40 mg of bexotegrast (“40 mg”); 80 mg of bexotegrast (“80 mg”); 160 mg of bexotegrast (“160 mg”); 320 mg of bexotegrast (“320 mg”); bexotegrast at 40 mg, 80 mg, 160 mg, and 320 mg (“ALL BEXO”); and placebo of Example B-4, where * p<0.05 vs. placebo and ** p<0.01 vs. placebo. RE, using the contrast agent Gadoxetate, is a measure of hepatocyte function. As shown in FIG. 36, all doses of bexotegrast showed an increase in RE compared to the placebo, suggesting improved hepatocyte function.

FIG. 37 shows the percentage of mean (SE) change in Gadoxetate time of arrival to common bile duct for a sub-study safety population at Week 12 in seconds for bexotegrast (“40 mg”); 80 mg of bexotegrast (“80 mg”); 160 mg of bexotegrast (“160 mg”); 320 mg of bexotegrast (“320 mg”); bexotegrast at 40 mg, 80 mg, 160 mg, and 320 mg (“ALL BEXO”); and placebo of Example B-4, where * p<0.05 vs. placebo and ** p<0.01 vs. placebo. Time of arrival of Gadoxetate to bile duct is a measure of excretory function. As shown in FIG. 37, all doses of bexotegrast reduced time of arrival to the common bile duct compared to the placebo, suggesting improved bile flow.

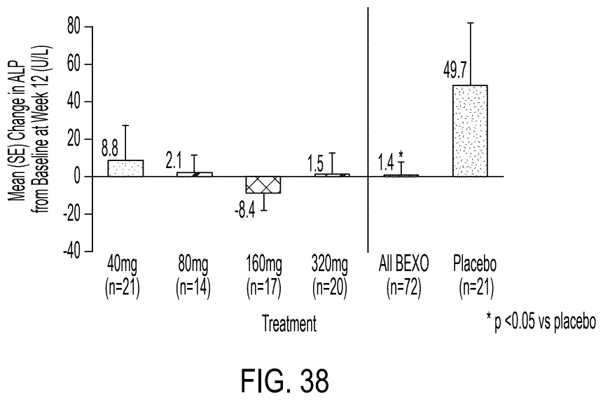

FIG. 38 shows the mean (SE) change in alkaline phosphatase (ALP) from baseline at Week 12 for the safety population (e.g., subjects with ALP>ULN at Baseline). FIG. 38 shows the mean (SE) change in alkaline phosphatase (ALP) from baseline for a safety population (subjects with ALP>ULN at Baseline) at Week 12 for bexotegrast (“40 mg”); 80 mg of bexotegrast (“80 mg”); 160 mg of bexotegrast (“160 mg”); 320 mg of bexotegrast (“320 mg”); bexotegrast at 40 mg, 80 mg, 160 mg, and 320 mg (“ALL BEXO”); and placebo of Example B-4, where * p<0.05 vs. placebo. As shown in FIG. 38, bexotegrast stabilized ALP relative to the placebo in the subgroup with elevated ALP at the Baseline.

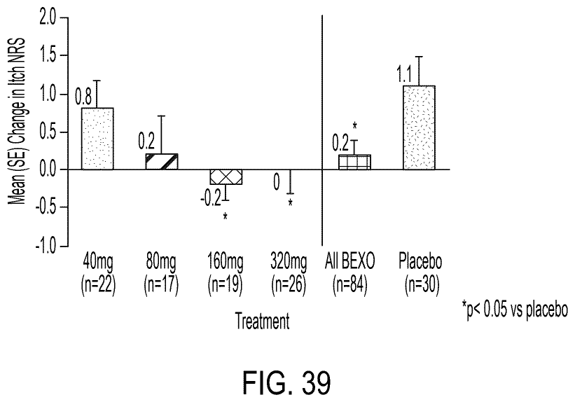

FIG. 39 shows the mean (SE) change in itch numerical rating scale (NRS) of a safety population for bexotegrast (“40 mg”); 80 mg of bexotegrast (“80 mg”); 160 mg of bexotegrast (“160 mg”); 320 mg of bexotegrast (“320 mg”); bexotegrast at 40 mg, 80 mg, 160 mg, and 320 mg (“ALL BEXO”); and placebo of Example B-4, where * p<0.05 vs. placebo. Itch NRS is a subject-reported outcome which assesses severity of itch, over the last 24 hours, on a scale of 0 (no itch) to 10 (worst imaginable itching). As shown in FIG. 39, bexotegrast showed dose-dependent reductions in itch relative to the placebo with statistical significance for the 160 mg, 320 mg bexotegrast groups.

### SUMMARY

Bexotegrast demonstrated a favorable safety and tolerability profile in the PSC patient population with suspected moderate to severe liver fibrosis. Of the 27 patients treated with bexotegrast at the 320 mg dose, 26 (96%) completed 12 weeks of treatment with no drug-related severe or SAEs. Most TEAEs were mild or moderate in severity and consistent with PSC disease symptoms. In addition, AEs of pruritus and cholangitis occurred less frequently on all doses of bexotegrast relative to placebo. Patients in the trial who had concomitant inflammatory bowel disease (IBD) saw no change in their IBD symptoms as measured by partial Mayo Score while on treatment.

The exploratory efficacy endpoints assessed changes in the liver fibrosis markers, ELF score and PRO-C3 levels, as well as liver biochemistry and MRI of the liver. Consistent with the results from the lower doses tested, bexotegrast-treated patients at the 320 mg dose showed a reduction in both ELF score and PRO-C3 levels relative to placebo at Week 12. Bexotegrast-treated patients also showed stabilization of ALP levels, relative to an increase on placebo at Week 12. In addition, MRI imaging continued to show evidence of improved hepatocyte function and bile flow with bexotegrast at the 320 mg dose relative to placebo.

### Example B-5-24-Week Data for Randomized, Double-Blind, Dose-Ranging, Placebo-Controlled, Phase 2a Evaluation of the Safety, Tolerability, and Pharmacokinetics of (S)-4-((2-Methoxyethyl)(4-(5,6,7,8-Tetrahydro-1,8-Naphthyridin-2-Yl)Butyl)Amino)-2-(Quinazolin-4-Ylamino) Butanoic Acid in Subjects with Primary Sclerosing Cholangitis (PSC) and Suspected Liver Fibrosis (INTEGRIS-PSC)

This example shows week 24 topline results for 320 mg vs. placebo of the study in Example B-3 and Example B-4.

The 320 mg cohort enrolled 27 patients in the active arm and 9 patients in placebo arm. The 320 mg treatment group met its primary endpoint of safety, demonstrating that bexotegrast was well tolerated up to 40 weeks of treatment. Pruritus and cholangitis occurred in lower proportions on bexotegrast than on placebo.

The bexotegrast study group had n=27, while the associated placebo group had n=9. Patients were screened on Day-28 and Day 1 was the first day of treatment. The study was stratified for the use of ursodeoxycholic acid (UDCA). Due to the trial design and enrollment trajectory, the longest treatment duration was 40 weeks.

The primary and secondary endpoints were safety, tolerability, and pharmacokinetics (PK) of the bexotegrast dosage. Exploratory endpoints included changes in transient elastography (TE) at Week 24; changes in liver fibrosis markers, enhanced liver fibrosis (ELF) score and PRO-C3; changes in liver imaging; and changes in liver biochemistry. Inclusion criteria was that a patient be at-risk for moderate/severe fibrosis defined by at least one criterion of ELF≥7.7; TE≥8 but≤14.4 kPa; magnetic resonance elastography (MRE)≥2.4 but≤4.9 kPa; or historical biopsy.

The total number of subjects screened was 52. The total number of subjects randomized and treated was 36, with 27 subjects receiving(S)-4-((2-methoxyethyl)(4-(5,6,7,8-tetrahydro-1,8-naphthyridin-2-yl)butyl)amino)-2-(quinazolin-4-ylamino)butanoic acid and 9 subjects receiving placebo. Out of the 27 subjects that received(S)-4-((2-methoxyethyl)(4-(5,6,7,8-tetrahydro-1,8-naphthyridin-2-yl)butyl)amino)-2-(quinazolin-4-ylamino)butanoic acid, 18 subjects (66.7%) were stratified for the use of UDCA and 4 subjects (14.8%) discontinued treatment. Out of the subjects that received the placebo, 6 subjects (66.7%) were stratified for the use of UDCA and none discontinued treatment. Four subjects in the treatment group withdrew for the following reasons: adverse event (n=1), withdrawal by subject (n=2), or other (n=1). All discontinuations occurred prior to Week 12. No subjects in the placebo group withdrew. UDCA=ursodeoxycholic acid.

Baseline demographics of the study subjects are shown in Table D-1. Duration since diagnosis at screening is calculated from the first reported date for preferred terms of PSC. The partial Mayo score is only reported for those with active inflammatory bowel diseases (IBD) at Baseline. BMI=Body Mass Index; NRS=numerical Rating scale; SD=Standard deviation.

TABLE D-1

| | Bexotegrast | | | |
|---|---|---|---|---|
| | 320 mg | | Placebo | |
| Characteristic | (n = 27) | | (n = 9) | |
| Male sex, n (%) | 13 | (48.1) | 7 | (77.8) |
| Age (yr), mean (SD) | 47.1 | (14.47) | 44.1 | (10.04) |
| Race, n (%) | | | | |
| White | 26 | (96.3) | 7 | (77.8) |
| Black | 0 | | 1 | (11.1) |
| Asian | 1 | (3.7) | 0 | |
| Other/Not Reported/Unknown | 0 | | 1 | (11.1) |
| Time since diagnosis of PSC (yr), | 9.4 | (11.20) | 6.7 | (5.37) |
| mean (SD) | | | | |
| Concomitant UDCA use, n (%) | 18 | (66.7) | 6 | (66.7) |
| IBD, n (%) | 13 | (48.1) | 5 | (55.6) |
| Ulcerative colitis | 6 | (22.2) | 3 | (33.3) |
| Crohn's disease | 8 | (29.6) | 2 | (40.0) |
| IBD Other | 0 | | 0 | |
| Partial Mayo Score, mean (SD) | 0.8 | (1.17) | 0 | (0) |
| Itch NRS, mean (SD) | 0.9 | (1.77) | 0.9 | (1.05) |

Baseline disease activity markers for the study subjects in Example B-5 are shown in Table D-2. ELF=Enhanced Liver Fibrosis; PROC-C3: neo-epitope pro-peptide of type III collagen formation. PRO-C3 was quantified using Roche COBAS® platform (assay reports approximately 2× higher concentrations than previous generation PRO-C3 ELISA) (COBAS® is a registered trademark of Roche, Inc., Indianapolis, Indiana, USA).

TABLE D-2

| | Bexotegrast | | | |
|---|---|---|---|---|
| | 320 mg | | Placebo | |
| | (n = 27) | | (n = 9) | |
| Liver Biochemistry, mean (SD) | | | | |
| Alkaline phosphatase (ALP) (U/L) | 190.6 | (91.29) | 318.6 | (282.73) |
| >ULN, n (%) | 22 | (81.5) | 6 | (66.7) |
| Alanine aminotransferase | 60.4 | (37.76) | 85.8 | (70.79) |
| (ALT) (U/L) | | | | |
| Aspartate aminotransferase | 44.6 | (24.69) | 58.2 | (50.91) |
| (AST) (U/L) | | | | |
| Total Bilirubin (mg/dL) | 0.53 | (0.208) | 0.76 | (0.424) |
| Direct bilirubin (mg/dL) | 0.16 | (0.062) | 0.33 | (0.341) |
| Markers of Fibrosis, mean (SD) | | | | |
| ELF Score | 9.0 | (0.84) | 9.5 | (0.93) |
| PRO-C3 (ng/mL) | 46.48 | (19.536) | 60.18 | (39.630) |
| Transient Elastography (kPa) | 8.7 | (3.14) | 8.6 | (2.85) |

Bexotegrast 320 mg was Well Tolerated Up to Week 40 with No Drug-Related Severe or Serious Adverse Events

The primary endpoint was the evaluation of the safety and tolerability of bexotegrast. Bexotegrast at 320 mg was well tolerated up to 40 weeks of treatment with no treatment-related severe or serious adverse events (SAE). Most treatment-emergent adverse events (TEAEs) were mild or moderate in severity and consistent with PSC disease symptoms. The adverse event profile for the study subjects after 24 weeks is shown in Table D-3. The abbreviations used are: AE=Adverse Event; TEAE=Treatment Emergent Adverse Event; SAE=Serious Adverse Events. Adverse events were coded using MedDRA v. 24.0. TEAE is defined as any AE starting (or worsening) on or after the date of first dose. The footnotes for the table are as follows: 1-Cholangitis/Enterobacter bacteremia (n=1); 2-Cholangitis (n=1); 3-fatigue (n=1), Oropharyngeal pain (n=1), Syncope (n=1), increased ALT/AST (n=1), cough (n=1); 4-cholangitis (n=1), Increased Pruritus/Ocular icterus/Fatigue/Upper abdominal pain (n=1); 5-Increased ALT/AST/ALP (n=1). Table D-4 shows the adverse event profile for the study subjects after 12 weeks. The footnotes for the table are as follows: 1-Cholangitis/Enterobacter bacteremia (n=1); 2-Cholangitis (n=1); 3-Syncope (n=1); 4-Increased ALT/AST/ALP (n=1). The abbreviations for Table D-4 are the same as for Table D-3. Table D-5 shows the serious adverse event profile for the study subjects. No serious adverse events were related to the study drug. Abbreviations: SAE=Serious Adverse Events (adverse events coded using MedDRA version 24.0); PSC=primary sclerosing cholangitis: ERCP=endoscopic retrograde cholangiopancreatography. Table D-6 describes treatment emergent adverse events (TEAEs) leading to withdrawal of the study drug in the study of Example B-5. A TEAE is defined as any adverse event (AE) starting (or worsening) on or after the date of the first dose. Adverse events were coded using MedDRA version 24.0. Table D-7 shows that the most frequent treatment emergent adverse events (TEAEs) were consistent with previous findings. Cholangitis was reported in 3.7% of bexotegrast participants and 11.1% of placebo participants. As in previous tables, a TEAE is defined as any AE starting (or worsening) on or after the date of first dose, and adverse events were coded using MedDRA version 24.0.

TABLE D-3

| | Bexotegrast | | | |
|---|---|---|---|---|
| | 320 mg | | Placebo | |
| AE, n (%) of Participants Reporting | (n = 27) | | (n = 9) | |
| TEAE | 23 | (85.2) | 5 | (55.6) |
| Related to study drug | 0 | | 0 | |
| Serious TEAE | 1 | (3.7)¹ | 1 | (11.1)² |
| Related to study drug | 0 | | 0 | |
| TEAE of CTCAE Grade 3 or Higher | 1 | (3.7)¹ | 1 | (11.1)² |
| Related to study drug | 0 | | 0 | |
| TEAE Leading to Interruption of | 5 | (18.5)³ | 2 | (22.2)⁴ |
| Study Drug | | | | |

| | | | |
|---|---|---|---|
| TEAE Leading to Withdrawal of | 1 | (3.7)⁵ | 0 |
| Study Drug | | | |

| | | |
|---|---|---|
| TEAE Leading to Early Termination | 0 | 0 |
| from Study | | |
| TEAE Leading to Death | 0 | 0 |

TABLE D-4

| | Bexotegrast | | | |
|---|---|---|---|---|
| AE, n (%) of Participants | 320 mg | | Placebo | |
| Reporting After Week 12 | (n = 27) | | (n = 9) | |
| TEAE | 16 | (59.3) | 5 | (55.6) |
| Related to study drug | 0 | | 0 | |
| Serious TEAE | 1 | (3.7)¹ | 1 | (11.1)² |
| Related to study drug | 0 | | 0 | |
| TEAE of CTCAE Grade 3 or Higher | 1 | (3.7)¹ | 1 | (11.1)² |
| Related to study drug | 0 | | 0 | |
| TEAE Leading to Interruption of | 1 | (3.7)³ | 1 | (11.1)² |
| Study Drug | | | | |

| | | | |
|---|---|---|---|
| TEAE Leading to Withdrawal of | 1 | (3.7)⁴ | 0 |
| Study Drug | | | |

| | | |
|---|---|---|
| TEAE Leading to Early Termination | 0 | 0 |
| from Study | | |
| TEAE Leading to Death | 0 | 0 |

TABLE D-5

| | | | | Any | | |
|---|---|---|---|---|---|---|
| | | | | alternative | | |
| | SAE | | | cause or | | |
| Treatment | Preferred | Standard | Treatment | confounding | Action | |
| Group | Term | Toxicity Grade | Related | factors? | Taken | Outcome |
| Placebo | Cholangitis | Grade 3 | No | PSC | Drug | Recovered/ |
| | | (Severe) | | | Interrupted, | Resolved |
| | | | | | ERCP | |
| | | | | | performed | |
| 320 mg | Cholangitis/ | Grade 3 | No | No | Dose not | Recovered/ |
| | Enterobacter | (Severe) | | | changed, | Resolved |
| | bacteraemia | | | | Hospital | |
| | | | | | Admission | |

TABLE D-6

| | | | | Any | | |
|---|---|---|---|---|---|---|
| | | | | alternative | | |
| | AE | Standard | | cause or | | |
| Treatment | Preferred | Toxicity | Treatment | confounding | Action | |
| Group | Term (s) | Grade | Related | factors? | Taken | Outcome |
| 320 mg | ALP | Grade 1 | No | No | Drug | Recovered/ |
| | increased/ | (Mild)/ | | | withdrawn | Resolved |
| | ALT | Grade 1 | | | | |
| | increased/ | (Mild)/ | | | | |
| | AST | Grade 1 | | | | |
| | increased | (Mild) | | | | |

TABLE D-7

| | Bexotegrast | |
|---|---|---|
| | 320 mg | Placebo |
| TEAE, n (%) of Participants Reporting | (n = 27) | (n = 9) |

Most frequent TEAEs (≥10%
in the 320 mg group)

| COVID-19 | 5 (18.5) | 1 | (11.1) |
|---|---|---|---|
| Nasopharyngitis | 5 (18.5) | 1 | (11.1) |
| Diarrhoea | 4 (14.8) | 0 | |
| Colitis ulcerative | 3 (11.1) | 0 | |
| Fatigue | 3 (11.1) | 2 | (22.2) |
| Headache | 3 (11.1) | 0 | |
| Pruritus | 3 (11.1) | 2 | (22.2) |

Bexotegrast 320 mg Continued to Demonstrate Antifibrotic Activity in a PSC Population with Suspected Moderate to Severe Liver Fibrosis at Week 24

The exploratory efficacy endpoints assessed changes in liver stiffness as measured by transient elastography (TE) at 24 weeks, changes in the liver fibrosis markers including Enhanced Liver Fibrosis (ELF) score, as well as liver biochemistry and magnetic resonance imaging (MRI). Bexotegrast at 320 mg demonstrated improvement in liver stiffness compared to placebo at Week 24. Bexotegrast improved markers and symptoms of cholestasis including alkaline phosphatase (ALP), MRI, self-reported itch, and common adverse events associated with PSC. Bexotegrast-treated patients showed decreased ALP levels over 24 weeks, compared to increased ALP on placebo. MRI of the liver demonstrated evidence of further improvement of hepatocyte function and bile flow with bexotegrast at the 320 mg dose from Week 12 to Week 24.

### Bexotegrast Stabilized Markers and Symptoms of Cholestasis

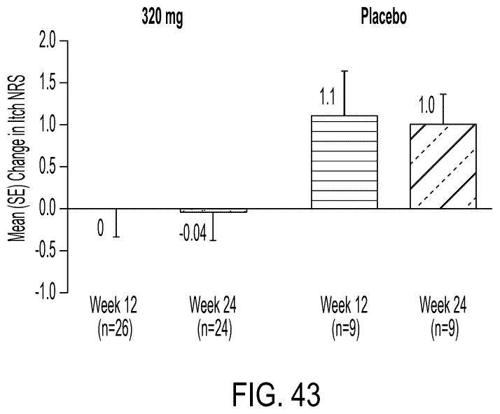

Pruritus and cholangitis are common symptoms of cholestasis in PSC patients (Karlsen TH, et al. J Hepatol. 2017 December; 67 (6): 1298-1323, which is incorporated by reference herein in its entirety). Adverse events of pruritus and cholangitis occurred in a lower proportion of bexotegrast-treated patients at 320 mg compared to placebo. As shown in FIG. 43, bexotegrast-treated patients demonstrated a stable score on the Itch Numerical Rating Scale (NRS) relative to a numerical increase on placebo over 24 weeks. Itch NRS is a patient reported outcome which assesses severity of itch, over the last 24 hours, on a scale of 0 (no itch) to 10 (worst imaginable itching). These findings are consistent with previously reported data from Week 12 across all doses.

### Stable Enhanced Liver Fibrosis (ELF) Score Observed; Reduction in ELF Score at Week 24 Observed Compared to an Increase on Placebo in High-Risk Subpopulation

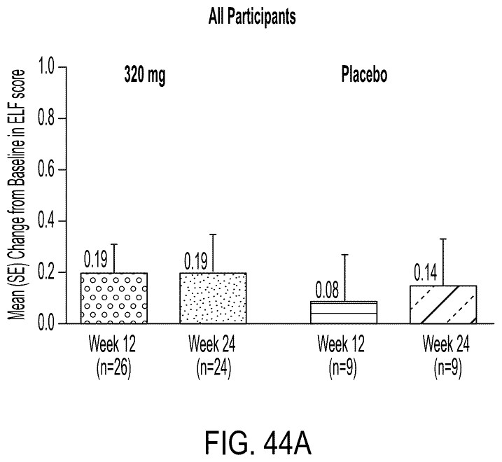

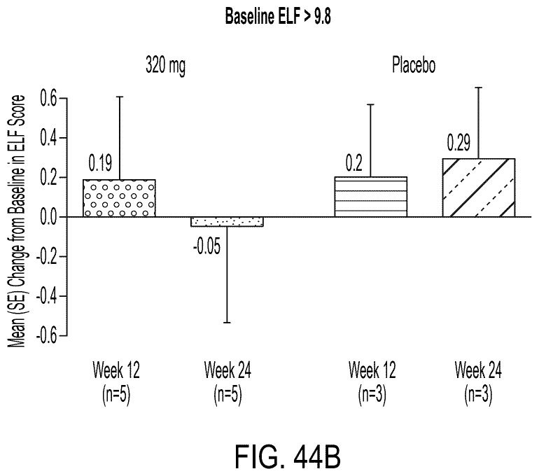

A stable ELF score was observed from Week 12 to Week 24 in the overall bexotegrast-treated population compared to placebo (FIG. 44A Importantly, in patients at high risk for disease progression (baseline ELF >9.8), bexotegrast-treated patients showed a reduction in ELF score at Week 24 compared to an increase in ELF score in the placebo group at Week 24 (FIG. 44B).

### Bexotegrast at 320 mg Demonstrated a Reduction in Median Liver Stiffness at Week 24 Compared to an Increase on Placebo

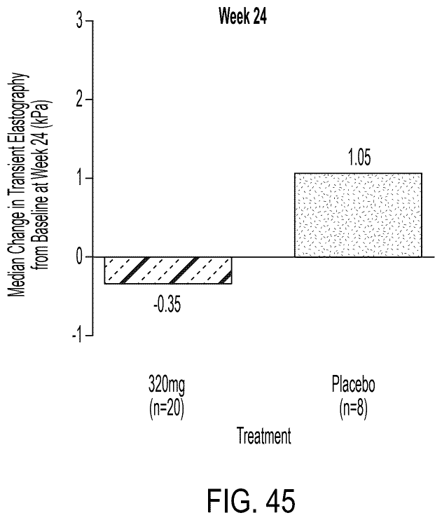

Bexotegrast at 320 mg demonstrated a numerical reduction in median liver stiffness at Week 24 compared to an increase on placebo, as measured by transient elastography (TE) (FIG. 45). Liver stiffness is a marker of liver fibrosis that increases over time in patients with PSC. Measurement of liver stiffness by TE can be used to predict the severity and progression of liver fibrosis (Corpechot C, et al. Gastroenterology. 2014 April; 146 (4): 970-9, which is incorporated by reference herein in its entirety).

### Statistically Significant Reduction in Alkaline Phosphatase (ALP)

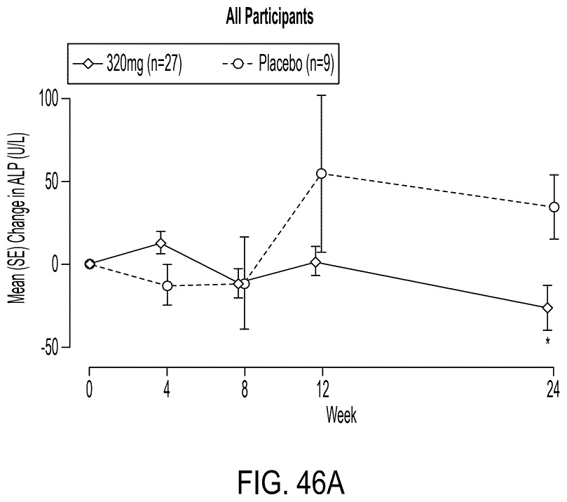

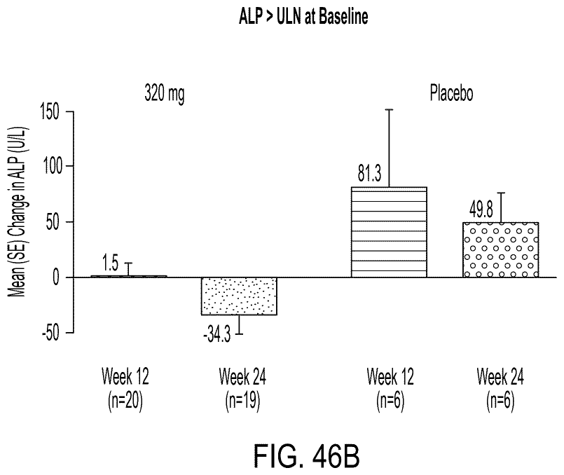

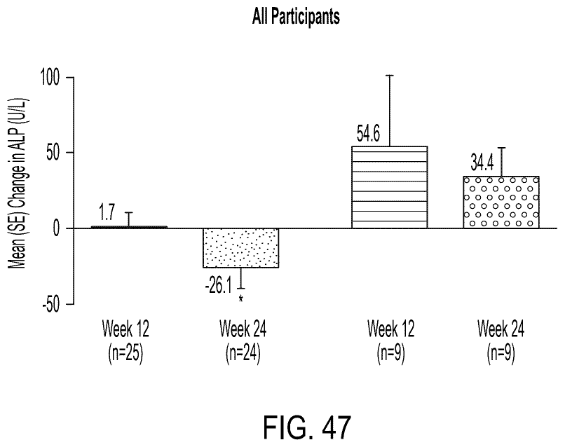

As shown in FIG. 46A, FIG. 46B, and FIG. 47, bexotegrast-treated patients demonstrated statistically significant reduction in ALP over 24 weeks compared to increased levels on placebo, with greater reductions observed in bexotegrast-treated patients with elevated baseline ALP values. (The asterisk * indicates p<0.05 versus placebo. ULN=upper limit of normal.)

Further Improvements in MRI Parameters-Continued Improvement in Hepatocyte Function and Bile Flow by Contrast MRI Imaging Observed from Week 12 to Week 24

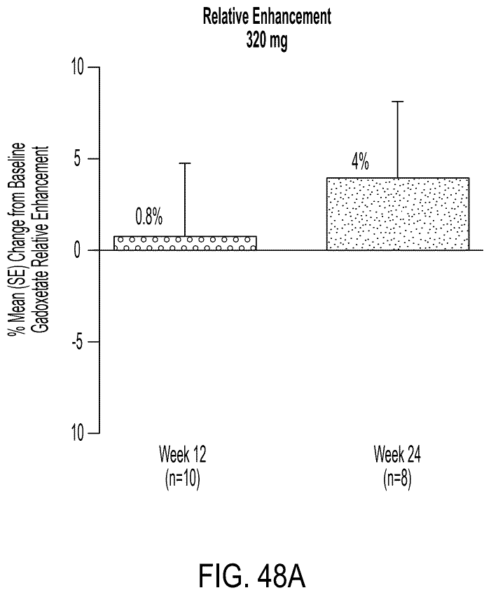

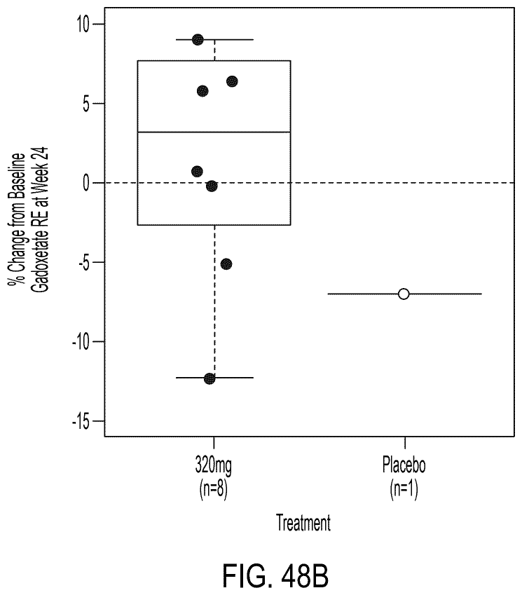

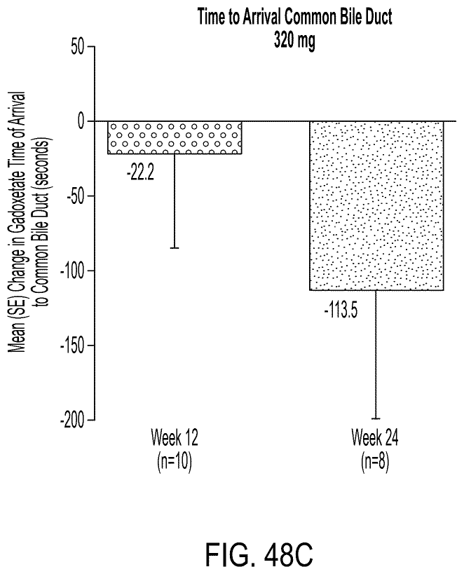

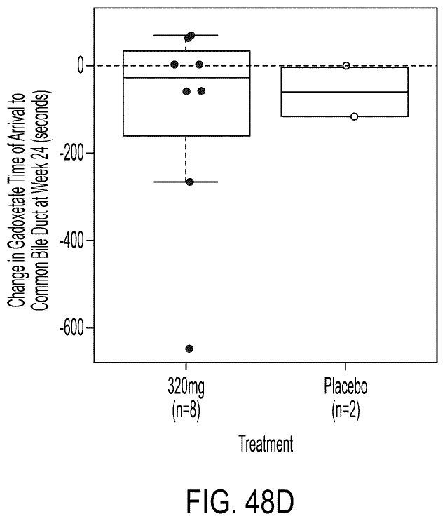

MRI was an optional sub study to the main study. At Week 12, in the MRI sub-study, bexotegrast-treated patients showed increased relative enhancement compared to decreased relative enhancement in the placebo group. (Relative enhancement (RE), using the contrast agent gadoxetate, is a measure of hepatocyte function) At Week 24, bexotegrast-treated patients displayed a further increase in relative enhancement, suggesting continued improvement in hepatocyte function from Week 12 to 24; see FIG. 48A. The values for individual patients are shown in FIG. 48B (only n=1 placebo participant had an evaluable Week 24 MRI scan for relative enhancement). At Week 12, bexotegrast-treated patients showed decreased time to arrival to the common bile duct compared to placebo, suggesting improved bile flow (Elkilany A, et al. Abdom Radiol (NY). 2021 March; 46 (3): 979-991, which is incorporated by reference herein in its entirety). At Week 24, bexotegrast-treated patients showed faster time to arrival to the common bile duct, suggesting further improvement in bile flow from Week 12 to 24; see FIG. 48C. The values for individual patients are shown in FIG. 48D (only n=2 placebo participants had an evaluable time to arrival). Time of arrival of gadoxetate to the bile duct is a measure of excretory function. Interpretation of placebo findings at Week 24 was limited due to the small number of placebo participants (n=<2) enrolled in the MRI sub-study of the 320 mg cohort.

All references throughout, such as publications, patents, patent applications and published patent applications, are incorporated herein by reference in their entireties.

Although the foregoing has been described in some detail by way of illustration and example for purposes of clarity of understanding, it is apparent to those skilled in the art that certain minor changes and modifications will be practiced. Therefore, the description and examples should not be construed as limiting the scope of the disclosure.

1. A method of ameliorating pruritus in a human who has fibrotic liver disease comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

2. The method of claim 1, wherein ameliorating pruritus comprises reducing pruritus in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

3. The method of claim 1, wherein the fibrotic liver disease is primary sclerosing cholangitis.

4. A method of ameliorating fibrosis in a tissue of a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein the tissue is liver tissue, gall bladder tissue, or bile duct tissue.

5-10. (canceled)

11. The method of, wherein the human has primary sclerosing cholangitis.

12. A method of treating primary sclerosing cholangitis in a human in need thereof, comprising administering to the human(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof, wherein primary sclerosing cholangitis in the human is ameliorated.

13-17. (canceled)

18. The method of claim 1, further comprising amelioration of an enhanced liver fibrosis score.

19. The method of claim 1, wherein the human has one or more of the following risk factors for fibrosis prior to a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human:
a. an enhanced liver fibrosis score greater than or equal to about 7.7,
b. a transient elastography score between about 7.7 kPa and about 14.4 kPa,
c. a magnetic resonance elastography score between about 2.4 kPa and about 4.9 kPa, and
d. a historical biopsy.

20-38. (canceled)

39. The method of claim 1, wherein the (S)-4-((2-methoxyethyl)(4-(5,6,7,8-tetrahydro-1,8-naphthyridin-2-yl)butyl)amino)-2-(quinazolin-4-ylamino)butanoic acid or the pharmaceutically acceptable salt thereof is administered in an amount equivalent to from about 40 mg to about 400 mg of (S)-4-((2-methoxyethyl)(4-(5,6,7,8-tetrahydro-1,8-naphthyridin-2-yl)butyl)amino)-2-(quinazolin-4-ylamino)butanoic acid.

40-45. (canceled)

46. The method of claim 4, further comprising reduction of pruritus in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

47. (canceled)

48. The method of claim 46, wherein pruritus is reduced as measured by a change in itch numerical rating scale from between about 0.8 and about-0.2 at about 12 weeks following a first administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof to the human relative to baseline itch rating prior to treatment.

49-52. (canceled)

53. The method of claim 4, further comprising reduction in collagen synthesis in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

54-63. (canceled)

64. The method of claim 1, wherein the administration results in amelioration of an alkaline phosphatase level in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

65-77. (canceled)

78. The method of claim 1 wherein the administration results in stabilization or decrease in total bilirubin in the human as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

79-85. (canceled)

86. The method of claim 12, wherein the human is concurrently being treated with another therapeutic for primary sclerosing cholangitis.

87-89. (canceled)

90. The method of claim 86, wherein the other therapeutic comprises a bile acid.

91-94. (canceled)

95. The method of claim 1, wherein the method is not accompanied by a serious treatment-related adverse event.

96-98. (canceled)

99. The method of claim 4, wherein hepatocyte function is improved in the human following administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

100-101. (canceled)

102. The method of claim 4 wherein bile flow is improved in the human following administration of (S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof as compared to a human who has not been administered(S)-4-((2-methoxyethyl)(4-(5,6,7,8-tetrahydro-1,8-naphthyridin-2-yl)butyl)amino)-2-(quinazolin-4-ylamino)butanoic acid, or a pharmaceutically acceptable salt thereof.

103-108. (canceled)

109. The method of claim 1, wherein the (S)-4-((2-methoxyethyl)(4-(5,6,7,8-tetrahydro-1,8-naphthyridin-2-yl)butyl)amino)-2-(quinazolin-4-ylamino)butanoic acid or a pharmaceutically acceptable salt thereof is a phosphate salt.

110-132. (canceled)
